SEQUENCE LISTING (89 sequences)

SEQ ID NO: 1 - DNA, 82731 bp, Artificial Sequence
  Description of Artificial Sequence Synthetic polynucleotide
cggacagaatgctaatcgaatcccaaaagcttagtgggttcttgaaaatgaagggctgaaatgctactaaagcaagtcattcaaaatcccgtcttgactgtctcaatttggccgattgcgatctgtcgtaaaacacttggaaatgcagaatgatggcatggcattgacgccatacattttgcgggcgttgagtccgtgtcgacagcaccccagtcgattctgtatttacgaattatgggacagtgaggtagggcttgacctggcgaatcatgaagaaatgactctataaaacacgcgtcacatgattttatccgctaatacccattctcgaattcttacggtcattcaaatagtttattaagttggctattggtgtagttattgctcttcatctacaattgcatgcctattcggaacatcaacaaaaagccagtatctaaatattggctttttgttgtgccggatgaatcaattagttatctgaaacaggataggcatgaaacagcgtcttttagtaatgaatggccaacggatactgcaaggcgaaagtgatggggcatggacgagtcagaaggtagacaaagcgggcactctgaagcctggaatatacaatatttacacggcggagccggtggataagtcgcagcggtatgatggcgtcatcgtttatgcggacaaaaatagtgtgtatcagcaaataggaaaaaaattcgttatgcatgctcggcaagatttcgatatagtgcctacttatgggagtgtaaaaagcattaattacaacgatcatggtaaagcagtcatatcgtccgagactgccaaactaagtcgaggacgctcccgataatttttagccgctaaaaatttggaggcgtctttgaacaagtcagttggtgaacgcggccctcggagcaatttccccagggttattcgaaacggtgatttaggaattctaaccgacgacacagtgggtcgggttgagcgattggatgagagcggaactcaagcgtcgccgcgatctgtgagaaaggcgacagagataagatttgccgggaacaatattgtcgatgatgtgttgttagagcgcactcagggcttggagcaagaacaacaagctctaatagaagccgctccccttgagcaaacgtatcaagaagcattggcgctgtacgctcaagctaaacacgcccaggttgaacagatcgaggatcggattgaaaacttgatcgatcgtcagcaagcgcgtctacaacacacgcaggccaatacgcccggtttgttgtccatgcctagcgcgaaacgggcatggcaggcccggcaaacacaacagcaagcacgtctgcacaatttgcataatcgtcttgagacagtgcgcgaaatcaaggaagggatggggattcatgccccaaaaattgaagaactggccacccgcaagatgcgagcggagaatcccgagcttgcgtctgattgggatgctatgcgcgaggcggcacggcgtcatcaagccctcatgcgcaagcaggagagggagcgcaaacaaacccatgagcagcggcaaggccgctctcaatccttaggactgtcaaagcccctataaacgatagcccggtcctccgagttatgtctgttgtctttacatcgaggcggcccaaaggaccagtccagccgctatgatcgtcatgccggcggcgatcaggttcatgagggccacgcgctttgcttcgcggatcggcgctgcgaactgtgcggatgccgctttaatctcgcggcgaatcaattccgccgccgacttagcgccttcctgcatcgtctgggtcatagtctcgcggctggccgtcaaggccgcgttgagcgtcctttccgccttattttttgcatcatcgctccatcggtgcgcaattgcctcaagctcttccttgaagtggtctaggatctcttgctgggcggctgcgctgtcctgcatcaatcgctcattgatggtttgaaggatcaggattggatcgtcgcggcccacagcgatgccatgtttaacagcaatttccttgatggtttcctcgattttgtcagtcgccattacagcaccgccgcgttctcaagatgcccaaaaatctgggtgcgcacgatcttgagccgctgtcgggtcatgatggttagcgattgcatggctagggcttcgtcgaaggtcagtcgggcttgcagcatttccgagaaatcccgtccgtaggtttcttccttgagcgtcggaatctggataatggccgatacacgatttttgttgtctttgtaggccttcatttgttcaaagcccttgccttcgtgctcgataggtccccaataaggattaagccagacgacaaacacacattcattcggaaactggccggccaattgagcaaatccgcttaccgtgtcctgaagggcctggccgccggtgatgacggtatgaacaaccatctcatgacccatgtccttgagcagtgccggcacttggttgctgatgaggtaatgcgagagcgggacgaacgagctggccccattgtcgataatgacatcgctttgggtcggcgctattagttcaaccagggcatcgaagttgcgggtgttgatttcatcgccatccatgatgttcagacgacgcgcttttaatgccttgtatccctcaaacgtcgcattgacaggatcggtatcaatgcatagaggcgcttgccccttgcttgccttgtattgagcaattgcggccgcgatcatcgacttaccgaccccccctttgccttgcaaaaccatatgaattttagccattaaaataaatcctctttgttgagttttggattgaatacgaagccagcctgagaagaaggcggtgtcgttgttttttgtgtcttgcttgtttttgatttcgtagcgttattggcagtggctgttgattcggtttcggttgtacgagctgcctgaggtcggcgtatcaagcggtttacgtggtttaagaatgtgtcgtaaccaaattcgatccgctgggtatcgaccatgttggcccatatcgttttaactgcgtaacccgcatctaatgctgcttgcacatcgtttttaaccgccaagaatgccactaagttcttgtctcgttttgttgattgccgttttttgacccattcgccaagttgttcgggatagctgttagccatatcgatttttcatccatcgttttcctattaattgtcaactttgtgtcagctcaatccggttgtgttcgattatacgtcaattatactcaatattctctatatcgaaatttaattaggatattttccagtctttatgtatgataaatcgaaactgcagggggctcctattcttggcgtaaccgtccgttaggacagggcaggatgtgtgaagtaggcccacccgcaagcgggatgttcctacttcactgaccttattcgcgctacgcactcaacggacatcctgctctgcgaggctggccggctaccgccggctttagattgagaggaaaccatggaacataaaaagaaaacacggaaaagcacgccacccatcaaggtgtattgccttccggaagagcgggagttgattgaaggcaatgctagagcggcagggatgagtctgtcttcttttttgatgacggtaggacagggttataaaatcacaggcattgtcgattacgaacaagtgcgcgaattggcacgtatcaacggtgatctcggccgtttgggcggcttactcaaactctggttgaccgatgacgtgcgtaccgctcagtttagcgatgccactattcgggccctgctgaaccggattgaggcgacgcaaaatgaaatggggcgggtcatgaagtccatcgttcaacccagagctgagtcatgatttttttagcggctaaaattctaaggatcgtgtcttgattgccaagcatgtccccatgcggtcgcctggtaaaagtgactttgcaggcttagcggattacatcaccgatgcgcaagaaaaaaccgagagattgggggcggttcaggtcaccaattgcgaggccggtacggttcaagccgcaattgccgaggcactggcaacccagcatctcaacacgcgggccaagggcgataaaacctaccatctactggtcagctttagaacaggcgagaagcccggcgtagataccttaagggccattgaagagcgcatttgtatggggcttggttatggcgaacaccagcgaatcagcgcagtgcattacgacacggataatctgcatattcatattgccatcaataagattcatccgaaacgccacacaatgcacgaaccgtattatccgcataaaacgttggccgaattatgcacaattcttgagcgagattaccggcttgagcccgacaatcatattccgcgcaagagcggttccgaaaatagagccgccgacatggaacgccatgcaggcgttgaaagcctgttgggttggatcaagcgggaatgtctggacgagatccgaagcgccgcgacgtggacggacttgcatcgagtgatgcacgaaaacggtctggaattgcgagagcgtggcaatggctttgtcattgaggcgggcgacggtacgagggttaaggccagcaccttatcgcgcgagttgtcaaaatcgaaactcgaggatcgtttgggaccattcgaggcatcacctcagcgacaagcccaggccaagcgacactatcaaaaaagcccgattcgtctacgggttgatacggttgagttgtacgccaagtataaggctgagcagcaaacgtcgacggcagcgcgtgccgccgagttggacgaggcgcgaaaacgtaaggagaggcggatagaggccgccaagcagtcgagtcggctgcgtcgaacagctatcaaaacattgaccgaagggcggttggttaagaagttgctgtatgcgcaagccagtcaagcgcttcgtaacgaaatacagtcgatcaataagcagtactggcgggagcggcaaggtttctatgatgcgttccggagacggacatgggccgactggctcaagcaggaggcgatgcagggcagtgccgaggcattgaccgccttgcgtgcccgtgaggcctcgcaggggctcaaaggtaacactgttaaaggcgatggacaggccagaccgggttccggccaagcgcctaccctggacaacatcaccaaaaaaggcacgatcattttccgagccggcaggtcggccgtccgggatgatggcgatcgcctgcaaatatcccgagaagcttcccatgaaggattgcaacaagccttgacgttagcaatggaacgctacgggagccgtatcaccgtcaacggcgccgccgagttcaaggcgcagatcatccgagccgccgtcgattcgcgtttacccatcacctttgccgatccgattttggaaagccggcggcaagcgcttttaatcaaggagcacagtcatgacaaacgaacagcacaacatcgagtccgagctggacgcggcattggccgctctgaatcaggagtcgctgccgaccggcacgctacccgatcagaaccttatggccgagcaggcggcggtcgaggaacagggggaaatgttagcgccggcgtcagccggaagcccgacattggaggcattggacgaatcccgccgccccaaagccggcaccgtttgcgagggctgtcccaactcggtgtggttcgcatcgccggcggaagtgaagtgctattgccgcgtgatgtttctggtcacctggagcagcaaggagccaaaccagataacgcattgcgacggggagtttctggggcaagcgtgagccctgaacaaatcactgccgctaaaaaatatattgacgaaagagagcaaaagaggcttaaaataaacgatataccgaagcataacttatataatggacaaaaaggagcctttgcttatgcaggagtcagaactgtcgagggtcaggctttggcattattgaaacagggcgatgacgttatggttatgccgatcgatcaagcgacgaccaggcgattgaagcgcgtcgggataggcgatttcatcacgattaccccgataggatcgattaaaacaacatcaggcaggagtcgatgactacatgagtacaaaacaccataatgcagtgggtccccaggtacggcgaaaaaagcaaggcaataaactgttgccggctttaggggtgctttcgctgggcggcggtctacaggcggccacgcagtatttcgcagctgcattcaactatcaagagagtttgggcgctcacatcaatcatgtatatgcgccctggtcgattttgcaatgggggacggcatggtacagccagtatccgaatgaattcatgcaagccggaagcgttggtatattggtttcaacgacgggtctgctgagcgtagccgttgcaaaagttatcgcttcgaattcatcgaaggcaactgagcacctgcacggttcggctcgatgggccggggagaaggatattcaggcggcgggtctattgccacggccgcgcacttttcttgaagcggtgaccggtaaagaatcgccatcctctaccggggtgtatgtcggcggttgggaagataatcagggtaaattttactacttgcgccataacgggccggaacacgttctgacctatgcgccgacccgttcaggcaagggtgtcggacttgtggtgccgaccctgctttcatggccggccagcgcggttatcaccgacttgaaaggcgagctgtgggcattgacagcgggctggagaaagcggcacgccaaaaacaaggttctaaactttgcgccggcatcggtcagcggcggcgtatgctggaatccattagatgaaatccgcaccggaacggaatacgaggtgggcgacgtacagaaccttgccgccctcattgtcgatccggacggcaagggtctcgaatcgcattggcagaagaccgcttttgcgctgctggttggcgtcatcctgcatgcactctacaaggcaaaacaggaaggcacgcaggccacgttgccatcggtcgacgccatgctagccgatcctaatcgtgacattgccgagctctggatggaaatggtgacctacggccattaccagaacggcgagaaccatccggccattggatcggccgcccgcgacatgatggaccgccccgaagaagaagccgggtccgtcctttccacggccaaatcctacttggccctgtatcgagatcccgtcgtagccaagaatgtcagtcattcagagtttcgcatcaaagacttaatgaatcacgatgaccctgtgagtttgtacatcgtgactcagccgaacgataagtcccgtctgaagcccttggtgcgcgtgatggtcaacatgatcgttcgtctgctggccgacaagatggaattcgagaacggtcgcccggtggcgcactacaagcatcgtttgctcttgatgctggatgagtttccaagtcttggcaagctggaaatcctgcaagagtctctcgctttcgtagccggctacggtatcaagtgttatctgatctgtcaggacattaaccagctcaagagccgtgaaacaggctacggtcacgatgaacagattacctcgaattgccacgttcagaacgcttatccacccaatcgcattgagacggccgagcatctttcgcgcttaaccggacaaaccacggtgattaaagagcagatcaccaccagcggacgccgaacggccgccatgatggggcaagtctcacgcaccattcaggaagtgcagaggcccttattgacggccgatgaatgcctacgtatgccggggccgcgtaagaatgcggcgggtgagattgaagaagcgggggatatggtcatttatgtagccggatatcccgccatctatggaaaacaaccgctctattttaaggactcgatttttcaagcgcgggcttcgatcccggctccgctgttgagcgacaagcttattcaggtacacaacgaagtggagattacgatatgaagcgcttgaaacgctttacggatagtgttgcgagagcaggtattgccgctctggtcgttagtggatgcgcctatgctgccggagtacgcattaacacgacgaaaagcattccggttggattgtatctgaccagcaatgctcaagtcgcgaaaggcgcctatgtgctgttttgtccgccacataatgaaatgttcacaatggcgaaggcaagagggtatatcggggccgggttttgtccgggggggtatggttatctgatgaaacgcattttagcggctaaaaacgatatcgtcgtgattgatgaagcgggcgtacgagtcaatggcgagcttcttcctttcagtgtaccccgcagggttgatagtgccgggcgggcattgccgcatttccatgtagagcactatagattaggcgacgacgaattactgctgatgtccgatgttagcggcacgtcctttgatggccgctacttcgggcctgtcgatcgctcacaaattaagaccgtgatactgccggtaatgacgttgtaaggagcgggtatgaatctatttattgcagaaaagccttctgtggcaaaagccattgcgggggaattggggatgacaggcaaaggcgatggctttatccactgtgggagcgacaaagtaacctggtgctttggacacatgctcgaacaggccgaaccggatgattacacaccgagtgatgtaccacgcggccacaatggcaagaaagtctggcgagttgatgaactgcctatcattccgacgacctggatactcatgcccaaggaggatgcaaagaaacaattgaccgcgatcggtaagttactcaaagaggcagacgtcattgtgaacgccggcgatcccgaccgtgaaggacaattgctggtagatgaggtccttgaacacttcaacaataccaagcctgtacgtcgtttctgggtgtctgcgcaagactctgtttcagtccaacgtggactggctgccttgaaggataacacccattttgccggctgggccgatgccgccaaggcacggcagcgtgccgactggctcatcggtatgaaccttagccgcgcattcacgttgcgggcgcaacgtggcggttctcgagcgctgctgaccgttggccgagtacagacgcccaccttggccctagttgttgcgcgggatagagaaatcgaaggattcaagccggtcccgtaccatactatccgagccgagattcagcatgcgggaggttcctttttcgccgcgtggaaggctaaagaagatcaagccggccttgattccgaaggccgacttgtcgataccgctattgccaacgcgcttgtcgagaagatagccggcaagcccggcatcattgccgagtataaggaggaagccaagacacaaagccagccgctggcttttgcgctgtccgacataacgctattagcctcccataaattcggctatagcgctgaagaggttctgaaagcctgccaggcgctttatgaaacgcacaaactgacttcctacccgcgtaccgattgcgcttatctgccggaatcccaacacgccgatgcagccggtattttggcagcgatcaaatacgtcaatcccgagctgggcgggatcgtggacggtgccgatccacgcatcaagtccaaaacgtggaacgactcgaagatcaccgcgcaccacgggatcatcccgacgatgcacaagggaggaaagtccggccttagtgacaaagagcgcactatctatgagctgattgttcgggcttacctcgctcagttctatccgttgcacgagtacaggcaaacaaccgtgagtgcagaagtcgagagtgaaaccttcaccgtgaacgggaaggtcatcacgcgcaacggctggcgagatgtgtttcaagaagctgacgaggacgacaaacacgattcggaagaaggcaacaggcaacgcttgccggccatgaaaaaaggcgacgatgtgacctgcggacaggccgctagacgggatgccaagacgaagccgccggccaggtttaccgaaggcacgttaacccgtgccatggagaacatacataaatttgtatcggacccggatcataagaaggtgttgcgtgatggcgatggcatcggtacatcggccacccgagcatccatactttcggagttaaaacgccgagagttcctggaactcaagggcaaacatattattagcacgacattgggtcgcagcgtcatcgatgctttgccggaagtcgtgaaaagtccggttttgactgccttgtatgaacgaatgttgaaaggtatcgagcaaggcaataccgagatgggcgcatttatagcccggcaagaggattttatccgcgaacaagtggcgaaggcgaacgcgggagccgtgcgcattgccggcggtaaagaggggactccggtatcttcgctgcacaagtgcatggcgtgcggtagcggcctttcacgtcgacccagcaaaaagccagggcatttttggtggggatgcagcaacttccctacctgcaagcaaacctatcccgagctaaaaggacgacctgactatagcaagggccgcgacaacaatcagacagtcattaaggggtaataccatcatgaataatgaaactaatagccgttcaaacaccgaaatcgagatagcggtcaagccgactgacgcactgattgaaaagctgagcgacgcagaaacggcgccaggctttgaagtggagtttgatccggatgaggctgagaaagccggcgcatttgaagaggatgctatcagtgaatcagacgcgttagacagtagcgcggacttaatctcgggcagttaaatatggcgaagaaatcgaatccggatcaatgggatttatttaacgactggctggttgtggttgatgcgccgatatcgccgattgttcattcaaaagaagaggcggacccatcaccgatatcgaaagatcagtctcgttatgttctcgcgttacgactcgctaaaaagctcgataaggacggtgaaatcacgacaaaattcttaactgatgaggccaatcgcgctttcggcggaacgatggccgaaggggtttattcaacgaaagacgcttatgatgcgatggaaacggcattcaacatccatctaggcaacaccgagaaagccgattggactgaacaggatgccgcatgggcaaccagcaaaactaaagaactcaccagtcgaatccagaaactgcccacgcagacgcggcgggatgaggaaatggaagagtttcagcaattcagcacaccgcctgctttgtcgtttgttgccaattgggtggcgaacgtcaaggcctatgatgtgatgggcgaaccctccgcagggactggcgatttagcgatctggtcaaagattgccggggccgaaatagttttaaatgagttatcgccacgccggcaggcgttactttcctcactgtttccaaaggcgagactgttcaaggaaaacgcggagcagctggacaacgtattaccacccgacatcaaacctaccgtcattgtcatgaatccgccgttttcttcgaccgctggccgagtgcgggggcaacgcgacacatcgaacggcgctagacatatcgaacaggccttaaaacggttacaagacggcggccggctggtggctgtggtcggcaatggaatggccgctgatcgaccggcattcgccaactggtggaaggagatagagaaaaagtacaccgtgcgtgcgaatgtcggcatagccggcaaggagtatgcgaagtacggtacgacgttcgataaccaactcttggttatcgataaaaccagaccgacgactcagcccgtcttaacgggtagcgtcgattcggtcgccgatttacccacattattagaaggcattagaaatgatcgtcagcacctacaacgaggcttcaatcaaccagcaatcgatgaagatactccaacagtaccggatagcttacaacccagcgacggagctcgcggcattagttctgatacgggaagctctcgagcggaatctattggacacgggggagattcaggagcctctgctactgatagccaagctggaggcgaatccgcagttagcgatgtcgttaatgacggaatcggagccgggagtggcatttcaaatggaactggcggaaacgctggaggaatcggcggcgcagatattggaggaaatagtggcatcagcgaaagcaaccctgtcggcgccgttgcaatagaggttaagagcggagaagtcagcgagttcaccgattccgtattcgcatcttacagcccgcagcgccttactattccaggggcaaaaccacaccccggaaagctggtgcagtcagcggcaatgtcggccgttcagccgctcacgccgacttatgcgccggttctgcccgtgaatgtaatacaagatggcctgctgtctattgcgcaacttgagtctattgtttacgcaggacaagcacattccgaattgttgccgaacggatcacggaagggttttttcattggcgacggcaccggtgtcggcaaaggccgggaaatatccggcattattctcgataacctgatgcagggtcgaagcaaggccgtctgggtttcgttcaacgagggattgattgaggatgcgagacgggacttcgcaggcgtaggcggcgatcccagcaagattttctttcagggcaaaaccaaggccggtaatgaaatcactcaaaaagacggcattctgtttaccacttattcaacacttcgcgggggcgagaaaaaactagccaatgacttagggcaaaagggcggcaagacccgtgctcaacagatcattgactggctgggcaaagattttgatggcgtgattgcatttgatgaagcgcatagcatgggtaacgctattgccgtcaagggcaaacgcggcgttaaaaagccttcgcaacaagcgattgccggtatcaatctgcaaaaagcgctgcccaatgcgcgtgttacctatgtgtcggctaccggcgctacggaaattagcaacttgagctacgcggatcggctaggtctgtggggcgaaggcacgccattcgccgacacgactgcgtttatcgataatgtgtctaaaggcggtattgcctcaatggaactgattagccgtgatatgaaggcgatgggcatgtatctcgcccgttcgttgtcgtatgacggcgtgagctacgaacggctggaacatacgctttcagacttgcaggaagacatctacaacgaactcgccggcgcttggcaagtggttcaagacaatgtggatcaggcattggaaatcacccaagccggtaagagcggcgcagggaagagtgcggcaatgtcccaattctggggcgctcatcagcgttttttcaatcaaatcatcacggcgatgcaaacgcctcgcgtgattgacgatattcgcgaacagcttgatacaggccatgttgccgtcattcagttggtcaacacaaacgaagcggcccaagaacggatcattgccgatgcggcggccaatgacgcagcccttgaagacttggattttacgccaagacagatgttgatggactacgtgcgcaacggttttccggttgcggcctatgaagagtctcaagacagtaacggtaatactatttatatccctgtccgtgatgcagaaggtaatcaggtctttgatcgcgaagcgattgccttgcgcgacgctttactggaaacgctgcatcaaatccgggtgcctgagaatccgctcgattccatcatcaatgcctttggttctgacacggtagccgaagtcaccggccgtagccgcaggtttgtccaggttcgtgatgatgaaggcaatctaagggttgttgaagaaaagcgcggtaagaacgcatccagggccgatgccgaagcattccggaacaacaaaaaagatattttggtgttttccggagcgggtggcaccgggtattcattccatgccgacaataccgcggaaaatcagcgtaaacgggttcactacattttgcagccgggttggcgggccgattcagcggtacagggcttcggccgaacccataggactaaccaggcgcaagaaccgcattatgtgttgcccacgaccaacctgaaggcgcaaaagcgtttcgtatcatccattgcccgcagactggatcagttaggcgcattaactcgcggccaacgtgaagcgaccggtcaaggcatgtttacggcatccgataatcttgaaagcgaatatgcctcgactgcgctgaagaatttctttagtgatctttatcgcggtcaaaccggcctgtcatttcatgaggtcaccaagcagatggggttaaatctgctcgatgagaatctatcgttgagtgaaagcaagatcccggcaataccgcagtttctgaatcggttgttgtcgctgaaaacggacatgcagaacgctgtttttggcgagtttgaggataggctcatagaggccgttgaatatgccaagcagcgggggttatatgatgttggtttgcagaccatgaccgctctgagcatagtcaagacgcgtgatgatgttgcttatgaagataaaaataccggagcacagacccgctatgtcgagttggctgttaccaacgagattaaatataactcatgggaagatgccaaaaagttcgtgcgcgaaaatccaagggataacgacatcagcggatggtttgtcgctgagttcggaaaaaacaaaggagatgtcttttttctttccgatataggcgaaagactcaattcagaaggcaagacggtacaccgaggcgtcgtgcatacgatcagaaaaaacgatcatcggtacatcgataatgccgatgcgattagtcgtggctatgactacagatccgttgtcactcatggcgtgggtagttatcagaaagtgacgctgaccagggcgatcgatgagaaggaagccgaaagcctttggaatgaacaactttccaatgcgccgaaaactgaaaccaaaactgagcggatgatagtcggcgtgatcttgcctatttgggatcgtgtcgagggatcggaaaccatcaagcggttgcaaaccgatgacggcgagcagctgctgggccgcatgttgggaccaaaggccgccaagcaaacgctcaaaaatttgggactggattccggtttgtccaatatgtcggccagcgatttgtttaattcaatcaagaacggcaacaaggcgattctttcaaacggctgggaaatcgcaaccgccaaggtcaaccgcgaggatcggatcgagatcaaagggcgcggctatctaacggatgccgaaaaacgtgtgctgaaagagcagggcgcatttatcgagcgtatcagttgggtggatcgggtctttattccaaccggtagcaacggagttcgggtgtttgaacggatcaccgcatcgaaacctgtggttgatttgatcaagaaaagccggaacaaggaaaacgatgacgttgagcaagaacatggcattcccaaagtaatcaacgaatcggccattcacccggtgaatagtgagtcggccgtcatagcgaaagctgtcagtaataccgcagatgtacatagccaaagtgggggcatttccatgaatcagaagaatcagaagaaaccgtatcacgaaaccgttgccgagaatttaatcaagcaactcgaagccgggacagcgccgtggcaacgaccgtgggaggccggagcgtcaggatctttcttgccgtataacccaactaccggcaatcgctacaagggaatcaatgcgctgcacttaatggctcaggatcgcatcgatcagcgttggatgacctataaccaagcggcggccgagggtgcgcaagttcgcaaaggcgagaagggcacgggtattcagtattggaagttcaccgaagaacagactaagaaagatgagtatggtaaacccgttttagacgccgaaggaaagcccgttaaagtgactgttaagcttgaacgtcctcgggtgtttttcgcgaccgtattcaacggtgagcaaatcgatggattaccggagcttgagaaaaaagaacaaacatgggatccgatcgagcgggcggagactatattgaacgcatccggtgccaatattcagcataacggcggtgaaagagcattttaccgatcattgaccgatagcattcacttgccggataaggggttgttttcaagcgcagatcgctattactcgacggcattacacgagttgggccactggactggtcactcatcacgcctagatcgagacctggcacatcctttcggcagcgaggggtacgccaaggaagaacttcgggcggaaatttccagcatgattttgggtgatgagcttgggattggacatgatccaggtcaacatgcggcttatgttggatcgtggatcaaggcgcttaaggaagatccattagagatattccgagcggccgctgatgccgaaaagatacaaggctatattcttggtcttgaacaacagcacatccaggagcaggagaaagcgcagacacacccttcaacacttgccgatcttaccactaaggaataccagcgcatgcaggctgcggaggaaaactatcatcaggagttagtccgtgtgtacggtgaatccagcgcaaatgaggcccgttaccaattaagccatgaggatcccattgttcaagcctccaaagaagcgtttcttgccgcatctgaagcatggcacaacgcaattaatgacgcacggaaatcaatagatggaggcaatgtatcccaagcctacgaggataaagcgatgacagaataccaagatgagcttgcacgattgttaaaactttcgacattgaaaccaaagttgtcggcagaggcccgctccgaatcattcagcaaacgggaattgttggatgagattgtgtcaatggaacccccggaaacgaatagcaaagaatttattctcgccagcgatcaatttacgttttggaacggtgatggatggcgtggactcgatgaagccaaacgttacaacgatcctactacagcattcaaagacgccctttctttagatgaaagtgagcgcgattatctggaagagagtgcggttatgctttatgagcacgatcacaatgcaaaaacgttcaatctaacagtaacaaccgcattgatgagcaacaaaccggctttaagagatcgatggattaagcacttcggtgaagcctgtccggaagatgttttagcggctaaaaccgaacgcagcaatacagaaaacattacgcagggagatactgattcaacgtctgattccaggacctatatcaatgttccctatcaggaaaaggatgacgcgaaaaagcttggtgctcggtgggaccgtcaagagcgatcttggtttcttccccctggcgttgatcccgaaccgtttgctaactggacaacacataaaagcgacgagtcggttgatcgacccattgattacgaacccgtaagcccgatccaaaatgataaacgcgagtatttagcagtgccgtatggtgaacgtaacgccgccaaatccgccggtgcgttatgggataaagttgctaagtcgtggtacgttggccctaatgcacagagggatgagctggaacgctggaagcctgataatgtccctaatcaacaaagtccggcaatatcccccatggaggaattcgcagacgccatgcgatcgatagactttattgtggccgacggacacccaattatggacggcaaaaaacaccgaatcgaagtggggggcgataagaccggagggaaatccggattctatgtcggtcacttggatggtcatcctgccggctacattaagaacaaccgcaccggcattgatatgaaatggaaatcaaagggatatttgctcgatccacaggaaaaagcaaaattgcaggccgaggctgccgagaaattggcagccagggcggctgaacaggaacgcgtccatgaagctactgcgcaacgagtaagccgtcaaacggctaatttagtgccggtattcgagccaaccccctatctgcgtgataaaggcattcagccacatgccggcacactcaccgacgccggtggccagaaaacgtacattccggccactgacgtcgacggcaagcaatggacgatgcagtatattcaagaagatggaaccaagcggttcgccaaagatagtcgtaaagaaggttgcttccatgtgattggcgggattgaatccctggatcaagcaccggccttggtcatttccgagggttacgcgacggccgccactaatgccgaggcattgggtttcgccactgttgccgcatttgattccggcaaccttccggttgtggccaaggcgttgcacgaaaagtttccggataaacctgttatcatcgccggcgacgacgataggaaccggctcctcacggatggtattaatccaggtcgcagtaaagccgaagaggcggccaaggctgttaatggcaccgcgatatttccaatttttgcgccgggtgaaaatgtttatccacctaatcttgaagcgtttactccccaacagtatagagagcatgatcgtgcaatgcgccgtctcgaagccgctcagaaggagccggaaacggtaaaactttcggatcaggaagtgattaaactgcaaaatgctttgctcagttctgaacaaattgatgcccttgataaaatgaaggcacatacagattttaatgatcttgccacacggagccaattagggcgaaacggattggaacgccaggttagggcggcacttagccaagctctacaagcccaaaaacaacgccaggaacggaaacagaatatagagcaaactaaggaactaaaacgagtacatgtaagagctcgcatttgaaaacggtcggactcgcatttatcgccggtttcagctcgaattaactgcaaataaaaatcctgccgagctcagattaaatgcaaatgagcttgcatttatcagtgccggctcgcgtttgattttctcaggctcactttatctgcaaatgaaatccggtaacgcccgttcaaatccggatgtcctccttgaacgatcaaaaccgacccctagttgacattcgaagctttgcgctcaatgtcggcagggattcaattagctgccattcaacgtaaaggtaaggggcgcgccgctgaaggaagctatacgaagccattgaacccgataaaaaggaagccttcaaaaccgccaacggacggcgcgtccctttgaccgttgtgttaggcaattactatctccaaattgtcaatcaaatcagaaataacagcattttccccagtgcgcgattcaaagaaaatttggttcttatcaccgacgttaatctgaaactcatctgtcaaattggctggagttctttgtttaatggagagggatttaaaacgtccgatatgaaattttccaccagaacactcggcaaattcatttttaagacgttcaatccagccgctagtatcaaagtgatatttaacaagaaaaaccgtgggaaacacctggtgttcttcgataggaagagttgtgtgggcttggcattcctctccgatccgataacttatttcagaagcgttcagtctctttatggactcttctagagaatccgcggccttaaatgcaataccactgaaacctctgaatgccgtaccttcaattccaaatctgactctaaaatttatgaattcgatattgatatcaccaaaattcacaccaatgctggtgtactcatcttttttcattaaaggccatgcaaaaggcaaacctagccgttccgctacattattagcaactttcaaagggtctccgacttcaatcataagatggtctatttcaaacattctgtatttatcctaatttacctcggtcaacgagtgaggccttacgcgacttgttatcactgccttagtcatactcagcattctgaggcagatcatccgtaatctcgaaccaaggagctttactcgccacgaatacatgacgccctggttttacttccgggtcggtgtcgagtgtgccgagtggaaaaccatagacttcaggagctgcgtcgaatttcgtgtaaaggctggagccgcagtttgagcagaagcctttgtgctcaccaggagacgattcgtagaacttcagcaactcctgacctttgagagtacgccaatccgaggattgaaccttcgcgcgggttctgaaagcagaggcatggagctttcggcacattgagcagtgacagtttagaacgtcggtcagcggcccgtcgatttcgtattgaatgcctccgcagagacagctacccgtttgcttcatttgtgcaactttcttgtgtgataacgtaaaatttaggggcaggccgcttttggcctgtcccgctcgaatgccgggttaggcttgattctcggatagccaagccatcacctcaacgacatttttatctggcggaaatacaggataaaagcactttttgatcaccccatctttaagaatgagcgtcagccttttgatcaattttgagccgccaacttcaagcaggggcagcttcaaagcagatgaaaaagaatgattagcgtcgctcaaaagcagataaggcaactccaagcgactagaggcttctttttgttcctccgttgattgactgcttatgccgtaaacgccaatgccattgcgtttgagttcagtgtgattgtcacgatatgagcaggcttgaggcgtacatcctgcagcacctggaattttaacccagccttctgggatggaaactccggggcgacctgtcattggataacagaaaattacattccaaccagacaagccggatagattgatttcttgatcatcggttgaccacagggaaacattcggtaccagcatgttttccaaatgctcgcatgcgccatcatccaaaggaactggcaaatctagaggaatcgaaaaaattacatcaggtggaggctgaatattttgcatcgtgattatctaaaaagccgtcgctgtgcctgcgattgttatacgattgagacttttacaagcgctgattcgtcaacaatagaaacaacactctcttcaaatacgcagggatggtcgtctgcgctgaacgactctaatgtgatcgtagcatcaagctttaatgccttcctggctatatcgcgtactgtgttcgtcacagatttttcatcaaaggagcctctgtcatgcctagagccatcagcattccatgacacctgtttagatttactgctcgtatgcttcttcctcgcgatatgaacatgtctttgttgaggaatattagggtcttctgggtcgattctcatcatgtagtctttatagcttgaaggtttccagtaactctctaatgctaggcaatcactctgaagtagtgattctggaaccttaacaaatatatgctttatctgatcatcgtctgcgccctcatctggctcatcgaaatagcctttaaggatgtcttttattctcatatgtcacccacacttagtttcatataacgccaagctcagccgaggaaggccgcgatgcggcctgacgtcggctggagcaatgtgttagccatttttagcgacgatgtgggtagggatgccgtcaatgctcatttggttcttctcctcccagtattgcttcaaaccgtcatcacctttctttgttgcccaatcagagagcaattctcgatcaccggcatacgaaaaataggggacagccatcccacaagaagtttgcacgaggtcaattctaacgtcgaaaacttgtcgagcgccaggcaatgcgacaaatcgaccaaaaagatcatcccactcggggtcagtcctgtgcacaacctttgccgttccataaagccgcaagatcagaggcggaccatcaaaggcacagaacatgatggtcattcgtgggttctgctgcacatgagcggacgattcattaccgctaccggttacatttagccaagcaatacgcgaattgcttagcaccctaaacgagtccataccttttggtgagatattcacgtgactatctgcggttgctgtcccgacaaagaaaatattttgtcctttgatgaactggatatgtttatcggataattccttaaattgttgacccattgggcactccgtgttgttgggcatcaaatgtctgcttgatcgaaaattcagcgtcatctgacggcacccccgatcagcgcgagaagagacctcaactcatcgaccatctgtctaggcgccggcagactcatgccgttcaatctcatcgggtatcggaagtaatagggcttatcgtgcgccgaattcaacagcgtgcaccactgaggcggttgcgctggtatcgccagcccacctgccactgcttccgcccacaatgcttcgaggttgtgacggatagagggtgaccgcaatctcttttccgtcgcccctgaactggcaagaaatgcctttaacgcacattcgagcgtttgcgcagaaagaaaggcgcaggagaggagttttggcgaagtggatattgccaatgcctcggctccttcaaatagtccacgcgctgcaatcagatagctttgagcctccgtaggaggaaccatcgtaagcggcggaagcgataccgtcggcagggtggcttgcatggaagcgtggatgatctttgtcatgcccaacgccatgctctggggcaaaccgaagcgcagcgtaggtttgtccctagcagcactttgttagccgatgatttgctggcgtacacaattacaccgcctctaaaatcaaccatgtccaggtaaccccggcagcttggttacttttttgtactgattcagcttcactatgggatataacattgaaatcaaatttttgagctaaaccaatcataccccctacctctactggatagcattttcgattgggcgctcccggtccatttcgtaacgagatagcaactctacctttacgcttaacgattgtacgcaagtttccaagtgcgatttttctgtcatcgtcttttatatgctgccagactgatgcgatcaaaacaaaatcgaagactttcccaacatttagtaatttttttagtgcaggtagataatccttcatccattcaattcccgaaacggcgtgaatagattttccagccaacctaaattcatcaactggttctactgcaagaacattatgccctaacgaagcaagccactctgcatctcttccagttccagcccctatatcaacaatatcggctggaacttctggaaggaaggctgagatatgagataacaacacagctgaatcgatagcctcaaaggcctgaatcaaatagtcactatccgatacatatccatttattacgctttgatccatgctaatttcacgatacccctgagcgccttgttgaacgacaactcatagctttgcacccaggcaacgaagtgctcttcatgcttttgcaatcagaaccaaaccgcatccgatcatcagtgcccctccaaatttgcatggcacctcggaggcaactcgcccacgaagactatcaacgagtttcgaggcaaatatcacataccccagcttaacaccaccaacggtcacggttgtgattgccataataactaatatgtcccatagatctatccctccaatatcaaccaacgtcggaaaaaggctggcgtagaacaaaatggctttgacatctccaagggttagaaatagccctgccaagaaatccgccgcaaacgaagacgcccgacttgtagtgcgctgaactgttggcggagactttgacttcagcagactgaagccaagccaaatcaagtaagcgctcccgcaatactttactacagagaaaaaaacaccaagccattcggctagaaggctcatgccaattaatgcgattgccacgaaaaccaaatcgcccgcgacaatgccaagcgcagaaaacacgccacttacccggccagcgcttacggatctggcgaccaccagtgcaacactagtactaggcattgcagccaggggcagcatcaccaggaacagcgttactgcttcaattatgctcatcgatgtgtctgcccccccgaacaggttctcgtcgcccaacgtttcattcagccgacacgtccgcgcccaactgcaatgagttgttggccgttacctctgcaatgaatataacggaggcgggcttatgggtctttgggtggacgggctcactgacctcaagtaaccgaaatccgctgctctggaacagtgtcacccaactgcttaatgtcctgaaataccatggagccggatccgtgaaatcagggccaaatccgtcccatgagccttcccgccaaccatcctgatacggggaattgccacacgaaaccaacggatggagtgtttgtacgataaaggctccgttcggtgccaagatagacggcaccgccttaaacagtgtctctaccgattccttaccaagaagagagaaattacagacgacaacgtcggataaaccatcaagcgcacccctggcaatttcatcgtaggaagccacatagaaactgccacctccggcgttctgcgcacgttcgatgagaccgggaacgacatctactccggacacatgcgccaccttgtcagccaactcgcggacaagccatccctcgccgcatccaacatcaagaacagattgggggggccgtttgagaatagcctcaacaatcgcgtcattggttaccaaccgacggctctcaattttccctttacggacggcctcgctccactggcaggcattcttagaccatgaatcaaatatctgttcatcactgaacgggtacataccgctctgtccttctattgtccaacgctgctaatcagccgcgcggctttttgcgtcggctgcattagccttgttgggcaatcttgccattcgaagggtcgcctcctcgcgttctgtaacgatgaagcctagccgctcatacaagcgccttgcggggttgtccgaaaatgtcgtaagccgaatggcctcttcgcaatgtcgattcaacagttgctcaagaatctcagtgccgatacccctgccgtgcaggaagggcgacaattggatattttcaatgtatacgccgcaatcatcgcgcttcacgctcagatagccaactcgtcgcccgtgcatgaccaccatggagatatggtcaggattgaaactcttgcgaaattcgcccgaagcccatccgccccaatggcgacagaatagatcaaacatgttccgcttagtgagcctgtagcagtactgatagtctgcatcgcaggcgggacggatcgaatatttcattcgtgcaccatcggtcatccagcagccaacaggttattgtcccgatctggctaacatacggagtttcctgctttattgtcaagaatgctgcttcaacgctaaatattttatatagtataacctatatttcattcatgataaaccgatcgggataattctaaagcctcgttctgaataaccgcaattaccaactcaagcgaaagtcaaacatctaaattaagagacaggtttagggaagtgcaaaatcatagccgacaggatgtgctctgggtcgaaaccggtagcttctgttcacatgctctcgttagtcctcattagtagcccgcttctttctggtgtcgaaaggcaagtacataaagagtattatcatccggttcatagcgatagagtgcaacaaaaccggaatcgccgaatggaatgagtaattcacgtaattctggaagatcttcccacggcctgccgatttcgggttctgtttctaggcgctcaaatcgctgtgcaattgcccgcgcggctcttgccgcggccaaaggatttttttcggctaaaaagaggcgacagcgttccaaaccttgagccgccccttcggtaataattattcgtggcactcagggctctcttgctcatgctctgtaccccaggtgctcagccaatgacgcgcttcttggcccgttagatgtttgcccgtttcttggtaagcctgccaagctgacaaggcttcttgtttgaaactttctcgggcttcttcgcgttcaacatagtcgcgaatggcttcgcgcataatccaatgcgatgatcgattcctcagatcagccaggtgctgaactcgattcttcaagtccgcatcgagtttaacagatgttgccatagcgatcgcctcgttagtaaaaggtagtacttttaaatactctagcataaggagatagcatttttccaatgaaatgtcagtgaagctttagctttggctctgcccgctttaggtcctataatgtattttataggacgtaaaataacaccctaaaacacagcctcaatttcgcccctatctataaggaataaccgcgatgaccgacttgctgctgaaaaatcccgaacgcgcactgaccgcgaaacagtttcagcaattatccgacgtgccgccggaactcgaatggttcgccaacctcagttcggaaaaaacccgcaaggcctaccaaaacgacattcacgatttcgcacaatttgtcggcatcagccaaccggctgagtttcgtatcgtcacccggtcccatgtcattgcctggcgaaaaaccattgaagagaaaaaattctccgacgccaccatccgccgcaagctctctgctctgtcttccttatttgagtatttatgtgagaaaaacgccgtgacccataatccggtcaaaggcgtcaagcgaccggccgcgaataacaatgaaggcaaaaccccggccttaagtaacgaccaggctcgactgctattggaagccccgccggaagatacggtaaaaggacaacgagaccgggcgattttagccgctctgctctatcatggcctgcgccggcaggaactgtgcaatctgagtgtcaaagattatcagcgccgtagcggcgtgatgatgtttcatgtccacggcaaacgcaaaaaggaacggtttgtaccggtagaaccgaaaacccagcgcttgattgccgaatatctggagctggctggccacggcgaacaactggatagcgccctcttccgccctgtgaaaaataacgcgaacggcatccttgacaaaccgctcaatccgcgttcggtctatcgcgatgtggtgatgttttatgccaagaaagtcggcatcacgctcgacacccatggcttttgtgtgcattcattgcgggcgaccgccgccaccaatgcattggatcacaaggccgacatcgccaaggtccaggaatggctaggtcatgccaatgtgtcgaccacccggctctacgataagcgaaatagtcggccagaagagagtccgagttttagggtcgagtattaagttacctaccagccaaactgcttggcgaaaaaaaagcatagtgcggcgaagcgaaattcatcttttttggggaaagtggagtaccttatttgcatcttcaaattcaaaggcaccattttttgttttgaccagaggccagtgaggttttcgtccctcaaacatgtcctggatttcctccattttaaaggtattcttaagtgtggcggcccgataacataaaacacaggaaccgataacatccgttttttgggaccacattagctgagaatatcggagcactctggcccacattaaatgagaattagcccagattaattgagaacagcccgataacatccgccaggaaccaaattaattgagaacgactaaagtaaccagtgcgtcttaactcttcgacattaatagcagcatcttcccgattgattgaagacctatctcttcctcaagcatcaatccagattgctcgaaaagtgcctgccattcagtaagcgttctttccctgcctcgggtagccatgaacatctgtatgtcgaaagcagcgatggaaaaatcggtcttcaactccggcataaccaattccatcagcacaatgcgggcaccagtgcccgtactggcgatggccaaattacgcagtactttgatgcaatcctcgtcatgcataccatgcaggatggcgctcaataagtagatgtctttgtcgtctttggctgcgggaaccgattcaagcatattcccagcttggtaggttaatcgggaaagcaggtctggcgattctttgccggtccagtttgttgctgctgcttgtatcacctgattccggtcgaacactaaagcagtgagatgtgaataacgctttaatatggccaaggatttacttcccttcgagccgcccacatcaatgatgcgatcaaatcgtccccaatcgaagtcggtggcaaaactgtcacccgtcaatgcctcaacacagtccatggcagaggcaaacagcgcatcgaattcggcatgctcatccatgtaggaataaagctcccgtccatgagaaagtttgaatgggacttccccgctgcgcactccctgttcaaactgctcataccaaggccggctcatctcaaccgagttatgcatcaagatcattgctcggacgttcttcgggttgtcttctcgcaaatatgcagaaagcgcattgtttttgaagacctgaggaaacacttcttcaaatatccccaaagccgccagcatgcgcagcaaacggtagatggcgtccggttgtgcagaggcgcgtgatgcgatgacttcgaccgtaagagattcatcccccagaatagatgcaatatccaggcgtacggcaatatgaagcacttgcgactgccaaaatgccgagcctatctggataagcctaaatggtgccggcgtgaccttgttgggaatgttttgcagccaagcggcaaacttcatcagttttgcaaagcgacgaaccgcgccggaatttttttgtaacgacatcgcaagtctcctgctattcagtttttgttggcgccaggccagtagcgaacaggtggccattgaatgcagccagtttttctaggaatgctgtgtccatggttcgccccatctgttcctcaaaaatatctttcagcaggatgggcatcattgccaaactgacgaatgccatacgcacgatgtcagggtccaatgaagaacttactaggccggcggacttcaaatcttcgaccttcctggcaccacgtgtgcggccgcgctccaatagttgttggataaaacgccgcccaggcccttggttaagtgctagaatttttatgatcagtttcggaaattcagggcgtttggacatcgtgtcatagtacagatggaggaagtccttaaacccaatgacggaatccaacatctgtccatccagcaccttaagcagcgggttcatggtgtcgcagatcatctcctcgtataacccctccttgttaccaaagtagtaacggatcatcgagatattggcgtcggccttttctgcgatctgacgggttgtgactttatggtattcgtcggatagaaaactgtcgagagctgctttgatcaaccgttcgcgcacgggatcgagagcaatctcctcaggggggctaatcattactttctccaatatcggatttaaaaagatagattagcagtctgcaagtcggtcctgctagaagtttcgtcaatcaaatcaggtggtttgattgaccttaaaattcactgttcaggaatgcgaattaattctgaatcggtccaagcacacaaatttcagcttgttccggtcacgtcattttttagtttgcagttagttcgacaaaacgatcattttgtcgaacacctgcagcaaagccattttgtcattttcagaagacgactgcaccatttcattggcctagagccgccttgtgcacaggactattgagcacacataaatcagtagtcatctgcaccgttcttgtttatgcttaaaactcctctgcacaagaatgagatttgggcatggatctcgataccccctccctttgttgaacgtggtttactgacgctgcctgatgctgcctgggaactggccaatcggcgagctcatttcattgcgccgcttttgcgcatccaaagagtagatgagccatatagtcatttgttccattctgcgctttgcgccgtccacgcaggctgaatttattgctctgatcttttctgactgagcgccagcctaccacgctatagctgcaaccttgccagtctgcgcaaacctatcgtcggtgacgtgccaaaaatcttgcggtattcatggctgaagtgagcggaatcggtaaagccggtagcatgcgccgcggtcgtaaagttgtgcccgcgtatgatctcgcgtatcgcttggcgcatgcggttccaccctcggtaacgccgaaacggcacgccgatttgctggctaaacaagtgctgaaagcgcgacggcgataaacccacctgagccgccagagcggccacggaagtcaggtcgtcgccgtgttcgaacaaataaccgacgatatgggtcaggcgtggatcgataacctcggcttgggctcgccgcttggaatagcccagcaaatcctgtaacgcgtccgttgtccagtctgacttgtagcggtcttcgtacaaattgcgcatgcagtttatttcaccgccgttaccgaccaatgcgccgttaagcgtccggctgttgttgatcagcggcttaaatacatcggcaccgccgacaaccgattcgatatataacacggcaatcggctcgccgccgacatccaattcatgcaaaacgccagccggtatgatcgcagtgcggcacaacgaccagtcgccgccgtgcagacgtaactgaaacttgccgtacaagcccgccaaaaacaccggcgcaccgtgttggtggctggcgttgtagtacaacggcccggtaaaaagggtgcaattgtcgcggatgtcccacagcgcgtccaattcgccattttccgcagcacgttcgtacaagcacggtggttgcctcatcgcttatactccttttttcacaagctgttcaggtttgaggcagtctagccgaaaaccgctcgggctggcaatgccaagccacgctcggccgctggctatggcaaaccattacatcaacgggcatacggcccaccacttgcagaggaaaccaatgaagcaacaaccgactatttccacgcctaatctgattcttctcgggttcgccgggctgtgtttggccatcgccctggctatcgcctgggtgctgggcgtgactttgttttttccagacggcatgttggccggtcatttggcggagcgcgacgacgtcatccgtgcgcatgtcgattatctgatgatggcacagtttctgttcatctttttcctgctgttccggcaatatgctatcgagccgccgctatggctggtagtggcctgctgcttcggtgcgttcttcaatccgctagccttcttgatcaggggcctcactcctaaagccgttgaggtaatgccggtcgaaccgcattttccgctacaggccttgctgagtttttcactgacaacggttgggtttctgggcgcagtgctgctggtgatccgcgccgtatggcaattacgtagacagccgaataacagcagtcagaaaacctctaacccgcccatgcagggtttccgttaaagctccaacccctatatggactccttccagcgttttaatcgccaccacgcgcttgttcggccgggtggcggcatcgaagaatcgcctgcgcatcgttaaaatcattttttattgtccaccacgaagtttttgacgtcatctctttgatcgcctgtctatggtgttcttcggtttttatgtcacatattatctatttttaaccctttgcgagtcttacattaaagaacgggttaagtgacacattttgacggctcagccgttttgttgcacactgtcacttaatcgaacgtataagtgacaggagcagtatgttaattggttatatgcgggtgtcgaaagcggatggttctcagacctgtgacttgcagcgcgatgcgctgttggcgaccggcgtcgatccccggcacctttatgaagatcaggcttccggcagacgcgatgataggcccggcttgacggcttgcctcaaggctttgcgcgaaggcgatacattgctggtgtggaaactggatcgactgggacgcgatctgcggcatttgatcaatacggttcatgatctggccgggcgaggtatcggcctcaaggtgttgaccgggcatggcgccgccatcgacacaaccaccgctgccggcaaactggtattcggcatctttgcggccctgtccgaattcgagcgggaactgatctctgagcgcacagtggctggactggcttcggcccgcgcacgtggcagaaagggcggacggccgttcaagatgacagccgccaagctgcggctggccatggcagccatgggccagccggagacgaccgtcggggctctctgcaaggaattgggtattacccggcagacgttgtaccgacatgtttcaccgaaaggagaactgcgtccagatggtgtgaaactgttatctcatggctaaaaccatcgagggggacttccttcactggcagtctcctgttactattgagcaattattaggccatggcatggagtgcgagcggcgaactatggattacgaacttagacccgcgacatgcgatgattacacctactgttaccgcctcaccaaaaggaacatgcaggatttgttctgtaaacactggggaggctgggttccagccgagttctgtaaaggattcgtggttgaaaacatagccatggtgatcattgccggtaaacgagcgggctacctcaatataataagagaatccaccttcatttacatagacaacatccagctatctcccgcttggcagaatcaagggatcggcaccagtcttttgaatcgtctactcgacaatcattcaagagtcagcattcgtctgacaacctttgaagacaatccagccaagcggctatatgagcgaatgggatttgtcgttttggaaagaaatggcatgaccgttaaaatggaaaaaaaagcccaacaagacggtataccggaaccgctaacacggactggcgacctttaacgttagatcagtgaagaaacaattctcttatgcgccactcagtagttattgccgccgcttggatggctggtacgttgatctcgttttccgcaatggccgtggcggtacgcgagctctcagctgattttggcacgttccagcttctagccatccgcagctttatcggtttgctagttatctcggccatactcacaagagtgggctggacgcaggtctcagtgcggaatataaaaacgcattttgctcgtaaccttgttcacttcggtgggcagttcggttggttctatgctattgcccttattccactggccgcggtgtttgctattgagttcaccgtgccgctttggacaatgatatttgcggcgcttcttctgaatgagagcatcagcgcgagtcggttgctagccattgcctttggcataaccggcgtcatcgttgtcctccgccctggttggtcagtagtgcatccagccgcctttgtcatgctgtccggcgcctttgcgtatggtctttctcatacgctaaccaaaaaactggccgagcaggacacgccgctatgcatcctcttttacatgatgttgattcaacttcctctgggtctagtccctgcagtctttaattgggtcactcccgcagcagcgcagtggccctggttgattgtggtcggcattacggggctcagcgcgcactactgtatggtgcgagctctttccttggcagatgcatcggtggtggtgcccttggattttctccggttgccacttatagcgctcgtcggctatgcactttacagcgaacgcgtagatgagtttttggttttgggcgctggcttgataatggcaggcaacttaattaacctgatttgggagcagaagagaaatcagtcttttcagcccgtccagcaggtaagcaccaattctgactctaggccaatccggcgttgaacatttcggtaaatggccaacatctgcgtcatatttgatttggatggtacgctcgtcgatagcgagggactgtgcaatcaagcgtttctcgatctgcttccgcagttgaacgagacagtggcgtcattgacccaacggtaccggggcaaaaaattggctcaaattcttgccgatcttgaacgccgtctttgtcaggaattgcctgatgcttttgaacaacagtatcgccaacgtgttgcagaactcttttctcacgagctgaaaccgatacccggcgttcctgaaatgctcgaaggcatgaatttccccaagtgcatcgcctcaagtgggccgcccttgaaaatccgtcaggcgctacaagtcagcggcctcgcaccttattttggcgataacctcttcagttcgtatgaagtgggttcctggaagccagagcctgggttattccaatatgccgccaaggcgatgggctttctgcctggccaatgcatagtgatagaagatagtgaagtggggattgaggctgccgcagcagccggcatgagagcattccagtatgtgcgaaatggcgaaacttcgccgcggcgagccgaagacgtagagctatttgacgatatgttgcagctgcctcaattgctaactcatacccaacttattgatccaaaggacgcggggtgatggggctgcgtcgcgcatggcggcgtcatctgaattaggtcccatttgcaagctaaatcaacataagatgggctggatattgcgaaacaagggattaggtctatagtcaatggcatggtgtcgcttctgcacttgagcgggacactgcattgaggggtagccgcagaattcggaaaatattgtacgttaagactgaatctgtcagattgaactttaagaatttatgcgagttttgtcagaaaataccggaacataccctattctgacgtaattttcagcccatgcctgacgtcttggttaaggtatagctcagtcggacacttaggggagaaaacccaaaatgccacctttgaccatcggcaaacttgccaaacaaaccgaagttaccatcgaaaccattcgtcactatcaacgtattggcttactgacggaacccgcgaaacccgaaggaggttatcgagtttattcagctgattcgattactcaactccgcttcatcaaacgcgctcaacaggcaggatttaccttgaaggaaattgccaccttgttatcgcttgacggggcgcattgtgccgatgtacgacgacacgccgagcaaaaatgccagcagatcgatcaacaaatcaaggatttaaaggctctccatcaggtattggaaaccctggtcaatgattgtcaacagacagcttcaaccgcgcgttgcgccattctcgatgcgttttgcgatgatgcatcaatcaaatcctgattgtaaataagtcaccatcgaccacttgactctgttcttaacaacagggtttaagcttcttgccagcattgaacattgtttggttgaggagattcgcatgaatacagaccctgaaacacccattaacaccccatcatggttaggtatcggcgcgctcttggccgctataggggcttcggcatgctgtgtcggaccctttttacttgtatctttaggtattggcggtgcatggatgagcacgttgacgggactggaaccggtccgtccatttttcatcatcctgaccctggtctttatggcgttagggtatcgaaaactctatctaatgcctcaccactgtgaagaaggtgaaaactgtgccacatccgacattcaacgccgacaacgcctgatgttttggttgagttccgcattcattttgatactgctggcctttccctggttagcgccttttttcatggcttgagaggattccgatatgaccattaaatccagtttgtttattttaagcttgtcatcaagtgtgttgtggatgccggcagcacaggctgaaacgactgttgcacagccaaaccggcagcaaacggtaacgctgaacatagaaaacatgacctgtgccatgtgtacagtcaccatcaaaaaagccttgcagaaagtcgaaggggtacaagaagtgactgtcgattacgattcaaaaacggccaccatcaccttcgatagccaaaaaaccgatagtgcagccttgatcaaagccacgacggatgctggttatccgggttcccttgccacacctgttgctcgataaagcaaggatacatctatgtctgattgctgctgccccaccaatacatccaccaaaactaaacagatttgtcctgagtgtggcgccacttgtaaaagtgtcggtatgtctacgctttaccatcaagtccggtttccagagaatcaggcgctcatcactgacagctattatttttgcctggtaaaaacttgctcaattgcttacttctccaacgcaggcaacactttacccaaacagcttttacgaagctgtcaagcgatccaaaacgacgcgctttgttactgttttgatattgacgccgagcaatatttatcggcgttaaaagctcagcgtgcggaaccgataaaggattttgtcatgcaacgcacccaagctggggaatgtgcctgtgaaatcaggaacccgtcaggccaatgttgtttagcaaattttaaatatctggaaacaagccattgagacacgctcatatagggataactccgaactgatgcgaacaagccaaaaacttatgctatagattcactccgaagtcttagcgtactattttttccgaattctgcggctatcccttatacgtagctaaatttagttcattaactttctattagagttcaaatgcccattgcggtactccttggtctagtaattctcgtttattgttcaggaatagctggtgcgctcgcggattgccagacgcagcatccgagcaatcgatatgccagtgccgaagaaaatatcccaccaaacaaactctcaaggtcacatatttcgcatcttcaacaatgtcatagtccatgcgaatcgtctctggatgttcggctttaggatgcggtataagctttagacacacttcattgttccattcagggtcttgatccagtctaatttcagataagtcgccttcaatggtggcactaaatctcgctaagttgtagtcgcgaaactcttggtgctcatgagcatagcacctgacgtgccaacgcaaaccatcattcacaagcgcaactggagacaaagtacgcgtctttctacctttagaaagagatccgtactcagcagacattttgcgatgctggtaaatgcatctggtaatagtagcaaccacagacaaaggcaattttcgcttgatgctgaaatttacccaaataggaagtcgtcctgcatgctctgtatcaattgctatggcacgctcaccggccaacgagtacaacgcttgatcgatgtcatgagtaaagtgtggctttccattcgagaagacataacactttttcttcagatcgtagtgcaatacattcggcgcaagttcggtgtagagagaaaggtcttttgtcgctgccggctcgctgattccgaatcgtgaaaccaagtcttttcggctaacccgacctaaaaataaagctaaaaactctaagtagaacaatcgttcacgttgaatatctcgataatgagacataactcaattcctgataaataataaatcataccgcacatagatataaaagatgtcatgatatttattacgaccacgtaaaaaatatcttaattttaagatatttttaagatgtctggcttatagtaaccctgtgttagagataccactgagttaacaccataatgttttattaaaccaaccttgatccaaaggagctttatatgaatcacccaaataagcctggccacccagaacatccggatgttccgccagggccacctagtaatcgtcccaatcctggacacggagcccccaaaccgcccaatcaccgtccggtgggctgatatgaaaggatggtggcatcgttaccccgaagcttttgaggctgagaaagccgccctcaatgcccttgggtgtatctgggaaattgataaggtagccatggagcgtggccgccttgttattcatattgtgcttctgcaagaggtgccgtctaatctgactgctgaatatccagatacttacccctactttccacctcatgtttttctccagggtcaactgcttcaacgtcatcaacatcctgttggaagaaatttatgtttattggctcgggagggcgaagattggctccccggccaggatacgttggcaggactactgcaagaacagtttccgaagatattggccgtgaatgcagtcgatctatcttctccttttgtagccgagatagaagatcatgtcagcgagcctctctcttcgtttctgaggtatccgccagattgtgctatcgtagttcccgatgtgtgtccatctacagatattcctgccggccgtttaacgttgaaggtccgcccctatcaaaaggacaatttaaaggaggctccatccgtcagtggcatggtgacaatgattaccgatatgccccggaagacgttgttcacttcttcgataatcccctctggttactctgaatcactagaaggtttttggttgcgcttaccagaacggcctcctttggaagagattccaaatctagcgaactttttgctgaagcgtatggaagctgacgttcctgcggttggaaaggcgctcaaagctggtaagcgtgggcaggtttttatcgcaggctttgtttatcaggatgagatgacatggcgggccacctctgatgattggctcttcgtagctatcagaatccagcatccagcaaaaggcccacattccgcacaagcacgtttacagctcattcgcactgattggggaggagaaaaagcttggatgcaacgtgtgccggctctccgacctgtacgtgcaaagtcggctttgattgtaggattgggtagtctcggatcgccgctaactttgcagttggcacgcgcaggaattggagtactgcatcttattgatttcgaccaattacaggtaggcaatactgtacgttgggcattgggttggggatctgccggtctctccaaaaacttggcactacaagctcatatcctcaatgattatccttatacgcaagtgcatacttatgacttacggatcggggcatctaatcatgtagatgagcataccctttctgattatgatcgcgttcgcttggtgtgcgagcaggtgaatttgataattgacgcctccgccaactaccgggttagccactttttggcagatctcgctcaggaacttggtaaatcttatttgtggttgacaacgacccacggagcatctgggggcgtggttggacgtattattccaggtaaaacagaaggttgctggcactgttttcagcataaactcgctgatggttcaatacgtcggccggcagatacgggaggagaagaaatccaaccaggcggctgctcacaaccaacctttatcggtgccggagtcgatagcgatgaaatagctctgcttgctgcccgtcttgccatttctaccctcaactgcggagaatccaatggctaccctgatttttcatgggatgtcgcagtcgtcgatctgcaacgggccggcctatcgatcgtgcctgactggacgccgtataccctaaaagtttccatcgcatgcaccacgtgtaatttgagatgagtcacctttggattagtgaaacggcatttcgggatatggttgaagaagctaatcgcgcatgcccgatggagacaggtggggtattagtgggctattttgcagagtttggtgaacctgtagtttttgctgccattggtccaggtcccaatgcgatccatctcaagaatcggttcatgcctgatcatacttggcaatgcgaacaacttgaaggcatctttcagaaatcttcaggaacatgggtgtatttgggagattggcacacccatccggattgctcagctaggatgagtcggctggaccagcggacgcttcgaagcattgcaaaattcaaacaggcagagaatccaaaacctttgatgttaattggcggctattcctcgtccgattgggattgtgcgggttaccaatacctgggtaaccgcctatttcgatttttggtttcatataaacagtgcagtttgcgcttgtttccaatccttaatatttaatatgaattttccgtgtgagggaaaaatggcaagaagaaaacgtaccagtcttattgaagatttacttgaattatcttttgaatttacaagttttttttggcaaataggagcaatgataacttgtttattatttattggtggtttatacagcgtagatactctaataataaattacagtccacctactgaactgattgctaaaataataaaaaatataccatgggtaatttatttattacccatgttgttatttttattgtgttttgtttttggtgtagcaacctactctgcttataagaaacaaaatttgtaactactcgccccatagaataaatcaaaaaaagcttgacaggttcttggcgtctaaaaagcgaaattgtgcagccctgatttaattgcagccttattggcagctcagtgcgcgcaacgtggatttcgcgcggttcaaggagtttagcctcaaaaattcggtttacagcgctcatacgcggccagcggaggccgaaaaaacccatgatatgatgagtttgtcgaaaacttaccctatcaacgtggatggccaacctcgataaagcctcggttcgaactgaagtcagtcgcttaaaagcggacttcgagaagctgtgtgccgaaggcaaaatcacctgtgaaagccaggctattatgactagcttgttcctggtggttgaattgatactggccatttttctggaacgcaccacgaagaaggatacgaccaattccagcaagccttcgtcgcaaaccggcaaagatgaatcggctttagggcgggctggcagtaacggtaaaggcaaagccgataacaaggctgccgcgcaaaataggcgtaccgtggaaactgtgacgctgtccaaggccctgacctgtgatgtctgcggcgaagatttgaccgacgtcccatgttcgcacgttgaaaggcgcacccagatcgatatcgtgttcgagaaagtggttgaacacgtcgatgccgaaattaagcaatgcccaacctgcgaggccacggttaaaggccggtttccagccgatctgcacggacctttgcaatatggcgatggcttaaaagcctttgtgatcaatctactggtgtgccaaatggttgccttgaatcgggttcagaagctgctggcatcgataatggatgtagtgattagcgaggccaccttactgaagttcgtattgcgcttgcatcaggctttggaagcctgggaaatgcagaccgtcgaacaaattctgaaagcgcccgtgatgcatgtggatgaaacctcgctacgggtggaccggaaaaatcactggatacacgtctattcagctggcgacatcacgctcaaatttttgcatcgaaagcgaggcaaggaagccatcgagtcgattaatctgattccgcgctatggtggtgccatcatccatgattgttggtcatcgtacttcgcctatcaacactgcgggcatggcttgtgtggttcacacttgctgcgcgagctcacttttattgtggatgcgcatggctatcgctgggcaaaaaacatgaaacgcttattgcaggaaacctgcgttaccgtctcaaaaagcgccgacaagaaactgacagtcatagaactgaccaacctgcaaaagcgctaccgggccatcctgacccgcggcgaaaaagagttgcccgttattccacccaaacccagtggcaaacgcggcaagctggccaaatccgatgcgcacaacttgtgggagcgcttcaaggttcatgaagccgccatactgctatttgccaaagaaccgcatgtggcgtttaccaataatcgtgccgagcaggatttacgcatggccaaagtcaaacaaaaggtctccggctgctttcgtgccgaagagtatgcccatgcttattgccgaatatccagctatctgcaaaccatggctaatcgaggctataatccgctcatcgcaattcaaatggctcttgctgggaacacggctatagggcgcgagtagttaccaaaatttttactaagtacacaaactttcaatttaaaattaagtgaaaaaatggtttgccgtgattgtccaaaatatccttgatttatgaacatgagattgtcgaaggcataaagcaaagcctgtatacacaggcagattatctgtgttcaaaatatggatcagattactttgtatcactatgtcagcttcatggtagcgtaaagccctttttcctggaattgacggtccacttattgatgctatatcagatgatgagcgcggacatcacaccggatgtatcacgctcgtcaccacgccccaaatcaccgtatccatttcctcacgcagcacaatatctttataagccgtgttttccgagcctaacaaccactggccggcccggcacttcaagcgcttgacgacgagctcgccatcgagcgcgcaaatcacgatcttgcccggcaccggttccagggaccggtcgacgaccagagtgtcgttcggatggatgccggcgccgagcatcgattcgccttgcgcgcggacaaagaacgtggccgccggtttctgtattaacagatcgttcaggtcgagcgacttttcgacataatcgtccgccggcgacgggaagccggccgagactttgccggaaaacagcggcaatgataccttgctgggagtttgggctggcaaataagcggaatccaaggcattagcgattttttgggtcttatccttacgatcatcaagcatggctttaatgctgggcagcagactttccggaacgcgaatagctttagtggattcgccgtaaggcccggttccggttttacgtcctgcgccgggccgcctgccgccatgtttatttttggttgtttctggcatgataaagtttatcttgcatcttgattattgtacaataaacaaaaatcaggcaatggtcaaccagccgtcatgggcagcgatttccgaataggagggtttatggacttggtaccgaatacatccgttcagtttcttcatgggagtctggtggaggcaaaacagtcgatattgtcgagttatcggatggcagagttctcggcattacggacgagtccgtggttctgtatccgagcatagaggctttggaagagggggaaaccgataacacggtaggcacaattgtgctcgcttgagctaacagggttatcatacccagttagatgattaaaaatcagcagatacaccggctgcaaccgatgtatctgcctaaccactattctactgtgatggagtagagacaatggctattgagcatgatatgacactagaagcccgcagcgaaaaaattagccgatcaaaaggcgctccgaaggctaaccggacaaaaaaagtttctgaaaagcggattaatagtaaagctaaatccgcttttccattgcggactattgatcttttttgcggcgccggtggtatcaccgagggattcaggcaagccggataccatagtctatatggtaatgatgtaatgcctgaggctatcgaatcctttaaatataaccacccggaagcgatcgcggattgccgttctattgagggagtcaatcccgcagaaatacgggagcgccttggtttgaaaccaggagagttggatgttcttgttggtggccctccatgccaaggtttctctatcaatgcaccggagcgttttttagacgatccacgaaacaagctattcacgcactatgagcgatttttggaagagttccagcctaaaacgtttttgtttgaaaacgtaccaggcttgctttctttaggtgatggaaaagtattccaacaaattattaaagtttttactgagctaggttacgcagtaacggcaaaaattcttttcgccgcacattatggggttccacaagagcgttggcgattgattttactcggttccagattttgcgaggtatcacatcctgtaccaacccactatgcctgtggacgtgccaactttcggggcggtaactcgatgacattccagcttagtgacacggatcgagaacgtttgccgccaacagccacagtgggcgatgcaataggagatttacctcgccttgagatgggggaaggagccgaagtcattggctatactatggatgcgcacagccgttatgcgcaagaaatgcgtaactctgccgaagttactttcaaccactatgctgcaaagttatccaaacagaatacggagcggatgaagcatgtaaaacctggtggatcgtggcgcgatattcctcacgagcttttgcctaagggaatgcagcgtgctcgtaaatcagaccatactaaacgttatggtcgattacggaatgacggtttatctggaaccgttctaacaaaatgcgatcctcactggggaactgtgtttttaccggatcaggatcgaacccttactgttcgcgaggctgctcgcttccagtcgttcccggatacttacagatttcttggttcccgtgtatctcagtatgagcaggttggcaatgcagtccctgtattgttagcaaaggctattgccaagcaactgcatctgcacttagaacaacagcggccaattgttacgcgcgtggaggttgagacaggggttgaacaagcagcttaatcctaagtattgccggtattatcgccttctattgaaaacagatcagtttctagttgtttcgctactggataaatgattttttcattaatgcgagtttcaaacatcgatagtggaaccggcttgccgccggttaggccttctaccgagcgttcaagagtagtgtagcgtatttcatcgcgcactatacgtgtccgcttttgtccatcgacctcttcaaatctaactataaaccatgcaatgtccgcatttgaaatatcggtcacattgtccatctcacctatggagtcgaaaaaagcgcgatctaccacaacagccatttttttaccccagcgcctgagtgttggcaccttaatttgaagctgtggcatcaaccgtttcgggccgctgcttctatagtcaggtcgacggcggccagctgggaaaatgacccaatccaccgacgcatcattgaaagcttcaaattctcccttcatcgcattgccggagaaatatactgcttgtatttcaagagcagcccacgccattggtgcgccctgagtggtcttgctgctaaccaaaaccatatctatacggccaacatcatctccaccttcattgtcagttgtggcacctgcttcgagaaagccaacctcaccaacaagcagcggttcagggtcatcaagtatggtttcaccaacccaattaaatacgtccaattcctcatgaaatcgataggggcatgtagctctcagttctccttgtttgccatcgacaggtactcccattgcccgatctgttcctggatgcttagcataagaataaagcctaagggaacacactccgccctcctttgaacatatcgcgtcttttttgcgtgcttgaaacgggcaaggttggggaattcgttcctttttcgtttttaggacttcttctgcaatctggcgacgttcttcaccggtaagttgggtaaggttatggccgtaccattctccgattccaaatcttggggtaaggtcttttagtttttttgtttttggcttcatttccatagcctatcaaggtaatacatagaataacagcaagccaaaaatagtaattatggcaactggcaagccgctacaccaaatagctcaaattcaatttgttgttggtcgctttttaaccatttggataaaagactagcaattctgtcattcgagtaattgtattttcctataataatgcgcattcccatattattgccactctccaacccaagttatatagttgtgttttgatttgtatatcctgttctctattttttgaaattttccctacccaatattcgcttcttctcttgggaatattcgagatgtgacaaccatgtccatgccaaaaacaaccattaaccataatgacagctttatatttagggagaactatatctggacaacctggtagttttttgacatgtaaacggtaacggaatccaagagcatgtaatatttttctgattatcatttcaggctttgtatctcgggcctttattttggccatcatccgacttcgaacctctgggcttactttatcagtcatgggtatgcttgccctatttattttgtgacgtaaatgattttaattgcttttagggatattggttgtcactagagcagattgataaccttttttctgctcaaatgtcggctaccaacttatataagcgctcctgccccgcatgcgtgtaaatttgcgtagtagcaatactttcatgaccaaatagcgctttaatatcaatcaattgcacatctttttctaacagccgcgtggcgtaagtgtgccttaatttatgcggcgtgactttcttctggatgccggctttgtcgatcatcttcttaagatacgccctcgcggcatgttggccaatcggtcggtagcccggttttttttgcgaacaggaattcatcgcggtatttgtcggcgatcgacaacgccaacacccgaccgaaactctccggaagcggcacctgccgctccttgttgcccttcccgattgctcgtaccgatttggctgcaccatcgaccacgcgcacatcggcggtttttaggctcaaggcttcgctgatcctcaatccggattctgtattaactcaaacagtaattcataacgctgacgaatcttgattgacgagggtttgctacggtcgtaaatgttgtccggaatgtcgtcgtaattgcggacggcttttgtaaacacatcctgctcttcttgttccaaccaaccaaacaggcatccgccgggggcgtttcggtttctcgacctggtagatcggatattcttcgacgtgattgttccgtttcgcccatttgtagaatgtggacaggcacgaaatcttacgggcgatcgcatgggggccgactttgtttttcgccagataggaaacaaagtgctcggtatcgatcggctggcatttgcgccagtcaaggccctcttcggtcagccaggcgatccagatgttcaaatcgctgtggtagctgtagcaagtcgtctcggcgtgacccttggtaaactttaaaaactcaagaaactctttttggattttcttgaacttcagctcaatcgaatttgccatctctcctccatccgctcgttcgaaacctgttggcggttttcgacggcgttccggtcgtttcggccgggaaccgaatttactaaaattagtattttatagcatcagacttaaaatgcaaaaacgtaaaagtctaggtttcaagcaaaaaatcgcctattagggaccacaaaaacacaaaaccggaagtccccaggcataaaaaatccctagaaattatgttgtagtaattttatgcccgattttcccctattcccaaccaaaattggcaaaaccgatgcagtcggctaacattggaatttattcgcacagcagattatcgctaacctttccatagttaccgataacactaaataccgaacatggccatttcaattcgagagaagcaaaaacgatttcaaccggctatgccgacgtgatcggcgaggctttgaatcgcttggaaatcgaaatggccccgcccggtacgaaggatatgatccggtatgggctgctctatctggacaaagaaacgaaaatcgaacgatgatacccacgacaatccggtgcgctgatttatgagcctcaaagcggaacgtaataggaaaaaacttgccaagaaagcgcgtaagggttttaccgactaccctgccgcgaccgttgccttttatggaccaaccgataaacaggcaactaaagtcacggttagcattattcctagtaaagattctccgcctgaacgcttaaccagctggtattccgagcatgacgtcaggcgtgatgaagagataggccaaatgctactggactatatggcggaacatggcatcaaatccgtcgtgatgaccgatcgcattattggctgtcctcatcaggaaggcatcgattatcccgaaggcgaaacatgcccagtgtgtaccttttggaaaggtcgagaccggtggaccggcgaaagaataaattaatgcaaagctatattattgaagactgttatccccaaaatctgtggataaccttgtcattaactggtcatatttcctccgaagccccctttcctgctggcgttgaatcgaattgaacaaaaattggtcggcactttttgatcatggttttgagcctccgatgacagtcggcgaggcagtcgagcgtctgatggaaatactgagcggcgaggaaaagctgatgatttcgatgatgtcggaagacgaactcatcgatctgcgtttcggccttggcttggcggtccggaatgcgtttggactgcatgagcctgggagcaaattattgtcttcgtgcggcacgtctcacccggatgatgcatcgaacgtgataattaaatcattgtggcgcaacttgagtgctttatgagaatagaactcgacagtagggataaagcacttatccaaaagtattggtacaccgccccagagaaaataaaagctcagcttttgaataagcggcgcaaaaccattgatatcgaacccgatgagattgaagatttgatcggatatttgtcgctagagtgcaatcattgcaagagtaatgggttggctttcgagcttaatgagctatgcgagaggcttgaatgcatagttttgccgaccaaaagaacaggcttcatgttttgaaaagaatgctacacaatgaaatcattcgataaacctatcatgcagaaagttattctaaccactggccttatattttcagcctttgcctggtcggcgccgagcgaatgcgaaaccgagcctcacccgatcgatgtcaaatacgaggcctgcatggaaaatacaaacgattatccggcgatgatgaattgcgttgaaatagccaattcggaatggcaaaaagaaatgtctgtgatctatgggaaactggaaagccggttacaaccgaaagaccattcgacattggagaaggcccaaaaggcatggcaggcataccatgatgcagaatttgctctttataatgcgatgcaggctggcgaagaagttgacacttttatgttcgccgaaaagaaaatgcgagtgatcggcgatcgggcgaaggagttgcagaatttcagcgattatgtgcttttggcagacgcagaaaaataaccgcccttttactggaatatcccgatatggacataaatcaaatactgatccaagcttttgccccattaataagcgtttattggtggctaattaccctgctctttcttctcgccttcctgaaatcgccatttatgaaaggtattttaggtgaattcctggttaatctggcggctaagttttttctcgacaaaaatatctaccggttatttaagaacgtaaccctgccaaccgaggacggaaccacgcagatcgatcacgtcatcgtatcccggtatggcgtattcgtcatcgaaaccaagaacatgaagggctggatctttggcagttcccagcaaaagacctggacccagaaaatttaccggcacaccagcaaattccaaaacccgctccaccagaattacaagcacacccagacgctgcaatcagcgctggaacttgatccggaaaaaatgttttcgctggtcgtctttgtcggcgacagcacttttaaaacgccgatgccggacaatgtagtctacgccgtcgggtatatccgatttattaaaagcaaaacgcaaccgatcataagcgatagtcaggttatggctatctgcgacaagattgcatccggccggcttaagccatcgctcaaaacgcaccgggagcatgtgaggcacgttaaagagatcgttgaggaaaaacagcgcccgattgatgataattcatgcaccaaatgcggcaagccaatggtgctaagaactgcacgaaacggcgacaagcagggcaagcagttttggggctgctcaggattcccgaaatgcagagcggttaggcaaatatcataagaaatcggctatatcagttcggtcagttgcagacggtgacctgatatttagcttaaggctatagaatgagctcaaaattccaatatcattgcagtgtaacaatataaatttattaatgtcattatgtctgtttattatttaaagcctattgtatggaataccgaaaactaccagagaccaagtggggtggattttaaatctggctatccaatggaaaatggttatggccatgaagaatggaacaactctccgggctttacttttaaagagaacaatcaaaattatcgtgtttttcactcagagggtttaggaaatcaatctttagatgattttgcaggagaaatttttgttttaatgattgcatctcataatggcaatcaatatttggttggcgtagcgggcggagctacaagcttacattctgaccaacataggaaaatgcgcgaacaattggtcaatcgtttggctattaatgcaagatggcaagaagcatgggctatcaataatgttcaatcttgttatagacatgaccaaaatgcctttcgtaaacactggcaaactgatgttcattggatacctaattggaagtgccctgcacatttgttcttatggttacaagaaccacatcttcttattcctcaagatatcactgggaaaaatcgcttagtaacgatgtacggtagatatcaagaaatcagcagaaaaaatgctttagatatacttaaaagtatcgacaacttaaataatgatactgatatcctggaaaatttaattcatatctgttctacggatgaattagatgctattactgatatcactcaaatcgaaaatagtaataacttagataaaacaacaaaagaatctttaattcaagcaagggtcggccaaggtaggtttcgccaaagtttaatgaatatatggaactcatgtgctgttacaggttgctctgttccagaacttcttcgtgcctctcacattaagccttggcgcagctcaacaaataaagaacgacttgatccgaataatggcttacttttggaatctaattttgatgcattattcgactcagggttgatttcattcgaagataatggcgccatgttagtctcagaaaacttgccagaaaccgagcgagagagactacgaataccagctaatctaagaattagtccgaatcaatatctctgtaggtatttagattatcatcgtaataaagttttcagaaaaggcagcagatgtttaggacaacctacatatcaatgactgtttagagatttgacactctgattccaaactctatgcattaacccgcaaatcgtccggtccggcttaaaagcatacatccctgtacgcttttaaactcggcccacccgaattgctcacgccctcgagccggaagcgcccgggatttagccatccctggcctcgcggcacacacgtccgtgtaagtgccgccccacctttggccggcacggttcgacgcccccggtacttgaccatcgcacgccgggagcctggccgcgtcaaatccgccactcgtttggccaagcgcaatacgtccctgtattgccccaatggccaaacgaatggctcagtctttccttttccgaacgtgcgcgggtatttgccttccatgacctcgcggcgaaaacgtccctgtcatcgccgcccgaagtcttgacgctctgtttatcgctcgccggtaaccaagcgttgctatacctgccctcgccggcgccgaccgtttaccgtcttaaccggtatcgcacttggccgtctgggtaagtttcttcgacaagttcaatttcacttccgactaccggcaattggccggtgatcatgacccgcggttcgatatcgccaacccgaatccgagctgtgacaaggctcacccgactccctgtcttccagctttggctttcgccttggttgacctcgataaccgtgccggtcactgttcgaacttccggttttccggacgggaacaacgcccatgcgctaaacaaggctatgagcagcattatcagtgcgaaaattgctctgttcaggatgtaattttgtatggaggattaccggcaattccccgctttccggaatcctgctgctcgagcctcggcctcgctcgaaaattcgacgatgttcttcggcttcatcgcgtcatagctcgggcatccggccggcagatggtagaccctgctgttgcggttgccccggatcgtgccgcccgctgcagccattaccgttttgggctcggatgacgccggatgactcgccgtgatcggcgtgacaatgccgtcggcggtgttcttgtggtttagcgaccatttccgttcgccggtcacgaacgggttgctgtgccccatgcgggcggcaatgcgtctatcacgatccctttcccattcgctgaccggaaattgcttgtcccacgccagcaataactgttgctgctgctgggacatccgcatgttgtagcggtcgtgcatgtagaaatagatacgcgcgatttggcctttgacctcatccctgggttcggcggttcggcccttgaaatcgactttgaaatcgcaggccccgtgctgtttaggcgtcgacggcagcatgccgaaattgtaattgctgcgattggcgttaatttcgccaaccgacggcgtcaggttgtgaagatcggcttccatcgcgttgaacaccgggtcaacggagttgcagttcttccgtccgccgttctgccagcatagccgggcccggtcgaagtttgaggccggaacgacatgctcccattcggtccggattgcccggtttgcctgtgaccggatttggtagccgcaggattcgagatcgacgcggccgccggatcgtccgacccattcccagttacagccgcaatagaaggtacccgcttgggttcggtcgtggtagacgtattgccgggcttcggctttggcttgctcgaaggtttgaggtgccgcaaaggtttggagagcggcaaagaaaagtagaaggccaatggttctggctgtattgatcaatgttgttacccgttatccgaaaaaaacgggtaatcgtacaggacaataatgtgttgttctaatcggaaatacagcgcacagaatccgccaatcgtccgggcgagcgcaaaacgtccctgttttacactacgccctccagaatggctcacgccttcctatcccgaacgtgtgcgggtatttatcttccctggccgcgcggcgaaaacgtccatgttatcgccgcccaaagttttgtctatcttctgatcgttcatagtgtccgacaggttgctttcccggcgcttcgcaatcccagcgatgcgtgacttcggcagattggaggggaaatggtgatgtaaaaagtgtctcaacttgagacactttctaatggcgttgcgaaaaatccctactcttcgctttggatcaggtcttcgagaaaatctttcagcttctgctcttgttcggcgttcaagacgcttgcgttgagctttacagtgatgtgcttttcggttctggcgatattaccgactttcttgccgtccctgatcaaattgtgcgcatcctggatatggctgtctcttgtttcatggcctttgagcttgcgcgcgacgtaagccgtgattttcgtttggtcgaggtcgcttttcaataattgcgcccaggcttcggccaaataatcgttgaccgagggatgatccttgttttcgttcaaaagccgcttgatgtcgcttgctgcggatctcgaaagcaagcgcgggttttcattgagatcggcgatgatgaaatccggcaaatcgtcgtaggcgtaataccggtacatatcctggcgactcaagcccatcgattcggccagttttttcttggtcgggaaggctttctcgacttgcttcagggcaatgccgatttcgtagtcgctcaaatcctcccggtcgatgttttcggccagcgcgaataccgccatgtcgctatcggatatctccatgaccacagcatcgatatggcgccgttcgatttgcttatgcgcccgccagcggcgctcgccggcggcgatttggtagctatcaccggttttcctgagcaagatcggctccagcaaaccaacttcggcgatcgactgggccaatttgtccaattcgtcttgcgggaaaatgcggcggggctggtaagggttcggctcgatctttccgaccagaaccgagaccaattcccggccttgatcaaaattgacggattgttccgccgaggcatgacgctgcgtgttctcctgcagctttttctccagcatttttttaatgtctttagccattgagccggttccttatacaagattgagttgtttggcgatcacgtcggccagccgtttaaactcgcgcgagactttgcaggttcggtctatgtcatgaacgctcgtttgcgctatcgccgactgattaaccttagtgctggtcgatatgcggacatcgatgagtttgccgatttgctctttcgccgaagcctcgatcagtttgcaaacggtttgtcgggcgtcatgacggatcagcaaggcgccgatcaattcgagctccgggttgatgcgcctgattttttcgactaacttcatcaggtcggtcacgccgtacatgccgtactgcgagccggactcgatcgggatgatcagatgcgtagctgccgccaatgcattgctggtcagtagcttgaggctgggcgggcagtcgattaggatcacgtcgtagatgccgcgaaggtggtttattttttcgcgcagttcctcggaaggccggggggcttcgtccttcaattcgtcttcggccttgccgagattgagcgagccgtagatcagagatacgccctcgatgttagtgtcatcgtagatcgattgcggcagtttctcgacctcggacaacagcagttccgcgcaggtcaccggcacttcgctcgggtgcttcttgcctatatgcaggctggcgttggcctgcggatcgaggtcgatgaccaggacggaaaggtccatcttcgccaattccgctgcaaggttgactaccgtcgtggttttgccgcacccgcccttgtgattggcgacggcgatgacggctgtttttttgttctcggttgacttcatttttgtgttcgttttatttaattttgtatccagtataaagattatatctggtaatgcaaacctggtatttttggtataaacagcttatttattacaaatatatgctcagattatcgctcgattgagtttggattgcctttttcgaacataaaatcgtgtacgatcaatccaaaaccgtattcattgttatcaaaaaagtgtcgcaagttgcgacacttttttgaacgataaccctggaaaggaatatgccaaacctaaaactcctggccttcatgctttgcgcttcatctctttcggcggcgccgttagatgccattctgaccggcatcgagccgggcacgatagtattcatcggcgaaagccatcatcgagccgaatcacctttgctgatcaagcgactggtcaatgagctgatagcaagcaatcgatgcccgatcctcgcgctcgaaatcggcagcgaccaacaatcggtgatcgaccaagtgatggcgagcgaaaaaccggccggcgcgatcgagatatccgacatcatcgatcatgagccttatcgcttgatgatccactatttcgccaaacggaaagcccggggttcctgtttaggcgtggttgcgatcgacgcaagcaaggaggcaaccgtcgaccgtaacaaatggatggcggctcggctttcgtcattgccgaccgacaggcctgtggtcgtattggtgggtgcattgcatacgctcaaaaaggtcgactggacggtgccgaccggcaagccttatgctgccgaaatactggccaatcagggctttcgcgtgaaatcgtttacgcagcgctggataccgaaggaatgtccgagtaattccattcgtcaacaacgttttgtcgaggcatccgatccggaggcgttgacgattctcaacgaaaccgtgttgtcgctgctcaatgcggagacggcggaatcggccgagggcgtggtggacgggtttatcgtgtgggagtgcgacagctgacgcgacaatctgtcacagttgcgccggtgcgtttagcggatattctgtaagcactgatctttttcctggatcagccatatatccttactctaatgcggaggcttagctgtccgcatttttttgtctgaaaaactgccattaatcgctgcgcaaggctatcgccaagcttgcttttgaaagcgtccctgtgagaacgcggtagtcgtccatgacaaccgcgtccagccgttcgacgttctgaatccgccaatcgtttgaccaagcgcaatacgtccctgtcatcgccgcccgaagtcttgttgctcttacggtcgatcgccggtatccaagctttgctatccctgcgcgtcctgctgtcccgatagcgcacgtccttgtgctttcatgcgctacttagcggatgtcgatcgtaccggttgtcgccaccgaatccctgccgatcgcttgcgtgttggtcagcgtgttttttgaggcgttgatcagagtcgatttctcaactttcgagccgactatgctgatagaacctgtgtgagcggcgctgttgcggccgatcgcctgtgtgttggtcaacgtgtttttggaagcgttgattatcgtggatttttcgattttcgagccttcggccatggctgtgccggcgacgactgacattaaaacgactgctgctaaagatactgttttcatgatgatttccccttggtttttgttggtatggttcgttcaagcgtttcgttgaacttgtgtacagtatcccgatttctggagaaaaaggcggtgatcgagtcacgccgattgtgtgatttattcaccggccggcgaattgagagggcatcgcttgttgaatcagccaatcattcggatgtcgtctgcgcttgccttcggcaaattggagaaggcatggtgatgtaaaaagtgtcgcaagttgcgacactttttcatggcgttttggaaaaatcctcactcttcgccttggaccaggtcttcgaggaaatccttcagcttctgctcttgttcagcgttcaaggcacttgcgttgagctttacggtaatgtgcttttggccgtgacgatttatttgttaacgtagcaccgtcggttgccggaatgagagtcttgagggttattgcaggttttcttaatcatcggttttgaaggaaattgagcgtggcacataatttttctggctcagggagcggaccaaagcctgcttcgccgccggcgaagacaaaggagacgaagccgtggtttgctgggttacacgttgtacttctatcggttgaaacggcatcgaaggcgctgtatccgctttggttacatctagcgtcacgaaatgggcttccaaccagtcagcccacttcgacgccttgcgaatgagatcggcgcggtacacggcattgtatcgcggcgtgcgcgagtgatagttagcggccttggtccacagatcgcccttgtcgttgcggatgtgcatccgcaatcgccaagcggccagatcgaacgaatagcaaccagcggccgctacatcgttcgctgtgattccgtaccgttccaggtcgcgcaggtatgcggtattgaattgcatggagcctacgtcatgggtgccgttcgtgttgcgcacccactgccacggcttgccggcttctttttctgccacagccaacacaatgttggccggtacttcatatttgacggcggccgagattgcgcacacgacgcgctcttgttcaaaaggtgtaaggtcggcaataaacggcagcataacgcccctttgggttaagtcggtgcgccgctattgctgcggttcacgaaggccgcaacttcggaatgagcatcgttttccccactgaggctgttgccggcgaattcgatgtttgtttcgggcgaactgccgccccttatggcgccggcaatcttgcccggtgtcgtatcagcaatgcgctcgttggctttggccttggttacatcggcaatacccttagccagattaacgccagcatcgacggcaatttttccggaagttgccgctgccgccatgaaaccaccgctttgcgtgtctgttgattgccggttaccgccttgcccggtttttgccgagctggttgattcggtcgagccgctaccctgtttatcgtcactcttcgcttggtcggagcctctttcggcttgccattttgcttccatatcttcgccgcggccccacatgctgtccgagctgttgctgaatcctgctgctttagcaaatggagtgccgccggtattactgctatcgctgcttacgccaccggcggcgctactgccccacatgttggtcaggacatcggacccgctggaaacgttttcgctggccttggagaacgcggccatgaccgcctgcgcacctccggcggcattagccgcgccggctgcaaccatagcgcctcctgttgccgctgctgccgtggccattccagccgcaccgattgcagctcctgaaccgaattgaccaatgccggcaccacctatgctcgcgccggtgatgatgccagaaagcaatggtggaatgcgattgacaagcatcattagggcaaaacagaatactagcattacgcctagctcctcaaagttgagcgtgcccttgttcatattgtcatagaatgtagaaagcaaatcattgccaatgccgaccaggagcaccatggcgaaaagctgaacggcaacgccaaggactgtcttgtagtagttgatcgccatgtcggaagtccaccgagacccgccgaagcccaagaagaatataccggcatacatcaaaatccacgcggacaccaagatcaacagcatattcaccgcgactgccgcaagcagcaataagataccggcgctcaaggttaggccaaccaagctgtcgataggttgccacgcggacatatttttaattgcctgcttaaagatcataaagcccacgttcacaatccctgacggagaaataccggtctttcctatcgcgttctcgccaatctgtcgcagcgatttaatgatggaatcggcgtattctggcccgttcctgagtaaccagaggaaaaaaccgaaaaacatgatgaaacggaaaaactctgcaaagaattctcctatgtctgctttacgcaatatcatcattccccccgtccataccagtgaaatcgtgcccagtgtccagaacagccacagtgccgcattcatgacaaaggttcgccatcccgacgctctcgtagaaaattcggtaattactttatcaaggataccctgattggtgccagagtcatgaaaggccaattgagccgctgcatcggtcgattttagtaatacaaatgcggccacaaccaccgctggtttaatcatcgtgcgcataatagcatcaccactttttcggattagcaggcaacgtttccgccccggtagaacgacgcgaacacaacccgctaaattgttcgcgtgttgttctgtccgctatctgcattatcttttctagctggcaattttcgtcgttgacttctggcatcgcttgctcggctggctctggatcacagcccgccaggaaagctggcatgacggttaccaaaatcaaaattagagcttttgttttcatgatcgtactccttcaccattttttcggattggtgggtagtgtgtcaggccctcgcccttctgtcgatgtggcgtgtgcagcttgctgcatagcctcaaggtccgcctccgcctgcatcctggttgcaattgcgttctgctgcgcgatcaacagtccacgtatctgtaatagctggtttgcctgttggctcgcaagctggttggcatagccaatggcttccattcgtccgccggcgctttgagccgctgattgcagttgctctaatcgtcttgagtcggacttgagcgcatcttgttgtttatccagtccacgaataagcgcatcgtttgcctttttttgtgattcggatgccaagcgccggttttcgctcattgccgcgaactcggcgtcactgcatccctcattcgagaaacacggcgagttacggtaataagaaacgtcttggaatttactcaggtagctatcgagactgccgacttgattcttataatagttgagtgtgtcggtagcggttattagaccatcgatcgtcgattgtgcctgatcccagatataagccgccggcgctaccgtgttccgtatttgatcctcatattgccgcaattgagtcgcatattggtcaatttgtttcagtgtttgcgcgacctcctctcccgctgacatagctgattgaatcgcagtttcaatattttgcgttaaattggtcatgtcaattacagcggtttgcgcgtgcgcctgctgcgtaatcgaaaccaccaaagtgatttcgactgcggccagaaggatttttttagccgctaaaattttatgctgcatcgatgttgtcaaagaactctttctcattttattctccagtgttttgcatagccggccgctcaacccgacccgctacgctcggcggttagggttagggttagtaatcaaacggttgataaggttttgaaaacgtcatgtctttcgtgacaatcacattgaatcgatagccagggcgaatctccagagttggcgctatgttcatgtttttggcaatcaattgtgcggtgacttggccaagttgctggcctaacgcctcactcaacgcgctactggcatccggccggccaaatgtcgcactactctgattttgtcgctggctgtaggttgtcgccgccgttacggccgacattagcagcgccgagctaaagagtcggacgtaatgatgattttccagatcattaaatccagcgtaccccgcgctgtccgctcccggcatcgcaccgatatccagcgctttcccatcggggaagacaatacgttgccaagcgacaaggacacgggcttggccataggccacatcgctcgaatacgtgccgaccagacgcgatccttgcggaatcaagaggtattttccaacgggtgtgtcgtacacgttctgcgccacttgcgccatgatctgtcccggtagatcggagttgattccggatattagcgttgccggcaccacaaacccggctcgcagttcatactgtgaacggggggcttgcgtagtcgattcgagttgccaacgatcaccggcattattcgcaaattgcgcgtaatcattaccggttccttcgcttgaggttccggtttgtggtgaagcggatgaattcgccggcatcccgccggtactattcaggctatgcctactgcgaagatgatttaatcgcttttgatacgccgttgatgggtcctcatgctgtatcgagtctatttcttggcgcacggcggataacctagccagcatggcttcgcgcgttttgggcgtgttagcgcccaaattggcatgactggatgacgagccggagctttttggcgcaatgacttgcacgccggttttggctttcaccgcttcttgaaacatttgcagttttgcttggcggatacgctccgcttcatcatcatgcggcggctgcatagggggagccggtggcctgtccaatgtttccggacgcgcaatgagtaccggttcgggtttcagcatggtttcttcagcgggcggtatcaaaggcgtgattaatccgtccatgtgatcgccggcaatttccttagcgaacatcgacgtgttgccggtttttagatgccgatcttccgcatcagtctgctgggctgcacgatccgccgcgacaatccccatcataattaaaaacgccgtcaataccccgccgagtagatacatgggtaaattgttgacgcggcgcacgccagcgcttggcgatgcgtccggttccatgttgtcgatatcttctgtcatcatttactccttatgcgtccaatagccgcccggcatcgccatgccgttttgctcgatgtacgggcgagtaatggattcagtaccgacccatagggtcaaatggtataagttgccgttttctcccttgacttgactgagcacatagcgcaatggcagtagggtattgtcctctgtcgtgtttgggctgaactccatgacggcatagccgctttcgcgcaagttttttaccagcgcattcccgaaaacatccggtgtcgtttgttttagctcaaattgtgtttttgccggcggccatagcgtggcgagctgcttgacggcatcgtcagccagtttttgttgatctactgaaacattacgtagatggttgccgtatggcgatgtcacgcatcctgcgagaaatagagcggccaaagccgctagagatattgtgcgcatacgattattgctcccgtctgatggttactcggtcttggcttctgccaacgccggaaatcaagatggcctggtcaaataccgtgtccacgatgtagcggtcgccttgaacacggtaattcactatgacggtctcatcatccgagaacaggccgccttctttgcgcaagaccaacagcgtcggcgcttcggtctgcgccatcgccttcggcatttgtaagatggttttacttccatcgttaaagacgcgaatcggacgccatgcggcatcaccgtccagctcatagttgaaattcagatcgcccaggtactcgcctgtttgcgggaggattttttgttcgcgcgcttgtgtttcccgcgtttggatcgcatcccatttggcgagagcatcttccgggtaggtgaacgcaacctgcggcatgtactctgtgcggtgcgagcgcaacttgaaatgatacgtgcggcggtttgtcgtcacgaccagactggtctccaatcccacatccatcggtttgatgatgagatgttgcgtttcggccggtccgctgccggtgatggccggttccacagtccagcgggccgtgtcgcccagatggatcgaattgaccagttctccggcttgtagggccacgtcacagacctgaagaaccgcgcaaacgatactgggctgttgtgcgccgtatatgaaatggatggccccattggcaccggatactggctttatgccgttggattttccggcttcccagcgtttcgcaatcgcgagagccgtcttttcttgtgcggttagctttgggttgtttttcgaaaaatatttgtcggccagatcgtcgccgggaacggccaggatgggttccgacaccgccatagcggcggccagaagggtgatcatatgttttttcatggtgaatagtctcattatcattatttcgaggaagtgatgatcgaccaggaataatcacgcacatagataccgagcggattgttgcgtaattgctcgtctgttgtttgagatgtaggcgaggcgatatagacattaactaatgcccgcatattgaccggctgactttttagaacgccttgccgatcgcgaagggtttctacccattcgacctgccaggtatccggcgattgcggcagcacggacttgatttcggtgctgaccatttccttcacggcacgtttgaacgggctggaatcgggcgttccgttcagccattcgttcattttttgggttgcgggatcattaggcgataacattgcgtagacgcgaaataccgctttccgctgtaatgcgacatccggcgtgaccatacgggcattagcgatgaaatcggctatagacgcatgaataacgcggggatcggcggtagaacccgctgtcgtcggtccgacggcaatggtctggccatgcttgtcaacctcgataatataagggatgaacttagattgactgcctatatggatgattccaccaacaccggccagcgcaatcaacaacgagagaataccgacaacttgccaggtctgccgtgacgacacgaccgaaccgacatgctcattccatgtgcgccgggcagtcaggtacgggttagtgtcctcgtttttgggtttattgataggccgagagtcaactggttttttcttggatagaatcaggctcatattgcgggctccaggtagtcattgagtttcagtccacgcccggcgagccattcatgaacccaccgatcgccgaatttggtttccaggctcttgatggcggcaacggaatccttgtccgatgcaccggcaagtgacagggcaagcggtccgagagcgaggtcgtagagacggcggccatattcggaaacgtagtaatattgctgtttcggaatggcggtcgccaaaatttcgatttggcgggcattaaggcccatacgacggtagagggctgccgtgtcttcgtcgcgggcatacacgttgggcaggaagattttggtggctgtcgattcaacgatgacatccaagatacccgagttggccgcgtcagacaggctttgcgtcgccatcaagacaaggcaattggctttgcgcaagactttgagccattcacggattttgtcgcgaaagaccggatgcccaagcatcaaccaggcttcgtccaggatgatgacggccggttggccgtggagtgatcgctctattctgcgaaatagatacaacagcacggggagcgcgaatttatcgcctaatcccatcagctcctcgatttcaaacgtcgtaaatgaggacagagagagtccatcctcttcggcgtccaacagatagcccatagatgaaccgatagtgtattgctgtagcgcctcacgaatcggtaagtcttgtacggtcgtgctgaaatcggatagtgtgcgggactgagtgccgctcatgctcaggatggcatggcctattttattgcgttgctcgggcgtggttatcaaaccgttcaacgccaggatcgtatctatccattccattgcccaagcccggtcgccccgcgtttctaagaattgcaatggacaaaatgcgagtgattcgccgtcccccgccactgaaaaatgcagcccaccaacggccttggtaagcggataaattgacatacccttatcgaatgcgtagatcgacatgccggcataacgtcgtaattgtgcggcgataatggctagatgcgtcgatttacccgcacctgtgggtccaaacataaaagtatggccgacatcgcggacatgcagattcagtcggaaaggtgtggccccgtgagtcacgcagtgcatcagtgcctgagacatgggcggatacagcggacaaggggctgttgcactacccgtccagatcgttgacgtgggcaacagatcggcgaggttcatcgtgttgatgaggggccggcgaacattctcgacaccgtgacccggtaagctgcctagatacgcatccattgtgttggtggtctctatacgcgcgacaaaaccgagacggtttatcgccttttctacctgccgtgcgattgcctcaagtcgcgttctatcctcatgcatcaaaataacgacactggtgtaatagcccatcgcgacaaagccgctgttcacctcggctatcgctgattcggcatcctctaccatcgacagagcatcctgatcgataggtccggtattggtattaaaaacttggtcgaagaatccgcgcactttttggcgccactttttacggaatttgtccaggtgggcaaccgcctcatgctgatccatgaaaataaaccgggatgaccagcggtagtcgcaggataattcgcccagagcgcttaagattccgggagttgactccagcgggaacccttcgatcgcgacgacttgcataagagagcggcctatttttggcacgaccccggtacgcagttcttgcccgccaatgagagcatccagatacatggggttgctggggagctgtacgggatgatgcttgccggtaatgcaaaattgtatccagctcaaaaaatcatcatgcgtaacagacgttccctcctccgtaacgaccttttgacctctgagccgtgtcatcgtaatagccgttgacaggcgttcctctatcgacgttatttcacgtttgaattgagtgatgagccccgtggtacgggctctgtgattcggcgccggcgtgttgtcatcgaacatgagctcgacaaatttacgttgcgctagaaccggcggaaaccaggtcagcgttaatacgaaataaccctcgtacatcgttcccaagcgctcgaatagcctgcgtcgctcttcatcgatcgcggcggataccggatcggggaaatgcgacaaacccaattcagcgtaattaggggctgggcggcgcaccgcatcgacgtgtaccatccacccactgccgaggccgacaagggcttgattgatgcggaaagacaccatttcgcgctgctcatcggtgctgctcgcgttgtcatcccctttgtatatccatgccgccataaatgagccgtttttaccgacaatgacaccgtcatcgacgacggcggcataattaagcagatcgcccagcccggcgtccttggatcgatgctttttgagcttcagctccgcatcgaccgaccggatgcgggcgaacagcataagcagcaggacggcaccgagtacagcaatcataatcgcgagtacttcgatcatcattgatattgctccccttgggtattggtattgtcacgaaacggcgtactacgcggcgggtaatacttcttgtagcgacggctccgcagataaacgtagcgcagcttaggatcgctctttgccatgagacggaacacaaagagtgcgccgaaccaaagacagacgccgaataccgtagccctcatgtcctgagcactgaaaataagcgctccggcgagcagtcccgagaacatcaccaattcccgatccccgcccatgaataggttgtctctattgccggcccgacgaatgggaatcgtgcgaccggccatgttacacagcccttccggtagcgatagatgccgcgttgtgagcgtaattgactaaaccgtcagacagtaaggctatttcggcgccgcgaccaaagaaggtacccatcatattttgggcgccgaccagtaacgccatgaccaggacgataaaaatcaacgttctgaaaaaaccgttcagttcgccgccgaaaataagcacgccgccggcaaccacaatgccgataatcgagagcgcaaacgccaccggaccggtaacggaattgcgcagattcgtcagccaactctcatagggcagcgagccgccggtgccttccgacgcaaaggccggttgcggcaaaaagataacggccatgagcagtacgaataggctcaggtacaaaacgctgttacggtcgttctgaagcgatggaattgcgaattgcatagatgtttctcctaaagtgatcttgtgttgtattggccgccgtggtatccggaaacctcaataatttcctgaatacgacgaccttccggggttctggcgatatgcacgacaacatgaaccgcctcgccgatgagcggttcgatgggtttgggggaatcgggatgcatgctgatgagcatggccaaccgatcaagtccggctctggcgttattggcatgcagtgttgccgccccgccttcatggccggtattccaggccattaacaaatcgagcgcttccgggccgcgcacttcaccgactaaaatccggtccgggcgcatacgcagcgtggttttcaatagcgccgtcattgtgacgtcgatgctggtgtggtactggacgtaattctccgcagcacattggatttcgccggtgtcctcgatgatgaagacgcgttcagtcggatcgttagccaccatctgattgataatcgcattgaccagagtcgtcttgcctgagcccgtgccaccgataaccagaatgtttcggtgtgcggcaactgattcaataatcacttggcgttgtccgggggtcattattccgccctcgacatattgctctaacgtgaaaatggcaaccgccttcttccggatggcaaaagtcggggccggaacaatcggtggaagctgaccggcaaagcgagaaccatctaacggcagctctccttcaagtatcggtttttgacgagtcacttctttgccgtgataaccggcaatggtttcgatgatggcttgcgcctgcgcaacgcgcatggtgccgatacattgcatgggctcgccaagccgctcttgccacagcttgccgtccgcattgagcatgatttcaacagttttcggatcgtatagagccgacaagagtgacgctcccatgtcgcgttccaacttacgtttggctcgatcacgggtgctgtcatggttatcgtttaatgaggtcatcgttatcctgcagatctgcggatttaggtcatgtcttttgatagatacgatataccgaaacaataaaaaatgcaacggtatagcgaaattttttcaggttgtggtatctttgagatgtctggatatagtcatgaaccgggatggtgtcatcccggtgaaatgcgaattttagcggctaaagtttccttattagctttggatgaaggtttattgtattggcgttatattaagattgtcttattttagccctggtttcttcgccccattttttgacgataaaggctttgacttcggggagaataacagacacgcgctcgtatcccggtggcaggctatgcatagccgtaccgcccgccagctcatcgagcgctgccttgtccaagtcagttgattcgagcagaagaggcaacggaacttctaacgcctttgctatagcctccattaccttaagcgatggattgccggtacccgatgttagatcggataaaaacgagttcgacactccggataggtcatgtaattcttttttggacattccacgctcgtccaaaatacgaagaatgttggtaaagaatatgtagttgtacactgagtttaccttgttaagtttggcctattttagccgctaaaataatattgacgataattaaaggtttagctaaacttcaattcgataaataagacgcctctctgtaagtcataataatcgatcaacacgaaggaccaacaaaatgcgaatcattaaacaccaaaagataggaaattgccatgattgaggagaccgtggtcaagctgaaagcgcttgagagcaccgcactgcgacgcaagatagaggccgttgccaggccaggcgaaacttatgagcaagctgcggctagaattctgcaaaacgaagaaaacaccattcctcataggtcacatacacatcaagcaccttcaagcgacagacaggctgatattttcgtgccgacacgctatgatgttggcacgcgtgacagccgaagcatcatggacgtcgcagtattccggctgtccaagcgagagaagcgcgcgggagatgttatccgatacgatctgccggacggatatgtcgaggtcaaggccggtccggacggcatggcgtcgatttgggattacgacatcgtgctgatgttgatctctcatttgaccgatgccatgaaccgctattgtgaagggaaggaagacaagccaggccgcacattccgtccacacgttagcgaaattctcaagttctgccgaaaaggcgacggtggccgacaaattgaagaagtcgaaaaagcgctggatcgtctgagaggaacgaccatcaagagtgtgcgcgagcgacccgccatcggagggaagcgcggcttacgcgaaatcaaggctgaaggcttaatcggggattacacggtgctctcctacaccgaaaccggaaagattgcgtctgttgaaatagagatcccgaaatggctgcaccgcgaagttacagatcggaagcgtccggacgtattgactgtccaccctgattatttcctgatagaacccggcatcggccgattcatctatcgcttggcacgccgcgccgcaggcaaaagcgtggctaagtggtcgtttcaactcatctaccagcgcagcggtagcacgggtacgttcaagaagttcacatttaccttacgcaagctgatcgaaactaacgacctgcctgaataccgactcgcagaggaaagcgggcaggaaggccccttattggtcataaaacataaagagtcattaatcgaaaattaatagggttcctaatagacggcataaaaaccacagcggctgataatagttgttattgcgtgcttttgcatcctgtcggcaaagctaaactaagccataatctccgatataaacttgtttcgaccgtaacaagccttaaactgtgtatagcctgtctattaggaaccgaaatccgtctattaagaaccggaacccgtcttttaggaacccaaatccgtctattaggaaccaataggttcagtaaggtattgattatgtggaataaataggcgcttttttggcctaaaacttattaatactcttaaatctcttaaaacccgacttctccagtgctgctgcaacataactaatgattggaacaacacttaactcatagacagcatcttgttttggctcaaaaaagctttaacgccaagaaagagaaggggcataataaaaatatattattcgttataatgaaattcgtatagctaaacttattgactagcaacgatatttcatttagtatcgtttttgaaggccgggaattccgttccggtcactaccgtaaaattgccgagtcacgataatcatgaacgtgcagaatttcgtgaatggccagaattcgcgaacaggggagcgagggcgtaacccattggaagacgggttgctgtgagtacaaagcagatgcgtattaatcgttggtgccgggtggagattgccccggagtactccggggcgtttggcttggcgctgtaacaccgaagccttactttcccagggtgggagaagtcgtcggtattggacacgttaaagcgtggagagtcaagcatgaacataatcaagtttcctcaaaaggggagaccggccggcaaacaagcgacggttccgactcctgatcttacgcgtaagtcggttaaacccacgagagtaaaaatcgtcctcgacaaaatggtgagttttgtttgggtgatgacagtgcttgtctggccatttgttaaatgggtgctgtcaatcgagattttcttccagatgctgcgcatgatctaccattggaacacgcccggcgtttatgccggttggacttttctgttgcatttctctgtattggtagcgctgacctggtttgtcgatttttatagaccgcaaggcatgaagcaacaataaattttagccgctaaaaaaattgataagcaatagtggcttagcaaaacttttaaatattggcggctgtaaccgccatggttcaacatcagggtggcgaatatatgcgtggatttcatgatagagagactaagcgaagctcaattcgaagaggccataaagggattgaatgtcggtcggcagaccatcgacatcgcgcggggggtgctggtagaaggaagaccgcagtccgagtttgtggcggcactagggatatcgaagggggccgtgtcgcaagcagtcagccgcgtaaaggcggcggtccaacaagagaagatcccggagggcttcgagcgggtgtcggcggtcctaccgaaacatcaagcgtttatcgtcaagaaatgggcagaggacgcaaccaaaaaattggagacaaagaaatgaaaacactggtcacagcgaaccaaaaagggggcgttggcaaaacttcgaccctggtacatctcgcgtttgacttccttgagcgaggtatgaaagtggctgtgatcgaccttgatacacaggcaaacgcgtcttatacgctgcaagaatttgaaagcggctctcttgccagtcagatgttcacggctgacgatgacaaattgcgtgcttgtttcaacaatctgaccgccgaaccagctctgtgcttgataagttccgatgcggctatggcgaacatggaaaagctcaccatgacggaagcggcaacaagctttaagcggaatatcaaagtattggctgaatgtggttttgatgtctgtttaatcgacaccgctccgtctcttggcgtcagtatggccgccgctctttttgcggccgattatgttttttcgcccattgaacttgaagcgtatagcatccagggcatcaagaagatggtcacgacaattgtcaacatccgcaaatcaaatcccggtttgcaatttttgggcatggtgccgagtaaggtcgatggccgaaacccacgccacggtcgacacctggaagaattgaagcgcgcttatccccaattgatgatacctgtcggtattggcctccgaagtagtattgccgatgcgttagcgtcaggcatgcccgtctggaaaattaagaaaaccgccgcgcgcaaggccgcacaggaagttcgtgcgtttgcgacacatatttttgaaaaaatggagattacacaatgaacgccaagaagaaatcgagcttgggcctagagagcataggcgatctttcagaacttttggaccagccggaaataactaagagcttcggcccgctggaactggcgctagaactgattgatgaagatccggatcagcctcgaacagccgaaaaccccggtttctcatccgagagtattgcagagatagggcagacgatcaaagatcgtggcgtgaaatcccccatatcggtaagggaacaccccgagatagagggccgctacattatcaatcacggtgcgcgtcgttaccgtggatccaagtgggccggaaggaagaccatcccggttttcattgacaacgattacaatcacgcagaccaggttattgagaacctgcaacgaaatgagctgactccgcgcgagatcgctgattttatcggtcgcgagctggcgatgggtaagaagaagggcgaaattgccaaagaaatcggtaaatcggcggcatttgtgacgcagcatgtcacattgctggatttgccggagccgattgcactgatattcaacaccggtcgcgtcagggatgtaaccgtcatcaatgagctggtgactgtttacaaaaagaatccctctgaagtggaaacatggctggacgacgacagcctggacttgacgcggggatcggttaagttgttgcgtgaatacctcgatgacaagaaacgtcaacaactagaggatcaagacagtgacgaagagtattatccagatacagtggatacgctaaccgacaagaccgatgccgaaaccgtcaagacttataccgagacagtcgatagagaagaggagcacgagacagaagagcccaaacataaaagggaagagaaggagaacgattcggacaagctcaagaaagcgattgtacgggtactgcatgacggtcgtatcgcccgattggttctatcgcgacgaccgactcaagacggttacgcatgggtgaaatatgatgatgacgggaatgagttcgaaactgaccttggtagtgtgcagcttattgcagtgctagaaggataactcactctccccttaaacccaccaaccccgccctgtgcggggttttttttgcctatttgctaggataatttatttgtttgtttgtaagtttattagtttgtttgttaatttatttgtttattattctatttattgctatactaaaagcgtcaaataactgttgagaatgccgccatgagacccgtagaatttgcccaagaagacatcatcacagccggccaagaactgcaagaatccggtcgcaatatcaccggatttgccctccgcaccaaggttggtggcggcaatccaagcagattgaaacaggtctgggatgagtacgttagtagtcagtccgttgttaaaactgagccggtggcagaactgccaattgaagtcgccgacgaggtggcggccgtcactaaatcgcttactgatcgattggcaagtttggcagtagagttaaacgataaggccgttaaggccgcagagcgccgcgttgcagaagtcattaggaccgctggcgagcagcgcgaacaggccgagcgcgaacttgctgacgcatcgcagacggttgatgacctggaagaacagcttgatgagctacgggctgagctggaagctgctcaggaacgcattgcggatatgtctagagaaaatcaggtacaagccgtagagcttgccaaggttcgcgagcgactggcggctaccgagcaaagcgctagggcggctgccgagcaaaatgcggcagcgataagtaaactgaatagcgccttggaatccgctcaatcgacaggtgtgcagttgaagcgtgagaacgatgcgcatgctgtagagctggctcagttgcgcgagcgactagcggcaagcgagcagcgtgctgtgatggcgaccgagaagagtgaggcagaggcgagtaagttgaatagcgccctggaagccgctcagagcgagaagaaccaagtcgctgagcagcttgctgtatttaaggctcagatggtcgctgagcagaaggttcagcaggagcagcgcaaagcaagtgctcaagaagcttctcgtcaggccgaacgctacaccaaggttcgcgctgagcgggatgaggcagttaaggatgctgctaaagttcgcgaggaaatcgcttctcttcgtggacgtattcaggcgttggaggaagtaatggctaccctatcgaaggatataaatacaaattctcctaaatagggtgaccgtaaggctttgtgcgaaaaataagcattaggggagacatcaagctcaaagaccgtaaaaccagggaaacagaaaggaaagaacgccggatagtaaagactggtaccgcgtataagacgtttggaatgcttgatgaagccgtcgcgtatgcttggaagatgtagactattaagggagtcattgactgtattttagggtcgcgggccgataacatgaagcacagaaactgattacatctgtttttcgggaccacattaactgagaatgggaccggtggtcatgccagcggcgccctaggatggctcgggctcagaaatgggcagtattggcgattacgactccaccagctttgcaatcgcttgcattggcagtatcagtcgaagagagtctggtaagcaaataaagccattagcctcgtagaaggcagccgcctgttcgctgatggcatctacgatgagcatggaagatccaatagaacttgcgctcgcataagcccgcttcaccgcatccgcaagcaacagtgcgcccaatccctgtctttgttgatgacttgcgaccgccagacggcctataagggttgcgctgaccaaaggatagcgcgggacgtgtttgcgggcctctactggtacgtccccttgcgcgagcgcggtcgcacacaaagtgtagtagccaagtacttcagtcggttcgcgcggatcgacgagaacgaatacgccgttgattttgcgcttcacatcctgattggcttgtgttttgaaatagcgatccaggctttcgacgccgcaggcaaagccagcacggttgtgatgggagccgaatggttcgatttttaagtctggcgatatcgcctcagtcatttatgaaatgatccgccgtttgtgctcggccagtgcgcgaacaagtcgttcacttgcttgcggtgggctcatgagcgtatcgaaaaatacctttcggtcaagctcggaaagcataagtgtttcatgctccgcaatggttcgtcgtgcagcatcggtaagtgccgctatacaaaagtcagtgagttttcgtgattccagatgagctgcgcgttcgattaagtccttggtttgttcatccaaacgaaaaccaagcctttctttgcgtactgggttttgtgctaactgcaagttcttgctggacatgggtaacctcatcggtcgtgttcttcatattggttgatattgtacgtcaatatgacgacttttcatagtcataccatttcttcaaatttattgttcgatgaaactgcattcgatttactattctctgatgagcctaatggtagaatcatttattcatgtttcgccaaattcgcccatcaacacgatacttaatgagaagagaagcatcaaagaaatcgagaaatggtggggaataccgtttattgttaccgatacggttgaagatataggcgatccagactatgaaacgcttatggatcgattgagcgaatggtcagatatcaaaatcaatgaccgagaaacgtgggaagcgatggttcgagtagtttcctagcggaacaaggcttttgggtcaaaggcttggatggctgttgagatcggtcgaccgaatacgaggcattttggaacgattgatgaatctgtgaagtatgttcggaatatgtaagctattaagggagtaattgactgtatttagggaaaggacattttcattttcctgatatgactacagattgatcgtcgatacccttattttttctttttttaccttttttctttttttaagagcgttggaagcctttatttataagggatacagcacgaatatgactacaaattgatcgctatatgactacagattgattgtcatatagcgacaaattgatcgtttagaaactacagattgatctcttaggactacaaattgatcgttaataactacaccgttattaatttgtagttgttttggctattaaacgggcgtatagtcgaggcacactatcatattgtagccagatactaatttggacaagcccacttacttaaccgtcgttaaatcgaacaaggtcgtcgaagccagctatatgcttacgttggctgaacagcgggttttgttggcgtgcattgcgcaggtcgattcgacggcggaattgtccgaaaactttcggttcgaggttaccgcttctggcgtagcggacctggccggtctggatagtctttcgaacgcttaccgggatctcaagaaagcggctgaaaaactctacgaacgcagcgtgattatcgacgacccggatccgggtaacccgcaaatcacgctgcggaaaactcgctggatcagcagcatcgattacgtgccaggagaggggaagctggtgctcagcttctcggtcggcatcattccttatctatcccagctctcgaaagaatttaccaaatacaaattgatgcatgtcgcacggttcgaaagcgtttattcgatccgtctatatgaattgttagtgcaatggagttctgccggtgagcgtgaaatcgaaattgagtggctgaaaaaacagttccaggttgaagataagtatgatcgtttaggtaatttaaaaaaacgtgtcattgacccggccgttgccgagatcaacgaacattcaaatatttgggttagatatggtcaacgaaaatccggacgaactgtgacgcattttcaattcaaattcggattgaaggatcagccgaagccgaggcaacaattgaccgacgccgatattgaacgcgaggcgaggccgggagagacgagggcggctgtgatcgcgcgtttgaccggtaattcggtggccaaggacgccaagccgggggagacgttcgatcaggccttgcagcggaagaaggaattggcggatgttaagcggaagttgaggtaaattggcaggttccgacccatcaacgacacacaactgttttaatcaaacgacgcctgcgctacagtttgcggtcatttaaacgtcagtgtgatcagtgcgcaagatcagcatttcgattcccacatttctcttggatagctatcgcaaatataggcaacgcacagtattctgtggtcttgtctttcaaagatcaattctttaacttttatcagattcatgtcacccaaagcgagcaagcaaagcaaaagataccaaccgctggaaaatctccgtaatcaactcttaaagttagaaccagcgggaccaaacggctttgagggtttggttgctactgcattatccgatttaaccggattaacctttcgcctcgcaaagagtggatcgcaatttgggcgagatgccagcacgcccagcggacgttttgcaatagcaatggaagccaagcgttacaaggaatcgctccgcctcgaagatgttgccggaaaaatttggcttgcttccaatgaacttgcctctgatgttgatatgtgggtactttgcgccacttccgaaatgggggacggagttcttcagaagcttgaagatatgctggaagaaaaaggcattactctgctgatgctcgattggacggaggcaccgttacctcgtttggcggcactcttggcagctacgcaatcatcgatcatacgatggttcaccaattacacgtcaaaggaaattgctgaagctattgatgcggaacttaatacccttaaagcggataacaactttatccccgctcgcaatcaactaatcagagatgcaagtgctagttattgcggactggcggcgctcaggaagatcaaccaggattggattaatcgagtattttctaatcgaattaattcccgaaccgcgtttggacaatacctaaccatactggacgagcggtatccggtaatttcccgttgccaactcaatacggctctttccgaggcggttaatgagtcatcctgcgttgccattctcggcccggagggggcaggaaaatcctggttagtcgccaattggtgggccgcttgtgatgacaagcctatcctggtgatgggaggctcctggatggcggatcggatcgattcaagagatcctttactaacattggctaggttgttggctgctcaaagcgaagatggaataatcgattcgagcgatcattggctaaggcgtttgaaacgttggagggaacatggtccagtttccaacgctgaacaactcagattcttggtcgttcttgatggcctcaacgaaagaagtgggatgcattgggcagacaccattttgcttctaaaagctgaagttgaaaaactcggcggttgcctagttacaacctgccgcgaagggttttggggtcgggaagtagcgcctcgattagctggggtcacgattaggattgtccgagtgggcggttatacgccggatgaattaggtgatttgctcaagcaacgtggcatagcaattgacacgatccccggtaaagtgcgtgactttatccttaatccgcgtatttgttcgattgccatcgattttctggaaaacctttctctgcaggttgatgcattaaccgttgagcggcttcttcttgaatactggcggcggcgacttgaggaacgaggcgacttgaccggtcatagccttccagattttgagaaattgcttagatcccatgctaaacaatttcgcaatgcacagggcgttcaatttgatcgcgacaactggcgtgagcattctggtgcggcgaggcgttcggacggtcgctcgctagaaaatgacttatcggacattgaagaaggcgcatttttgcgaatcgcccctgacagggacggattttacgagttcaaaccggatatagtcccttttgcattgggattgctgatagcgcaagaattacaagacgaacttcgaaaaccgaaccaagaacccagggaactaattgatcgcatcgttgatgagattcgcggttttgatttagtcggagaagcattacgcgcagccgctggtattggttgttttaaagaaaattatccagcggggggacgagctgcactcatttcggcttggcttgagttgcaaaatattgacgattcagcgtatgacgctcttgttgcctatgtttccgcttgtccagaagccgttttggataccgtcgaaatcgaattcgatgaacgttctaacggtagtcggagagattggcttgtcgatgcgctattacagaaaggcggaaatcaaaatgtccattcagcaatagataaacgtattagtcgatggcttggtcgatggtcccgagtaccacaaatcttgggggtatcagatgagcaaacgcaatcccaacagagtgagaaggctaagcctattttggataagttggcaaatctgacaccaatagaaaaggagttcttagcagccgcatgttttgaagtagattcaccagaagccgctcagctagattcggtcgctgccaagttgatggctggatactctcaagcggaatatgccgaagcggttttagcttgggctttcactctggccataacaagtggctaccggtacggcgatgccgatttgtgctggagcgttcggttgaataccaccgattttgaagcgtttgaactcaaactcaggcaagctattgaccgactacttaccaagtcgcactccgaggttagcaaaaaagccgcagcgattgcgctacgtctgatcggtaccgttaatggtgcactggaagctgaacaattaaacccacgcgaatatcgaaaaggatggcgagcaatcgaaaattattgcgcgactgatccatacgatcctgattctgatagaccggaaaacttagaaaacgcaattaatttggcaaacactttaaatcctgagattctttggacgcaattctctgtgactcaggaaaaccatcagctggaattgataactcctgctttggttcgtttccaaccggatctgattgttcatatagttcgagaagtatgcaaaacacttgaaactcgaagtgaactcccattgcgccaacttagttggaatctaccaagattcagccccctttttgacgaagatacgcttaaatccgttgtaacaggctatgaacggatagtagtgcagcccgatcttgtggatgaaaacgataaaacattcgttactggatttattctactaagtctattgccgcactattctgcgcacgaacaattagcgctgtttctgagactgcccgctgtagttggcgaaatgtatacgtttcgcgatgtgttcaaggcacttgatgagacggaaattgaaatggctttaatttttgctcagttcgatccggggaaattgagacgaacttgtttttttatatccgctcatcgtccgacgctaacaaatcgcagtcgtgaaattttgggtttggctctcacagataatgatcctcttgtcgttacgtgcgcttccgatgttgcctttatcgcgcaggataaatttcttgatgaattggtgattacggcagctaagcagcgaggcaccagcaaatatactagggaaacatttcaccgagacagggctgttaccgccgctgtagtgtcactcgatctttgcgaagcgatttctttaattgcgccacgttttttgggtcgtgcagcctcgaagctcaacggcgaattggccgacagaattgaagatgaaattgaacatgcagttaatcgtttgttaaaacctgttctggctaacgtaccaagtttggggactttaaatctcgatattgaccaaactgcacgagatcctatgattcatgtcgaggaacgtgtccgagaatttacgggcaatgcagatgacagtttgcaaacctttgtaaattcctgttcattggataaacattcagagagacttagactccgggatctggaattcagaagctacaccgagcagctcgaaatggaaggggctctatgcctaatccaagaacctgaccgtatcgcgttgacgcaacttagcagacgaaatccgaataaaacaatagccttggcgaatcggattctcgaagaagtaaatcctgaaagattgagtgccgttcgaagctttgccctcgctcttgcggaatcattggcagataccaacccgactactacgtctgatttattcaaccatctttccagtgtcgacagcatagtaaatatcaatatcggtgacgccaaaattcctcaagaagcagttactttgtttgctgggccagacgttgaggttttatctaaattaagaaaaaaagcgctggtacaagcaaaaactgatgccgagcttcaaactctgatctacgcagccgaaagatccgggcatattgactggcttgaacactggatagctaacgaagtagcaacagaaataccaggccgcattgcaaggggattgatggttgaaggtcttagaaattttggttccgtaacatcgccatttctatcgcgtgactggggtcccggatttttgggcgaggttgcaaaacaagctcgtttttcccatgaacgaaatatctggtctcgaacttgggcgaccaaggcattgcaagcaaataattctttagacttttggcgctggggagagcttactgtgggtattgttgatattcgggcttttcattggtttgacctgaacgtagataacccgctggtaaaacaatttggtaaagagcttttcggacggatccgaagcgagtctgagaaaaggaccaaaaaaagagaagagacgctatttggcataaagaaa

SEQ ID NO: 2 - DNA, 31 bp, Artificial Sequence
  Description of Artificial Sequence Synthetic primer
gaggacgtccacagcggctttgactggatcg

SEQ ID NO: 3 - DNA, 33 bp, Artificial Sequence
  Description of Artificial Sequence Synthetic primer
gagggtaccgagtttaccgaggtggatttcgcc

SEQ ID NO: 4 - DNA, 32 bp, Artificial Sequence
  Description of Artificial Sequence Synthetic primer
gaggggcccgtggaatagtagtgctaaacaat

SEQ ID NO: 5 - DNA, 33 bp, Artificial Sequence
  Description of Artificial Sequence Synthetic primer
gaggagctccgtcaaaggacgccgtgagcccag

SEQ ID NO: 6 - DNA, 30 bp, Artificial Sequence
  Description of Artificial Sequence Synthetic primer
gaggacgtcgtattgaatcaggcggctcgt

SEQ ID NO: 7 - DNA, 31 bp, Artificial Sequence
  Description of Artificial Sequence Synthetic primer
gagggtacctgctcgcttgagaggtgtcggc

SEQ ID NO: 8 - DNA, 29 bp, Artificial Sequence
  Description of Artificial Sequence Synthetic primer
gaggggcccgcgttacgattattcctgga

SEQ ID NO: 9 - DNA, 30 bp, Artificial Sequence
  Description of Artificial Sequence Synthetic primer
gaggagctctggttcgctcggcgctgcagc

SEQ ID NO: 10 - DNA, 29 bp, Artificial Sequence
  Description of Artificial Sequence Synthetic primer
gaggaattccgccaagcatactccacacc

SEQ ID NO: 11 - DNA, 30 bp, Artificial Sequence
  Description of Artificial Sequence Synthetic primer
gagggtacccttcacgcatgattaacaccc

SEQ ID NO: 12 - DNA, 29 bp, Artificial Sequence
  Description of Artificial Sequence Synthetic primer
gaggggcccataatcctgagcagccggag

SEQ ID NO: 13 - DNA, 30 bp, Artificial Sequence
  Description of Artificial Sequence Synthetic primer
gaggagctcctgcaacaacataaagccgac

SEQ ID NO: 14 - DNA, 33 bp, Artificial Sequence
  Description of Artificial Sequence Synthetic primer
ggtggtgacgtcacgctgctctaaataccttgg

SEQ ID NO: 15 - DNA, 33 bp, Artificial Sequence
  Description of Artificial Sequence Synthetic primer
ggtggtggtacccacgaacaactatctcaagcg

SEQ ID NO: 16 - DNA, 33 bp, Artificial Sequence
  Description of Artificial Sequence Synthetic primer
ggtggtgggccccctttgcactcggtatcatcg

SEQ ID NO: 17 - DNA, 34 bp, Artificial Sequence
  Description of Artificial Sequence Synthetic primer
ggtggtgagctccgtgttatccaaatcgaatccg

SEQ ID NO: 18 - DNA, 32 bp, Artificial Sequence
  Description of Artificial Sequence Synthetic primer
ggtggtgacgtcatttacgcgaaccttggtgg

SEQ ID NO: 19 - DNA, 32 bp, Artificial Sequence
  Description of Artificial Sequence Synthetic primer
ggtggtggtaccgcagcggagaagctaaggat

SEQ ID NO: 20 - DNA, 32 bp, Artificial Sequence
  Description of Artificial Sequence Synthetic primer
ggtggtgggccctttgttcgggcgtttgttcc

SEQ ID NO: 21 - DNA, 34 bp, Artificial Sequence
  Description of Artificial Sequence Synthetic primer
ggtggtgagctcggtcagcaattcccagtttgag

SEQ ID NO: 22 - DNA, 30 bp, Artificial Sequence
  Description of Artificial Sequence Synthetic primer
gaggacgtccacgacggcatcacaatgaac

SEQ ID NO: 23 - DNA, 31 bp, Artificial Sequence
  Description of Artificial Sequence Synthetic primer
gagggtaccgcttaattcctgttgctgtgtc

SEQ ID NO: 24 - DNA, 29 bp, Artificial Sequence
  Description of Artificial Sequence Synthetic primer
gaggggcccgccgaagccaagtgtccaag

SEQ ID NO: 25 - DNA, 29 bp, Artificial Sequence
  Description of Artificial Sequence Synthetic primer
gaggagctccgttgccgttggttgaacac

SEQ ID NO: 26 - DNA, 33 bp, Artificial Sequence
  Description of Artificial Sequence Synthetic primer
ttagatctcggccaaaattggcgatgagttggt

SEQ ID NO: 27 - DNA, 34 bp, Artificial Sequence
  Description of Artificial Sequence Synthetic primer
taccatggacgatgacggcagccggcttatcgac

SEQ ID NO: 28 - DNA, 30 bp, Artificial Sequence
  Description of Artificial Sequence Synthetic primer
ttccgcggtcggcaatggcaattttaaaga

SEQ ID NO: 29 - DNA, 29 bp, Artificial Sequence
  Description of Artificial Sequence Synthetic primer
tagagctcgcccaatccggttcgatttca

SEQ ID NO: 30 - DNA, 28 bp, Artificial Sequence
  Description of Artificial Sequence Synthetic primer
gagacgtcgctcaaccgactcattgacg

SEQ ID NO: 31 - DNA, 28 bp, Artificial Sequence
  Description of Artificial Sequence Synthetic primer
gtcagctgggatgaatcggcctcattcg

SEQ ID NO: 32 - DNA, 28 bp, Artificial Sequence
  Description of Artificial Sequence Synthetic primer
gagggcccgtggaatagtagcgctaaac

SEQ ID NO: 33 - DNA, 33 bp, Artificial Sequence
  Description of Artificial Sequence Synthetic primer
gagagctcgatcaaaatccggcgtcgggggtcg

SEQ ID NO: 34 - DNA, 30 bp, Artificial Sequence
  Description of Artificial Sequence Synthetic primer
gtcagctgggatttcgttcaattgccgatg

SEQ ID NO: 35 - DNA, 31 bp, Artificial Sequence
  Description of Artificial Sequence Synthetic primer
gagacgtcctatccgcgcgtctccttcacgg

SEQ ID NO: 36 - DNA, 26 bp, Artificial Sequence
  Description of Artificial Sequence Synthetic primer
gagggcccgctcggcatagaatgacc

SEQ ID NO: 37 - DNA, 29 bp, Artificial Sequence
  Description of Artificial Sequence Synthetic primer
gagttaaccgctggcaagtggagctggtc

SEQ ID NO: 38 - DNA, 29 bp, Artificial Sequence
  Description of Artificial Sequence Synthetic primer
gagggcccgttcggcgcttaagagtttcg

SEQ ID NO: 39 - DNA, 28 bp, Artificial Sequence
  Description of Artificial Sequence Synthetic primer
gagagctcgttcgcaactagcacctttg

SEQ ID NO: 40 - DNA, 29 bp, Artificial Sequence
  Description of Artificial Sequence Synthetic primer
gaccatggcacaacggcatattggattgc

SEQ ID NO: 41 - DNA, 27 bp, Artificial Sequence
  Description of Artificial Sequence Synthetic primer
gagaattcgctgtcggcatctttgatc

SEQ ID NO: 42 - DNA, 2589 bp, Artificial Sequence
  Description of Artificial Sequence Synthetic polynucleotide
cacagcggctttgactggatcgattgccatgatgtcgaacaatcgattatcagctaccgccgcaaaggcaccaacgatgatttgatcatcatacttaacttcacaccgattgttagagaaaactatcatatcggcgtgcctttcgaaggggtttattttgaaatctttaattcagactctgcctattatgaaggcagcaatgtcggcaaccgcgagatactatcggaaccggagccttggatggggcaccagcaatcgatacacttgactttacctccgttaggcggaatcatcttgacccggcaaacgaaaaccataaacagctctgctcactaatatagggacctgcttgagaaagtgagcccagcgtgccgttgcgcaccgcaaattaactgacgacggcctagcttataaaaaattcgcttcgatatttatacgtaataggcgcaactacgcgagaaatgcccgcaaaatagaactaccgccgccccgaacgcaaacttaaactgatttttcaataatgtcggacgatcctactacagcagacagtgagccaataaacactcaatcggctattttggcgaaatccacctcggtaaactcaattctttaatatacctagaaccgaattttgactgcctttaaatttaattatgaaacgaattctttttgttaccagtgaagcacaccctttaataaaaaccggcggcttggcggatgtttcaagcagcttacctaaggctttggcggatctaggccaggatatccgtatcatcatacctaactatcaggctataaaaaaaaccgaaaacgtccaacatcggtgtacgctgagaatcaataattgcgatgtcaatattctcgaaacccgcttgccggaatcgaaagtaattgtatggctgatcgattgtccccagttttttgactacccgggcaatccttatcacgatgaatacggtaatgcctgggcaaacagtgccgatcgcttttcgctgttctgccgcataaccgtggaagtcgcgatgaacagagcctacttagattggaaaccggaaatcgtccactgcaacgactggcaaagcggcctggttcccgccttgttaacgctggaatacaatcgcccggcaaccatttttacgattcataacatggcctatcaagggatctttccctattcgacctacaatgcgcttaatcttccaagacaactttggaacccaaatgtacttgagtattacggcaacatgtcgtttttaaaaggcggcattgcttgctccgatcgagtaacaacggtaagtcctacctatgccaaagaaattcaatcatccgagtttggttacgggctagaaggcttgttaacccatcgcaaggaatttctatgcggtatactcaatggaaccgacaatgactggaatcccgaattcgacaacaacatcgtccagcgctacagctataaaacgctccatcacaaacaacccaataaggccgcattacaagaacggctctcactgcctctcgagccgtcgattcccattttcggattaattagccgtttggttgaacaaaaaggtatcgacttgctattagaatgcttgccggaattattaagtatgccagtacaatttgtattactcggtagcggcaacaaaagcttcgaacaacgtttatataattttgccgaagcctaccctgaaaaaatgtcgattactatcggctacgatgaacaattggcccattggatagaagcgggaagcgatattttcttgatgccatcgcgcttcgagccctgcggattaaaccaaatgtatagtcaacgctatggaactttgccgatcgtaaggaaaaccggcggcctagcagatactgtagtcgatgctctaccccatacgattgacaacaaaacggcaaccggaattacttttaatgaatcgacccccagttcgctactggaggccatcaagcgcgcgttgattttatacagagtcccggaaatttggacacaactgcaaaccaatgccatgaggaaagacttttcgtggaatagtagtgctaaacaatacttatcgctctatgaccatttgtaactcgattaaggatttggtcgtaaataacttccctgttttatggagagttcggtaactcgattggcaggtgtcggcggcagggcagattttttgctcctgcaaaatctgcattcatgccatccatggcaatcagtcgccgcggtcaagcctacacggacgtattcacggcgtcctgcaaagcgagttaccgaaccctcaacaaagctcatagttccaagacgttatttatcacgaaatcctaaggcgggctataactccaaaaagagttatttatcttttaccatgaagagcatgaaggacttgaagatacagcatttacaattaaatgtaagtgactgtccattaacagcctatgccatcattattgccacttcagtctcgccattgttttgatattttttagtcattatacctttcgcacttcaaatttctgcagcgcctaaagaggcaccgggttcctctcctaacgctaggtgagaagctgggctcacggcgtcctttgacg

SEQ ID NO: 43 - DNA, 4142 bp, Artificial Sequence
  Description of Artificial Sequence Synthetic polynucleotide
agttggggcgaacaaggctattcaggatactggctcaatgaaaccaatgcttggatttatcctgttttacacaaatcggcgatgttgatggaggaattggccgatgagtttcaacaagttccccgttattccttggaggaacgggtattgaatcaggcggctcgttcgttattattggcacaggcctcggattggccgttcatcatgaagtcaggaacgaccgtcgagtacgctaaaaagcgtattctcgaccatttggctcgatttaattatctgcaagaaagtgttcgcactcatacaattaacgaacactatttggcagcgttagaagtgatggatgatatttttccgatgcttgattttcggacttataaaacacaaacgcggacaacatactaagctaagaatttcgcgataaatatacccgtcgtagatcaaaattcggcgccggggtgttcgctaaaggacgccgtgaatacgcacaggtaggctctatgccagctccatgctggcaaagcctttgtcggacacccctgcgcctcctcgggcgctgccgaaatttgaagtgcgaaaggtatagcttccgggggttatgagctttttcaagggttcgataactcggttggcaggactttgtgaacccggcacctaaattatcgaaggcagttagccatagccaacaggctatgtaatttagccgctgggtgaatactttcgtgaaggcttgacggcggccgacacctctcaagcgagcaaccgaaccctccataaaagagggaagttatttatgagcaaatcctaagggcagcgggatgataaatcttttaattggcatgataggtctgatttatggcaaagcaaaatacaactaaaaccagaagctcaacaaaaaaagcaacaacgactaaggcttccgaaaagccgacgacagagagtcaagcggcgctaacgtctgaggcggaggcggatgcggtgccggagatacagaattcaagcgctgctgtgtctacggtaaaagacgcccctaaagcagagtctccgacaatggaaaagtcgattttggcggatgatgcgccgaagcctaatctatcgtctgagtccaaaacggcaccgaaatctaaagcaaccccgaaagcaaaagcggcttcttcggaagtcacttcttcgcctaagacgacgattacaaaagtggcgcaagcggagccggagaaaaaagcagcaccaaaagttgcgagtccggtaaagcctcctgcgactccaaaagctaaagccgctccaaaaaacaagtttgctgaaaaagccaagactgcgcctaacgtcaagacgccgactcaaactaaggatgaagagaaggctaaagccgaaatagaagcgaaggattctccgaaaactaagtctgcagcggaagttaagccggccgaatctgatcggcagattaaagcagcggaaacgaatgcgccggaatcgacgtcattatcgccggcaccggtgttgcccgttgttccgcaaaacgagacggcgctcaatattgttgcttcgaaggggaaggctacgcctagccaaccctccgcaaaaaccgtccctaaacagtccgattccgctggcgaaacgcaagtcaaaacgccctccaatgtcgacgaacccttatacttggagcccaagacgacatttatcacgccttttcctgaggtcgctcaagcaacctccgtcagccctgatataccgtcggaaaagcttcatcgacagaccgattacagcaaggcagagacaatagcgcgcacaccggaaccttcggcgccggagcccgttgccgacatgatgtcgcaactcgagcatgcaccataccacgacaaacccggtcccgactatccgcatatgtttgtcgtgcagattactccggaattggctcctctggcaaaggtcggaggtttggcggatgttgtgaccggtttgagccgcgaactggaattgcgaggacacagtgtcgaaattattttgcccaaatacgattgtatgcattacgatcatatttggggcttatgcaaaacctatgacgacctttgggtgccctggtataacggccatattcattgcacggtctggttcggtttcgttcatggccgtaaatgctttttcatagagccgcactccccggagaacttcttcaatcgcggcacgatttacggctttaatgacgacgtgctgcgttatgcgttcttttgccgcgccgcattagaatttctttggaaatccggcaagcatcccgatgtgattcattgccatgattggcaaacggcattggttccggtgtttttatatgaaatctatcagaatttaggcttatggcatccgagggtatgttttacgatccacaattttaaacatcaagggttgacgggcgacttcttattgaatgcgacgggcctgcatcggccggagtattattttcatcatgatcgcttgttggataatcggattccgcatgcgttgaatttgatgaaaggcggtatagtctattccaacttcgttacgacggtatcgccgcgttatgccggtgaaaccaaggacggcggtcaaggattcggtttggagccgacccttcatacgcatcattataagttcggcggtgtggtcaacgggatcgattatgatgtctggaatcccgaaatcgatccgcatatcgcggctccctttgctgtagacagggtagacaataaatatctcaataaacgggctttacgcgatcgcttgttgctagcggataatgaaaaaccgattattgctttcgtcgggcggctcgatcctcagaaagggttggacttggttcgtcatgcattgttttattcattgaatcaccgggcccagtttgtgttgctcggctccagtcccgagcgagatatcaatagctatttctgggagttgaagcggcatttgaacgacagtcccgattgccatatcgaagtcggcttcgacgaaggcttggcgcatctgatttatgccggtgcggatatgatcgtcgtgcctagtttattcgaaccttgcggtttgacgcagttgatatcgatgaaatacggcacggttccgatcgttcgcgaggtcggagggttggccgatacggtattcgataaggatcatgcgcatcataaagccttgcacgagcgcaacggctatgtttttagggattacaacaatgaagggctggagtcggcattgtcgcgcgcgatcgattgttattatcaatttccggatcattttcgggagttgatgaaaaatgcaatgcgttacgattattcctggaaatatcccggtcaacattacctgaatatctacgattatattcgtaacaaataaagaggccgattgatgaggatgtataacttgttccccctgttggcaggtcacttcgggcagtgggaagttcatttggaaagggccgcggcgatgcggttcgattgggttttcgtcaacccgttacaaaaaaccggggcatccggcagcctgtattctattgccgattattttcagttaaacccgctgttggtcgatccgaaatccaaaaaaaccgcggagcaacaattgcaggctttgattcaacaggctgagaaaaaataccgaatccgctttatgaccgatttggtattgaatcactgtgcgatcgattcgcccttgttgaaacagcaccccgaatggtttgttcacgagcatggtcaggttcagcgccctttttgtctcgaagacggcggccataaggtggtatgggaggatttagcacaattcgatcatggccgaagtcacgataatgggctttatgcctattatcgtaaaattatcgagtcatacatcgatctgggttttaaaggctttcgttgcgatgccgcctatcaattagcgcccgaactttggcggcaattgattacggatacgaaacaaaaacatcccgatgtggtgtttaccgctgaaacgctaggctgcagcgccgagcgaaccaagcaaaccgcgcaagccgggtttgattatattttcaatagttccaaatggtgggacttcgaaagcccttggttaccggaacagtatcaattattgcgccaagccgccccttcgatcagttttcccgaaagccacgatacgcctcgtttatttgc

SEQ ID NO: 44 - DNA, 3732 bp, Artificial Sequence
  Description of Artificial Sequence Synthetic polynucleotide
cgccaagcatactccacaccctttttgatctccaaattgggaattgctgttattgttcatcttgtttgacgcattctaacaatgaatattgaatgatgaatagcgtaatcgccacggggaatagcaaaaaatgttcaataagaaacaaacaggcaactgaaaaagccattgttaccatagacagtaagcgcttgcgttcggctatctttaacatgaccttcagcgtttcatgatctatgtttttccgctgtccgacaaaaagtattaccgttatcagtgaaatgaaagccagaaaatatgggaaataaatgaacatcggcgcatcgggttcgaaaaatcgatgccaataagtaaaccatacgaataaggtgctcgatgcaaaaaaatcatgccagttgagatagcttggtttatcgatcaatacgccgattccgctaagtaccattaccgtacccatgatcggaatgacaaacggcgacaatagataagcaaccagcggatgattgttatcggcaaggtagcccagtgataggcaggataggatgaaaataggcataaatgagcacatgcttcctaagaaaagtgacgtcatagaggattgtatctcaaagacggtattcgccaatgattgtattgcttgggtgttaatcatgcgtgaagattgatgatcaaagggctacaatgatttagactgttgagtaaaaaccggaggaacgcacatgccagaaaaattcaagtacatccgttggtttgaggaactaacgatcgacgatattcccttggttggcggaaaaaatgcctcgctgggcgaaatgtacctcgaactagccgccgaaggcatccgcgtgccgaacggcttcgcgattactgcggaaggttaccggcacatgctcgataaagcggatgcctgggaagccttacatgaggcgctcgataccttgaatccggacgatgtgaatgacttggctaagcgagccagaaaagcgcgagatatcgtttacgcggcgccgctatccgaagacttggagcatgaaattctgattgctttcgatcaactgcagcgacaatacgatgaggaattgaccgttgcggttagaagttcggcgacggccgaggatttgccgaccgcgagctttgcgggtcagcaggacacctatctgaacgtgcatagcggacaggctctgctcgatgcgtgtaaacgctgtttcgcgagcctgttcaccgatcgggcgattcattatcggatcgatcaaggtttcgatcatttcaaggtgtcgttatcgatcggcgtgatgaaaatggtccgctcggatttggcatcgagcggcgtaatgttctcgatcgataccgaatcgggcttcaaagatgcggtattcattaccggtgcttacggcctcggtgaaaatgtcgtgcagggttcggtcgacccggacgagttttatgtgcataagcctaccttcgagcaaggtcatcgttgtgtgttgcgacggtcgctgggcgcgaaaaaaattaagatggtctatagcgaaggccgtacgcgcgagcaaacttgtaatgtcgtgacgtcagccgaggagcgttcgcaattttgcttgagcgacgacgaggtgctgactttggccgattacgcgatcaagatcgagaagcactacagcgcgaaggctggcatgcccaggccaatggatatcgagtgggcgaaggacgggctggacggacaactctatatcgtacaggcacggcccgaaacagtcgcatcgcagttgagcgggacgacgctcgaacaatacgaactgaaacaaaaggccgaggcgattgtgacaggacgagcggtcggcagcaagatcgcggtcggtaccgcgcatgtgatcaaaaatgtcagccaattgaataccttcaaacccggcgaagtgttgattaccgacatgaccacaccggattgggaaccggtcatgaaaacggccgcagcgatcgtgaccaatcgaggtgggcgcacctgtcatgccgcaatcatcgctcgcgagctgggtgttccggccgtgatcggctgcgacaatgcgaccgaaacgattaaaaccggtacgactgtcacggtatcctgcgccgaaggcgatgccggcaaggtttatgacggcgagttgagtttcgatgtcaataagaccgatctttccggattgaagcgaccgaaaactaaaattatgctgaatttgggcaatccggaattggcattcaaactcagctttttgccgaatgacggcgtcggattggcgcggatggaattcatcattaccgagtttatcaaggctcatccgatggcgttgattcatcctgaacgcatacaagatgccgaagaaaaagcaaagattaaacgcttgacccgctattatgcgcagccggaggattttttcatcgagcgtcttgcggagggagtcggtacgatcgctgcagcgttttatccgaagccggtcgtggtcagaatgtccgacttcaagactaacgaatatgcaaccttgctcggcggtcgcgggttcgagcgcgacgaggcgaatccgatgatcggtttcagaggcgcttcgcgttatgtgcatcccgattataaggaaggcttcgcactcgaatgccgagcgatgaagcgggttcgcgaagacatgggtttgaccaacgtgattcttatgattccgttttgccgccgggagcaagaggcggtgcgcgttttggattatatggccgagctcggtttgaaaagaggggagaacggactcgaaatttatgtgatgtgcgaaattccgaataatgtgattcgtatcgatgcgttttcgaaactgttcgacggtttttcgatcggttcgaacgatttgacacaattgacgctcggcgtcgatagggattccgaaatacttgccgaagatttcgatgagcgcgatccgggcgttaaagaaatgattcgtatggctgtggaaggcgcgcgccgtaacggcaagcattccggcttgtgcgggcaggcgccgtccgattatccggaaatggccgaatacctagtcgaaatcggcatcgattcgatgagtttgaatccggatacggtgttgcagacaactcagcggattttgaaaatggaagaacaattaaaagggtgataatcctgagcagccggagcccatcgataccctttgcgagtcaaatttggtagtttaaccaagcagaaaatgaagttcatgaattaactcaagaggttgcctgagtatccttgttaacctctcgggtaagcgagagttttacacaaatgagtaacgagttattgaattaacttgttactcttcaaagtgaaatgaatttttaggataatcaagtcccgcaaaacgtttgataaacacgttcgtcgggttgaggcggttcttgatagtgccggcaaccgggttgttcggtaaatggtttgttgagtacggcgagcaatgcttcaaactttgaaaagtcctcgtcttgaaccgccgcgctcaatgcttgttcagccttgtggttacgcggaatataagccggattgactcgacgcatggcttcggctcgttgtgccggagactccgtttcttgatcgagacgggtttgccagcgctctaaccaattgtcaaagtccggcgtttgtggcagataatcattcagggtcggttctgtagcgggtccctcggcgatatggctcagtgctcgaaaaacattggtgtagtcggctttatgttgttgcag

SEQ ID NO: 45 - DNA, 3666 bp, Artificial Sequence
  Description of Artificial Sequence Synthetic polynucleotide
acgctgctctaaataccttggaaattgttggtgttcataagttataaagcgccagttactatctctaaatccataaaacagtcattctttctgacctgaaaatcggtaaatttcggcgttttattttttggtcatgaaaaaataatttgatgggcatttaagaatcattttgtcgttctctttttcgattgcggcacaggtttccattctggagcggcccggtgctgcttgtttgactaatttgccgtcctcgaccgaatagttcagtactgctcgcagttgatcgattctggcgtgagcgtctgattcttgagattttccttccatcgtaccgtcttctcggaatgtccaaatcgtattcaagcttcttgatttagtgccttcgcggtcgacagattccttatcgatttgccaggagccgattaggtccgcttgagtcaattggacatcggcattcacggaaaaagagaagaataggattgctaaagcagaaaatttaatgaattgcatgattgaaaccttttggggtcgtttttagaggcgagcattgtagcatggaaaggcctttctatgagtagggtaaataaataataacctagtatattcataggtttatggtgggtgttctcgtgattatcggtaggagtcctgattgcgttcatgggtaaaaattcggaagggtaaagtggatcggtataattctagcgttagagtcgcttgagatagttgttcgtgaatgagagaataaaggctatatccagctgaagtgagtacgatcatgacaagcaaaaaggttgagcactacattcttttgatcagtgttcatggtctaatacgcggacgaaatttagaactgggccgcgatgccgataccggcggtcagaccaagtatgtcgttgatttagcgagggctttggcgtgtcaaccttcggtaggtcggattgatttagcgacccgtttaattgatgatcccgatgtcgatgccgattaccgcgaagcgcttgagccgctggataaaaacgcgcaaatcgtccgaataccggccggccccgaaggctatatcaaaaaggaagagctttgggattatctcgatattttcgccgacaacttattggagtggttgcgtctgcagccgcgcatgcctgatgtgattcatagccattatgccgacgccggctatattggcgtacggttgtcgcttttgaccggtatcccattggtgcatacaggacattctctgggcagagacaagctcggtcgtttgcttgcaatgggcttatcgagtgatttgatcgagcagcgttatcatatttccaaaagaattagcgctgaagaggatgtgctggccaatgccgaattggtcgtgaccagcacccgcaacgaaattgccgagcaatacgaactctatgattattatcagccggatcgaatggtcgtgattccgccgggtaccgatctggagcaattctatcctgctgaaaacaaaataaaaatagctttcgccaaaacgctggagtcttttttgacccaccctaaaaaaccgatgattctggcgttgtcgcgtcctgacgaacgcaaaaacattgtttcgttgatacaggcctacggtgaatcccccgaattgcagaaattggcgaatttagtgatcgtggccggtaatcgcgacgacatccgcgaaatggacgaaggcgcgcaagcggtgttgactgaaattctgttgttgaccgattactatgatttatatggccgtatcgcgattccaaagcaccataaacaggaagaagttccggatatttaccgtttggccgcacaatcgaaaggcgttttcgtcaatccggccttgaccgaacccttcggcttgacgctgctggaggcggcggcatgcggattgccgctggtcgcgaccgaaaacggcgggccggtcgatattatcggtgcttgccataacggtattttggtcgacccgctggatagcggcgagatagcgagagcattgctggaagttttgaacagcccgaaaaaatgggaaaaattttccgaaaaaggccttaaaaatgttcggaaattgtattcctgggatgcccatgcgcaaaagtatcttgaaacgatacggcctttattggagcatcatccggagtttccgaaaataccgcgcgtgcgaggcgatttgagttatcgcgaccgtgccgttttttccgatatcgatcaaagtttattgggcagtcccgatggcttgaaagcgttcgccgcttatttggccgagaatcgaaaaaaagtttttttcggtgtcgcgaccggccgaagactcgattcggccatagcgattttaaagagaaacggcctgccgacaccggatgttttaatcaccagcttgggtaccgaaattgtctattcgccgcagatgacgccggacagcgcctgggcaaggcatatcgattatcattggaatcccaaggcgatacgacgcatcatcggcgatttgcccgggttgactttgcagtccaaaagcgagcaaagtcgcttcaagatttcctatcattacgatgccgagcaagcaccgacggtggaagaaattcaaacgctgttgcgtcaggaagagcaggcggtcaatgtgtatttatctttcggtcagtttttcgacatcgttccggtgcgggcttcgaaaggtttggctttgcgctatttcgctcagcagtggaggttgtcgttggagcggattctggttgccggggggtccggtgccgatgaagatatgatgcagggcaatacgctagcggtcgtcgtcgccaatcggcatcgcgaggaattgtcgggtttgcgcgagcctgagaggatttattttgcggcgcaaccctttgcactcggtatcatcgaggcaatagaacattatgatttttaagtgactgagagcagaattatgactgatcggcttttgctttgtaccgacctcgatcgaaccttattgccgaacggcaatgtgccggaatcggcgatggcgagggtttatttcaaggccttggcgcggcatcccggatgtacgctggcatatgtgaccggcaggcatcaagccttggtgcagcaagcgatagccgagtacgatttgcctttgccggattacgtgatcggcgatgttggcacgacgatttaccaagtcgtcgatcatgaatggcaagaatggtcgcaatggcgtcatgaaatcgcgccggattgggccggattcgaacatgccgagttgcatcgtttgttcagcgagctgtccgaattgacgctacaagaaagcacgaagcagaatcgtttcaaactcagttattatgtttcggagtcgatcgatccggagcctttgttaatcgaaatgagaaaacgcttggcgtcgcactcggttcgagcggcactcgtctacagtgtcgatgaaactacgcacactggattgttggatgttttgccggcgtgcgcgaccaagtttcatgcggtcgaatttttaatgaaacaactcggattcgatttggataacacggttttcgccggcgatagcggtaacgatcttccggtgttaaccagcccaataaactcggtgttggtcgccaatgcgaccgacgaggtacgcaagcaggcgctgcagttggctcgagagcaatcgaccgaacggtcgttgtatttggccgaagg

SEQ ID NO: 46 - DNA, 3536 bp, Artificial Sequence
  Description of Artificial Sequence Synthetic polynucleotide
cacgacggcatcacaatgaacaaggaccgtctgctggccgacgcaaaagcgcgcgtcctgacaatgatcgccgactaccgaccgccggaaccttatgtttattatttaccgggcgcttcggcccgcgcggcgcttgacatagccgtgcgcaacttagcgctctcgggcaaagcaaccgcctacgaccgggaaatcgccggccaactggctttcgtattgagcggcggagacaccgactccctcgatccgcttagcgaacaagacatattgaacctggaacgacaagcctttttgcacctggtcaagcaacccggcactgcagcaaggctcgaacacatgctaaaaaccggcaagccgttgagaaactgacggggcaatcgagcaacgtaacgttcatgaaggcctggccatcgccccggcaaatgaaagtagtctggtgacggagggtgcggtcactcgcccgacaggacgccgtgaatacgtccatgtaggctcgacggcggctgtcgatcgagcaaccgcaccctctccggaaccggcattgccagaacggagttttgcatatcgaaaaattagataaagaacccaaacctacgaactgacacagcaacaggaattaagccatgaccgaattcattatcgccgtatcgagcgggcttatcctagccggactcgttgcctatgctccgatacgccgcagtctactgacccggccgctgttcaaggtcatgcgccgcaatctgccgccgatgtcgcaaaccgaacgcgaagcgctcgaagcaggcaatacctggtgggacgccgaactgttcacgggccggccggactgggccaaaatcgcggcgctgcctgcggcgcgtctgagcgacgaggaacgcgcctttatcgacggaactgtggaaaccctgtgcgcgatgctcgacgactgggacatcaccttcaagcgccgagacttaccgaaggaagtctgggactatatcaaacaacataaattttgcggcatcatcataccgaaacgctacggcggtctggagttttccgatttcgcgcattcgcaaatcgtcatgaaactcgccagccgcagcacgaccgccgcagtcacggtcatggtcccgaattcattgggaccggccaaattattgctggcctacggcaccgaacagcaaaagaatcattatctgccgcgtttagccgacggcctcgaaataccggcattcgcgctaaccggcccgctggccggcagcgatgccggcgcgatgcccgataccggcatcgtctgttacggcgcgttcgaaggcaatgaacaagttttgggcatacggctcaattgggagaagcgttatattacgctaggcccggtcgccacggttctgggattggcgttcaagctttacgatcccgaccggctgttgggcgaaaccgaagacatcggcattaccgtcgcgctaattccgaccgatacgccgggtatttcgatcggccgccgccactttccgctcgattcggccttccagaacggccctaactggggcaaggatgttttcattccgctcgactggatcatcggcggcgcggcacaagccggtcaaggctggaaaatgttgatgcaatgcctcgccaccggccgcgcgatttcgctgccggcgctgagcgtcggcgcggccaaattcatctgccgcaacaccggcgcctatgcgcggatcaggcaccagttcaatctgccgatcggagccttcgaaggcattgaagaggtgctggcgcgcatggccggcagcgcctatctgatggacgccgctcgccaagtaacctgcgcggcgctcgatgccggcgaaaaaccggcggtgatctcggcgatattgaaataccaactcaccgaaggcatgcggcggatcgtcaacgacggcatggatattcaaggcggttcgggcatttgtctggggccttccaacgcaattggccgtttgtatcaagtcgtcccggtcggcatcacggtcgagggcgcgaacattctaactcgcacaatgatgattttcggccaaggttcggtgcgctgccatcccttcgtgcagcaagaaatacaatcattgaatcgaaccgatacggcccaagccttgcgtgaattcgactcggtagtgtggcggcatatccgtttttttctaaggaacatccttagagggctctggttcggcgttggcggttcgcgactgaccgccgcgcccggtaaccggcatacgaagcgttattaccggcaattgacgcggcttagcacaggctttgcgctggccgccgatgtcgcgctgctgacgctgggcggccgactcaaacgcaaggaacgcatttccgggcggctggccgacgtgctgagccacttatacttgtgttcctgcgtactcaaacatttcgacaatcaaggcgcgccctcggacgacctgccgctgttgcattgggcctgccaagacagcctgtatcatgcccaacaatcgttatcggaagcacttcggctactgccggcggcactttcggccggcttgctgcgtgccctgttgtatccgctcggaagtccctgcgcgccgccgcgcgataccttgacaggccctctcgcaaaactgctgttgagcgacaaccctgcgcgcgaacgtttaaccgacggtatttatatcaacaccgacccggacgatcctaccggccgcatcgagacggccttcaaggcagtactcgaggccgcaccggccgaggcaaaaatccgcgccgcgcaaaaacaaggagcgttgcccaaaggaatgccgtcatccctactgcagcaggctttggcatcggaattgatcgacaaggacgaatatgcattactcgaaaaagccgagcaagccaaactgaaagcgatcgcggtcgacgatttcgcgcctgatcaattgagcggtggtacttaacttcacctgattagcaagatgcttcagcttgccgaagccaagtgtccaagctggggttagtcgtagtcgcaaaaactaaaatatgctgttacccaagctccagcttgggtaacctgttcaggaagctctagcttcccgataatcaagaaagattgaacgagcgagcgagtcgggggtgattgagaatgcgcggaggttgtgtcggtctcaagaagctggagcttccggtgtgtctttcccaagctggagcttgggaaagagcgtgaaaatgaaagcgatcgcggtcgacgatttcgcgcctgagcacttgaccggacgctcttgactttacgccaggattttcgtcatgtaccgcacgcccaaaagcgcgggtgggtcagtattcatagcgtcctttgacgggcatttgactagcccagaagatatttagagtcttaatcaatacataatgctgccgggaaaattactttcccggccaatcgttttatcaaatccctttttgttccttggtcaattgcttcaaccagccttccacttgcggactcaactctttttttccgcttaatttcaaggtttgcgccaaatagccgtgttcaaccaacggcaacg

SEQ ID NO: 47 - DNA, 2121 bp, Artificial Sequence
  Description of Artificial Sequence Synthetic polynucleotide
atgacgagcaagaaggtttcgcattatattcttttgatcagtgttcacggcctgatacgcggacgagatttagaactgggacgcgatgccgataccggaggtcagaccaagtatgtcgtggatttagcgagggctttagcttatcaaccttcggtaggtcgggttgatttagcaacgcgattggttgacgatcccgaggttggtgcagattatcgcgaagcgcttgagccgctggataaaagcgcgcaaatcgtccggattccggccggccccgaaggctatatcaagaaagaggagctttgggattatctcgatattttcgccgacaatttgttggagtggctccggcagcagacgcgtatgccggacgtgattcatagccattatgccgacgccggctatgttggtgtgcggttgtcgcttttaaccggtatcccattggtgcataccgggcattcactgggcagagacaagctcggtcgtttgcttgcaatgggcttatcgagtgatttgatcgagcagcgttatcatatttccaaaagaatcagcgccgaagaggatgtgctggccaatgccgaattggtcgtgaccagtacccgcaacgagattgccgaacaatacgagctctacgactattatcagccggaacgcatggtcgtgattccgccgggaaccgatctggagcagtttcatcctcctgaaaacaaggtaaaaatagccttcggcaaatcgctggagacttttttgaataatcctaaaaaaccgatgattctggctttgtcgcgtcctgacgaacgcaagaacattgtttcgttagttcatgcttacggcgagtcccccgaattacagaaattagcgaatttagtgatcgtagccggtaatcgcgacgatatccgggaaatggacgaaggcgcgcaagcggtgttgaccgaaattctattattggtcgattactatgatttatacggccatattgcgatccctaagcaccacaaacaggaggatgttccggatatctatcgcttggccgcgctatcgaaaggcgtgttcgtcaatccggcattgaccgagccgttcggtttgactttactcgaggcggccgcatgcggattgccgctggtcgcgaccgaaaacggcggcccggtcgatattatcggtgcttgccataacggtattctcgtcgatccgctagatagcagtgcgatagcgaatgcgttgctggaaattttaagtagcccgaaaaaatgggaaaaattttccgaaaaaggccttaaaaatgttcggaaacgctattcctggaatacgcatgcacaaaagtatcttgagacgatacagccattattggagcatcatccggagtttccgaaagtggcgcgcgtgcgaggcgatttgagctatcgcgatcgagccgttttttccgatatcgatcaaagtttattgggcagcgccgaaggtttaaaagcatttgctgcttatttggccgagaatcgaaaaaaagtttttttcggtgtcgcgaccggccgcaggctcgattcggcaatggcaattttaaagagaaacggcctgccggtaccggatgttttaatcaccagcttgggaaccgaaattgtctattcgccgcaaatgacgccggacagcgcttgggcaagacatatcgattatcactggaatagcaaggccatacgccgcattatcggggaattgcccggtttgactttacagcccaaaagcgagcaaagccgatttaagatttcctatcattacgatgccgagcaggcgcctacggtagaagaaattcagacattgttgcgtcaggaggagcaggcggtcaatgtgtatatatccttcgggcaatttttcgacatcgttccggtgcgtgcatcgaaaggtttggccttgcgttatttcgcccagcagtggaatctgccgttggagcgtattctagtcgccggcggttccggcgccgatgaagacatgatgcagggtaataccttggcggttgtcgtcgctaatcggcatcgcgaggaattgtcgggtttgcgcgagcccgagaggatttattttgcggcgcaaccctttgcgctcggtatcatcgaggcaatagaacattatgatttttaa

SEQ ID NO: 48 - DNA, 1956 bp, Artificial Sequence
  Description of Artificial Sequence Synthetic polynucleotide
atgtacgaacagatttcgcattccttattaaatgaaattttagatgatctcaagccggaaatccgcaggcaggatttgcggcatttttacacgcgtctcggcgcgaatttttatgcgattcataatctgtttcatcatctatacggccgccgcgacgatttcaaagagcaaatgctcaagctggtcgaaaccttggctcgacaatatattcagcgaccggaagagttggaaaaactcgatatcgcgcgggaaaacgatcataactggtttctgaatcaaaaatgggtcggcatggctttgtatagcgccgggtttgccgacgacttggccggcgtggcggataatacctcttattttcaagagctcggcatcaatttggtgcacatcatgccgatactcgagtgtcctaccggtcgcagcgacggcggttatgcggtcagtaatttccggcagatcgatagccgtgtcggcaaattagaagatttgcagcgggtttcaagcgagttcagaaagcgcgatattctgttgactttggacgttgtgttgaatcatacctcggacgagcatgaatgggctcagcgggcgaaggcaggagagcaaaaatatcaagattattattacatcttcgataatcgtgaaattcccgatatgttcgagcaaagtatgcccgaaatttttccggaaaccgctccgggaaattttacctgggacgaaactttgcagaaatgggtaatgacggtcttcaatgattatcaatgggatctcaactacagcaacccttcggtatttattgaaatgctcgacgtgattctcttttggatcaatcaaggcgccgatattatccggctcgatgcggttgcatttttatggaagaagatgggcacgacttgtcaaaacgaacgcgaagcgcatctgttgctgcagttgatgaaagactgttgccaagtaacagcgccggggtctttgttcattgccgaagcgattgtcgcaccggtcgagattactaaatatttcggcgaggacgcgattatcgcgaaagagtgtgaaatcgcctataacgcgactttgatggccttgctttgggatgcgttggcgacgcgcaatagcaaattactgtatcagggcttgaaaagcctgcccgataaattggagcgagcgacttggttgaattatgtgcgttgccatgacgatatcgggctaggtttcgatgacaacgatgtgcggcatgtcggttatgatccggtcgcgcatcgtcgttttttggtcgatttctatacaggggcttatgaggattcgatcgcgagcggcgtgccgttcatgcgtaatgagaaaaccggcgatgcgcggattgccggatcgctggcttcgttggccggccttgagacggcgattcagtccgatgacgaacatgcgatcgatattgcgttgaacaaaatcatattaatgcatagcgtgatattttcattcggcggccttccgcttctgtattacggcgatgagatcgcgacctttaatgatcacagttatctcgatcaacccgataaggctaatgacagtcgctggattcaccggcctaagatcgattgggagcgggccgagcaacgtaagaaacaaggtacggtcgagtatgccgtatttagcgcattgaaaaaaatgatcgcgattcgcaaggaaaccacggcttttgccgacttcaataatcgacagttgatcgaatgcgaaaacccgcatattttttcgtttatccgttacgatcataaccggccttcggaacaagtgctggtgatttgtaatttcagcgaacaacctcagcgtatcaagatggaaaaatttaatacggcggggtttggcatttacggacaagtcagcgatttatacaccggcaagcgtccgactgaattcgaccagcaactggtcattcagccgtgccagttttattggttaactgagtattaa

SEQ ID NO: 49 - DNA, 4880 bp, Artificial Sequence
  Description of Artificial Sequence Synthetic polynucleotide
ctatccgcgcgtctccttcacggaaccgaaaggggctgagcaatggttggtcaaatccgaaagcggtgaaggcggccacggtattcgcctgtttcgaaacggcatgccgacccaaagaaatatgtactggcaaaaatatatcgaagggacagcctgctccgctttatttcttgcagacggaaaaaatattcgaattatcggtttccatgcgcagtggtgcgaaaacctgaatgccgaccaacccttcctatttgccggcgtcatcaatcgctgccccctgtccgccgagcaagccgatatcattcattcgtggatcaccgcccttgtgcgggcaatcccgcttaaaggcctcaacacgctcgatttcattatccaaggtgagcgaatttatgtattagaggtcaatccccgcccatcggccagcatgcaattatatgatggcgatttattcggggcgcatctcaatgcctgtcggggcatcttagacaatacgaactttacgagtcgtacggtttgcgcttatcgaatcatttacgccgacagaagcatcactattcccgatgccttttgctggccgaaagcctgccgggatttaccgattgccggctcgataattcgcaaaggacagcctatatgcagtatcattgcccgcgatataaacgttcggaacgttcatcggcaattgaacgaaatccggcaaaccatttttcaatcattcaattaaaggtgttttaggtcatggcatcaaaagcgagcgttaatagactagcgcagcctctggttagagaactcatcgaaaatgcggctaaattacgagtcggcgtagaaaaattggcgaacggctgcaccatcatcgacgccggcatcgacatgccgggcggtttggaagcaggccgaatcattgccgaaatttgtttgggcggcatgggctcggtctctatctcgcacagttgttacaccgcgaattggccattgacagtcaatgtgcataccggtaacccggtcttggcctgtctcggcagccaatacgccggctggagtttgtcgcatgaaaaatattacgcgctcggctccggtcctgcacgggccatggcggtcaaaatcaaggatggccaacaagagcctgtcgaggagctatacaaagaattggcctatcgcgacgatgcggaaaacaccgtattggtgatcgaaaacgatgcgatcccgccgctcgaaatcgtcgaaaaagtagcaaacgcttgtcaagtagacccttgcaacttaaccatcatcgtcacgccgaccagcagcctggcaggcggcgttcaggtcgtcgcacgggttttggaagttgcgatgcacaaggcgcatgccttgcattttccgctggaaaacatcgtcgatggtagcggcagcgcgccaatctgcccgccgcatccggattttatcaaggcgatggggcgcaccaacgatgcgatattgttcgcggggcaagttcatattttcgtcaaaggcagcgacgaagcagccgagaagctcgcaaaagagttgcctagctcgacttcgaaggattacggcaaaccgttcgcgcaagtcttcaaagaatacaattacgactttttcaaggtcgatgcaatgctgttcagccctgcaagtgtcatcgtgaccgcagtcgaatccggtaagagtttccgcgccggcaaactcgacaatgcgttgctcgatcaatcgttcggcgcttaagagtttcgacacatgggtagaattgccgttttcacagacgatccgggttggcacggcaaagcattgcgtcttgcattcgccgctcgcggctattcggcggactatgtatcgttaactgagtgccgcttgaatctggtttcggaaggacttcctgtttcgattccgggtttcgagcatgccctgcccgacgccgtgtttgtccgcggcgtaccgggcggctccctcgaggaagtcgtgttttatctcgacatcctccatgcgctgaagatcatcggcgttccggtgtacaacgacgccggagcaatcgaacgcagcgtcgacaaggcgatgaccagcttcctgcttcggcaaaacggtttgccgacgcctccgacctgggttttacgcgatagaaaagaagcattggccatcgccgaatgcgaactcgcgcaaggccgcatgatcatcagcaaacctttattcggctcgcaaggcgaaggcgtccgacgcatcgaaaaaatgctggatttgttttggttaaccggcagccgcggcatctattatttgcaacgctttgtcgattgttacggcgaaggttattccgatacgcgagtattcgttatcaacggccgcgcggtcgccgcgatgcggaggaaaggtacattttggttgaataatgttgccaaaggtgctagttgcgaacgggtccaattggaatccgaattatcgaaattagctatcaagacagcggaagtcgtcaaaatggactatgccggcgtcgatatcattcgcgaaaaaaacggcgattacaccgtaatcgaggtcaacagcattcccgcatggaagggcctgcaaagcgtttgtgatatcaatatcgccgaattactcgtcgaggatttgctggacaggcatgtcggcgatcatcgccccgaattgcaaacggttgcctcatgatgactatcgaaacgcttgaagatttgtaccgacaagcttgcgagatcgaattacaggcttttaagccgggcaacgtcagtgttcatgcagacgggcacgacatgtccgtcgatgatttcagaatcagcgcaaaggtcagcgcaccgcatatcgcaaatccaaattactcgcttggcgaaaaaatttattatgcgatcaaggccacgcgcgatgcagtcggctgtaataccaatctgggcattgttttattatgcgcgccacttcttcaagccgcattattacgaaaaccgaattcgacgttaagacaagctttacacgacgtattaaataatacgtcagtcgacgatgccgactgggtcttcaaagcgatcgttttagcctctccgggcggtttaggccactcatccgaacaagacgtttcagagcggccggctgtcaatttgaccgaagcgatgcgaatagcggccgacaaggatcgcatcgctttacagtactgctcggattataaagatatttttgatttcggagttttaaggtataatgcttggctaactcgatgggctgaagctaattgggcggctgtagcagtctatgtcgggttgctgagcctttatcccgatagtcatatcgagcggaaatacggcaatcgctttacgctttcggtgacatccaaaatggacatggttaaccaagccctgcaagattctgataatccgaaagaaatcgaacctttactgcgtaggattgacgcggaatttaaatccgaaggcatcaatccgggtacgacggccgatttaacggtagcgacgattttagcggtatttctggaggatcatattagcaattcgaattgctaatgggtcttcaaaacggatttacacttaaagtaaataaattaattctcactgttcatattattcttcaaaggagacaactagcatggctaaaatcaacaacctgcgcgtaggcgaatcattggtaggtgacggcaacgaagttgcgcacatcgacttgatcatcggcccacgcggctcggctgccgaaaccgctttcgctaactgcttgactaacaacaaagacggtttctccagcttgttggctgttgttgcacctaacctgatggttaaacctgcaactgttatgttcaacaaagtcaccatcaaaggctcgaaacaagccgttcaaatgttcggccctgcgcaacgcggcgtagccatggctgttgcggattcagttgaaaacggcaccattcctgccgatgaagctgacaacctgttcatttctgttggtgttttcattcactggatggctgaagacgacgcaaaaattcaacaatacaactacgaagcgactaaagaagctatcgaacgcgctgttgccggctctccaacagctcaagaagttgttgctgctaaagccactgcaacacacccattcgctgccactaaaatttagctcggcatagaatgaccaaaaaccccggctagcccggggttttttttgcctaaaataaattggatagcggttagcagtgagtagtaagcggcagaatcagggcttttatcctactactccctttatcctgaattcggcagttttataccatgaagttcatgaaggacttcaagaaattaactaaatcttctcgctaaccattttggtgagcgaaagctctattcaaacgcgtaataagttattgaattaacttcctattcttcatgatcttcatggtgaaatgctttttctaggtttatactcaaaaatggctaactttatgaaggtatggggggggtgtggctcataagcgccaacttaagaatctaagtatttgattaagtaagactaacgccacctccccgaccttcaataatgagctccaaaacacgccccggcaaaccctgaagctttctcttgcgcggacgctcacagaggctttttgtgcagtcatcgataaatcgcttatttattggaaaacaagccgtaagtcctgccttgatctcattggcgatcgtgcgccataaacgccaatgggtaatcgaacgatcaccgtccattgtggtggccgcccggccgaatcggcgctgcgctattttttgagtcactgcagcaaacagtaacttgccatgcaaatataaatccgccagcttgctgtcttttcgggcgcgtagccggtcgatatcaagcaagctctttagccgctttatgaccagctccacttgccagcg

SEQ ID NO: 50 - DNA, 18991 bp, Artificial Sequence
  Description of Artificial Sequence Synthetic polynucleotide
gctcaaccgactcattgacgaaaatttcgatgacattcaaaaaattatcgccggctaccaggtccaacttttaccgatcccaaaacaaaaaagccgcaaagacgacgactaattcatcggatacattatttcacttggagcagtcaagcattcggttttatcctaagttcggtagtttaaccatgaagctcatgaaggacttcaaaaaattaccttaacattctcgctaaggaacgagcatgaaataacttcgacaaatgctgaaagggggttcggtggctcgattgacaggcgtcggcggcagggcagatttttgctcctgcaaaatccgcattcatgccatccatggcaatctgatagccgccgtcgagcctacatggatgtattcacccagcacctaaattccatagcccattggctatggttaactatcttggataatttaggtgctgggttcacggcgtcctgtcaagcgagtcgccgcaccaccacaatgcctaaaacttgtagaagtaaatttgtgcatattcctaaccttgtactctacacaaacgcagaacaccttattcgtcataatcttcatggtgaaatgcttttttctaggcttatagttgaggcggtaatcaaaatacggatataacatccaccaggctaaatcatttgaatttagcctggttttttcaggattaattctttttgggaggcgtcaacaccgcttgcggaccgggtgccgtttggctgtgttttaagacgacccctcctaaaggcggcaaggtgatatagagcgaatgaggccgattcatccaagcctgctcttgagtccaaataccgtccggcgaattaaccaccccgctgccgccgaattcctgcgcatcggagttaaggatttcgtcataacgcccagcagccggtacaccaatgtgataatactcccgcgggaccggagtaaagttcagcgcgacaaccaaaaactctcctgtatcggcagccttgcggatataaatcagcaccgagttatgggcatcatggcaatcgatccactcaaatcccgcatcggtaaaatcattggcatgtaaagccgtctccttttgataggtgtgattcagcgtcttaaccaaatgctgcagtccttggtggaaagggtaggccaacacataccaatcaagcacttgactgctgtcccattcgcgcccttgaccgaattcacaacccatgaagagcaactttttgccggggtgggtaaacatataaaaatacaccaaacgcaaattagcaaagcgctgccactcatcgccgggcattttatataacatcgattgcttgccgtgaaccacttcatcgtgagaaaacggcaacaaaaaattttccgtaaaggcataaatcaggctaaaggttaattctccctgatggtattggcgaaatatcgactctttttccatatagcgcaacgtatcgttcatccagcccatattccactttaaatcgaagcccaagccaccgacatacggaggcttggtaacctgcggccaagcggtcgactcttcggccatgattaacacgccaggaacttgctgatgggtaatttcgttcaatgatcttaaaaagtcgatggcctccaaattttcgcggccgccgtacttattgggtatccactcgccttcctcacgggaataatcaagatacaacattgacgctaccgcatcgactcgcaaaccgtccacatgatattcctcgagccagaatagcgcactcgagatcaaaaagttcttaacttcatagcggccgaaattaaagatcaacgtcgaccaatcacgatgttcgccgagccttggatcttcatgctcataaagcgatgtaccgtcgaaccatgcaagcgcatgcgcatcttttggaaaatgcgccggcacccaatcgagaatgacaccgatctcgtgctgatgacaataatcgacaaaccagcgaaaatcttcggggctaccgaaacggctggttggcgcatagtaccccgtcgtttggtagccccacgatttatcatagggatgctcagtaaccggtaacaactcaagatgagtaaagcctaaggttttggcatattccaccaaggccaccgccaattgccggtaattgagcaactcgccttcggagcctcgctgccaagaccccaaatgcacctcgtagatgctaatcggcttgtgctgccaatcgaacttctcacgctgtttcaaccatttttggtcatgccaattaaaattatcgtttgccgttacaatactggccgtattcgggcgaacctcataaaaccgaccgtaaggatctgtttttaataaaatatcgccattcttcgcgcgaatttcatacttataaaccaagccgggctctaagcctggaataaaaagctcccaaacgccattaccgcgcacccgcatttgatggcaacggccgtcccagcggttgaaatcgccgactacgctgactcgcgatgcattaggcgcccacacggaaaacagcacgccggcaacattttcgatttcgtgtacgcgtgcacccagcaaacggtaagcatgccaatgtttaccttcactgaataaatgtaaatcgtaatccgataattgcggcccgtagcaataaggatcgtaaccgatgtgctcgtaatggtcttgatcgcgccaaatcagccgatagcatggaggaacttgtgatgctaagccatgccattcgaaaagagccgtaccctcgattcgagtcaatggcaagtttccttcagccaaggccacggtttcggcatggggcaaaaaagctcgtaccacaacctgtttgtcatccttgggatggcggcctaaaacggcaaagggatcgctgcattttacatcgacaagactctgtaattctgtggacagttttgttttactcatctgcttttgcgggaagcccgcgaatttataccccttacttgtcaaatttcggtgccggggtgcccgtcaaaggacgccgtaaatacgtccatgtaggctctatgatagcatccatgctatcaaagcctttgccgaacaccccgacacctccatagactaatgccgaaatttaaagtgcgaaaggtggtatatcattcgggtggggatagctcagctcgcctcggcaagcccggttcccgctctcctgttttaatgtgctatcctggttttagcattgattcggcgtcgttattctggcacctgtaattgtaaaatcttaacttttaccgagccgtctactgtgtagtgaccatgacccgcaccatacccggcaataagtcgaaatgatagggactcggcacataagctagcggatactccggcgatagatcggtaaacggcgcgcgcgactgaacatattgatatagatcgggcgtcgaaaactcatggcgttgccaagggtctttatttaacaagatcaaagcttcttgttgtccggataggctggctttccagaaaaagagtatcggcgactgcggataaccgattaggttaaaaggcccctcttcctgaaatacagggtaatcggctttcaccgtgttgacttgagtaataaactcggtcaaatccacctcggtattttcccagtcctcggggcgagtatgcaccacatgcagcggtttactaaaaccgtattcgtaacccatcggcatcatcacgccgctgctgaacactgcggaaaacaagtaacgctgctttagggcttcggcattgccgttgctttcggcaaataaacgaggcgtatcgtgactttcgggaaaactgatcgacggggcggcttggcgcaataattgatactgttccggtagccaagggctttcgaagtcccaccatttggaactattgaaaatataatcgaacccggcttgcgcggtttgcttggttcgctcggcgctacagccaagcgtttcagcggtaaacaccacatcgggatgtttttgtttcgtatccgtaatcaattgccgccaaagttcgggcgctaattgataggcggcatcgcaacgaaagcctttaaaacccagatcgatgtatgactcgatgattttacgataataggcataaagcccattatcgtgactgcggccatgatcgagttgcgctaaatcctcccataccactttatggccgccgtcttcgagacaaaaaggacgctgcacctgcccatgctcacgaacaaaccattcagggtgctgttttaacaagggcgaatcgatcgcacagtgattcaataccaaatcggtcataaagcggagtcggtgttttttctcagcctgttgaatcaagccccgcaattgttgctcggcggtctttttggatttcggatcgactaacagcgggtttaactgaaaataatcggcaatagaatatagactgccggatgccccggttttttgtaacgggttgacgaaaacccaatcgaaccgcatcgccgcggccctttccaagtgcgcctcccactgcccgaagtggcctgccaacagggggaacaagttgtacatcctcatgaatcggcctctttatttgttacgaatatagtcatagatattcaggtaatgttggcccggatatttccaggagtaatcgtaacgcattgcatttttcatcaactcccgaaaatgatccggaaattgataataacaatcgatcgcgcgcgacaatgccgactccagcccttcattgttgtaatccctaaaaacatagccgttgcgctcgtgcaaggctttatgatgcgcatgatccttatcgaataccgtatcggccaatcctccgacctcgcgaacgatcggaaccgtgccgtatttcatcgatatcaactgcgtcaaaccgcaaggttcgaataaactaggcacgacgatcatatccgcaccggcataaatcagatgcgccaagccttcgtcgaaaccgacttcgatatggcaatcgggactgtcgttcaaatgccgcttcaactcccagaaatagctattgatatcccgctcgggacttgagcctagcaacacgaactgcgcccgatggttcaatgaataaaacaatgcatgacgaaccaagtccaaccctttctgaggatcgagccgcccgacgaaagcaataatcggcttttcattatccgctagcaacaagcgatcgcgtaaagcccgtttattgagatatttattgtctactttttccacgataaagggagcggcgatatgcgggtcgatttcgggattccagacatcataatcgatcccgttgaccacaccgccgaacttataatgatgcgcatgaagggtcggttccaaaccgaatccttgaccgccgtctttggtttctccggcataacgcggcgataccgtcgtaacgaagttggaatagactatacctcctttcatcaaattcaacgcatgcggaatccgattatccaacaagcgatcatcatgaaaataatactccggtcgatgcaggcccgtcgcattcaataagaagtcgcccgtcaacccttgatgcttgaagttatgaatcgtaaaacatactctcggatgccagaagcctaatttctgatagatctcatataaaaacacaggcaccaatgccgtttgccaatcatggcaatgaatcacatcgggatgcttgccggatttccaaagaaattccaatgcggcacggcaaaagaacgcataacgcagcacgtcgtcattaaagccgtaaatcgtgccgcgattgaagaaattgtccggggaatgcggctcgatgaaaaagcatttacggccatgaacgaaaccgaaccagaccgtacaatgaatatggccgttataccagggcacccaaaggtcgtcataggttttacataagccccaaatatgatcgtaatgcatacaatcgtatttgggcaaaataatctcgacactgtgtccgcgcaattccagttcgcggcttaaaccggtcacaacatccgccaaacctccgaccttcgccaagggcgccaattcaggagtaatctgcacgacaaacatatgcgggtagtcgggaccgggttggtcatggtacggcgaatgctcgacgtgcgacatcatgtcggcaacgggctccggcgccgaagactcgggtgtcggcgctattggctctgccttactataatagggttgccgctgaaacttttccgacggcacatcgggactgatggatgccgcctgagcaacctctggcaaaggcgtgataaatgccgtattgggctcccggtatacggattcgccgcccttggaagacgtatcgacttgggtttcgccggcggtaccggactgtttaggaacggctttttcggaagcttgactaggtgcagccgccaccttcgaagcaacattctctgccgccgctgtctcgttttgcggaacaacgggtaaaaccggaacaggcgacaacgactccgcttcggatttttcctgcgccgttttttcggatgttttaatctgccgatcggatttagtctgcgtatctgatattttgctcgctgaattacgcattagcgccgacttaagttcccctgcagacttagttttcggagaatccttagcttctatttcggctttagccttctcttcatccttcttagtttgagtcggcgccttgacgctagatgcggtcttggttttttcggcaagcttttttttgggagcggctttcgccttaggcttaacgggggattttgccgtattcgcagtttttgacgaagcctttttcacaggctctgcttgagccggttttgcagcctgcgctttgggtgaagatttaacttccggcgccgttttagctttcggaactgttttaggttttgctgccgtttcggattccgaagacaggctaggcttcggcgcatcatcggtctgagtcgacttttccattttggaagattcggctttagggtcgttgttaaccgtcgacgcggcagcgctcggctgctcttccgcctcaggcgttaccgcggattgattctctgtcgtcggcttttcggacgccttattcgttgttgctgtttttgttgagcttctggttttagttgtagtttgctttgccataaatcagacctatcatgccaattaaaagatatactttcccgtcgcccttagtatttcgtgacaaataacgtccaggaactatgagccttgttgagggctcggtaacttgtttaacaggactctctgccgtcgacatctatcaagcgaccaaccaaacccgcctatcctacattaaaattagggaagttttatgaccgaaaccttagcttagtatgttgtccgcagttgtgtttttataagtccgaaaatcaagcatcggaaaaatatcatccatcacttctaacgccgtcaaatagtgttcgtttattgtatccgtacgaacgctttcttgcagataattaaatcgagccaaatgatcaagaatacgctttttagcatactcgacggtcgttcctgatttcataatgaacggccaatccgaggcctgcgccaataataacgaccgtgccgcctgattcaaagcccgctcctccaaggaaaaacggggagcttgctgaaactcttcggctaactcctccatcaacatcgccgatttgtgtaaaacgggataaatccaagcattggtttcattgagccagtatccggaatagccctgttcgccccaactcgatgccgaaggcgttgcggtttgcaagttttgaaaacgcgacaaataatcgctaccactgaccgactgcattcccaacttattgccgccaaccaggcgtaataaaaactccagccattgcggtccctcgaaccaccaatgaccaaacaattcagcgtcgtagggagctacaatgattggcgatttcctcatccgatagctcaggtcgtcaagctgccgttgccgcttcaaataaaagtcttgggcatgttgcctcgccttcatcaatgcttgacgcggccgataggccgctttcgggccaccccctcccgtcactcgctgatatttgatgccgacgggtacgcgaatcgtttcttccaataaatacggagcgagataactctccggaatggtggaatcataaccgatatcgcaatgatattcacgatagtcgccgtctcccggatacccttcctgcgcgctccagacttgacgcgacgattcgggatcgcgggcaaaagccgccacaccgttagtacaggctataggcgcatagacgccattttctgattgcgggcttgcttgagtcaaaccatggctatcgacaaaaaaataacgaagatcggcttcgtcaagcaacgtctccaagcccggataataaccgcattcgggcaaccaaaatccttttggcataaaaccgaagcactgtttgaaagtttcaacgcccatgttaatctgattgcgcaccgcactttcggaaacgttcagtaacggcaaaaaaccgtgtgtggccgcgcaggtaatcaattccaggccgcctaattccgcctgttttttgaaagccgcaactaaatcgcaatcataatcctcccgataacgctgcaatgcggccttgaaattacgtcgatataaacgagccaaatactgatattcagcttgcttgcgagtccttgaaacttcctgctcggctaatctgatcaaaccttgcaaatggcgaacatagcgctgctgcaataactcatcattcaacatcgtcaacaaggtcggcgatagcgacagtgtcagtcgatatcgcacgcgatcccgattcaagcgctcgcacatgctgagcaagggcaaataacactcggttatcgcttcgaataaccagcgctcttcaaaaaaatgctcatactcgggatgatgtacgtaaggcaaatgggcatgtaatacaatgcataaaccgcccctatccatctcaagtacctaaggaacgagcatgaaataatttcgacaacagttgaaaggagggctggtcggcaattcccagtttggggatcaaaaagagtgaggaggatgcttggcgaggacttcggctgcagggagcagccgtcaagcctccatgaatgggtttacgacatcctgtcaagcgagccgccgaaccgacgcaaagcctactacttgtaagagttattttgtgcatattcttaagtgagttttattgctattctgcagacaggatataccggcactcgaataactatccatatcatggagctcaaatcgttctgctgaggtcaagtgaatcggagacgccggtgaaaacgggatatcttcggcatccggcttgctcaatgtttgccgaattttagcgtcaatatcggcttcattaaaggctaaaggccttgttagcgcggtcaccggcgtcatcgcagaagaaggtatactaacaggctgcgagcgaacatatacaataaagtccccattactgtcgcattgaccaatcgccgccgtataaattgcaccggcaaccggcaattgaattttagcattggcgtgcgtggataccacgccgacatcaaaccatggacgctgatgctcttgagtatccgcccaatcaacctgccagaacaagcgtaatattaacggattattcgtcgcctgcagcagaggagtaacgccggcaatacgccaataggcatgaagatgatagggatctatcggtaaaataataagctgtggaaaattaagcggttcagtctgactgaattgcaaaggatgataggtatgactaatggtttgactgatgtgttgaatttcttgagacgatatagtgagcttagggttatgcctggaatgccaatatctcattcattttttcctgatttcataaatcttttaaccacattcactgcatttgaagggtaaccgtcatatcggcatgatccataggcggattatcctgactgaaaccttaaagtgtcaattcaaattaggctttgccaaccagattttggcggaagacaacccaagcaaaccttcggcaacctttgcccagcttaatctcgcccgtataatttgatcgcacgcgccaaacgctgactgtgcaatggtaataattgatcccaagtaaaacgccacaaccaattgccttcagtcgtaccgggaatattcatgcgatgctcggtatctaactcaagaatgtcctgcatcggaatgatcgcgagattcgcaaccgaagctaaagcggtttgtattagcgtatagggcatcgagctagcaggctgacctaagtattcataaacataagcttgctcttgctcactgatctgacggtaccatcctaaggtagtgtcgttatcgtgcgtgccggtataaacgacgctgtttttatccagattggccggtaaataaggattactggggccgccgctgaacgcgaattgcagaatcttcatgcccggcaaagagaaatcgtccctgagtttttcgactttatcggtaatcacgcctaaatcttccgcgaccaatgcaataggtccgaacgcatcgaaaaccgtctttaacaaggcgtgccccggcgccttgacccagcaaccattcattgccgtgggttcggataccggaatctcccaggcggcctccaaccccctgaagtgatcgatacgaataatatcgaacatttcgacttgcgtcttgattcgatcaagccaccaatggaagccattttttttaagatatgaccagttgtaatgcggattaccccaacgctgccccgtttccgaaaaataatcggggggtacccccgccactaccgacatttcgaactgctcatccagtttaaacacctccggatgaacccaaacatcggcgctatcgtatgaaacgaaaataggaatatcgccaaacaacaatacaccatggcgcgccgcataatctttaagttcttgccactgtgtaaagaatacgaattgctcgaatttaatcgcttcaatctcagtgcttaggcgtctggaggcttctttaagcgccgaaatattgcgcgatttataccgttcaggccattgattccagctaagattattgaattcttttctgagtgctataaataaggcgtaatcatccagccaaaactttttgtcgatacaaaatttggtaaattcgattttttcgtgttcggtcgcgtgcagcttaaagccataatatgcgtgattgagtaaacactcccttgcaaatgacggactgccgccacaatcttgacaacgctcgccacggctcagccagcctttgtcaaccaacttatccaagtcgatcaaagcggtattaccggcatgagcggagatgcactgatagggagaaccatcatcatgaggcatgtttaatggcaaagtttgccaaaccgttacaccgacactatttagaaagtcgacgaaccgataagcttccgggccgaaattaccattatcacgattgcctggaagtgaagtaatgtgcaataaaattccggcacggcgtctatgtaagatatcaatcacaaacgtctttcctgactaaacgttttcaatacccggcctcatcgtaccgcccatggcgggagctccggacccctgagtgaacgataatgccagataagccggcggatcttctcctaacaacagatagagattactcaagttcaaacgaaactgtttttcaaagctgctgacagcctcccccgggttgtaatcgccgaaccaccaaaaccaatccgaaccttcacaaatagcaagctgatgctcggcaagagccaactgctcctcggtcaagcgcccggtcgacacgactttatcaaacgcccatttagcatcgcccagcatatcccaaccccggtttttatcgacatcgccgatccaggtagaaaacgttccatagacccaactgccagccatcaaattcggaagcgtttgaatttcaatgacttgatcgaggcattcggaaaatgttgttagctgaatctcggggtgatgactcaagcgtttataaagcgcgctgagaaagtgataaccgttttgcggaaaatactcccaagcattttcgccatccatgataatcgataccaccttatccgtagaagtatcatgcttatggatttgttccaagtgattgaccaaatcggcaaccgcatcgtcggcatgccatttggaatattcaaatccgatcaaatccgataagccatcgtccctaaaaaaacaaggtataccggttccgctagcgcgaaacggatgatggggactcacgctatcgatgcctgataatcgcaagctgttatgcaagacattaccgcctgttgcagcccaatcgaaaccgaactcggagagcatctttaatgtctcggtactgactgctccctcggatggccaacagcctctcggcgtgaatccgaaaaatcgtttaaaggtttcgacaccatgcttcagatgccatttcaccctttcctcgccccccggataatgatccagttcgggtagcggagcatccggcatcgcctgcaatgtacttttaatatcgattaacaaaggcataatcggatgcgcataaggcgttaccgaaagctcgatgcgtccttttcttgccaacgccttatatcgacatattgcagtggataattgctcaccgatgacctcgacgatttcgatccgttcatgcaatgtgtagcctgaacctttggcaatcaagcgcttgaccctgacatcggtcagcttgacactctcgcccatccaagccagatgataccacgctaaaagatcgctgatgaattgagaattcatatagctcaacgcatcatgattctgctccatccattcggccattttgaccagtttctggaaagtcggatatcgtccgatctgccgctctcgattggcacgcatgcaatccttgaccagttttagccgtgcatcgggatccgagggaatcgatggggcaaccagcgccgccaacaacggatctttaaatgagatgcgatcatgcagatagcgccccacttgcctggcataatcctcgatctgctctagaaggatcggcgcaaaattaacgaccgctttggcttccggcgccgcttcaagatgggcgatcatgtcgacataatccttcataacatgcaaataggtccaaggcagttgaaattcgccggtttgcaaatcgcgatactcaggctgatgcatgtgccaacatatcaccagcttcagttttttatctgacatagtgcctactctgtcctaacatatccggggtgaccaatacgactccgcccgggctgacatgaaatctttttttatcgtcctcgaggttctcgccgattaccgtgccttggggcacttcgcaacctttctcaatgattgccttggtaattcggcaatgccttgcaatattgacctcaggaagcaccacgacatcttgtaacgtcgtataagaattaacccggacattggaaaataacaatgagtgcctgaccttggcgccggaaataacgcaaccgccggacaccatcgaatcgaccgcctgcccccttcttttgtcgtcgtcgaagacaaattttgccggcggagtttgttcctgataggtccaaatcggccaagttttatcgtataaattcaaatcgggcttgacgccgattaattccatatttgccgcccagtaggcatctatcgtacccacatcgcgccagtaactttgatcgccttgcaaattcaggaatgggtatgcactgaccctatatttgtcgataaccgcaggaataatgtccttgccgaaatccctgttcgagcctttgctgtcggcatctttgatcaactgctcgaataaaaatgccgcattaaatacatagatacccatcgaagccaaggctttgtccggcttacccggcatcgaaggcggattggcgggtttttcgacaaatgcttttacccgtcgattttgatcgacgtccatgaccccgaactcggtcgcctcttcaaggcttacctcgagacaaccaatggtcaagtccgcattattggccacatgatcggcaagcatttcgccgtagtccattttataaatatgatcgccggccaatatcagaacatactccggccgacgggttcttagaatatcgatattttgataaaccgcatcggcagtcccctcataccaagacgtttcgatgcgttgttgagccggcatcaaatcgacatattcaccgaactcgccgcgtaagaagccccagccttgttgaatatgtcgaatcaaagaatcggctttgtattgcgttaaaatgccgatttttctgattccggaattaatgcaattggataacggaaagtcgacaatcctgaattttccgccaaaaggaactgccggcttggcacgccaatcagtcatattttttaaccgtgagccgcggccgcccgcaagaatcaaggcaattgtatttctagttaaatgactgacgaacctgtcttgaggttggttaattgactctgacattttcattccccttctggaattgactattacatctgttgcttaggtggtaacattctgataaaaatcattctactacattgaggcgtttcgaagtgaaaaagcaatccaatatgccgttgtgtaatgatttatgcaaaatcattgaagccaaacatcatgaccctttttccgttctaggccgcctcagcgataacaacctgaccaaggtaagactctatttgccttacgccgattcggttcgttttgctgaaaaaggccctgaaatacctcgactcgcaaaaacggacttttttgaatataccgctcaggccggcgaattaccagaacattatcaaatttcttggatcgacaaggaaggccgacaacacaccgattacgacccttatgactttcccgagcaattgccggaattcgatcgtcatttgttcggcgaaggcaaacattggcatatctaccaaaaacttggcgcacatcgatataaggtcgacggcatcgatggcgtattatttaccgtctgggccccaagcgcggagcgcgtcagcgtggtcggcgattttaaccgctgggacgggcgctgtcacttaatgcgagtgctaggtaacagcggggtctgggagctttttattccgggcctcgacgcgggctgtttatataaattcgaaatactgaaccgtcaaagccaagaaattttgattaaaacagacccttatgcccagcaatatgagcatcgccctaaaaccgcttcgattgtcgtcaaagaaggagcctatcaatggcaagatgcgaaatggatgacttatcgcaaaacccgcgattggcttcacgaaccgatgtctatttacgaagtgcatttaggctcgtggcaacgggacgatgccggaaatttcttgaattaccgtgaactcgctaaacagttaatccaatacgtcaaagacatgggctttacccatatcgaattactgcctatcaccgagcatcctttcgatgcttcatggggttaccaatcgacgggctacttcgccccgaccagtcgacacggatcacccgacgatttcagatattttatcgatctctttcatcaaaacaacatcggcattattttggactgggttcccgcgcattttccgaaggactttttcgcactggcgcgattcgacggtagcgcgctctatgagcacgaagacccccgcaaaggcgagcatagagattggggcaccctgatctttaacttcgggcgcaacgaagtcaaaaatttcttactggcgagcgcgatattttggctcgaagaatttcaccttgacggtcttagggtcgacgccgtcgcatcgatgctttatctggattattcccgcgatgaaaacgactggattcccaacatgtatggcggcaacgaaaatctcgaagccatcgcttttttgcgcgaactcaataccgtaacgcatgaacaacaccccggcacggtcgtgatggcagaggaatcgaccgcatggcctcaggtaacccgcccgacttggaccggaggcctgggtttttcgataaaatggaacatgggctggatgcatgacattctcgattatataagccgcgacccgatacacaggcgttatcaccacgatcaactaaccttcggcctactttatgcctttaccgagaacttcgtccttccgttctctcatgatgaagtcattcacggcaaaggttcgttactcaacagaatgccgggagacgaatggcgaaaatttgcgaatttacgcctactttataccttgatgtttacctacccgggtaaaaaattattattcatgggttgcgagttcgggcaaggtacagagtggaatttcaaccaacccctggattggtatgtactgcagtatcctcaacaccaaggtttgcaaactttggtcaaagatcttaatcatttgtataaaaactacccggcacttcaccaatacgacttcaatcacagcggctttgactggatcgattgccatgatgtcgaacaatcgattattagctaccgccgcaaaggcaccaacgatgatttgatcatcatacttaacttcacaccgattgtgagagaaaactatcatatcggcgtgccttccgaaggggtttattttgaaatctttaattcagactctgcctattatgaaggtagcaacatcggcaaccgcgagatactatcggaaccggagccttggatgggacaccaacaatcgatacacttgactttacctccgctaggcggaatcatcttgacgaggcaaacgaaagccataagcagctctgctcactaaatagcgtcctgtttgagaaagatgatccagcgtggcattgctcaccgcaaattaactggcgccggcctagcttataaaaaattcacttcgatatttatacgtgataggcacaactacgcgagaaatgcccgcaaaatagaactgccgccgccccgaacgcaaacttaaactgattcttcaataacaccggacgatcctacaacagcagacagtacgccaataaacactcaatcggctattgtggcgaaatccacctcggtaaactcaattctttaatatatacctagaaccgaattttgactgcctttaaatttaataattatgaaacgaattctttttgttaccagtgaagcacatcctttaataaaaaccggcggcttggcagatgtttcaagcagcttacctaaggctttggcggatctaggccaggatatccgtatcatcatacccaactatcaagctataaaaaaaaccgaaaacgtccaacatcggtgtacgttgagaattaataattgcgatgtcaatattctcgaaacccgcttgccggaatcaaaagtgatcgtatggctgatcgattgcccccagttttttggctacccgggcaatccttatcacgatgaatacggtaatgcctgggcaaacagcgccgatcgcttttcgctgttctgccgcataacagtggaagtcgcgatgaatagagcctacttagattggaaaccggagatcgtccactgcaacgactggcaaagcggcctagttcccgccttgttaacgctggaatacaatcgcccggcaaccatttttacgattcataacatggcctatcaagggatttttccctattcgacctacaatgcgcttaatcttccaagacaactttggaacccaaatgcacttgagtattacggcaacatgtcgtttttaaaaggcggcattgcttgctccgatcgagtaacaacggtaagtcctacctatgccaaagaaattcaatcatccgagtttggttacgggctagaaggcttgttaacccatcgcaaggaatttctaagcggcatactcaacggaaccgacaaagactggaatcccgaattcgacagcaacatcgtccagcgctacagccataaaacgctccatcacaaacaaaccaataaggccgcattacaagaacggctctcactgcctctcgagccgtcgattcccattttcggattaattagccgtttggttgaacaaaaaggtatcgacttgctattagaatgcttgccggaattattaagtatgccagtacaatttgtattactcggtagcggcaacaaaagcttcgaacaacgtttatataattttgccgaagcctaccctgaaaaaatgtcgattactatcggctacgatgaacaattggctcattggatagaagcgggaagcgatattttcttaatgccatcgcgcttcgagccctgcggattaaaccaaatgtatagtcaacgctatggaactttgccgatcgtaaggaaaaccggcggcctagcagatactgtcgtcgatgctctaccccatacgattgacaacaaaacggcaaccggaattacttttaatgaatcgacccccagttcgctactggaggccatcaagcgcgcgttgattttatacagagtcccggaaatttggacacaactgcaaaccaatgccatgaagaaagacttttcgtggaatagtagcgctaaacaatacttatcgctctatgaccatttgtaactcgattaaggatttggtcgtaaataacttccctgttttatggagagttcggtaactcgattggcaggtgtcggcggcagggcagattttttgctcctgcaaaatctgcattcatgccatccatggcaatcagtcgccgcggtcaagcctacacggacgtattcacggcgtcctgcaaagcgagttaccgaaccctcaacaaagctcatagttccaagacgttatttatcacgaaatcctaaggcgggctataactccaaaaagagttatttatcttttaccatgaagagcatgaaggacttgaagatacagcatttacaattaaacgtaagtgactgtccattaacagcctatgtcatcattattgccacttcagtctcgccattgttttgatattttttagtcattatacctttcgcacttcaaatttcggcagtgcctaaagagacactgggtccctctcctaacgctaggtgagaagctgggctcacgacgtcctttgacgacccccgacgccggattttgatc

SEQ ID NO: 51 - DNA, 40 bp, Artificial Sequence
  Description of Artificial Sequence Synthetic primer
tggatgcatatgctgcagctgagttcgggcaaggtacaga

SEQ ID NO: 52 - DNA, 32 bp, Artificial Sequence
  Description of Artificial Sequence Synthetic primer
gaactctccaggcagtcaaaattcggttctag

SEQ ID NO: 53 - DNA, 33 bp, Artificial Sequence
  Description of Artificial Sequence Synthetic primer
tttgactgcctggagagttcggtaactcgattg

SEQ ID NO: 54 - DNA, 40 bp, Artificial Sequence
  Description of Artificial Sequence Synthetic primer
ccggttaacacgcgtacgtatgtggaattaccggaaggca

SEQ ID NO: 55 - DNA, 40 bp, Artificial Sequence
  Description of Artificial Sequence Synthetic primer
tggatgcatatgctgcagctagttggggcgaacaaggcta

SEQ ID NO: 56 - DNA, 33 bp, Artificial Sequence
  Description of Artificial Sequence Synthetic primer
atcggcctctatttatcatcccgctgcccttag

SEQ ID NO: 57 - DNA, 30 bp, Artificial Sequence
  Description of Artificial Sequence Synthetic primer
gatgataaatagaggccgattgatgaggat

SEQ ID NO: 58 - DNA, 40 bp, Artificial Sequence
  Description of Artificial Sequence Synthetic primer
ccggttaacacgcgtacgtagcaaataaacgaggcgtatc

SEQ ID NO: 59 - DNA, 50 bp, Artificial Sequence
  Description of Artificial Sequence Synthetic primer
attggtaccatggatgcatatgctgcagctacgctgctctaaataccttg

SEQ ID NO: 60 - DNA, 50 bp, Artificial Sequence
  Description of Artificial Sequence Synthetic primer
atatgagtaaacttggtctgacagttaccaccttcgccgatttggtttct

SEQ ID NO: 61 - DNA, 35 bp, Artificial Sequence
  Description of Artificial Sequence Synthetic primer
atctttcagctttggcattctaaatcccgttcgcc

SEQ ID NO: 62 - DNA, 35 bp, Artificial Sequence
  Description of Artificial Sequence Synthetic primer
acgggatttagaatgccaaagctgaaagatgaagg

SEQ ID NO: 63 - DNA, 51 bp, Artificial Sequence
  Description of Artificial Sequence Synthetic primer
cgtgcatcacgacaccgacaacctgcacattcgattctaatgagcctgttc

SEQ ID NO: 64 - DNA, 50 bp, Artificial Sequence
  Description of Artificial Sequence Synthetic primer
atatgagtaaacttggtctgacagttaccaatttattcaaactgcaaccg

SEQ ID NO: 65 - DNA, 36 bp, Artificial Sequence
  Description of Artificial Sequence Synthetic primer
aacgtgttgaagaataaaccagcatatccctagcag

SEQ ID NO: 66 - DNA, 37 bp, Artificial Sequence
  Description of Artificial Sequence Synthetic primer
gggatatgctggtttattcttcaacacgttaggtccc

SEQ ID NO: 67 - DNA, 50 bp, Artificial Sequence
  Description of Artificial Sequence Synthetic primer
cgtgcatcacgacaccgacaacctgcacatgggtgaatgggtggatgaat

SEQ ID NO: 68 - DNA, 45 bp, Artificial Sequence
  Description of Artificial Sequence Synthetic primer
tggatgcatatgctgcagcttctatcgaaagcaacaccaggaacc

SEQ ID NO: 69 - DNA, 44 bp, Artificial Sequence
  Description of Artificial Sequence Synthetic primer
cagcattgatgcgagcagtagatcatcttgcactaagccgcgta

SEQ ID NO: 70 - DNA, 44 bp, Artificial Sequence
  Description of Artificial Sequence Synthetic primer
tacgcggcttagtgcaagatgatctactgctcgcatcaatgctg

SEQ ID NO: 71 - DNA, 42 bp, Artificial Sequence
  Description of Artificial Sequence Synthetic primer
ccggttaacacgcgtacgtagtgatattcaggttgtggcggt

SEQ ID NO: 72 - DNA, 22 bp, Artificial Sequence
  Description of Artificial Sequence Synthetic primer
caaccgaacaccgaacatcctc

SEQ ID NO: 73 - DNA, 25 bp, Artificial Sequence
  Description of Artificial Sequence Synthetic primer
aagctgaagcatcttgctaatcagg

SEQ ID NO: 74 - DNA, 24 bp, Artificial Sequence
  Description of Artificial Sequence Synthetic primer
ggctgtgcttgataaagatcacgc

SEQ ID NO: 75 - DNA, 22 bp, Artificial Sequence
  Description of Artificial Sequence Synthetic primer
gacgattgcgcggctgtgttgg

SEQ ID NO: 76 - DNA, 21 bp, Artificial Sequence
  Description of Artificial Sequence Synthetic primer
caggatgcgtgccgaaataag

SEQ ID NO: 77 - DNA, 19 bp, Artificial Sequence
  Description of Artificial Sequence Synthetic primer
gaagaaccgacatggccgg

SEQ ID NO: 78 - DNA, 22 bp, Artificial Sequence
  Description of Artificial Sequence Synthetic primer
ggagtaggtgaaaggtgaaagg

SEQ ID NO: 79 - DNA, 19 bp, Artificial Sequence
  Description of Artificial Sequence Synthetic primer
ggcagggtaaggcggcgag

SEQ ID NO: 80 - DNA, 2992 bp, Artificial Sequence
  Description of Artificial Sequence Synthetic polynucleotide
gagttcgggcaaggtacagagtggaatttcaaccaagccttggattggtatgtattgcagcaccctagacaccaaggtttacatactctggtcaaagatcttaatcatttgtataaaaaccacccggcacttcaccaatacgacttcaatcacagcggctttgactggatcgattgccatgatgtcgaacaatcgattatcagctaccgccgcaaaggcaccaacgatgatttgatcatcatacttaacttcacaccgattgttagagaaaactatcatatcggcgtgcctttcgaaggggtttattttgaaatctttaattcagactctgcctattatgaaggcagcaatgtcggcaaccgcgagatactatcggaaccggagccttggatggggcaccagcaatcgatacacttgactttacctccgttaggcggaatcatcttgacccggcaaacgaaaaccataaacagctctgctcactaatatagggacctgcttgagaaagtgagcccagcgtgccgttgcgcaccgcaaattaactgacgacggcctagcttataaaaaattcgcttcgatatttatacgtaataggcgcaactacgcgagaaatgcccgcaaaatagaactaccgccgccccgaacgcaaacttaaactgatttttcaataatgtcggacgatcctactacagcagacagtgagccaataaacactcaatcggctattttggcgaaatccacctcggtaaactcaattctttaatatacctagaaccgaattttgactgcctttaaatttaattatgaaacgaattctttttgttaccagtgaagcacaccctttaataaaaaccggcggcttggcggatgtttcaagcagcttacctaaggctttggcggatctaggccaggatatccgtatcatcatacctaactatcaggctataaaaaaaaccgaaaacgtccaacatcggtgtacgctgagaatcaataattgcgatgtcaatattctcgaaacccgcttgccggaatcgaaagtaattgtatggctgatcgattgtccccagttttttgactacccgggcaatccttatcacgatgaatacggtaatgcctgggcaaacagtgccgatcgcttttcgctgttctgccgcataaccgtggaagtcgcgatgaacagagcctacttagattggaaaccggaaatcgtccactgcaacgactggcaaagcggcctggttcccgccttgttaacgctggaatacaatcgcccggcaaccatttttacgattcataacatggcctatcaagggatctttccctattcgacctacaatgcgcttaatcttccaagacaactttggaacccaaatgtacttgagtattacggcaacatgtcgtttttaaaaggcggcattgcttgctccgatcgagtaacaacggtaagtcctacctatgccaaagaaattcaatcatccgagtttggttacgggctagaaggcttgttaacccatcgcaaggaatttctatgcggtatactcaatggaaccgacaatgactggaatcccgaattcgacaacaacatcgtccagcgctacagctataaaacgctccatcacaaacaacccaataaggccgcattacaagaacggctctcactgcctctcgagccgtcgattcccattttcggattaattagccgtttggttgaacaaaaaggtatcgacttgctattagaatgcttgccggaattattaagtatgccagtacaatttgtattactcggtagcggcaacaaaagcttcgaacaacgtttatataattttgccgaagcctaccctgaaaaaatgtcgattactatcggctacgatgaacaattggcccattggatagaagcgggaagcgatattttcttgatgccatcgcgcttcgagccctgcggattaaaccaaatgtatagtcaacgctatggaactttgccgatcgtaaggaaaaccggcggcctagcagatactgtagtcgatgctctaccccatacgattgacaacaaaacggcaaccggaattacttttaatgaatcgacccccagttcgctactggaggccatcaagcgcgcgttgattttatacagagtcccggaaatttggacacaactgcaaaccaatgccatgaggaaagacttttcgtggaatagtagtgctaaacaatacttatcgctctatgaccatttgtaactcgattaaggatttggtcgtaaataacttccctgttttatggagagttcggtaactcgattggcaggtgtcggcggcagggcagattttttgctcctgcaaaatctgcattcatgccatccatggcaatcagtcgccgcggtcaagcctacacggacgtattcacggcgtcctgcaaagcgagttaccgaaccctcaacaaagctcatagttccaagacgttatttatcacgaaatcctaaggcgggctataactccaaaaagagttatttatcttttaccatgaagagcatgaaggacttgaagatacagcatttacaattaaatgtaagtgactgtccattaacagcctatgccatcattattgccacttcagtctcgccattgttttgatattttttagtcattatacctttcgcacttcaaatttctgcagcgcctaaagaggcaccgggttcctctcctaacgctaggtgagaagctgggctcacggcgtcctttgacgacccccgacgccggattttgatctacgatggttgggtacattagaaaacccttaggcgatttctttacaattaagacttttcgggtccttactctctaaacgggacatacgcttttttcgccggcgcgcattacttgctctttttttattcgaattagcgcttaatcttttgatatccagcttttgccgttgaacctcaatcgttttcaaatgttgaaaaatatcctccggcatgccttccggtaattccaca

SEQ ID NO: 81 - DNA, 4142 bp, Artificial Sequence; identical to SEQ ID NO: 43
  Description of Artificial Sequence Synthetic polynucleotide

SEQ ID NO: 82 - DNA, 3732 bp, Artificial Sequence; identical to SEQ ID NO: 44
  Description of Artificial Sequence Synthetic polynucleotide

SEQ ID NO: 83 - DNA, 3666 bp, Artificial Sequence; identical to SEQ ID NO: 45
  Description of Artificial Sequence Synthetic polynucleotide

SEQ ID NO: 84 - DNA, 4503 bp, Artificial Sequence
  Description of Artificial Sequence Synthetic polynucleotide
aaataccatgattccgaccggaggcgtcattccgatagcggtgtggggaacgcctgacggtcaacgtgcctacgtcgccaatctgttgggcacgccgccgctgttaagcagtttgacggtcatcgatattccgggcaagaagaaattgtccgacatcgacctggccgccgattacgatccgatatcgggaaaaatcagcggcgaggcatacgggttgttgccgatacaaacgccggttagtcctgacggtaagtatgtagtgacagcgaataccttgagcatgagcatcacgattgtcgataccgcaaccaataaagtcgtcaaaagcttgccttgcgaggcgggatgtcatggcgttcatttcggcatgaaaaaaggcggcgggtactatgcttatgtggccagcaagttcgctaataatctgttggttgtggatatggataagcttgaggtggccggcagtattttattggccgatgacaaagatagcagcataaaagcccataacggcatgggcgggcagggcgtgttgccgttgccgttggttgaacacggctatttggcgcaaaccttgaaattaagcggaaaaaaagagttgagtccgcaagtggaaggctggttgaagcaattgaccaaggaacaaaaagggatttgataaaacgattggccgggaaagtaattttcccggcagcattatgtattgattaagactctaaatatcttctgggctagtcaaatgcccgtcaaaggacgctatgaatactgacccacccgcgcttttgggcgtgcggtacatgacgaaaatcctggcgtaaagtcaagagcgtccggtcaagtgctcaggcgcgaaatcgtcgaccgcgatcgctttcattttcacgctctttcccaagctccagcttgggaaagacacaccggaagctccagcttcttgagaccgacacaacctccgcgcattctcaatcacccccgactcgctcgctcgttcaatctttcttgattatcgggaagctagagcttcctgaacaggttacccaagctggagcttgggtaacagcatattttagtttttgcgactacgactaaccccagcttggacacttggcttcggcaagctgaagcatcttgctaatcaggtgaagttaagtaccaccgctcaattgatcaggcgcgaaatcgtcgaccgcgatcgctttcagtttggcttgctcggctttttcgagtaatgcatattcgtccttgtcgatcaattccgatgccaaagcctgctgcagtagggatgacggcattcctttgggcaacgctccttgtttttgcgcggcgcggatttttgcctcggccggtgcggcctcgagtactgccttgaaggccgtctcgatgcggccggtaggatcgtccgggtcggtgttgatataaataccgtcggttaaacgttcgcgcgcagggttgtcgctcaacagcagttttgcgagagggcctgtcaaggtatcgcgcggcggcgcgcagggacttccgagcggatacaacagggcacgcagcaagccggccgaaagtgccgccggcagtagccgaagtgcttccgataacgattgttgggcatgatacaggctgtcttggcaggcccaatgcaacagcggcaggtcgtccgagggcgcgccttgattgtcgaaatgtttgagtacgcaggaacacaagtataagtggctcagcacgtcggccagccgcccggaaatgcgttccttgcgtttgagtcggccgcccagcgtcagcagcgcgacatcggcggccagcgcaaagcctgtgctaagccgcgtcaattgccggtaataacgcttcgtatgccggttaccgggcgcggcggtcagtcgcgaaccgccaacgccgaaccagagccctctaaggatgttccttagaaaaaaacggatatgccgccacactaccgagtcgaattcacgcaaggcttgggccgtatcggttcgattcaatgattgtatttcttgctgcacgaagggatggcagcgcaccgaaccttggccgaaaatcatcattgtgcgagttagaatgttcgcgccctcgaccgtgatgccgaccgggacgacttgatacaaacggccaattgcgttggaaggccccagacaaatgcccgaaccgccttgaatatccatgccgtcgttgacgatccgccgcatgccttcggtgagttggtatttcaatatcgccgagatcaccgccggtttttcgccggcatcgagcgccgcgcaggttacttggcgagcggcgtccatcagataggcgctgccggccatgcgcgccagcacctcttcaatgccttcgaaggctccgatcggcagattgaactggtgcctgatccgcgcataggcgccggtgttgcggcagatgaatttggccgcgccgacgctcagcgccggcagcgaaatcgcgcggccggtggcgaggcattgcatcaacattttccagccttgaccggcttgtgccgcgccgccgatgatccagtcgagcggaatgaaaacatccttgccccagttagggccgttctggaaggccgaatcgagcggaaagtggcggcggccgatcgaaatacccggcgtatcggtcggaattagcgcgacggtaatgccgatgtcttcggtttcgcccaacagccggtcgggatcgtaaagcttgaacgccaatcccagaaccgtggcgaccgggcctagcgtaatataacgcttctcccaattgagccgtatgcccaaaacttgttcattgccttcgaacgcgccgtaacagacgatgccggtatcgggcatcgcgccggcatcgctgccggccagcgggccggttagcgcgaatgccggtatttcgaggccgtcggctaaacgcggcagataatgattcttttgctgttcggtgccgtaggccagcaataatttggccggtcccaatgaattcgggaccatgaccgtgactgcggcggtcgtgctgcggctggcgagtttcatgacgatttgcgaatgcgcgaaatcggaaaactccagaccgccgtagcgtttcggtatgatgatgccgcaaaatttatgttgtttgatatagtcccagacttccttcggtaagtctcggcgcttgaaggtgatgtcccagtcgtcgagcatcgcgcacagggtttccacagttccgtcgataaaggcgcgttcctcgtcgctcagacgcgccgcaggcagcgccgcgattttggcccagtccggccggcccgtgaacagttcggcgtcccaccaggtattgcctgcttcgagcgcttcgcgttcggtttgcgacatcggcggcagattgcggcgcatgaccttgaacagcggccgggtcagtagactgcggcgtatcggagcataggcaacgagtccggctaggataagcccgctcgatacggcgataatgaattcggtcatggcttaattcctgttgctgtgtcagttcgtaggtttgggttctttatctaatttttcgatatgcaaaactccgttctggcaatgccggttccggagagggtgcggttgctcgatcgacagccgccgtcgagcctacatggacgtattcacggcgtcctgtcgggcgagtgaccgcaccctccgtcaccagactactttcatttgccggggcgatggccaggccttcatgaacgttacgttgctcgattgccccgtcagtttctcaacggcttgccggtttttagcatgtgttcgagccttgctgcagtgccgggttgcttgaccaggtgcaaaaaggcttgtcgttccaggttcaatatgtcttgttcgctaagcggatcgagggagtcggtgtctccgccgctcaatacgaaagccagttggccggcgatttcccggtcgtaggcggttgctttgcccgagagcgctaagttgcgcacggctatgtcaagcgccgcgcgggccgaagcgcccggtaaataataaacataaggttccggcggtcggtagtcggcgatcattgtcaggacgcgcgcttttgcgtcggccagcagacggtccttgttcattgtgatgccgtcgtgttcggataaatagagtaattccttcgccagcaaggccgatttggacgttttggcgagcgcgatggtctcgaaggcttgcgaaatcgccggcatcggtccgctcgggcggttaggcaagctaagccatcggcgcaataattccttacagccgccccagcccggcaccagaccgactccggtttcgacgaggccggtatacagttcggcatgggcttgcaccgcatcgcaatgcagcagaatttcgcagccgccgcccagcgccaaaccggacggcgcgccgacgaccgggaaaggcgcgtatttcaaggcttgataagtttgttgcccgagctcgacgattcgggcgacggtgtcccaatcttgccgcagcaatgccggggccagcagagttagattggccccggccgaaaaatgctcggcttcgttatggatga

SEQ ID NO: 85 - DNA, 3454 bp, Artificial Sequence
  Description of Artificial Sequence Synthetic polynucleotide
tctatcgaaagcaacaccaggaaccatgactaaatccaaatcttcgggcgttatttcctttccaggctccccccaccgttctttcggcggctcgagtatgccccacgtcccaggctgcaattcttgcaagtcctcgagcttccaaagacccaatcggttttgaccgtcggcatccttggtgcaataaggaacaactacctgcttatcgcccgacaactgttcgcgaaccctatcgcgcgtcctaacttcggagcgacaatgtaaataaaacatgaccgtcatggcccgccggtaagccgcatgctctaggaatttatcgattatctcccggctcaccgcatccttatctcgctgagcgttccgggcatcataagcttttcgccgctgttgggcttttgctgtttgcatcttggctcatctaattaaccttcgaatcggcatcgattataccggctgcaacgttaagaaacacagccattatcaaaccaatcacagcaaatcacctaatgatccaaaatataccaagcttaaatgctcagcaattcccaatttggagatcaaaaagggtgtggggtatgcttagcgaggatgtcggcagcaggacagatttttgctccatgcaaaatctgcattcacgccatccttggcgcttagctgccgccaagcccccatggatgggtttacggcgttcctcgacaggcataccccacaccctaaaatcggcgaaactgctcaaactaggaattgctgttaaatgctcgctagcaattcaacactcacaaaaagcgctaggcatttttaaattactttaatttcttttatactgaggtgctaaattttaaggaggggggaacccgtggaaaaaccatttattttacatatgctcacaaccgctaaaaacctaagcccgttcgacgtcaacatggcgctcgatgcgggttgggtttcggcaataccctatatcaatgtagaacccagcgaagtacgcggcttagtgcaagatgctattttttcacgcagtcctaaaagcttgaaaaaaaccggaatttttatcggcggcagggataccaaacaagcgctcgacatgcttaaaaccgccaaaaactcgatggtcccgccttttgaagtctccgttttcgccgacccaagcggcgcatttacgacggcagccggcatggtcgcggcagtcgaacacgaattgcaaaaaaccttcaacactactcttgaaggcaaaaacgtgcttgccttgggcggtaccggaccggtgggacaagcagccgccgttatcgccgctcaggccggcgcaaacgtaagaatcatcggacgccaactcgataaagccgagcgcattgccgagctttgcagcaatgaattcggcgaaggtactatccaaatcatggccggcgcggatgcggacaaagccgaattcatgaaaactgccgatgtcgtcttcgcgacaggtgctgccggcatcgaactcttaagtgccgagctgatcgcttcggcgccgcaattaaaagtcgcggccgatgtcaacgccgttccgccatcaggaatcgccggactcgacgcattccataacggtgcgcctctgcccggatcgaaaagcggcgcagtcggcattggcgcactggctatcggcaatgtgaaataccaagcacaaaacaaactgctcaagatcatgatcgacgccgaaaaaccggtctacttgcattttacccatgcattcgaagttgcccgggacttcatcaaagccaacgcttaacgacttaaattaagaggaaattcaatggagaagcgcagtatcctgcatatgctggacccgatgcccaataacagccctttcgatgtcaatatggctttggacgcgggattcgatgttttaataccttacagcaatgtcaaactggacggcgttcacggattgacgcaagatgccgttttctccagggggcccgccggtgtgaaacgcaccggtttgtttatcggaggaagggatatcggcttggcgatagacatgctgaacgccgcggaacaagcgatggtgccgccgtttgaaatctcggtattcgccgatcccagcggcgcattcacaaccgctgcagcattagtcggctgcgtcgaaaaagaactgaaaaccaagcataaccttgacttgaaaaacgccaaagcactggtgttcggcggcaccggtccggtcggcatagccacaggcgtcatcgcttcgctggaaggtgccgaaacaattttgatagatcatctttcagccgaaaccgccaacgatttggccaaagaatataatagacgcttcaaatctaatctaaaaggtgccagcgcttcgtctgacgaacaaaaagccgaactgatcgccgatgccgatatcattttctgtaccgcgaaagccggaatacaagtcatcaacggcaaagtactgagccatgccaaagcacttaaagtggccggcgatgtcaatgcagtccctccggccggtatcgaaggcataaaattaaacgatttcggccaacccatagcacatgcgccaaacgcggtcggtatcggcgcgttagccgtaggtaacgtcaaatatcaactacaacatctactgctcgcatcaatgctgacaactgataaaccattatttctagatttcagaaatgcttttgctaaagccagggaaattttataaacaacctttgtaactattcagcgcatgcggtaggttggtgtcaggcattgatttctaaagacgcgtgaatacatccctgtaagctctgactgcaacgtcctgttgcagacagccttataaatcaatgcctgacaccttctcatacataacttaggctgaataattaccaacctttctctagcccatgattccgtatcgctcaaatcgatgcggaatcgatataatcgaatttgatggcgagccgcgttaattcgacatcgttcttcacttttaatttatcgtaaagccgatagcgataagtgttaatcgttttatcgctaagcgtcagcatacccgacatttcttttatcgttttgccttgcaagattaatgagactacttccgactccctttgggagagctttgagaaaggagaatcattaccgggcaacttcatgaatgctaaattgttggctacctccgcgcacaaataacgctgcccggccataatttttcgaatcgcggcgaccatttcatcgaccggcgaacttttggaaataaagcctaacacccccattttcaaaagctgcaacggtatcggtccatcattacaaacagagagcacaataatcttcatgtccggattatcctgcagaattcgccgacaagcttcaacgccgccaataccgggcatgctaatatccatgaccaccacatcgggatgcagtgttgttaccgcctcgacgctctcttcaccggtttttgccaccgccacaacctgaatatcac

SEQ ID NO: 86 - DNA, 2882 bp, Artificial Sequence
  Description of Artificial Sequence Synthetic polynucleotide
cttcaagtcctgccaattcagattttttgttttgataaagctcgttatagtgccgccatattcggccttcgccgatttggtttcttaacgatggagggatttcaggatgcgtgccgaaataagcttgagtgacttgttggttcaactgttccacttcaagccgctctttcgccagcaattcccctttgatggcgataatgttgcttaaataatcctcgactttattgcggacctcgttgaggcgttggatatgaatcgtgtcttgctcggagatttcataaaatttgaaataagaaaacgacaccgatgcgcccaatgcaatcaacagcgaggcaagcacgctgaaataacgcaatcgattggcacgccaatgaatccctgcgatttcgaatgagcaaattacgatgatcgattgaatcgcgaccgtgatgatcagcgcgatccagggtgtgatgaaatgcgacatgccgtaatatgtcgtaaacccggaagccaaactgagcgcgagaacgatgggaatggtccatattcgcatcagactcacgaacatggtttgtatgatgttgatgagacgtcctaggccgccgcttttcatttttattattattcggttgtccatcgtctgactttatgttgttattcgagtaagcgtaatcctaaaaaatcagctggagcgcttggaatccgttcgtgctgagtccttcggactgcgctcaggagagccttgtcgaagtatgagcgaatccaagcgccacccattgggtgagcattctaaatcccgcccacccttcgactaggctcagggcgaacgggatttagaatgctcaactgattttttagcgttattgcggaaattagcaagattaaaaaaattctgctaatagtatcacattgaatttaaatagtttctattgaaaacaaatcaagcagttgtctttattccggctattgcgtgagggttttaaccgtatatttgagattgagtgtcaatcatgaaaatagccataatcggagccggccatgtcggttcgtcgttagcgtatgcgctggtgttgaaggggttgggcaattatttgttgttggtcaatcgtgatgcgaccaaagcgttgggaaatgcattagatttacagcatacactggctttttgcgaacgaccgatgcaaatcgagggcggcccgcttgaagaggcggtgggttgcgatatcgtcgcgatcaccgcttcgacgccgatggcaaaaggcatgacttctcgtatggaattaggcaaagcgaatgtcgagttattcaaatcgattatcccgatgctggccgaaaataatcctgaagctgtttttattatcatcaccaatccggtcgatgtgatgacctgctgtgctacgcggctttcgggattgccgtcgtcacaaatcgtcggaatcggcacgctggtcgactcggctcgattcagaaccttattgtccaaggccgagcagattcatcccgacgatttaagagcctatattcttggcgaacacggaccgaatcagtttccggtattcagccatgcttcggctggcggcgagctgataaccgatagtccgaggcatagagagatttttgaagaggtcaacaaggccggtttcgaggtctatcgattgaaaggttataccaattttgcgattgcttcagccgcttgcgaagtcatcagaaccatagttcacgacgaacatcgaacgatgccgctcagtacttgtttcgatgactggcaggggatcaaagacaattgtttcagcattccggtcgtactaggccgttccggtattatccggtatttgcagcccgacctaaacagtctggagatcgaggcgctggagcaatcggccagaacggtccgagccaatatcgatagtttattgcttggggtaggtgaaagaggaaaggtgaaaggtgaaagatgaaaggtgaaaggtgaaaggtgaaaggtgaaaggtgaaaggggaaaggtgaaaggggaaaggggaaaggggaaagaggaaagaggaaagaggaaagaggaaagaggaaagaggaaagaggaaagaggaaagaggaatggagtcttgtgggagcgatccccagatcgcgatttcgaagccggtaagcttggatgataataaagggtatcgaagtcacattggtcaaggttcctcgttaccgccgctccagcgtgggaatgcataccaaagctgaaagatgaagggagaaacgtggccgactcaagtatcgtgtttgtggcgcagtagcggccgaatatcgaaccacgttgcggtaactagataagctgcaaggcttgccgaagcgctgagccaggtgttgataagatagacttctgtcgcgaaatgatgggcaacatataaaattaaaccgaaggtgatatagccgatgatcttgcctagcctataagggaccggataatagcggcgcccgagtagccaagagatgacggccatgaatagatagcacgccagcgtcgcccaggcagaaccgtgataaccccaaatcgggatccaccaaatattcagccaagcggtcaaaccggcgcccgccaacgaaacccaagcgcccatgccggtgcggtcggtcaatttgtaccagatcgacagattgacatagatgccgagcagtagatttgcgatcaataagaccggcacaatatcgaggccttcacggaaatccgagccgataaaatattgaaagaaatcgatgaatagcgtcacgactagaaaaataaacacgccgaagattacaaaataattcatgatcaatgcataagacttttgtgcatcctcacgtccggctatgctaaagaaaaacggttcgccggcatagcggaaagcttgcacgaacaggctcattagaatcga

SEQ ID NO: 87 - DNA, 2901 bp, Artificial Sequence
  Description of Artificial Sequence Synthetic polynucleotide
atttattcaaactgcaaccgaacaccgaacatcctcacggtttaagtcatcgcgaattcgatagcctatttacggtcgacaaaccggtcattttcaatttccacggctatccctggttgattcacaaattggcctatcgcttcaagaatcatgaaaatctgcatgtgcggggctataaagaacgcggcaatatcaatacacccatggagcttgcgattctgaacgaagtcgatcgcttcaatctagtcatcgatgtcatcgaccgggttccgaaattgcaaatcagggcggcccatctcaaggagctcatgaaaaacgaaatcatcgagaatcttcgttatgcgcatcaacacggcaccgataaacccgaaatcaccggttggcaatggccgttttaacagtaatcctgaaaagagcagttggcctatgttgtagaccgttcgtgctgagccaagtcgaagcatgaagggtctacaatactttcaccggactggtgaagcctcaaactaccgttcacccttcgacaaggctctcctgagcgtagccgaaggggctctcaacagctctttttagggtaatgacaaacctgattagcaagatgcttcagcttgctgaagccaattgtccgagcagaggcataagtatctacacaatttttctgcgggacgggttatttaacccgtccccaacgtttcggtttgccctaaacatttcggctaacttcggccaaagtcaaaacgtttaggacgggattacaaaccccgtcctgctagggatatgctggtttttgggctttagctgaagaaacttgctaatcagatttttatttgtaaaacggcgtttaccaaaatcattttaaggatatgacaataccaaacggcaatattctcgttatcaacagcggcagttcctcaattaagtatcgattgattgctctgccgcaagagcaggtactggcagacggcttgctggaacgcatcggagaccagaaaagcaggatcattcatagagccgacgattcgggtcgtttatatgaaatcaagcagtcggtcgccgctgccgatcatcaccaagccttcaaggcagtcttcgagattttgggcgaaaattgcccggtcgatgcaataggccaccgcgtcgtacatggcggtgaccggttctccggccctgccttggtcgatgacgatacgatagcgtcgatgcgcgcgctctgccgaatagcgccgctgcacaatccggtaaacttgcttggcatcgagagctgcttggctcatttcccgggagtacctcaggtggcggtattcgatacggcatttcaccaaacgatgccgccccacgcctatcgttatgcgattccggaaacttggtatagcgattacggcatacgccgattcggttttcacggcacctctcatcattatgtggcgagacgggccgccgaatttatcgggaaacctttcgatcgcagccatctgattactttgcatttgggcaatggcgcgagcgcaacggcgattgcaaacggccgttccatcgatacatcgatgggatttacgcccttggaaggtcttgtgatgggcacgcgtagcggcgatttggacccggcaataccgctatttgtcgaacaaaccgaaaataccgacacgaacgtaatcgaccgggcattgaaccgcgaatccggattaaaaggcttatgtggtaccagcgacttaagaaccgtgctcgaacaaacaaatgcaggcgatgaacgagcccgcttggctctcgatctgtattgctatcgaatcaagaaatatatcggcgcttactacgcggtactcggcgaagtcgacgctctggtttttaccggcggcgtcggcgaaaacgcggccgaagtgcgccgtttagcctgcgaaggcctgtcgcgtctcggcatcactattgatgaagcggccaatagcgacgtgaccggagctatcgccgaaatcggacatcccgaaagtcgaacccgtattctagtcattaaaactgacgaagaattgcaaattgcccgggaaaccatggttgtacttgataaagatcacgcatgaaaatatttagactgaacggtttaccgggcacccaacaccatggaaaaattcttcaacacgttaggtcccataaaagcggccatgcattattatattccgccgctggatcgcatcgattgacctaaaattcaacacatagcttacgtaggatgggtagaggcggaagccgaaacccatcgtttcaaccccaccaccgaatctctcgccgaacggatttttaaatccagccgcaatagtttcctgccgtttgcggggtctccatgatgggtttccgcttcgctcctacccatcctactgcctctttcatctttcccctttcttctttcctctttaacctaaatcaaacatctccccataaccctcccgataagtcgggtatttaaaccgaaaccccaaggtcttcaagcgactattccgacatcgtttattttgatctggaacattatcggcaggttttgcgatcggcggttggctgttcatttgttgagcgagccatgaaatcacgtcccacatcggcgcgggattgtcgtcgctggccaaataaaaattgtccagatgccctccttcgatatgctttttcattaaaaaagccaacacagccgcgcaatcatcttggtgaattcggttcgtgtaataaggcgggtcgacttgaatggcggcttgccgcgacgccatttgtaaaaggcgctctctacccgggccgtaaataccggaaaaccgaacgacgaccgcgccgggatcatgctccaacagcttttgctcggccttacggatgatttgcccggttacggtattcggttgggcatccgaagattcatccacccattcaccc

SEQ ID NO: 88 - DNA, 2121 bp, Artificial Sequence; identical to SEQ ID NO: 47
  Description of Artificial Sequence Synthetic polynucleotide

SEQ ID NO: 89 - DNA, 18991 bp, Artificial Sequence; identical to SEQ ID NO: 50
  Description of Artificial Sequence Synthetic polynucleotide

(19) United States
(12) Patent Application Publication
(10) Pub. No.: US 2016/0237442 A1
(43) Pub. Date: Aug. 18, 2016

(54) MODIFIED GROUP I METHANOTROPHIC BACTERIA AND USES THEREOF

(71) Applicant: UNIVERSITY OF WASHINGTON THROUGH ITS CENTER FOR COMMERCIALIZATION, Seattle, WA (US)

(72) Inventors: Aaron Puri, Seattle, WA (US); Mary E. Lidstrom, Seattle, WA (US); Marina Kalyuzhnaya, Seattle, WA (US)

(73) Assignee: UNIVERSITY OF WASHINGTON THROUGH ITS CENTER FOR COMMERCIALIZATION, Seattle, WA (US)

(21) Appl. No.: 15/030,144

(22) PCT Filed: Oct. 20, 2014
(86) PCT No.: PCT/US14/61363

§ 371 Date: Apr. 18, 2016

Related U.S. Application Data
(60) Provisional application No. 61/893,071, filed on Oct. 18, 2013.
(60) Provisional application No. 62/004,036, filed on May 28, 2014.

(51) Int. Cl. C12N 15/52; C12P 7/40; C12P 7/64

(52) U.S. Cl. CPC C12N 15/52; C12P 7/64; C12P 7/40

(57) ABSTRACT

Described herein are methods and compositions relating to engineered methanotrophic bacterium and the production of carbon products from methane.

### CROSS-REFERENCE TO RELATED APPLICATIONS

This application claims benefit under 35 U.S.C. §119(e) of U.S. Provisional Application Nos. 61/893,071 filed Oct. 18, 2013 and 62/004,036 filed May 28, 2014, the contents of which are incorporated herein by reference in their entirety.

### GOVERNMENT SUPPORT

This invention was made with federal funding under Grant Nos. DE-AR0000350 awarded by the Department of Energy. The U.S. government has certain rights in the invention.

### FIELD OF THE INVENTION

The field of the invention relates to the modification of methanotrophic bacteria and uses thereof.

### BACKGROUND

Methane (e.g., CH₄) is a potent natural greenhouse gas that contributes 18% to the Earth's warming In terms of atmospheric radiative forcing, each molecule of methane is approximately 80 times more potent than carbon dioxide (CO₂) at a 20 year timescale. Methane is emitted from a variety of natural and anthropogenic sources: human-related activities such as fossil fuel production (e.g. underground coal mining, oil and gas production), agriculture (e.g. enteric fermentation in livestock, manure management, and rice cultivation), landfills, and municipal wastewater. As the understanding of global climate change has increased, a multitude of research activities have been directed towards reducing methane emission and optimizing low-cost methane mitigation technologies.

Natural gas and its renewable alternative biogas are becoming the most prominent sources of fuel that can substitute for oil in the future. Methane is one such natural gas. Effectively utilizing methane as an efficient source of fuel, however, requires its conversion into a liquid form.

A number of advanced gas-to-liquid conversion technologies are currently being developed. For example, three potential routes for converting natural gas include: direct, indirect, and physical conversion. Direct conversion focuses on the chemical transformation of natural gas to ethane, ethylene, acetylene, or methanol. Indirect conversion methods concentrate on the production of syngas (CO, CO₂, and H₂), which is subsequently converted to liquid fuels. Physical conversion techniques are centered on the conversion from natural gas to liquefied natural gas. All three approaches are complex and multistep with a high capital cost. As a result, only a small fraction of natural gas reservoirs (<30%) and virtually no biogas sources are large enough to commercially justify the expense of installing conversion and transportation infrastructure.

Scalable, movable, low complexity (preferably low temperature/low pressure), low environmental impact, and low-cost gas-to-liquid conversion technology has a high potential to transform the current landscape of the energy/raw material market. A number of advanced smaller scale gas-to-liquid conversion technologies are currently under development. No viable processes, however, have been developed to date. Accordingly, while a number of strategies for efficient utilization of remote/small methane sources have been proposed, venting and gas flare remain the most common way of handling waste-derived methane. Such venting and flaring represents both a significant loss in carbon/energy resources and an increase in harmful greenhouse emissions.

### SUMMARY

Aerobic methanotrophic bacteria (methanotrophs or MB) are a highly specialized group of microbes utilizing methane (e.g., CH₄) as a sole source of carbon and energy. MB function in nature by eliminating methane and retaining it in the carbon cycle. The biotechnological potential of MB has been of broad interest, ranging from bioremediation to large scale bacterial protein production.

Provided herein are modified MB that produce bio-fuel and a number of other value-added chemicals directly from methane including currently wasted/flared sources of methane (natural gas or biogas). The modified MB have the catalytic versatility to assemble complex biochemicals from methane. The assembled biochemicals can be converted into diesel range hydrocarbons and/or polymer/plastic materials.

### BRIEF DESCRIPTION OF THE FIGURES

FIG. 1A depicts an overview of the central carbon metabolism of Methylomicrobium (M.) buryatense 5GB1 (5GB1). FIG. 1B depicts predicted fluxes for Methylmicrobium alcaliphilum (20Z). Predicted fluxes are given in mmol g⁻¹ h⁻¹. The width of the arrows is proportional to the flux value.

FIG. 2. 5GB1R glycogen synthase (glgA1) gene (deleted region in modified form of strain underlined) (SEQ ID NO: 42); as used herein, “R” signifies rifamycine resistance as compared to wild-type strains.

FIG. 3. 5GB1R glycogen synthase (glgA2) gene (deleted region in modified form of strain underlined) (SEQ ID NO: 43).

FIG. 4. 5GB1R phosphoenolpyruvate (PEP) synthase (pps) gene (deleted region in modified form of strain underlined) (SEQ ID NO: 44).

FIG. 5. 5GB1R sucrose-6-phosphate synthase, (sps) gene (deleted region in modified form of strain underlined) (SEQ ID NO: 45).

FIG. 6. 5GB1R Acyl-CoA dehydrogenase, fadE gene (deleted region in modified form of strain underlined) (SEQ ID NO: 46).

FIG. 7 M. alcaliphilum 20ZR (20Z/20ZR) sucrose-6-phosphate synthase (sps) gene (deleted region in modified form of strain is underlined) (SEQ ID NO: 47).

FIG. 8. 20ZR, Alpha amylase (ams) gene (deleted region in modified form of strain underlined) (SEQ ID NO: 48).

FIG. 9. 20ZR, H4MTP-pathway gene mutation, Δmch-fae gene (deleted region in modified form of strain underlined) (SEQ ID NO: 49).

FIG. 10. 20ZR, glycogen biosynthesis pathway (deleted region in modified form of strain underlined) (SEQ ID NO: 50).

FIG. 11. Genotyping of kanamycin-resistant modified strains using internal primers to detect integrated allelic exchange plasmid (single crossover). Lanes 2, 4, 5, 8, 12: Allelic exchange plasmid positive controls. 1: glgA1 knockout strain AP1. 3: glgA2 knockout strain AP2. 6,7: Clones of fadE single crossover mutants. 9,10: Clones of pps single crossover mutants. 11: pps knockout strain AP3. 13,14: Clones of sps single crossover mutants. Strains were also genotyped to confirm the presence of the integrated kanamycin cassette between flanking regions (double crossover, not shown).

FIG. 12. Scheme for selecting an improved genetically tractable variant of Methylomicrobium buryatense 5GB1. During the first round of selection, a variant of the IncP-based broad host range plasmid pVK100 was used (Knauf & Nester, 1982). During the second round, a variant of the trimmed IncP-based broad host range plasmid pCM66 was used (Marx & Lidstrom, 2001). Following selection of kanamycin-resistant colonies in each round, clones were passaged on NMS2 plates with no kanamycin 2-3 times before checking for loss of plasmid as shown by kanamycin sensitivity.

FIG. 13. Comparison of mating efficiencies in Methylomicrobium buryatense 5GB1 and the more genetically tractable variant 5GB1S using the IncP-based broad host range plasmid pVK100 (Knauf & Nester, 1982).

FIG. 14. Comparison of mating efficiencies in Methylomicrobium buryatense 5GB1 and the more genetically tractable variant 5GB1S using the trimmed IncP-based broad host range plasmid pCM66 (Marx & Lidstrom, 2001).

FIG. 15. Comparison of sequencing reads of 5GB1 and 5GB1S, where 5GB1S shows no sequencing reads mapped to the plasmid endogenous to 5GB1.

FIG. 16. Comparison of relative genetic tractability of Methylmicrobium buryatense 5GB1 and 5GB1S.

FIG. 17 depicts a graph of ¹³C kinetic analysis of flux to pyruvate in the indicated mutants.

FIG. 18. 5GB1 glycogen synthase (glgA1) gene (deleted region in modified form of strain underlined).

FIG. 19. 5GB1 glycogen synthase (glgA2) gene (deleted region in modified form of strain underlined).

FIG. 20. 5GB1 phosphoenolpyruvate (PEP) synthase (pps) gene (deleted region in modified form of strain underlined).

FIG. 21. 5GB1 sucrose-6-phosphate synthase, (sps) gene (deleted region in modified form of strain underlined).

FIG. 22. 5GB1 Acyl-CoA dehydrogenase, fadE gene (deleted region in modified form of strain underlined).

FIG. 23 5GB1, H₄MTP-pathway gene mutation, ΔmtdB gene (deleted region in modified form of strain underlined).

FIG. 24. 5GB1, lactate dehydrogenase deletion, Δldh gene (deleted region in modified form of strain underlined).

FIG. 25. 5GB1, acetate kinase deletion, Δack gene (deleted region in modified form of strain underlined).

FIG. 26. 20Z sucrose-6-phosphate synthase (sps) gene (deleted region in modified form of strain is underlined).

FIG. 27. 20ZR, glycogen biosynthesis pathway (deleted region in modified form of strain underlined).

### DETAILED DESCRIPTION

### Methanotrophic Bacteria (MB)

Methanotrophs are a highly specialized bacterial group utilizing methane (e.g., CH₄) as a sole source of carbon and energy. Obligate aerobic MB can be separated into three major groups. Group I MB are gammaproteobacteria that have stacked membranes built mostly of C16 fatty acids. Group I MB use the ribulose monophosphate (RuMP) cycle, which converts formaldehyde (CH₂OH) into multi-carbon compounds for building cell biomass. The majority of Group I methanotrophs are grouped into the Methylococcaceae family. Group II MB are alphaproteobacteria, contain rings of particulate methane monooxygenase (pMMO)-harboring membranes at the cell periphery, generally accumulate C18 fatty acids, and use the serine cycle for converting formaldehyde into biomass. Methylocystis and Methylosinus species are typical representatives of Group II MB. Group III MB do not produce intracellular membranes (ICM), display a low growth rate, and assimilate carbon through the Calvin-Benson-Bassham (CBB) cycle. Group III MP are represented by methanotrophic Verrucomicrobia. MB are unique in their ability to synthesize lipids from methane. Group I MB particularly have relatively high lipid/biomass content (as high as 22% total lipid in 5GB1) as a result of formation of extensive intracellular membranes (ICM). Accordingly, Group I MB are the focus of the current disclosure.

The biotechnological potential of MB has been of broad interest for decades, ranging from bioremediation to biocatalysis, such as the production of specialty chemicals, polymers, and food-grade chemicals (epoxides, poly-β-hydroxybutyrate, ectoine, and astaxanthin) or single cell protein. In the past, major efforts have taken place in the UK, Denmark, and USSR to develop industrial-scale processes for converting methane into single cell proteins (SCP) using Type I MB for high yield biomass production. For example, in the USSR, production fermentors (up to 750 m³ working volume), were used to produce about 36,000 tons of dry biomass a year at a cell density of 20 g/L. SCP from a methanotrophic consortium (BioProtein, Norferm Danmark A/S), is a commercially established product in Denmark with the isolated protein product being approved by the European Union for use as a feed for salmon, calves, and pigs.

Despite the long-term interest in using MB for bioprocess applications, the majority of these efforts have not been successful due to the lack of a robust strain suitable for both metabolic engineering and process conditions. Further, little is known about the global metabolic and regulatory networks of methane utilization in MB. A few SCP strains such as Methylococcus capsulatus and Methylomonas spp have served as useful models for research and industrial process development.

Methylomicrobium buryatense strain 5GB1 (“5GB1”) and Methylomicrobium alcaliphilum strain 20Z (“20Z”) grow extremely well in pure culture, and are resistant to a variety of water chemistries and contaminants as well as typical gaseous impurities found in natural gas. All of these parameters make 5GB1 and 20Z particularly well suited for developing a modular system to be reproduced at remote stranded natural gas sources, making use of local available water resources (including freshwater, brackish, or marine water).

As an example, unmodified 5GB1 naturally produces up to 20% of dry cell weight as total lipids, which makes it useful as a biomass for a biofuel. Lipids are complex molecules typically defined in the context of biofuel development as fatty acid esters or isoprenoid-type molecules present in biomass. The proportion of the cells that are fatty acids reflect the overall ‘biofuels potential’ of the biomass. In the case of 5GB1, the lipids are mainly found in the cell membranes. Of the 20% lipid biomass, >80% are present as free fatty acids (FFA) and phospholipids, most commonly of the phosphatidylethanolamine and phosphatidylglycerol classes. As an example, carbon conversion efficiency (CCE) is 60% and standard batch culture cell density is 3 g CWW/L. Unmodified 20Z has a similar baseline CCE.

### Modification of Methanotroph Bacteria

Provided herein are modified Group I methanotrophic bacteria that can convert methane into commercially valuable products. As used herein, “methane” includes all C1 carbon substrates and particularly includes, without limitation, any carbon-containing molecule that lacks a carbon-carbon bond such as methane, methanol, formaldehyde, formic acid, formate, methylated amines (e.g., mono-, di-, and tri-methyl amine), methylated thiols, and carbon dioxide. Appropriate Group I strains for modification include without limitation, 5GB1, 20Z, Methylomonas sp. LW13 (LW13), Methylomonas MK1 (“MK1”) and Methylomonas sp.11b (“sp.11b”). In some embodiments, methane refers to CH₄. In some embodiments, the methods and compositions described herein relate to the conversion of CH₄.

CH₄ can be obtained from a variety of sources, including, by way of non-limiting example, natural gas, fracking, landfill emissions, livestock facilities, Fischer-Tropsch processes, coal seam gas, and the fermentation of wastewater sludge, manure, and/or solid waste. It is specifically contemplated herein that the CH₄ used in the methods described herein can be gaseous methane from one or more of the foregoing sources.

In particular embodiments, the modifications result in Group I methanotrophic bacteria with up-regulated lipid content, up-regulated methane flux, up-regulated carbon conversion efficiency, up-regulated carbon conversion into cellular sugars, up-regulated conversion of formate into formyl-H₄folate; up-regulated conversion of oxaloacetate, up-regulated regeneration of glyoxylate, up-regulated conversion of formate into acetyl-CoA, up-regulated flow of carbon into pyruvate, down-regulated sucrose synthesis, down-regulated conversion of pyruvate into phosphoenolpyruvate (PEP), down-regulated conversion of formate into carbon dioxide, down-regulated cyclic oxidation of formaldehyde, down-regulated carbon conversion into glycogen and/or down-regulated lipid degradation pathways.

The described modifications can be achieved by up-regulating activity of pathways that convert formate into cellular carbon, down-regulating the H₄MPT pathway for formaldehyde oxidation and/or down-regulating the pentose-phosphate pathway for formaldehyde oxidation. The described modifications can also be achieved by up-regulating methane monooxygenase, pyruvate kinase, acetyl-CoA carboxylase, formyltetrahydrofolate synthetase/ligase, methylenetetrahydrofolate dehydrogenase, formyltetrahydrofolate cyclohydrolase, and/or PEP carboxylase and/or down-regulating formate dehydrogenase, formaldehyde activating enzyme, methenyltetrahydromethanopterin cyclohydrolase, methyl-enetetrahydromethanopterin dehydrogenasegluconate-6-phosphate dehydrogenase, glucose-1 -phosphate adenyltransferase, glycogen synthase, ADP-glucose pyrophosphorylase, glycogen branching enzyme, PEP synthase and/or sucrose-phosphate synthase.

The described modifications can also be achieved by up-regulating pmoCAB, ftfL, mtdA, fch, ppc, pyk1, pyk2, accABC and/or tesA and/or down-regulating fae, mch, mtdB, gnd, glgA, glgB, glgC, pps, fadE, fdsABCD, fdhAB and/or sps. In some embodiments, the described modifications can also be achieved by up-regulating pmoCAB, and/or tesA and/or down-regulating mtdB, gnd, glgA, glgB, glgC, pps, fadE, ack, ldh and/or sps.

As used herein, “up-regulation” or “up-regulated” means increasing an activity within a bacterial cell. The activity can be the actions of one or more metabolic pathways or portions of metabolic pathways within a bacterial cell. An up-regulation of one activity can be caused by the down-regulation of another. Alternatively, an up-regulation of an activity can occur through increased activity of an intracellular protein, increased potency of an intracellular protein or increased expression of an intracellular protein. The protein with increased activity, potency or expression can be encoded by genes disclosed herein.

To cause an up-regulation through increased expression of a protein, the copy number of a gene or genes encoding the protein may be increased. Alternatively, a strong and/or inducible promoter can be used to direct the expression of the gene, the gene being expressed either as a transient expression vehicle or homologously or heterologously incorporated into the bacterial genome. In another embodiment, the promoter, regulatory region and/or the ribosome binding site upstream of the gene can be altered to achieve the over-expression. The expression can also be enhanced by increasing the relative half-life of the messenger or other forms of RNA. Any one or a combination of these approaches can be used to effect upregulation of a desired target protein as necessary for the methods and compositions described herein.

As used herein, “down-regulation” or “down-regulated” means any action at the metabolic pathway, protein or gene level that results in: a decrease in the activity of a metabolic pathway or a portion thereof; a decrease in activity of a protein; elimination of a protein's activity, translation of an incomplete protein sequence; incorrect folding of protein; reduced transcription of a gene; incomplete transcription of a gene, interference with an encoded RNA transcript, or any other activity resulting in reduced activity of a pathway, protein or gene. An increase in the expression of a pathway inhibitory protein or signaling molecule can also result in pathway downregulation.

A gene can be down-regulated for example by insertion of a foreign set of base pairs in a coding region, deletion of any portion of the gene, or by the presence of antisense sequences that interfere with transcription or translation of the gene. In another embodiment, down-regulation includes elimination of a gene's expression (i.e. gene knockout). As used herein, the symbol “Δ” denotes a mutation in the specified coding sequence and/or promoter wherein at least a portion (up to and including all) of the coding sequence and/or promoter has been disrupted by a deletion, mutation, or insertion. In another embodiment, the disruption can occur by optionally inserting a nucleotide or polynucleotide molecule into the native gene sequence whereby the expression of the mutated gene is down-regulated (either partially or completely). Any one or a combination of these approaches can be used to effect downregulation of a desired target protein as necessary for the methods and compositions described herein.

“Up-regulation” and “down-regulation” can be measured against a control condition including, without limitation, relative to the activity of an unmodified bacterial strain of the same species.

Embodiments disclosed herein include modified Group I MB that achieves precursor lipid content of 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%, and/or a carbon conversion efficiency of 60%, 61%, 62%, 63%, 64%, 65%, 66%, 67%, 68%, 69%, 70%, 71%, 72%, 73%,74%, 75%, 76%,77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Embodiments disclosed herein include modified Group I MB that achieves precursor lipid content of 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50%, and/or a carbon conversion efficiency of 60%, 61%, 62%, 63%, 64%, 65%, 66%, 67%, 68%, 69%, 70%, 71%, 72%, 73%,74%, 75%, 76%,77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. In particular embodiments, each of these numerical values is a minimum value (e.g., at least 20%, at least 24%, at least 25% . . . at least 98%, or at least 99%). In additional embodiments, the modified Group I MB utilizes a variant of the RuMP pathway which involves glycolysis. In additional embodiments, the modified Group I MB are modified 5GB1, modified 20Z, modified LW13, modified MK1 or modified sp.11b. In a particular embodiment the modified strain is 5GB1 with a lipid content of at least 22% and a carbon conversion efficiency of at least 65%. In particular embodiments, each of these numerical values is a minimum value (e.g., at least 23%, at least 24%, at least 25% . . . at least 98%, or at least 99%). In additional embodiments, the modified Group I MB utilizes a variant of the RuMP pathway which involves glycolysis. In additional embodiments, the modified Group I MB are modified 5GB1, modified 20Z, modified LW13, modified MK1 or modified sp.11b. In a particular embodiment the modified strain is 5GB1 with a lipid content of at least 34% and a carbon conversion efficiency of at least 70%.

As used herein, “genetically tractable” is the ease or rate at which genetic material can be received and stably maintained by a recipient strain from a donor strain. Genetic tractability can be defined, in part, by the number of recipient bacteria that receive and maintain genetic material from the donor strain.

As used herein, “relative genetic tractability” is the number of colonies obtained of the modified strain divided by the sum of the number of colonies of modified strain and the number of colonies of the remaining unmodified strain. In certain embodiments of the present disclosure, the relative genetic tractability of the modified methanotrophic bacteria is between 0.1-1.0. In certain further embodiments, the genetic tractability is preferably between 0.5 and 1.0.

### Metabolic Networks

All known aerobic MB use methane monooxygenase (MMO) for the first oxidation step that converts methane into methanol and the cells further oxidize methanol to formaldehyde, formate, and then into carbon dioxide. Two isoenzymes of MMO are known: soluble methane monooxygenase (sMMO) and membrane bound (or articulate) methane monooxygenase (pMMO). pMMO has a higher affinity for methane compared to sMMO, and pMMO is the most efficient system for methane oxidation.

Group I MB also use a more efficient pathway for assimilation of the C1 unit into biomass than other MB. In these strains, formaldehyde is directly assimilated to form central metabolic intermediates by the assimilatory RuMP pathway. This pathway allows Group I MB to generate the highest cellular yields from methane.

Genomics, transcriptomics, and metabolomics were used to reconstruct the metabolic networks of 5GB1. In addition, ¹³CH₄-based analysis was used to measure fluxes through the metabolic networks of strains 20Z and Methylomonas spp. LW13 (“LW13”). The results showed that these bacteria utilize a metabolic pathway not yet observed in Group I MB, a theoretically efficient variant of the RUMP pathway involving glycolysis. In this pathway, the conversion of 9 mol of formaldehyde to a three-carbon intermediate requires 3 mol of PPi, which produces both 3 mol NADH and 2 mol of ATP:

9HCHO+3NAD+3PPi+2ADP→3phosphoglycerate+3NADH+2ATP+4Pi

This equation was used to calculate the cell yield on methane, (carbon conversion efficiency), with a result of 65% (Table 1). From a catalytic standpoint, a direct high flux of C1-units into the glycolytic pathway opens new possibilities for metabolic engineering in MB and permits the use of this platform for technological applications.

TABLE 1
Summary of major advantages of Group
I MB Utilizing Modified RuMP Pathway

| Parameters | Comment |
|---|---|
| Genomic | 1.1 |
| Genomes | A number of genome sequences are available |
| Tools | A set of custom vectors are available |
| Metabolic | Central metabolism is modeled and refined |
| reconstruction/FBA | with respect to metabolic data/flux analysis |
| Metabolic | 1.2 |
| pMMO V_{max (app)}/Kₘ₍ₐₚₚ₎ | 220 mU (e.g. 185 mU)/0.2 × 10⁻⁶ M |
| Growth parameters | 0.23 h⁻¹/3 h/65% (calculated; |
| (μₘₐₓ/T_{d}/CCE) | observed is 60%) |
| Fermentation | Natural ability to produce, e.g., formate, |
| | acetate, succinate and lactate |
| High lipid content | e.g., 15-22% of cell dry weight (C16) |

Batch culture data.
pMMO—particulate methane monooxygenase; mU, nmol min⁻¹ mg protein⁻¹.

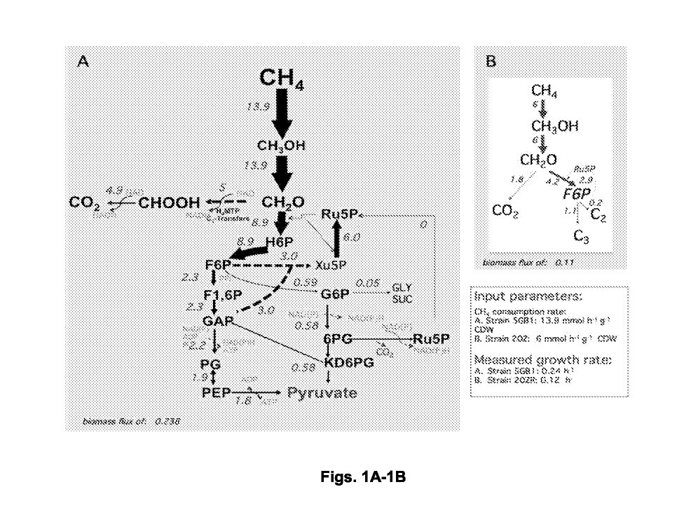

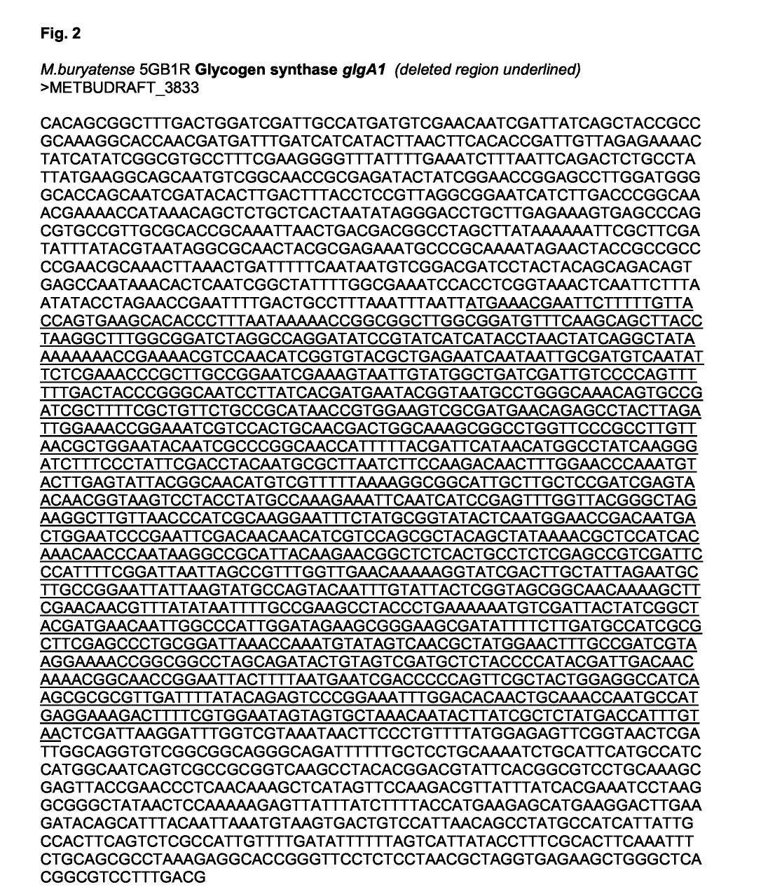

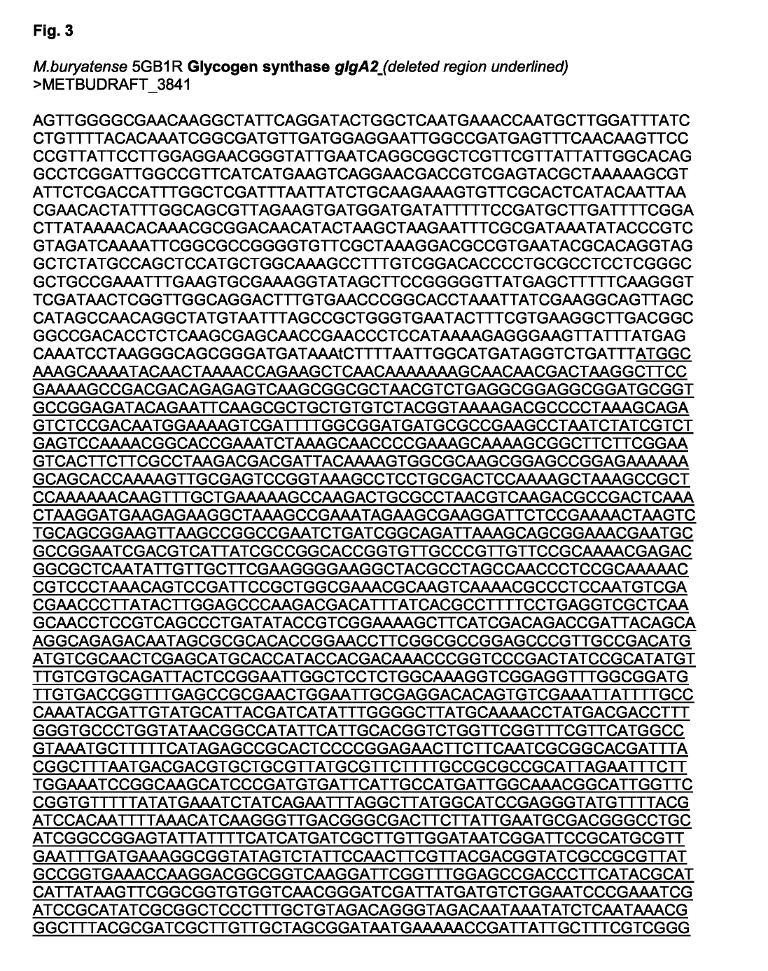

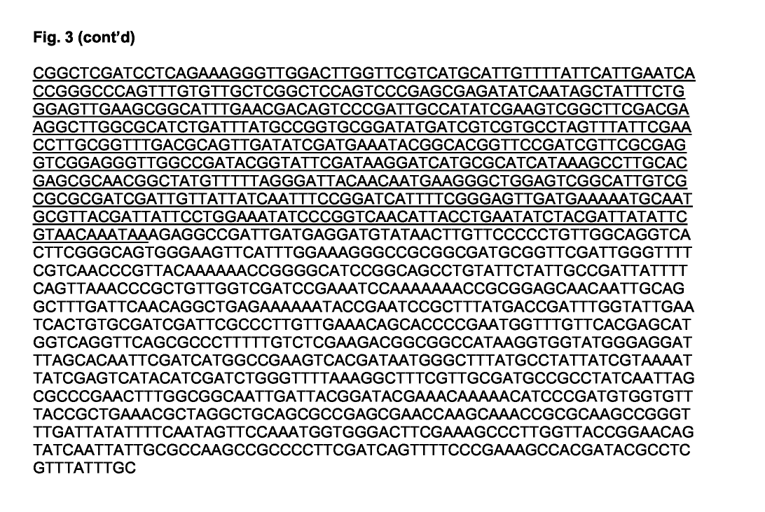

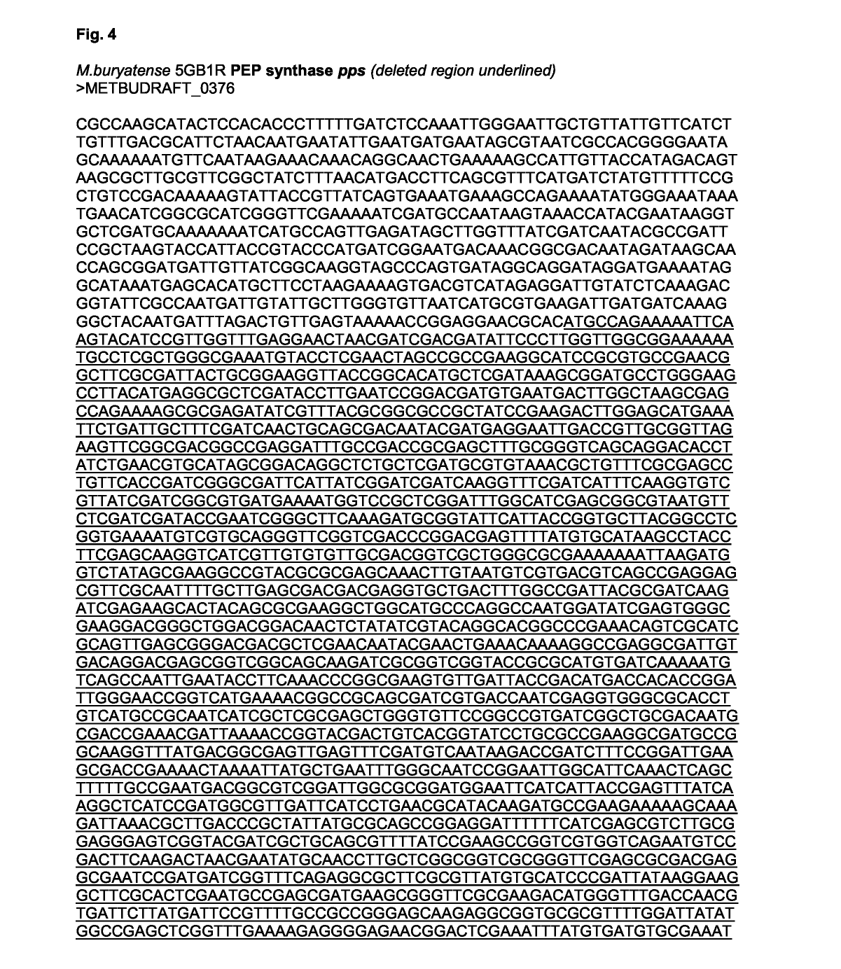

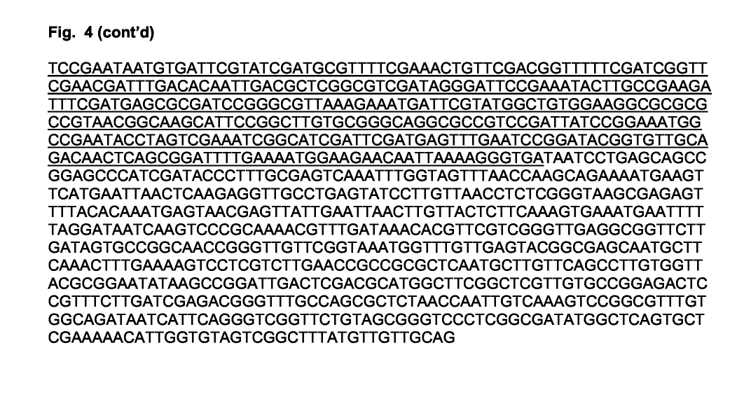

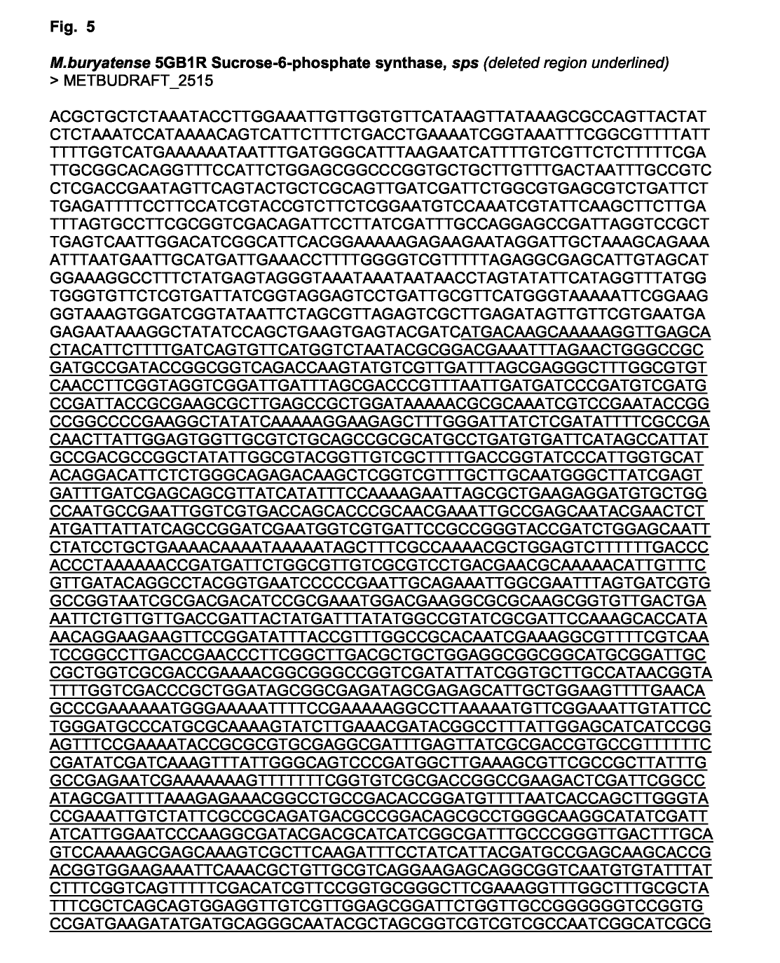

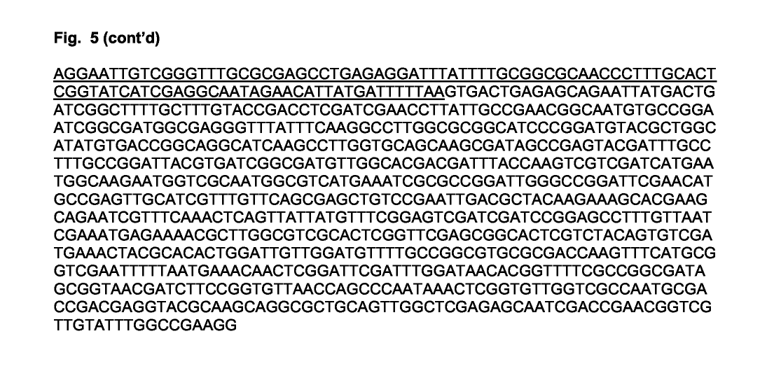

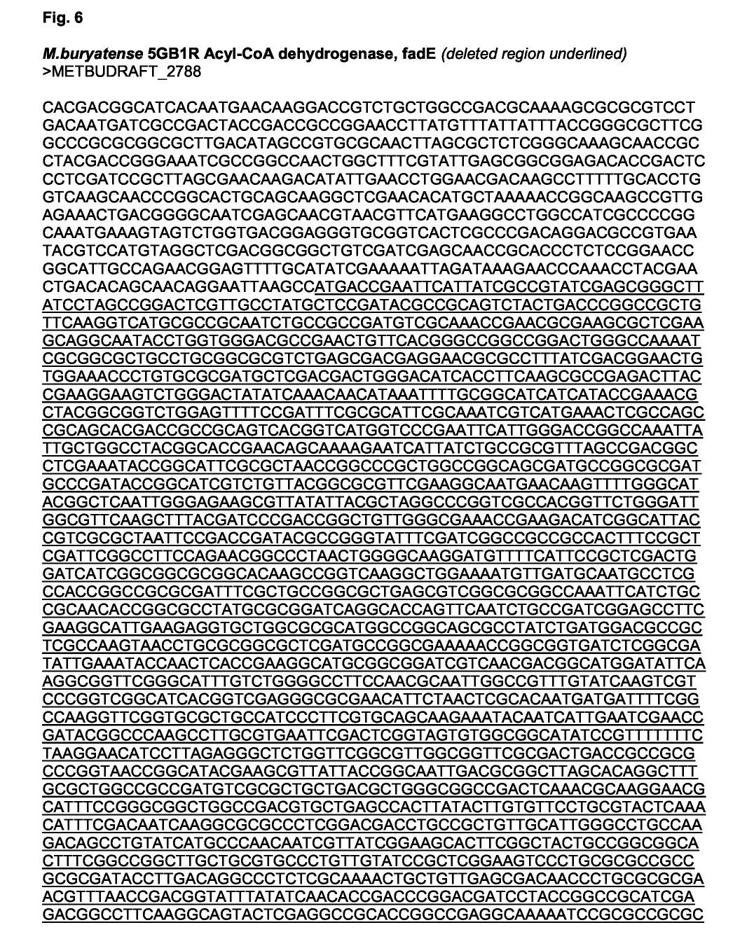

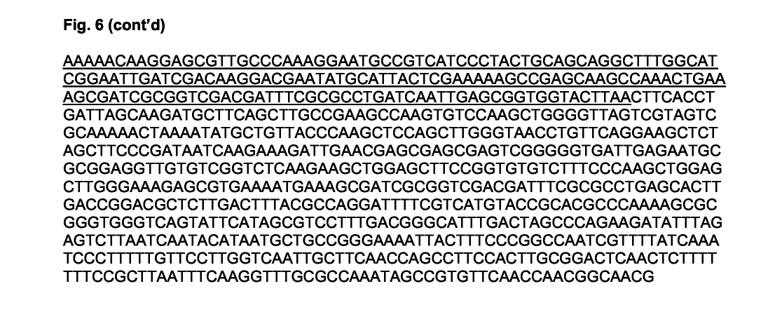

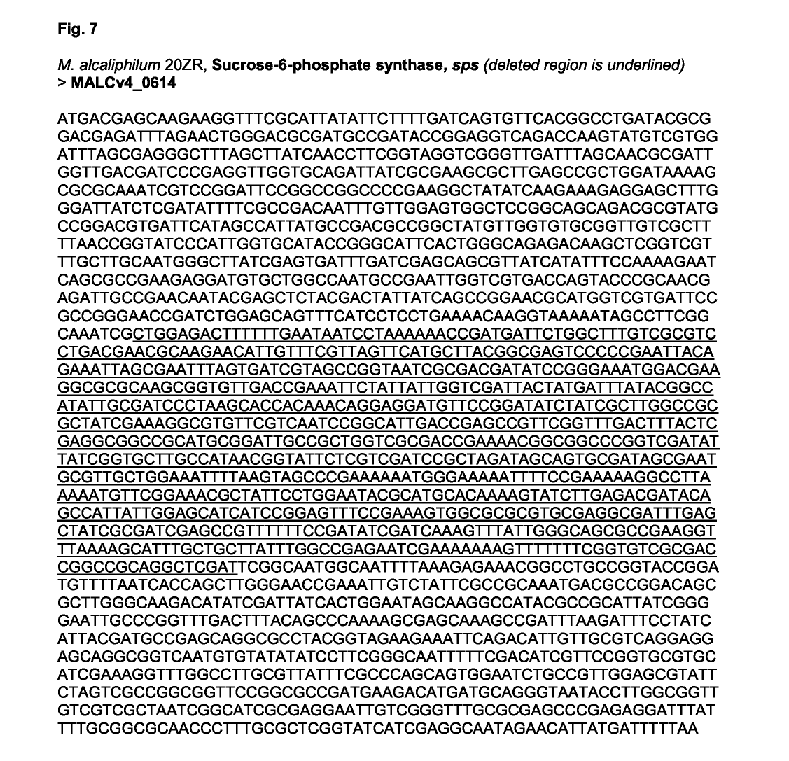

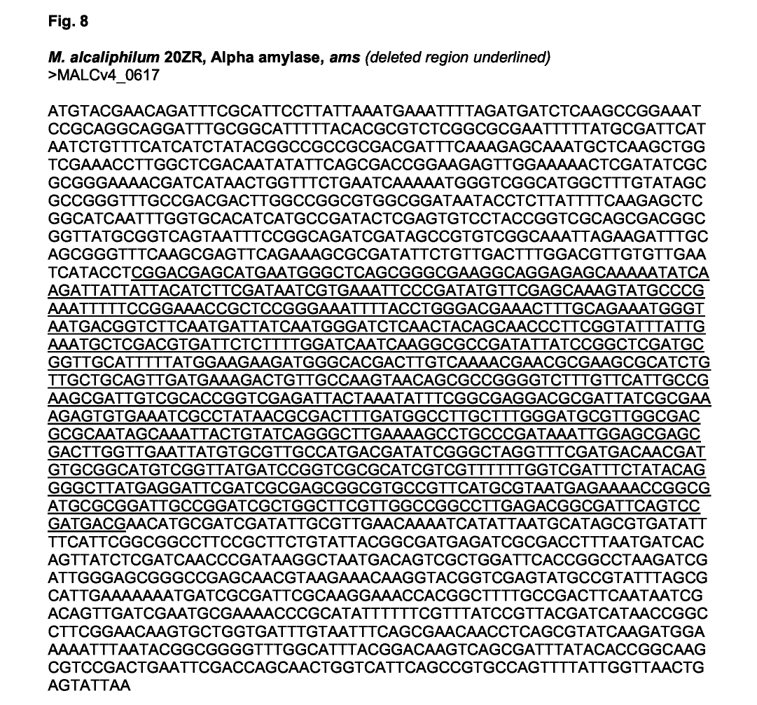

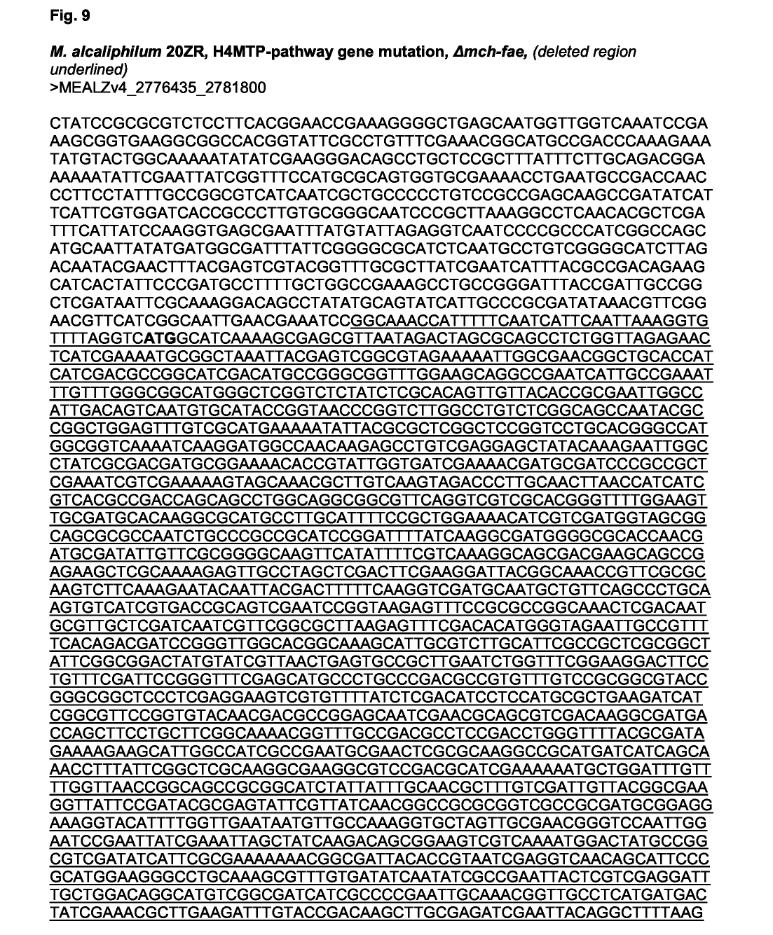

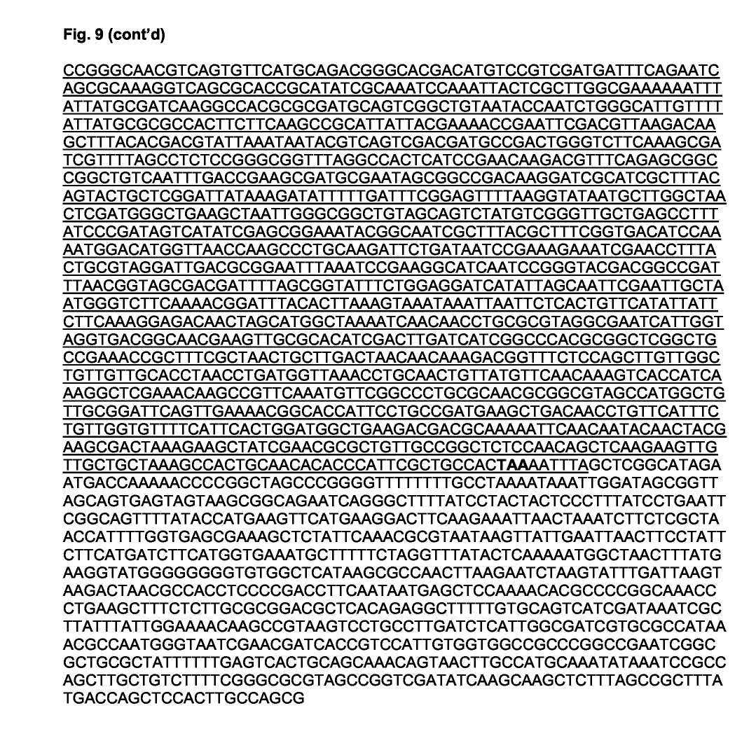

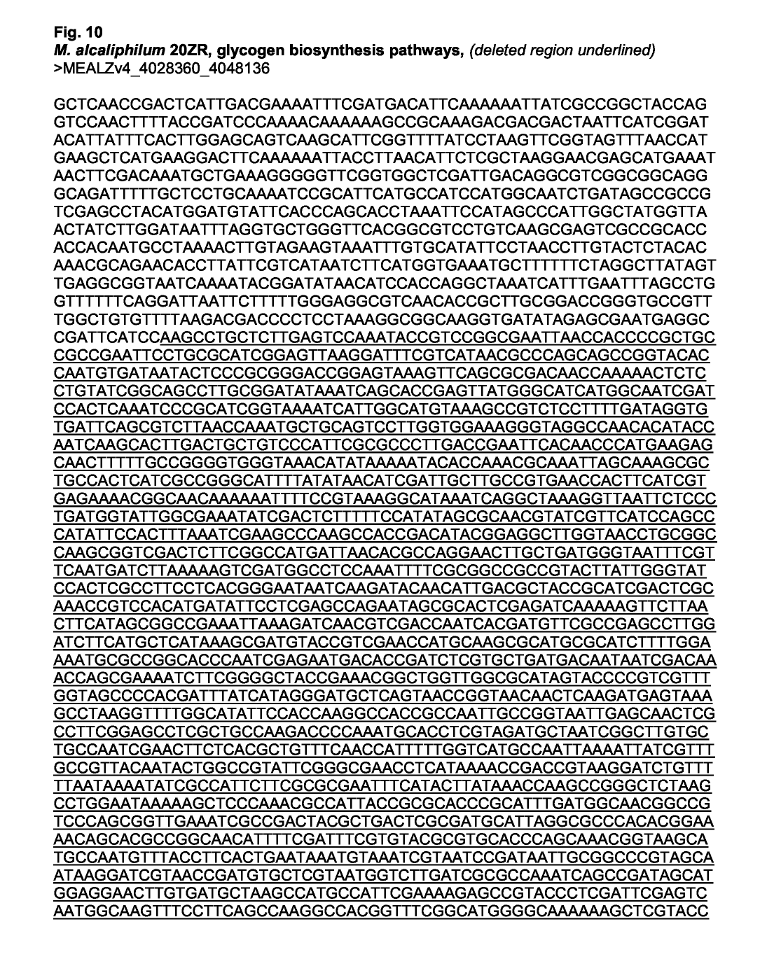

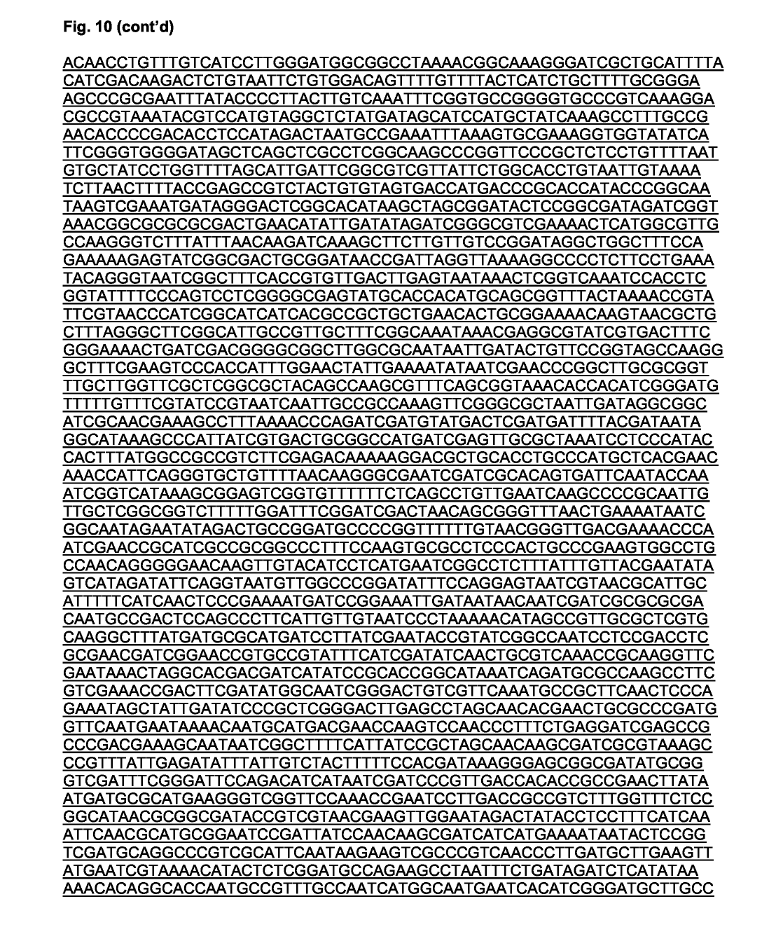

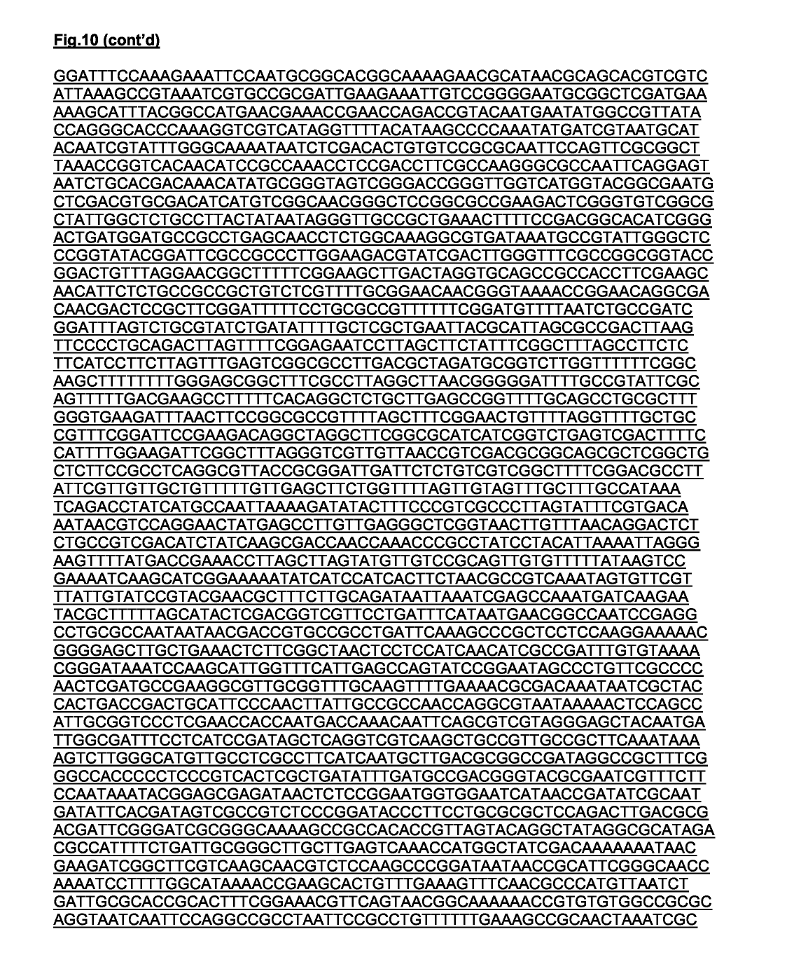

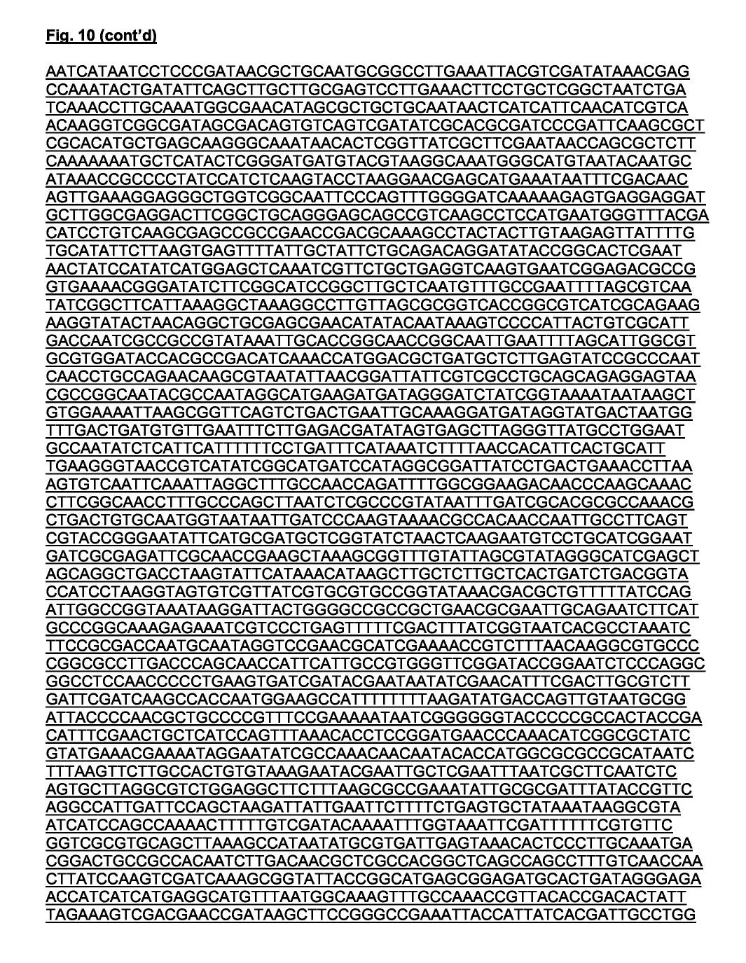

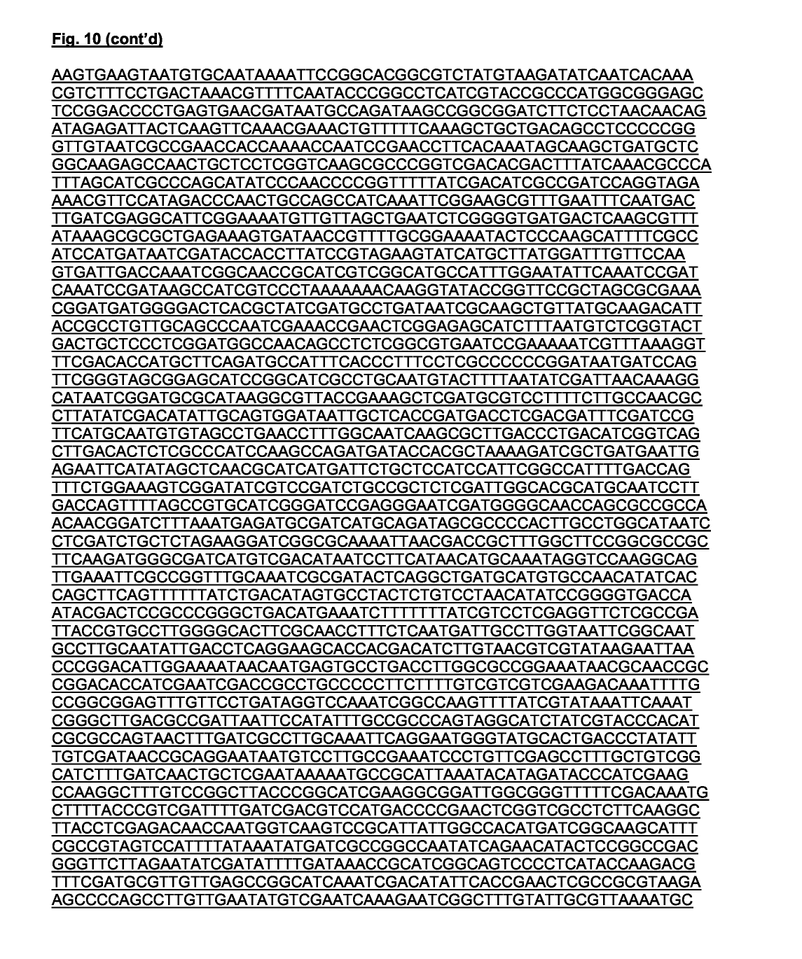

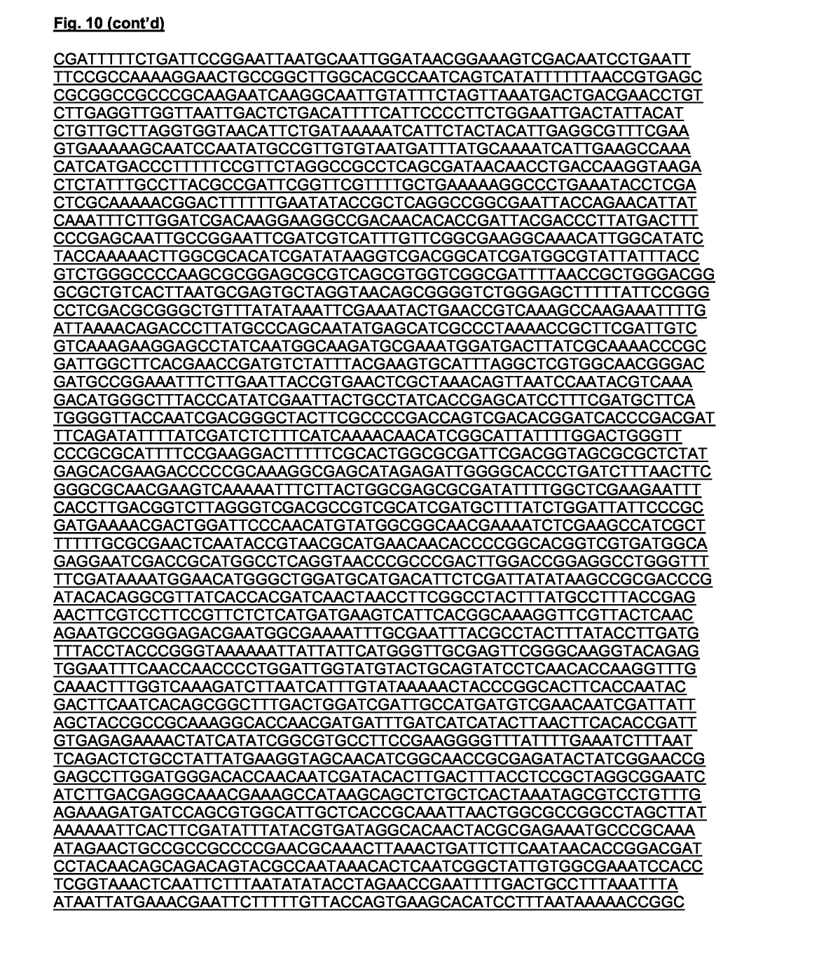

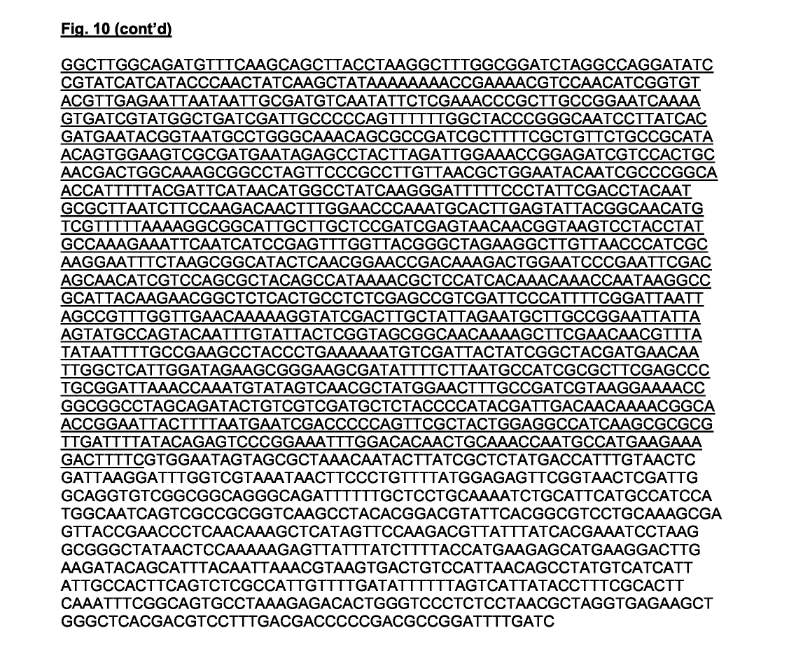

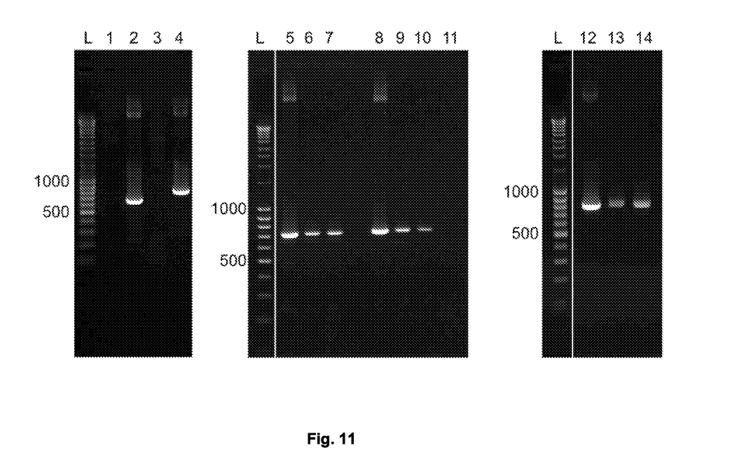

The described results were used to create flux balance models for methane metabolism for each strain (a summary of genome-based reconstruction and flux predictions are shown in FIG. 1). These metabolic models provided design platforms for metabolic redesign activities to modify the strains to produce value-added chemicals from methane. Analysis of central metabolic pathways alone with flux balance analysis (FBA)-simulations resulted in identification of a set of genetic modifications that result in increased flux of the carbon via C3/C2 compounds and increased carbon conversion efficiency.

### Modifications of Group I MB

Strategies for increasing flux into pyruvate (C₃) represent first and essential steps for efficient conversion of methane-derived carbon into biofuels, organic acids or amino acids. Pyruvate is a key intermediate in central metabolism of Group I MB and is a key precursor in biosynthesis. A set of strain modifications are disclosed for Group I MB including 5GB1, 20Z, LW13, MK1 and sp.11b that enhance the use of the strains to produce value-added products from methane. In is anticipated that these modifications or modification targeting the genes modified in these strains will provide beneficial results in other Group I MB strains.

A central goal of the strain modification approach is to increase flux of carbon into fatty acid biosynthesis pathways and to increase fatty acid synthesis for both membrane (phospholipid) and FFA production. Analysis of central metabolic pathways has resulted in identification of several metabolic steps for which elimination or activation leads to increased flux of carbon into fatty acid biosynthesis pathways. Identified targets for genetic modifications for improvements for lipid productivity include: increased gene copy numbers for methane monooxygenase (pmoCAB) to enhance methane-converting capacity of the cell and, indirectly, formation of membranes; pyruvate kinase (pyk1) to increase the flow of carbon into pyruvate, as data indicate that conversion of PEP to pyruvate may be a limiting step; and acetyl-CoA carboxylase (accABC) to increase the flux of carbon into lipids. In order to reduce the CO₂ footprint of the process and increase carbon conversion efficiency, a pathway in the cell to convert formate into acetyl CoA (“AcCoA”), the main precursor for fatty acid biosynthesis can be introduced. This pathway involves diverting formate produced during C1 metabolism into serine, which is then converted to AcCoA via a portion of the serine cycle that already exists in Group I MB strains such as 5GB1 and 20Z. Experiments indicate that cultures excrete a significant amount of formate (up to 25 mM), and diversion of formate into AcCoA synthesis increases carbon conversion efficiency.

The modifications can also involve deletion of formate dehydrogenase (fdsABCD, fdhAB) to decrease formate conversion to CO₂, overexpression of the genes for conversion of formate into methylene-H₄Folate (ftfL, mtdA and fch), which is the precursor for biomass synthesis (including AcCoA synthesis) in the serine cycle, and introduction of ppc (PEP carboxylase gene) to improve conversion of formate to acetyl-CoA. More particularly, this approach can involve diverting formate produced during C₁ metabolism into serine, which is then converted to phospho(enol)pyruvate (PEP) via a portion of the serine cycle that already exists in gammaproteobacterial methanotrophs. The metabolic engineering steps involve down-regulating formate dehydrogenase (fdsABCD, fdhAB) to down-regulate formate conversion to CO₂. Overexpression of the genes for conversion of formate into methylene-H₄Folate (ftfL, mtdA and fch), which is the precursor for biomass synthesis in the serine cycle and introduction of ppc (PEP carboxylase gene) to improve conversion of phospho(enol)pyruvate to oxaloacetate for subsequent regeneration of glyoxylate for serine cycle operation and production of acetyl-CoA, and/or overexpression of the pyruvate kinase to increase the flow of carbon into pyruvate can also be used to increase lipid biomass synthesis.

Another modification can include deletion of specific genes, including gluconate-6-phosphate dehydrogenase (gnd) in order to increase carbon conversion by down-regulating cyclic oxidation of formaldehyde through the pentose-phosphate pathway and redirect carbon into the Entner-Doudoroff and glycolytic pathways for pyruvate production; ADP-glucose pyrophosphorylase (glgC), glycogen synthase (glgA) and glycogen branching enzyme (glgB) to reduce the carbon conversion into glycogen; and phosphoenolpyruvate synthase (pps) in order to decrease flow of pyruvate to phosphoenolpyruvate (PEP). Reallocation of the carbon from carbohydrate metabolism into fatty acid biosynthesis can result in increased production of lipids. To further enhance fatty acid production, a set of modifications (knockouts and down-expression variants) in lipid degradation pathways (β-oxidation pathways, fadE) can be generated. A mutated form of the ACP-thioesterase (TesA) from E. coli can be used to redirect fatty acid synthesis excretion and increase the yield of free fatty acids (FFAs).

Identified targets for genetic modifications for improvements for lipid productivity can include, e.g., deletion of ADP-glucose pyrophosphorylase (glgC), glycogen synthase (glgA) and glycogen branching enzyme (glgB) to reduce the carbon conversion into glycogen; and phosphoenolpyruvate synthase (pps) in order to decrease flow of pyruvate to phosphoenolpyruvate (PEP). Reallocation of the carbon from carbohydrate metabolism into fatty acid biosynthesis can result in increased production of lipids. To further enhance fatty acid production, a set of modifications (knockouts and down-expression variants) in lipid degradation pathways (β-oxidation pathways, fadE) can be generated. A cytoplasmic form of the ACP-thioesterase (TesA) from E. coli can be used to delimit free fatty acid production, resulting in higher yields of free fatty acids (FFAs).

Increased flux of carbon into fatty acid biosynthetic pathways will result in production of more ICM rather than FFAs. In this case it will represent a strain with higher lipid productivity. Production of FFAs at a high level can also provide a production option but the FFAs will need to be recovered from the culture broth, which adds costly process steps. As an alternative improvement, increased fatty acid flux can be re-directed to the in vivo generation of fatty acid esters (FAMEs and others), a concept effectively applied in E. coli [Kalscheuer et al., 2006, Steen et al., 2010]. A strain capable of producing both fatty acid esters and membrane phospholipids is envisioned due to projected improvements in total lipid content as well as simplified recovery and conversion processes. Modifications begin with FAMEs and involve expression of the same heterologous acyltransferase WS/DGAT gene (atfA) in MB, e.g., 5GB1, that has been used in E. coli.

Other modifications include down-regulation of fae, mch, and/or mtdB to increase carbon conversion into cellular sugars by down-regulating the H₄MTP-pathway for formaldehyde oxidation; and deletion or downregulation of sucrose phosphate synthase (sps) to decrease sucrose biosynthesis.

Based on the foregoing, and without wishing to be bound by theory, a summary of modifications to Group I methonotrophic bacteria includes, without limitation, one of more of the following:
1. Up-regulating methane flux by up-regulating methane monooxygenase (pmoCAB), for example, by increasing pmoCAB copy number;
2. Up-regulating pyruvate kinase (pyk1) to up-regulate flow of carbon into pyruvate;
3. Up-regulating acetyl-CoA carboxylase (accABC) to up-regulate flux of carbon into lipids;
4. Down-regulating formate dehydrogenase (fdsABCD and orfdhAB) to down-regulate conversion to CO₂;
5. Up-regulating formyltetrahydrofolate synthetase/ligase (FtfL), methylenetetrahydrofolate dehydrogenase (MtdA) and formyltetrahydrofolate cyclohydrolase Fch to up-regulate conversion of formate into formyl-H₄folate;
6. Up-regulating PEP carboxylase (ppc) to up-regulate conversion of formate into acetyl-CoA;
7. Down-regulating gluconate-6-phosphate dehydrogenase (gnd) to down-regulate cyclic oxidation of formaldehyde through the pentose-phosphate pathway and redirecting it to enter the Entner-Doudoroff and glycolytic pathways for pyruvate production;
8. Down-regulating ADP-glucose pyrophosphorylase (glgC), glycogen synthase (glgA) and/or glycogen branching enzyme (glgB) to down-regulate carbon conversion into glycogen;
9. Down-regulating phosphoenolpyruvate synthase (pps) to down-regulate the flow of pyruvate to PEP;
10. Down-regulating Acyl-CoA dehydrogenase (fadE) to down-regulate lipid degradation pathways, for example, β-oxidation pathways;
11. Introducing mutated ACP-thioesterase (TesA) from E. coli to redirect fatty acid synthesis to up-regulate yields of FFAs;
12. Down-regulating formaldehyde activating enzyme (fae), methenyltetrahydromethanopterin cyclohydrolase (mch), methylenetetrahydromethanopterin dehydrogenase (mtdB) to up-regulate carbon conversion into cellular sugars by down-regulating the H₄MTP-pathway for formaldehyde oxidation (it has been shown that the formaldehyde oxidation pathway is not essential for growth of non-methane utilizing MB containing two pathways for formaldehyde oxidation (cyclic RuMP pathway) and the linear H₄MTP-pathway (Chistoserdova et al., 2000). FBA predicts that down-regulation of the H₄MTP-pathway in MB will lead to up-regulated flux of C₁-carbon via the formaldehyde fixation pathway (RuMP)).
13. Deleting or down-regulating sucrose-phosphate synthase (sps) to down-regulate sucrose biosynthesis
14. Deleting or down-regulating lactate dehydrogenase to reduce pyruvate conversion to lactate;
15. Deleting or down-regulating acetate kinase to reduce conversion of pyruvate to acetate.
   A subset of these approaches is summarized in Table 2.

TABLE 2
Non-Limiting Examples of Disclosed Modifications in Group I MB. The genes listed in
this table can be downregulated in accordance with the methods described herein.

| Gene | Strain | Locus tag | Rationale |
|---|---|---|---|
| Glycogen synthase 1 | Methylomicrobium | METBU | Decrease |
| (glgA1) | buryatense 5GB1 | DRAFT_3833 | glycogen/ |
| | | | extrapolysaccharide |
| | | | production |
| Glycogen synthase 2 | Methylomicrobium | METBU | Decrease |
| (glgA2) | buryatense 5GB1 | DRAFT_3841 | glycogen/ |
| | | | extrapolysaccharide |
| | | | production |
| Phosphoenolpyruvate | Methylomicrobium | METBU | Increase flux to |
| synthase (pps) | buryatense 5GB1 | DRAFT_0376 | pyruvate |
| Acyl-CoA dehydrogenase | Methylomicrobium | METBU | Decrease β-oxidation of |
| (fadE) | buryatense 5GB1 | DRAFT_3642 | produced fatty acids |
| Sucrose-6-phosphate | Methylomicrobium | METBU | Decrease |
| synthase (sps) | buryatense 5GB1 | DRAFT_2515 | glycogen/ |
| | | | extrapolysaccharide |
| | | | production |
| Sucrose-6-phosphate | Methylomicrobium | MALCv4_0614 | Decrease |
| synthase (sps) | alcaliphilum 20Z | | glycogen/sucrose |
| | | | production |
| Glycogen synthase cluster | Methylomicrobium | MALCv4_3507 | Decrease |
| 1. (glgABC1manBQamyC) | alcaliphilum 20Z | MALCv4_3508 | glycogen/sucrose |
| | | | production |
| Glycogen synthase | Methylomicrobium | MALCv4_3502 | Decrease |
| Cluster 2 (glgAB2amyAC) | alcaliphilum 20Z | MALCv4_3503 | glycogen/sucrose |
| | | MALCv4_3504 | production |
| Glycogen synthase cluster | Methylomicrobium | MALCv4_3502 | Decrease |
| 1 + 2. (glgABC1-glgABC2) | alcaliphilum 20Z | MALCv4_3503 | glycogen/sucrose |
| | | MALCv4_3504 | production |
| | | MALCv4_3505 | |
| | | MALCv4_3506 | |
| | | MALCv4_3507 | |
| | | MALCv4_3508 | |
| mch | Methylomicrobium | | Increase carbon conversion |
| | alcaliphilum 20Z | | into cellular sugars |
| mch-fae | Methylomicrobium | MALCv4_2425 | Increase carbon conversion |
| | alcaliphilum 20Z | MALCv4_2426 | into cellular sugars |
| | | MALCv4_2427 | |
| | | MALCv4_2428 | |
| Glycogen synthase 1 | Methylomicrobium | METBU | Decrease glycogen |
| (glgA1) | buryatense 5GB1 | DRAFT_3833 | production |
| Glycogen synthase 2 | Methylomicrobium | METBU | Decrease glycogen |
| (glgA2) | buryatense 5GB1 | DRAFT_3841 | production |
| Phosphoenolpyruvate | Methylomicrobium | METBU | Increase flux to pyruvate |
| synthase (pps) | buryatense 5GB1 | DRAFT_0376 | |
| Acyl-CoA dehydrogenase | Methylomicrobium | METBU | Decrease β-oxidation of |
| (fadE) | buryatense 5GB1 | DRAFT_3642 | produced fatty acids |
| Sucrose-6-phosphate | Methylomicrobium | METBU | Decrease glycogen |
| synthase (sps) | buryatense 5GB1 | DRAFT_2515 | production |
| Lactate dehydrogenase | Methylomicrobium | METBUDRAFT_{—} | Reduce pyruvate conviction |
| | buryatense 5GB1 | 3726 | to lactate/build-up pyruvate |
| | | | pool |
| Acetate kinase | Methylomicrobium | METBUDRAFT_{—} | Reduce pyruvate conviction |
| | buryatense 5GB1 | 1552 | to acetate/build-up pyruvate |
| | | | pool |
| Methylenetetrahydro- | Methylomicrobium | METBUDRAFT_{—} | Reduce formaldehyde |
| methanopterin dehydrogenase | buryatense 5GB1 | 1893-894 | oxidation/Increase |
| | | | formaldehyde conversion to |
| | | | fructose-6-phosphate |
| Sucrose-6-phosphate | Methylomicrobium | MALCv4_0614 | Decrease |
| synthase (sps) | alcaliphilum 20Z | | glycogen/sucrose |
| | | | production |
| Glycogen synthase cluster | Methylomicrobium | MALCv4_3507 | Decrease |
| 1. (glgABC1manBQamyC) | alcaliphilum 20Z | MALCv4_3508 | glycogen/sucrose |
| | | | production |
| Glycogen synthase cluster | Methylomicrobium | MALCv4_3502 | Decrease |
| 1 + 2. (glgABC1-glgABC2) | alcaliphilum 20Z | MALCv4_3503 | glycogen/sucrose |
| | | MALCv4_3504 | production |
| | | MALCv4_3505 | |
| | | MALCv4_3506 | |
| | | MALCv4_3507 | |
| | | MALCv4_3508 | |

While particular examples of genes for modification are described, the current disclosure also encompasses modifications to genes that hybridize with the specifically disclosed genes or are otherwise structurally and functionally related to the genes described. In this manner, for example, genes with functionally redundant counterparts can also be targeted to modify carbon flux in a desired MB. A gene or polynucleotide fragment “hybridizes” to another gene or polynucleotide fragment, such as a cDNA, genomic DNA, or RNA, when a single stranded form of the polynucleotide fragment anneals to the other polynucleotide fragment under the appropriate conditions of temperature and solution ionic strength. Hybridization and washing conditions are well known and exemplified in Sambrook, J., Fritsch, E. F. and Maniatis, T. Molecular Cloning: A Laboratory Manual, Second Ed., Cold Spring Harbor Laboratory Press, Cold Spring Harbor (1989), particularly Chapter 11 and Table 11.1 therein (incorporated by reference herein for its teachings regarding the same). The conditions of temperature and ionic strength determine the “stringency” of the hybridization. Stringency conditions can be adjusted to screen for moderately similar fragments (such as homologous sequences from distantly related organisms) to highly similar fragments (such as genes that duplicate functional enzymes from closely related organisms). Post-hybridization washes determine stringency conditions. One set of hybridization conditions to demonstrate that sequences hybridize uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. Stringent conditions use higher temperatures in which the washes are identical to those above except for the temperature of the final two 30 min washes in 0.2×SSC, 0.5% SDS is increased to 60° C. Highly stringent conditions use two final washes in 0.1SSC, 0.1% SDS at 65° C. Those of ordinary skill in the art will recognize that these temperature and wash solution salt concentrations may be adjusted as necessary according to factors such as the length of the hybridizing sequences. Other approaches based on hybridization include, for example, the expression of antisense sequences to target a desired gene.

Proteins and genes that share a % identity with the proteins and genes explicitly disclosed herein are also within the scope of the present disclosure. The % identity is at least 85%, at least 86%, at least 87% at least 88%, at least 89%, at least 90%, at least 91%, at least 92%, at least 93%, at least 94%, at least 95%, at least 96%, at least 97%, at least 98% or at least 99%. As is known in the art, “% identity” refers to a relationship between two or more protein sequences or two or more polynucleotide sequences, as determined by comparing the sequences. In the art, “identity” also means the degree of sequence relatedness between proteins or polynucleotides as determined by the match between strings of such sequences. “Identity” (often referred to as “similarity”) can be readily calculated by known methods, including (but not limited to) those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992), each incorporated by reference herein for its teachings regarding the same. Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp (1989) CABIOS. 5:151-153, incorporated by reference herein for its teaching regarding the same) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410, 1990, incorporated by reference herein for its teaching regarding the same); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. incorporated by reference herein for its teaching regarding the same). Within the context of this disclosure it will be understood that where sequence analysis software is used for analysis, the results of the analysis are based on the “default values” of the program referenced. As used herein “default values” will mean any set of values or parameters which originally load with the software when first initialized.

### Industrial Production

A variety of culture methodologies may be applied to the modified strains described herein. For example, large-scale production of a specific product made possible by the modified strains described herein may be accomplished by both batch and/or continuous culture methodologies.

A classical batch culturing method is a closed system where the composition of the media is set at the beginning of the culture and not subject to external alterations during the culturing process. Thus, at the beginning of the culturing process the medium is inoculated with the desired strain and growth or metabolic activity is permitted to occur adding nothing to the system. Typically, however, a “batch” culture is batch with respect to the addition of carbon source and attempts are often made at controlling factors such as pH and oxygen concentration. In batch systems the composition of the system changes constantly up to the time the culture is terminated. Within batch cultures, strain cells moderate through a static lag phase to a high growth log phase and finally to a stationary phase where growth rate is diminished or halted. If untreated, cells in the stationary phase will eventually die. Cells in log phase are often responsible for the bulk of production of end product or intermediate in some systems. Stationary or post-exponential phase production can be obtained in other systems.

A variation on the standard batch system is the Fed-Batch system. Fed-Batch culture processes are also suitable and comprise a typical batch system with the exception that the substrate is added in increments as the culture progresses. Fed-Batch systems are useful when catabolite repression is apt to inhibit the metabolism of the cells and where it is desirable to have limited amounts of substrate in the medium. Measurement of the actual substrate concentration in Fed-Batch systems is difficult and is therefore estimated on the basis of the changes of measurable factors such as pH, dissolved oxygen and the partial pressure of waste gases such as CO₂. Batch and Fed-Batch culturing methods are common and well known in the art and examples may be found in Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36:227 (1992) each of which is incorporated by reference herein for its teachings regarding the same.

Continuous cultures can also be used. Continuous cultures are open systems where a defined culture medium is added continuously to a bioreactor and an equal amount of conditioned medium is removed simultaneously for processing. Continuous cultures generally maintain the cells at a constant high liquid phase density where cells are primarily in log phase growth. Alternatively, continuous culture can be practiced with immobilized cells where carbon and nutrients are continuously added and valuable products, by-products, and waste products are continuously removed from the cell mass. Cell immobilization may be performed using a wide range of solid supports composed of natural and/or synthetic materials.

Continuous or semi-continuous culture allows for the modulation of one factor or any number of factors that affect cell growth or end product concentration. For example, one method maintains a limiting nutrient such as the carbon source or nitrogen level at a fixed rate and allows all other parameters to moderate. In other systems, a number of factors affecting growth can be altered continuously while the cell concentration, measured by medium turbidity, is kept constant. Continuous systems strive to maintain steady state growth conditions and thus the cell loss due to medium being drawn off must be balanced against the cell growth rate in the culture. Methods of modulating nutrients and growth factors for continuous culture processes, as well as techniques for maximizing the rate of product formation, are well known in the art of industrial microbiology and a variety of methods are detailed by Brock Biology of Microorganisms, 8th edition, Prentice Hall, UpperSaddle River, N.J. (1997) which is incorporated by reference herein for its teachings regarding the same.

Regarding 5GB1 particularly, it can be grown in a simple mineral medium (NMS) supplemented with salt and carbonate buffer. In some embodiments, in batch culture, optimal growth occurred at pH 9.0-9.5 and with 0.75% NaCl. Under these conditions, a doubling time of 3 hr was achieved, placing it among the fastest-growing MB described. In comparison, standard MB strains have a doubling time of 6-10 hr. Total lipid content ranged from 17-22%, with an estimated 80% present as phospholipids+FFAs, the majority as phospholipids. Fatty acid and polar lipid compositions in the strain were affected by the salinity and pH of the growth medium. Cells grown at high salinity and high pH contain increased levels of C_{16:0} compared to cells grown at low pH and low salt (Table 3). In some embodiments, in batch culture, optimal growth occurred at pH 8.0-9.5 and with 0.75% NaCl. Under these conditions, a doubling time of 3 hr was achieved, placing it among the fastest-growing MB described. In comparison, standard MB strains have a doubling time of 6-10 hr. Total lipid content ranged from 11-22%, with an estimated 80% present as phospholipids+FFAs, the majority as phospholipids. Fatty acid and polar lipid compositions in the strain were affected by the salinity and pH of the growth medium. Cells grown at high salinity and high pH contain increased levels of C_{16:0} compared to cells grown at low pH and low salt (Table 3).

TABLE 3
Growth Parameters

| Parameters | Comment |
|---|---|
| NaCl range (optimum) | 0.1-9% (0.75%) |
| T range (optimum/resistance) | 4-47° C. (30° C./80° C.) |
| pH range (optimum) | 6-11 (8.0-9.0) |
| Lipid content (major fatty acids) | 17-22% of cell dry weight (C16:0; |
| | C16:1) |
| Major phospholipids (% of total) | PA(4), PEA(63), PG(18), PS(8) |

The modified strains disclosed herein can convert methane to complex biochemicals that can generate, for example, fatty-acid derived diesel range hydrocarbon fuels. Fatty acid esters and fatty acids can be converted directly to hydrocarbon products in a single step process using non-sulfur based catalysts (Davis et al., 2009, incorporated by reference herein for its teachings regarding the same). In particular, catalysts have been developed for the conversion of triglycerides, fatty acids and fatty acid methyl esters to hydrocarbon mixtures that closely resemble jet and diesel fuels. Table 4 summarizes data for conversion of palmitic acid (C₁₅H₃₁CO₂H) to C₁₅ and C₁₆ hydrocarbons at 298° C. and 10 bar H₂. Table 5 summarizes data for the conversion of methyl myristate (C₁₃FI₂₇CO₂CH₃) to a combination of predominantly C₁₃ linear hydrocarbons at 270° C. to 295° C. and 10 bar H₂.

TABLE 4
Catalytic conversion of palmitic acid to hydrocarbons.

| | 0.3% Pt on | 5% Pt on |
|---|---|---|
| Catalyst | ferrierite | mordenite |
| Conversion (%) | 45 | 95 |
| Linear C15 hydrocarbon product (%) | 17.8 | 41.0 |
| Branched C15 hydrocarbon | 6.8 | 2.0 |
| product (%) | | |
| Linear C16 hydrocarbon product (%) | 23.7 | 55.0 |
| Branched C16 hydrocarbon | 41.7 | 2.0 |
| product (%) | | |
| Other products (%) | 10.0 | 0.0 |

TABLE 5
Catalytic conversion of methyl myristate to hydrocarbons.

| | | 5% Pd/C + | |
|---|---|---|---|
| | 5% | zeolite | 22% |
| Catalyst | Pd/carbon | (295 oC) | Ni/Al2O3 |
| Conversion (%) | 19 | 76 | 81 |
| Linear C13 | 13 | 59 | 78 |
| hydrocarbon product (%) | | | |
| Linear C14 | 0 | 3 | 1 |
| hydrocarbon product (%) | | | |
| Fatty acids (%) | 6 | 3 | 5 |
| Others (%) | 0 | 5 | 7 |

The modified MB disclosed herein can generate, for example, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5 or 3.6 g total fuel precursor lipids (FFAs+phospholipids+fatty acid esters)/L fermentation broth/hr, ultimately resulting in 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 55 mL diesel product/L/day. In particular embodiments, each of these numerical values is a minimum value (e.g., at least 0.5, at least 0.6, at least 0.7 g, etc. total fuel precursor lipids/L fermentation broth/hr or at least 15, at least 16, at least 17, at least 18 mL, etc. diesel product/L/day).

Described herein are compositions and methods relating to the bacterial production of industrially-useful carbon products from methane. In particular, the engineered bacteria described herein have been modified to increase the production of pyruvate and/or lipids while decreasing the flow of carbon to glycogen, formaldehyde oxidation, and carbon dioxide production. In some embodiments, the bacterium can have increased pyruvate flux. As used herein the term “carbon flux” refers to the number of feedstock molecules (e.g. methane) which proceed down the desired pathway and/or are incorporated into a target molecule relative to competitive paths and/or molecules per unit time. Thus, increased pyruvate flux refers to an increase in the amount of carbon (e.g. from methane) that is converted into pyruvate over a specific time period (e.g. per minute).

In one aspect, described herein is an engineered methanotrophic bacterium, the bacterium comprising a genetic alteration causing a modulation selected from the group consisting of: an increase in the conversion of methane to pyruvate and/or AcCoA; a decrease in the activity of a pathway that diverts formate and/or pyruvate from fatty acid biosynthesis; a decrease in lipid degradation activity; and an increase in fatty ester production. As used herein, “engineered” refers to the aspect of having been manipulated by the hand of man. For example, in some embodiments of the present invention, an engineered bacterium comprises an engineered polynucleotide, e.g., comprises a genetic alteration resulting in a polynucleotide sequence, copy number, or regulatory element not found in nature. As is common practice and is understood by those in the art, progeny and copies of an engineered polynucleotide are typically still referred to as “engineered” even though the actual manipulation was performed on a prior entity. As used herein, “genetic alteration” refers to a change or difference in the genetic material of a cell as compared to a reference wildtype cell, e.g. a deletion, an insertion, a SNP, a substitution, a gene rearrangement, a mutation, and/or the introduction of an exogenous gene or sequence. In some embodiments, the genetic alteration can be an engineered change.

As used herein, “modulation” with respect to genes, proteins, reactions, and/or pathways, refers to downregulation (inhibits activity) or upregulation (activates or increases activity) of protein activity or function. In one embodiment, the modulation occurs by directly inhibiting or increasing the activity of a protein, i.e. via direct physical interaction with the protein or a nucleic acid encoding the protein. In one embodiment, the activity of the protein is modulated indirectly, for example, in signaling, by inhibiting an upstream effector of the protein activity. In some embodiments, the activity of the protein is modulated by increasing or decreasing the level of the protein, e.g. by increasing or decreasing the expression of the gene encoding the protein. In some embodiments of this and other aspects of the technology described herein, activity of the protein is inhibited or lowered by at least 5%, at least 10%, at least 20%, at least 30%, at least 40%, at least 50%, at least 60%, at least 70%, at least 80%, at least 90%, at least 95%, at least 98%, or even 100% (i.e., complete loss of activity) relative to an uninhibited control. In some embodiments of this and other aspects of the technology described herein, activity of the protein is increased by at least 5%, at least 10%, at least 20%, at least 30%, at least 40%, at least 50%, at least 60%, at least 70%, at least 80%, at least 90%, at least 1-fold, at least 1.1-fold, at least 1.5-fold, at least 2-fold, at least 3-fold, at least 4-fold, at least 5-fold, or more relative to an un-activated control, e.g. in absence of activating agent.

A genetic alteration resulting in a decrease in the activity and/or level of a target gene/protein can include, e.g., a knock-down (e.g., a mutation in a promoter that results in decreased gene expression), a knock-out (e.g. a mutation or deletion that results in 99% or greater decrease in gene expression or activity), a mutation of catalytic residues that reduces enzymatic activity, and/or introduction of a nucleic acid sequence that reduces the expression of the target gene (e.g. a repressor that inhibits expression of the target or inhibitory nucleic acids (e.g. CRISPR etc.) that reduce the expression of the target gene).

A genetic alteration resulting in the increase of the activity and/or level of a target gene/protein can include, e.g., introduction of an exogenous nucleic acid sequence comprising the target gene (e.g. on a plasmid or integrated into the genome), a mutation of the endogenous target gene to increase expression (e.g. a mutation of the promoter sequence), and/or introduction of a nucleic acid sequence that increases the expression of the target gene (e.g. introduction of a transcription factor that increases expression of the target gene). In some embodiments, any of these changes can result in ectopic expression of a polypeptide.

In some embodiments, an engineered methanotrophic bacterium as described herein comprises a genetic alteration causing an increase in the conversion of methane to pyruvate and/or AcCoA. In some embodiments, the genetic alteration can result in the increase of pmoCAB (methane monoxygenase); pyk1 (pyruvate kinase); accABC (acetyl-CoA carboxylase); ppc (PEP carboxylase); ftfL (formyltetrahydrofolate); mtdA (methylenetetrahydrofolate dehydrogenase); and/or fch (formyltetrahydrofolate cyclohydrogenase). In some embodiments, the genetic alteration can result in the increase of pmoCAB (methane monoxygenase); pyk1 (pyruvate kinase); accABC (acetyl-CoA carboxylase); ppc (PEP carboxylase); and/or at least one of ftfL (formyltetrahydrofolate); mtdA (methylenetetrahydrofolate dehydrogenase); and fch (formyltetrahydrofolate cyclohydrogenase).

In some embodiments, the genetic alteration can comprise the introduction of an exogenous and/or ectopic ppc (PEP carboxylase) gene. In some embodiments, the genetic alteration can comprise the introduction of a pathway to convert formate to AcCoA. In some embodiments, the introduction of a pathway to convert formate to AcCoA can comprise the introducation of one or more genes that can convert formate to serine. In some embodiments, the bacterium can further comprise a genetic alteration causing a decrease in the activity of a pathway that diverts formate and/or pyruvate from fatty acid biosynthesis.

In some embodiments, an engineered methanotrophic bacterium as described herein comprises a genetic alteration causing a decrease in the activity of a pathway that diverts formate and/or pyruvate from fatty acid biosynthesis. In some embodiments, the genetic alteration can result in a decrease in the activity and/or level of one or more genes selected from the group consisting of: fdsABCD, fdhAB (formate dehydrogenase); gnd (gluconate-6-phosphate dehydrogenase); glgC (ADP-glucose pyrophosphorylase); glgA (glycogen synthase); glgB (glycogen branching enzyme); pps (phosphoenolpyruvate); fae (formaldehyde activating enzyme), mch (methenyltetrahydromethanopterin cyclohydrolase); mtdB (methylenetetrahydromethanopterin dehydrogenase); and sps (sucrose phosphate synthase). In some embodiments, the genetic alteration can result in a decrease in the activity and/or level of one or more genes selected from the group consisting of: gnd(gluconate-6-phosphate dehydrogenase); glgC(ADP-glucose pyrophosphorylase); glgA (glycogen synthase); glgB (glycogen branching enzyme); pps (phosphoenolpyruvate); mtdB (methylenetetrahydromethanopterin dehydrogenase); sps (sucrose phosphate synthase); ldh (lactate dehydrogenase); and ack (acetate kinase).

In some embodiments, an engineered methanotrophic bacterium as described herein comprises a genetic alteration causing a decrease in lipid degradation activity. In some embodiments, the genetic alteration can comprise the introduction of an exogenous and/or ectopic tesA (e.g. E. coli ACP-thioesterase (NCBI Gene ID NO: 945127)) gene. In some embodiments, the genetic alteration can result in the decrease of the activity and/or level of fadE (acyl-CoA dehydrogenase) (beta-oxidation pathways).

In some embodiments, an engineered methanotrophic bacterium as described herein comprises a genetic alteration causing an increase in fatty ester production. In some embodiments, a genetic alteration causing an increase in fatty ester production can comprise the introduction of an exogenous and/or ectopic atfA (actyltransferase WS/DGAT) gene (see, e.g., Kalscheuer, R. & Steinbüchel, A. (2003). J Biol Chem 278, 8075-8082; which is incorporated by reference herein in its entirety).

In some embodiments, the alteration can be selected from those listed in Table 2 and/or Table 9.

TABLE 9

| | An increase in the activity and/or | A decrease in the activity | |
|---|---|---|---|
| | level of: (to include introduction of a | and/or level of: (to include | |
| | plasmid or alteration of the genome) | knock-outs/knockdowns) | Introduction of: |
| An increase in | pmoCAB (methane | | |
| conversion of | monoxygenase) | | |
| methane to | pyk1 (pyruvate kinase) | | |
| pyruvate and/or | accABC (acetyl-CoA | | |
| AcCoA | carboxylase) | | |
| | | | A pathway to |
| | | | convert formate |
| | | | to AcCoA |
| | ftfL (formyltetrahydrofolate); | | |
| | mtdA (methylenetetrahydrofolate | | |
| | dehydrogenase); fch | | |
| | (formyltetrahydrofolate | | |
| | cyclohydrogenase) | | |
| | ppc (PEP carboxylase) | | ppc (PEP |
| | | | carboxylase) |
| A decrease in the | | fdsABCD, fdhAB (formate | |
| activity of | | dehydrogenase) | |
| pathways that | | gnd (gluconate-6- | |
| divert formate | | phosphate dehydrogenase) | |
| and/or pyruvate | | glgC (ADP-glucose | |
| from fatty acid | | pyrophosphorylase); glgA | |
| biosynthesis | | (glycogen synthase); glgB | |
| | | (glycogen branching | |
| | | enzyme) | |
| | | pps (phosphoenolpyruvate) | |
| | | fae (formaldehyde | |
| | | activating enzyme), mch | |
| | | (methenyltetrahydro- | |
| | | methanopterin cyclohydrolase), | |
| | | mtdB | |
| | | (methylenetetrahydro- | |
| | | methanopterin dehydrogenase) | |
| | | sps (sucrose phosphate | |
| | | synthase) | |
| A decrease in lipid | | | TesA (E. coli |
| degradation activity | | | ACP- |
| | | | thioesterase) |
| | | fadE (acyl-CoA | |
| | | dehydrogenase) (beta- | |
| | | oxidation pathways) | |
| Increased fatty acid | | | atfA |
| ester production | | | (actyltransferase |
| | | | WS/DGAT) |

In some embodiments, a methanotrophic bacterium as described herein can comprise a combination of any of the genetic alterations described herein, e.g. described above herein. By way of non-limiting example, a bacterium can comprise an alteration that results in an increase in conversion of methane to pyruvate and/or AcCoA and an alteration that results in increased fatty acid ester production. By way of further non-limiting example, a bacterium can comprise two alterations that result in increased conversion of methane to pyruvate and/or AcCoA, e.g. an alteration that increases the level and/or activity of pyk1, and an alteration that increases the level and/or activity of AccA.

In some embodiments, a heterolog, homolog, and/or variant of the genes described can be utilized in the methods and compositions described herein. A “variant,” as referred to herein, is a polypeptide substantially homologous to a native or reference polypeptide, but which has an amino acid sequence different from that of the native or reference polypeptide because of one or a plurality of deletions, insertions or substitutions. Such polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein. As to amino acid sequences, one of ordinary skill in the art will recognize that individual substitutions, deletions or additions to a nucleic acid, peptide, polypeptide, or protein sequence which alter a single amino acid or a small percentage, (i.e. 5% or fewer, e.g. 4% or fewer, or 3% or fewer, or 1% or fewer) of amino acids in the encoded sequence is a “conservatively modified variant” where the alteration or alterations result in the substitution of an amino acid with a chemically similar amino acid. It is contemplated that some changes can potentially improve the relevant activity, such that a variant, whether conservative or not, has more than 100% of the activity of a wildtype or native polypeptide, e.g. 110%, 125%, 150%, 175%, 200%, 500%, 1000% or more.

Amino acid sequence alignment of a polypeptide of interest with a reference, e.g., from another species can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. Such alignments are readily created by one of ordinary skill in the art, e.g. using the default settings of the alignment tool of the BLASTP program, freely available on the world wide web at http://blast.ncbi.nlm.nih.gov/. Furthermore, homologs of any given polypeptide or nucleic acid sequence can be found using BLAST programs, e.g. by searching freely available databases of sequence for homologous sequences, or by querying those databases for annotations indicating a homolog (e.g. search strings that comprise a gene name or describe the activity of a gene). Such databases can be found, e.g. on the world wide web at http://ncbi.nlm.nih.gov/.

The variant amino acid or DNA sequence can be at least 90%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g. BLASTp (available on the world wide web at http:// blast .ncbi.nlm.nih.gov), with default parameters set.

In some embodiments, the variant is a conservative substitution variant. Variants can be obtained by mutations of native nucleotide sequences, for example.

A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of the wildtype enzyme , e.g. 110%, 125%, 150%, 175%, 200%, 500%, 1000% or more.

The degree of similarity (percent similarity) between an original and a mutant sequence can be determined, for example, by using a similarity matrix. Similarity matrices are well known in the art and a number of tools for comparing two sequences using similarity matrices are freely available online, e.g. BLASTp (available on the world wide web at http://blast.ncbi.nlm.nih.gov), with default parameters set. A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity of a native or reference polypeptide is retained, or for that matter, improved upon. Conservative substitution tables providing functionally similar amino acids are well known in the art. Such conservatively modified variants are in addition to and do not exclude polymorphic variants, interspecies homologs, and alleles consistent with this disclosure. Typically conservative substitutions for one another include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

A methanotrophic bacterium which is engineered as described herein can be, e.g., a Methylomicrobium spp.; Methylmonas spp.; Group I methanotrophic bacterium; Methylomicrobium alcaliphilum; M. alcaliphilum 20ZR; M. buryatenase; M. buryatenase 5GB1; Methylomonas sp. LW13; Methylmonas MK1; or Methylomonas sp.11b.

In one aspect, described herein is a method of engineering a methanotrophic bacterium to increase pyruvate flux, the method comprising genetically altering a methanotrophic bacterium to cause a modulation selected from the group consisting of: an increase in the conversion of methane to pyruvate and/or AcCoA; a decrease in the activity of a pathway that diverts formate and/or pyruvate from fatty acid biosynthesis; a decrease in lipid degradation activity; and an increase in fatty ester production. In some embodiments, the genetic alteration can be as described herein, e.g., as described in Tables 2 and/or 9. In some embodiments, the method can further comprise measuring the catabolism of methane to pyruvate.

In one aspect, described herein is a method of increasing the flux of carbon from methane to pyruvate, the method comprising treating a methanotrophic bacterium to alter the expression or activity of a gene product as specified in Table 2 and/or 9. In some embodiments, the method can further comprise measuring the catabolism of methane to pyruvate.

In one aspect, described herein is a method of producing carbon catabolic products from methane, the method comprising contacting an engineered bacterium as described herein with methane under conditions suitable for carbon catabolism. Conditions suitable for carbon catabolism can comprise conditions under which a bacterium as described herein is metabolically active and provided access to methane as a carbon source. Examples of suitable conditions are provided in Table 3 and the examples herein. In some embodiments, the carbon catabolic product is selected from the group consisting of lipids; fatty acids; fatty acid esters; free fatty acids; phospholipids In some embodiments, the method can further comprise measuring the catabolism of methane to pyruvate.

In some embodiments, the method can further comprise the step of isolating, purifying, and/or concentrating the carbon catabolic product. Methods for isolating, purifying, and/or concentrating the carbon catabolic products described herein are well known in the art.

In one aspect, described herein is a method of fixing methane carbon in pyruvate, the method comprising contacting an engineered bacterium as described herein with methane under conditions suitable for methane catabolism. In some embodiments, the method can further comprise measuring the catabolism of methane to pyruvate.

Methods of measuring the catabolism of methane to pyruvate are known in the art and can include, by way of non-limiting example ¹³C-labeling metabolomics (see, e.g., Kalyuzhnaya et al. 2013; which is incorporated by reference herein in its entirety).

In some embodiments, a genetic alteration can comprise the introduction of an exogenous gene (encoding an exogenous and/or ectopic polypeptide) and/or an alteration of an endogenous gene. In order for the gene to be expressed, the nucleic acid encoding the polypeptide can be operatively linked to a promoter. In some embodiments, the polypeptide can be constitutively expressed. In some embodiments, nucleic acids encoding the polypeptide can be operatively linked to a constitutive promoter. In some embodiments, polypeptide can be inducibly expressed. In some embodiments, nucleic acids encoding the polypeptide can be operatively linked to an inducible promoter.

As described herein, an “inducible promoter” is one that is characterized by initiating or enhancing transcriptional activity when in the presence of, influenced by, or contacted by an inducer or inducing agent relative to such activity when not in the presence of, under the influence of, or in contact with the inducer or inducing agent. An “inducer” or “inducing agent” can be endogenous, or a normally exogenous compound or protein that is administered in such a way as to be active in inducing transcriptional activity from the inducible promoter. In some embodiments, the inducer or inducing agent, e.g., a chemical, a compound or a protein, can itself be the result of transcription or expression of a nucleic acid sequence (e.g., an inducer can be a transcriptional repressor protein), which itself may be under the control or an inducible promoter. Non-limiting examples of inducible promoters include but are not limited to, the lac operon promoter, a nitrogen-sensitive promoter, an IPTG-inducible promoter, a salt-inducible promoter, and tetracycline, steroid-responsive promoters, rapamycin responsive promoters and the like. Inducible promoters for use in prokaryotic systems are well known in the art, see, e.g. the beta.-lactamase and lactose promoter systems (Chang et al., Nature, 275: 615 (1978, which is incorporated herein by reference); Goeddel et al., Nature, 281: 544 (1979), which is incorporated herein by reference), the arabinose promoter system, including the araBAD promoter (Guzman et al., J . Bacteriol., 174: 7716-7728 (1992), which is incorporated herein by reference; Guzman et al., J. Bacteriol., 177: 4121-4130 (1995), which is incorporated herein by reference; Siegele and Hu, Proc. Natl. Acad. Sci. USA, 94: 8168-8172 (1997), which is incorporated herein by reference), the rhamnose promoter (Haldimann et al., J. Bacteriol., 180: 1277-1286 (1998), which is incorporated herein by reference), the alkaline phosphatase promoter, a tryptophan (trp) promoter system (Goeddel, Nucleic Acids Res., 8: 4057 (1980), which is incorporated herein by reference), the PLtetO-1 and Plac/are-1 promoters (Lutz and Bujard, Nucleic Acids Res., 25: 1203-1210 (1997), which is incorporated herein by reference), and hybrid promoters such as the tac promoter. deBoer et al., Proc. Natl. Acad. Sci. USA, 80: 21-25 (1983), which is incorporated herein by reference.

An inducible promoter useful in the methods and systems as disclosed herein can be induced by one or more physiological conditions, such as changes in pH, temperature, radiation, osmotic pressure, saline gradients, cell surface binding, and the concentration of one or more extrinsic or intrinsic inducing agents. The extrinsic inducer or inducing agent can comprise amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones, and combinations thereof. In specific embodiments, the inducible promoter is activated or repressed in response to a change of an environmental condition, such as the change in concentration of a chemical, metal, temperature, radiation, nutrient or change in pH. Thus, an inducible promoter useful in the methods and systems as disclosed herein can be a phage inducible promoter, nutrient inducible promoter, temperature inducible promoter, radiation inducible promoter, metal inducible promoter, hormone inducible promoter, steroid inducible promoter, and/or hybrids and combinations thereof. Appropriate environmental inducers can include, but are not limited to, exposure to heat (i.e., thermal pulses or constant heat exposure), various steroidal compounds, divalent cations (including Cu2+ and Zn2+), galactose, tetracycline, IPTG (isopropyl-β-D thiogalactoside), as well as other naturally occurring and synthetic inducing agents and gratuitous inducers.

Inducible promoters useful in the methods and systems as disclosed herein also include those that are repressed by “transcriptional repressors” that are subject to inactivation by the action of environmental, external agents, or the product of another gene. Such inducible promoters may also be termed “repressible promoters” where it is required to distinguish between other types of promoters in a given module or component of the biological switch converters described herein. Preferred repressors for use in the present invention are sensitive to inactivation by physiologically benign agent. Thus, where a lac repressor protein is used to control the expression of a promoter sequence that has been engineered to contain a lacO operator sequence, treatment of the host cell with IPTG will cause the dissociation of the lac repressor from the engineered promoter containing a lacO operator sequence and permit transcription to occur. Similarly, where a tet repressor is used to control the expression of a promoter sequence that has been engineered to contain a tetO operator sequence, treatment of the host cell with tetracycline will cause the dissociation of the tet repressor from the engineered promoter and permit transcription of the sequence downstream of the engineered promoter to occur.

In some embodiments, a genetic alteration is present in nucleic acid sequence present within the prokaryotic genome, e.g. the nucleic acids can be incorporated into the genome. For example, in bacteria, one can use homologous recombination to target genes to specific sites on bacterial chromosomes. In some embodiments, a nucleic acid comprising a genetic alteration is present within a vector. The term “vector”, as used herein, refers to a nucleic acid construct designed for delivery to a host cell or transfer between different host cells. As used herein, a vector can be viral or non-viral. Many vectors useful for transferring exogenous genes into target cells are available, e.g. the vectors may be episomal, e.g., plasmids, virus derived vectors or may be integrated into the target cell genome, through homologous recombination or random integration. In some embodiments, a vector can be an expression vector. As used herein, the term “expression vector” refers to a vector that has the ability to incorporate and express heterologous nucleic acid fragments in a cell. An expression vector may comprise additional elements. The nucleic acid incorporated into the vector can be operatively linked to an expression control sequence when the expression control sequence controls and regulates the transcription and translation of that polynucleotide sequence.

In some embodiments, a nucleic acid comprising a genetic alteration is present within a portion of a plasmid. Plasmid vectors can include, but are not limited to, pBR322, pBR325, pACYC177, pACYC184, pUC8, pUC9, pUC18, pUC19, pLG339, pR290, pKC37, pKC101, SV 40, pBluescript II SK +/− or KS +/− (see “Stratagene Cloning Systems” Catalog (1993) from Stratagene, La Jolla, Calif., which is hereby incorporated by reference), pQE, pIH821, pGEX, pET series (see Studier et. al., “Use of T7 RNA Polymerase to Direct Expression of Cloned Genes,” Gene Expression Technology, vol. 185 (1990), which is hereby incorporated by reference in its entirety).

As used herein, the term “viral vector” refers to a nucleic acid vector construct that includes at least one element of viral origin and has the capacity to be packaged into a viral vector particle. The viral vector can contain a transgenic gene in place of non-essential viral genes. The vector and/or particle may be utilized for the purpose of transferring any nucleic acids into cells either in vitro or in vivo. Numerous viral vectors are known in the art and can be used as carriers of a nucleic acid into a cell, e.g. lambda vector system gt11, gt WES.tB, Charon 4, IncPfamily plasmids such as pAYC61 (see, e.g., Chistoserdova et al. 1994) and sacB-based system (see, e.g, Sharpe et al. 2007 and Welander and Simmons 2012). Each of the foregoing references is incorporated by reference herein in its entirety.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The term “exogenous” refers to a substance present in a cell other than its native source. The term “exogenous” when used herein can refer to a nucleic acid or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in low amounts and one wishes to increase the amount of the nucleic acid or polypeptide in the cell or organism. A substance will be considered exogenous if it is introduced into a cell or an ancestor of the cell from which the cell has inherited the substance. In contrast, the term “endogenous” refers to a substance that is native to the biological system or cell (e.g. the microbial cell and/or target cell). As used herein, “ectopic” refers to a substance that is found in an unusual location and/or amount. An ectopic substance can be one that is normally found in a given cell, but at a much lower amount and/or at a different time.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level.

The terms “increased” ,“increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double- stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

As used herein, “donor bacteria” are bacteria containing genetic material which is capable of being transferred to other bacteria.

As used herein, “recipient bacteria” are bacteria that receive or are capable of accepting genetic material from another source, e.g., another bacterium.

As used herein, “broad host range plasmid” is a plasmid capable of replicating in more than one bacterial host. In certain embodiments, the broad host range plasmid can replicate in 2, 3, 4, 5, 6 or more different types or species of hosts. See, for example, Lale et al., Methods in Molecular Biology, vol. 765, pages 327-343, (2011).

As used herein, “methanotrophic bacteria” are bacteria that are able to metabolize methane as their only source of carbon. In certain embodiments of the present disclosure, methanotrophic bacteria include Methylococcus, Methylomonas, Methylomicrobium, Methylobacter, Methylocaldum, Methylovulum, Methylomarinum, Methylocystis and Methylosinus. In certain preferred embodiments, methanotrophic bacteria include Methylomicrobium buryatense and Methylomicrobium alcaliphilum. In certain further preferred embodiments, the methanotrophic bacteria are Methylomicrobium buryatense 5GB1 and Methylomicrobium alcaliphilum 20Z.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment of the invention.

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 4^{th} ed., J. Wiley & Sons (New York, N.Y. 2012); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 4th ed., Cold Spring Harbor Laboratory Press (Cold Spring Harbor, N.Y. 2012); provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. “About” can be from about +/−20% to +/−1%. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

As will be understood by one of ordinary skill in the art, each embodiment disclosed herein can comprise, consist essentially of or consist of its particular stated element, step, ingredient or component.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Ed. Anthony Smith, Oxford University Press, Oxford, 2004).

Contemplated herein are embodiments of the invention according to the following paragraphs:
1. A method for generating genetically tractable variants of methanotrophic bacteria comprising:
   mating methanotrophic recipient bacterium with a donor bacterium comprising a broad host range plasmid comprising an antibiotic resistance gene;
   selecting antibiotic resistant strains; and
   passaging the antibiotic resistant strains on non-selective media to enable plasmid loss to provide a genetically tractable variant of the methanotrophic recipient bacteria.
2. The method of paragraph 1 further comprising: mating the genetically tractable variant of the methanotrophic bacterium with a second donor bacteria comprising a second broad host range vector.
3. The method of paragraph 1, wherein the broad host range plasmid is selected from group consisting of pVK100, IncP, IncQ, IncW, pBBR, pMB1, p15A, and pUE10 plasmids.
4. The method of paragraph 3, wherein the IncP plasmids are selected from the group consisting of RK2, RP4, pVK100, and pCM66.
5. The method of paragraph 3, wherein the IncQ plasmid is RSF1010.
6. The method of paragraph 3, wherein the IncW plasmid is pSa.
7. The method of paragraph 3, wherein the pBBr plasmid is pBBr1.
8. The method of paragraph 1, wherein the donor bacterium is E. coli.
   9. The method of paragraph 1, wherein the methanotrophic recipient bacterium are selected from the group consisting of Methylomicrobium alcaliphilum and Methylomicrobium buryatense.
   10. The method of paragraph 9, wherein the Methylomicrobium alcaliphilum recipient bacterium is Methylomicrobium alcaliphilum 20Z.
   11. The method of paragraph 9, wherein the Methylomicrobium buryatense recipient bacteria is Methylomicrobium buryatense 5GB1.
   12. The method of paragraph 11; wherein the lost plasmid is that of SEQ ID NO: 1.
   13. A modified methanotrophic bacteria having an increased genetic tractability over wild-type methanotrophic bacteria.
   14. The modified methanotrophic bacteria of paragraph 13, wherein modified methanotrophic do not contain an endogenous plasmid that interferes with replication and conjugation functions.
   15. The modified methanotrophicbacteria of paragraph 13, wherein the relative genetic tractability is between about 0.1-1.0.
   16. The modified methanotrophic bacteria of paragraph 14, wherein the endogenous plasmid is that of SEQ ID NO: 1.

Some embodiments of the technology described herein can be defined according to any of the following numbered paragraphs:
1. An engineered methanotrophic bacterium, the bacterium comprising a genetic alteration causing a modulation selected from the group consisting of:
   an increase in the conversion of methane to pyruvate and/or AcCoA;
   a decrease in the activity of a pathway that diverts formate and/or pyruvate from fatty acid biosynthesis;
   a decrease in lipid degradation activity; and
   an increase in fatty ester production.
2. The bacterium of paragraph 1, wherein the bacterium comprises a genetic alteration causing an increase in the conversion of methane to pyruvate and/or AcCoA.
3. The bacterium of any of paragraphs 1-2, further comprising a genetic alteration causing a decrease in the activity of a pathway that diverts formate and/or pyruvate from fatty acid biosynthesis.
4. The bacterium of any of paragraphs 1-3, wherein the bacterium has an increased pyruvate flux.
5. The bacterium of any of paragraphs 1-4, wherein the alteration of a gene is selected from

Table 2 or Table 9.
6. The bacterium of any of paragraphs 1-5, wherein the alteration of a gene is selected from the group consisting of:
   an alteration resulting in an increase in the expression or activity of a gene selected from the group consisting of:
      pmoCAB(methane monoxygenase); pyk1 (pyruvate kinase); accABC(acetyl-CoA carboxylase); ppc (PEP carboxylase); ftfL (formyltetrahydrofolate); mtdA (methylenetetrahydrofolate dehydrogenase); and fch (formyltetrahydrofolate cyclohydrogenase).
   the introduction of an exogenous or ectopic ppc (PEP carboxylase); atfA (actyltransferase WS/DGAT); or tesA gene;
   an alteration resulting in a decrease in the expression or activity of a gene selected from the group consisting of:
      fdsABCD, fdhAB (formate dehydrogenase); gnd (gluconate-6-phosphate dehydrogenase); glgC (ADP-glucose pyrophosphorylase); glgA (glycogen synthase); glgB (glycogen branching enzyme); pps (phosphoenolpyruvate); fae(formaldehyde activating enzyme), mch (methenyltetrahydromethanopterin cyclohydrolase); mtdB (methylenetetrahydromethanopterin dehydrogenase); sps (sucrose phosphate synthase); ldh (lactate dehydrogenase); and ack (acetate kinase).
7. The bacterium of paragraph 6, wherein the alteration of a gene is an alteration resulting in a decrease in the expression or activity of a gene selected
   from the group consisting of:
      gnd (gluconate-6-phosphate dehydrogenase); glgC (ADP-glucose pyrophosphorylase); glgA (glycogen synthase); glgB (glycogen branching enzyme); pps (phosphoenolpyruvate); mtdB (methylenetetrahydromethanopterin dehydrogenase); sps (sucrose phosphate synthase); ldh (lactate dehydrogenase); and ack (acetate kinase).
8. The bacterium of any of paragraphs 1-7, wherein the bacterium is selected from the group consisting of:
   Methylomicrobium spp.; Methylmonas spp.; Group I methanotrophic bacterium; Methylomicrobium alcaliphilum; M. alcaliphilum 20ZR; M. buryatenase; M. buryatenase 5GB1; Methylomonas sp. LW13; Methylmonas MK1; Methylomonas sp.11b.
9. An engineered methanotrophic bacterium, the bacterium comprising a genetic alteration which modulates the expression of a gene product as specified in Table 9.
10. The bacterium of paragraph 9, wherein the bacterium has an increased pyruvate flux.
11. The bacterium of any of paragraphs 9-10, wherein the bacterium is selected from the group consisting of:
   Methylomicrobium spp.; Methylmonas spp.; Group I methanotrophic bacterium; Methylomicrobium alcaliphilum; M. alcaliphilum 20ZR; M. buryatenase; M. buryatenase 5GB1; Methylomonas sp. LW13; Methylmonas MK1; Methylomonas sp.11b.
12. A method of engineering a methanotrophic bacterium to increase pyruvate flux, the method comprising genetically altering a methanotrophic bacterium to cause a modulation selected from the group consisting of:
   an increase in the conversion of methane to pyruvate and/or AcCoA;
   a decrease in the activity of a pathway that diverts formate and/or pyruvate from fatty acid biosynthesis;
   a decrease in lipid degradation activity; and
   an increase in fatty ester production.
13. The method of paragraph 12, wherein the bacterium comprises a genetic alteration causing an increase in the conversion of methane to pyruvate and/or AcCoA.
14. The method of any of paragraphs 12-13, further comprising a genetic alteration causing a decrease in the activity of a pathway that diverts formate and/or pyruvate from fatty acid biosynthesis.
15. The method of any of paragraphs 12-14, wherein the bacterium has an increased pyruvate flux.
16. The method of any of paragraphs 12-15, wherein the alteration of a gene is selected from Table 2 or Table 9.
17. The method of any of paragraphs 12-16, wherein the alteration of a gene is selected from the group consisting of:
   an alteration resulting in an increase in the expression or activity of a gene selected from the group consisting of:
      pmoCAB(methane monoxygenase); pyk1 (pyruvate kinase); accABC(acetyl-CoA carboxylase); ppc (PEP carboxylase); ftfL (formyltetrahydrofolate); mtdA (methylenetetrahydrofolate dehydrogenase); and fch (formyltetrahydrofolate cyclohydrogenase).
   the introduction of an exogenous or ectopic ppc (PEP carboxylase); atfA (actyltransferase WS/DGAT); or tesA gene;
   an alteration resulting in a decrease in the expression or activity of a gene selected from the group consisting of:
      fdsABCD, fdhAB (formate dehydrogenase); gnd (gluconate-6-phosphate dehydrogenase); glgC (ADP-glucose pyrophosphorylase); glgA (glycogen synthase); glgB (glycogen branching enzyme); pps (phosphoenolpyruvate); fae(formaldehyde activating enzyme), mch (methenyltetrahydromethanopterin cyclohydrolase); mtdB (methylenetetrahydromethanopterin dehydrogenase); sps (sucrose phosphate synthase); ldh (lactate dehydrogenase); and ack (acetate kinase).
18. The method of paragraph 17, wherein the alteration of a gene is an alteration resulting in a decrease in the expression or activity of a gene selected
   from the group consisting of:
      gnd (gluconate-6-phosphate dehydrogenase); glgC (ADP-glucose pyrophosphorylase); glgA (glycogen synthase); glgB (glycogen branching enzyme); pps (phosphoenolpyruvate); mtdB (methylenetetrahydromethanopterin dehydrogenase); sps (sucrose phosphate synthase); ldh (lactate dehydrogenase); and ack (acetate kinase).
19. The method of any of paragraphs 12-18, wherein the bacterium is selected from the group consisting of:
   Methylomicrobium spp.; Methylmonas spp.; Group I methanotrophic bacterium; Methylomicrobium alcahphilum; M. alcahphilum 20ZR; M. buryatenase; M. buryatenase 5GB1; Methylomonas sp. LW13; Methylmonas MK1; Methylomonas sp.11b.
20. The method of any of paragraphs 12-19, wherein the method further comprises measuring the catabolism of methane to pyruvate.
21. A method of increasing the flux of carbon from methane to pyruvate, the method comprising treating a methanotrophic bacterium to alter the expression or activity of a gene product as specified in Table 9.
22. The method of paragraph 21, wherein the method further comprises measuring the catabolism of methane to pyruvate.
23. A method of producing carbon catabolic products from methane, the method comprising contacting a bacterium of any of paragraphs 1-11 with methane under conditions suitable for carbon catabolism.
24. The method of paragraph 23, wherein the carbon catabolic product is selected from the group consisting of:
   lipids; fatty acids; fatty acid esters; free fatty acids; phospholipids
25. The method of paragraph 23, wherein the carbon catabolic product is an organic acid.
26. The method of paragraph 23, wherein the carbon catabolic product is an alcohol.
27. The method of paragraph 25, wherein the organic acid is selected from the group consisting of:
   succinate; acetate; butyrate; lactate; malate; fumarate; citrate; glycerate; formic acid; stearic acid; 3-hydroxybutyrate; propionate; and mixtures thereof.
28. The method of paragraph 26, wherein the alcohol is selected from the group consisting of propanol, isopropanol, ethanol, or mixtures thereof.
29. The method of any of paragraphs 23-28, wherein the method further comprises measuring the catabolism of methane to pyruvate.
30. The method of any of paragraphs 23-29, further comprising the step of isolating the carbon catabolic product.
31. The method of any of paragraphs 21-30, wherein the carbon catabolic product is a lipid.
32. A method of fixing methane carbon in pyruvate, the method comprising contacting a bacterium of any of paragraphs 1-11 with methane under conditions suitable for methane catabolism.
33. The method of paragraph 32, wherein the method further comprises measuring the catabolism of methane to pyruvate.

### EXAMPLES

The Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### Example 1

Construction of mutants was performed as previously described (Ojala et al., 2010). The following cloning vectors were used: pCM 184, as a suicide vector (Marx& Lidstrom, 2004); pRK2013 (Ditta et al., 1985) as a helper plasmid, pCR2.1 (INVITROGEN™) for cloning of PCR products. E. coli strains JM109 (34), S17-1 (Yanish-Perron et al., 1985) and Top 10 (INVITROGEN™) were routinely cultivated at 37° C. in Luria-Bertani (LB) medium (Sambrook et al., 1989). The following antibiotic concentrations were used: Tet, 12.5; Kan 100 μg ml⁻¹; Amp 100 μg ml⁻¹, Rif, 100 μg ml⁻1.

Data from the Methylomicrobium spp genome projects were used for designing primers flanking upstream and downstream regions of targeted genes. Representative primers include those listed in Table 6.

TABLE 6
Representative Primers used for amplification of flanking regions

| | | | | |
|---|---|---|---|---|
| | | | SEQ | |
| | Primer | | ID | |
| Gene ID | name | Sequence | NO: | Description |

5GB1 (See, e.g., GenBank/EMBL under the accession numbers AOTL01000000, KB455575, and
KB455576)

| | | | | |
|---|---|---|---|---|
| Glycogen | AP-1 | GAGGACGTCCACAGCGGCTTTGACTGGATCG | 2 | glgA1- |
| synthase, glgA1 | | | | up/F/AatII |
| METBUDRAFT_3833 | AP-2 | GAGGGTACCGAGTTTACCGAGGTGGATTTCGCC | 3 | glgA1- |
| | | | | up/R/KpnI |
| | AP-3 | GAGGGGCCCGTGGAATAGTAGTGCTAAACAAT | 4 | glgA1- |
| | | | | dw/F/ApaI |
| | AP-4 | GAGGAGCTCCGTCAAAGGACGCCGTGAGCCCAG | 5 | glgA1- |
| | | | | dw/R/SacI |
| Glycogen | AP-5 | GAGGACGTCGTATTGAATCAGGCGGCTCGT | 6 | glgA2- |
| synthase, glgA2 | | | | up/F/AatII |
| METBUDRAFT_3841 | AP-6 | GAGGGTACCTGCTCGCTTGAGAGGTGTCGGC | 7 | glgA2- |
| | | | | up/R/KpnI |
| | AP-7 | GAGGGGCCCGCGTTACGATTATTCCTGGA | 8 | glgA2- |
| | | | | dw/F/ApaI |
| | AP-8 | GAGGAGCTCTGGTTCGCTCGGCGCTGCAGC | 9 | glgA2- |
| | | | | dw/R/SacI |
| PEP synthase, | AP-9 | GAGGAATTCCGCCAAGCATACTCCACACC | 10 | pps-UP-F- |
| pps | | | | EcoRI |
| METBUDRAFT_0376 | AP-10 | GAGGGTACCCTTCACGCATGATTAACACCC | 11 | pps-UP-R- |
| | | | | KpnI |
| | AP-11 | GAGGGGCCCATAATCCTGAGCAGCCGGAG | 12 | pps-Dwn-F- |
| | | | | ApaI |
| | | GAGGAGCTCCTGCAACAACATAAAGCCGAC | 13 | pps-Dwn-R- |
| | | | | SacI |
| Sucrose-6- | AP-43 | GGTGGTGACGTCACGCTGCTCTAAATACCTTGG | 14 | AP43_sps- |
| phosphate | | | | up-fwd- |
| Synthase, sps | | | | AatII |
| METBUDRAFT_2515 | AP-44 | GGTGGTGGTACCCACGAACAACTATCTCAAGCG | 15 | AP44_sps- |
| | | | | up-rev-KpnI |
| | AP-45 | GGTGGTGGGCCCCCTTTGCACTCGGTATCATCG | 16 | AP45_sps- |
| | | | | dwn-fwd- |
| | | | | ApaI |
| | AP-46 | GGTGGTGAGCTCCGTGTTATCCAAATCGAATCC | 17 | AP46_sps- |
| | | G | | dwn-rev- |
| | | | | SacI |
| Sucrose-glucan | AP-47 | GGTGGTGACGTCATTTACGCGAACCTTGGTGG | 18 | AP47_sgg- |
| glucosyltrans- | | | | up-fwd- |
| ferase. | | | | AatII |
| Sgg | AP-48 | GGTGGTGGTACCGCAGCGGAGAAGCTAAGGAT | 19 | AP48_sgg- |
| METBUDRAFT_2512 | | | | up-rev-KpnI |
| | AP-49 | GGTGGTGGGCCCTTTGTTCGGGCGTTTGTTCC | 20 | AP49_sgg- |
| | | | | dwn-fwd- |
| | | | | ApaI |
| | AP-50 | GGTGGTGAGCTCGGTCAGCAATTCCCAGTTTGA | 21 | AP50_sgg- |
| | | G | | dwn-rev- |
| | | | | SacI |
| Acyl-CoA | AP-13 | GAGGACGTCCACGACGGCATCACAATGAAC | 22 | fadE-up- |
| dehydrogenase, | | | | Fwd-AatII |
| fadE | AP-14 | GAGGGTACCGCTTAATTCCTGTTGCTGTGTC | 23 | fadE-up- |
| METBUDRAFT_2788 | | | | Rev-KpnI |
| | AP-15 | GAGGGGCCCGCCGAAGCCAAGTGTCCAAG | 24 | fadE-dwn- |
| | | | | Fwd-ApaI |
| | AP-16 | GAGGAGCTCCGTTGCCGTTGGTTGAACAC | 25 | fadE-dwn- |
| | | | | Rev-SacI |

20Z (see, e.g., GENEMBL under accession numbers FO082060 and FO082061)

| | | | | |
|---|---|---|---|---|
| Sucrose-6- | SB-1 | TTAGATCTCGGCCAAAATTGGCGATGAGTTGGT | 26 | sps-up-F |
| phosphate | SB-2 | TACCATGGACGATGACGGCAGCCGGCTTATCGA | 27 | sps-ud-R |
| synthase, sps | | C | | |
| MALCv4_0614 | SB-3 | TTCCGCGGTCGGCAATGGCAATTTTAAAGA | 28 | sps-dw-F |
| | SB-4 | TAGAGCTCGCCCAATCCGGTTCGATTTCA | 29 | sps-dw-R |
| Amylosucrase, | SB-5 | GAGACGTCGCTCAACCGACTCATTGACG | 30 | ams-up-F |
| alpha-amylase, | SB-6 | GTCAGCTGGGATGAATCGGCCTCATTCG | 31 | ams-up-R |
| ams | SB-7 | GAGGGCCCGTGGAATAGTAGCGCTAAAC | 32 | amp-dw-f |
| MALCv4_0617 | SB-8 | GAGAGCTCGATCAAAATCCGGCGTCGGGGGTCG | 33 | amp-dw-r |
| Δ mch-fae | MK-1 | GTCAGCTGGGATTTCGTTCAATTGCCGATG | 34 | mch-up/R- |
| MEALZv4_2776435_ | | | | PvuII |
| 2781800 | MK-2 | GAGACGTCCTATCCGCGCGTCTCCTTCACGG | 35 | mch-up/F- |
| | | | | AatII |
| | MK-3 | GAGGGCCCGCTCGGCATAGAATGACC | 36 | fae-dw/F- |
| | | | | ApaI |
| | MK-4 | GAGTTAACCGCTGGCAAGTGGAGCTGGTC | 37 | fae-dw/R- |
| | | | | HpaI |
| | MK-5 | GAGGGCCCGTTCGGCGCTTAAGAGTTTCG | 38 | mch20z- |
| | | | | dw/ApaI |
| | MK-6 | GAGAGCTCGTTCGCAACTAGCACCTTTG | 39 | mch20z- |
| | | | | dw/SacI |
| glgABC1- | MK-7 | GAGACGTCGCTCAACCGACTCATTGACG | 30 | glgA2-dw- |
| glgABC2 | | | | AatII |
| MEALZv4_4028360_ | MK-8 | GTCAGCTGGGATGAATCGGCCTCATTCG | 31 | glgA2-dw- |
| 4048136 | | | | PvuII |
| | MK-9 | GAGGGCCCGTGGAATAGTAGCGCTAAAC | 32 | glgA1-up- |
| | | | | ApaI |
| | MK-10 | GAGAGCTCGATCAAAATCCGGCGTCGGGGGTCG | 33 | glgA1-up- |
| | | | | Sac |
| | MK-11 | GAGGGGCCCGTGGAATAGTAGTGCTAAACAAT | 4 | glgA1- |
| | | | | dw/F/ApaI |
| | MK-12 | GAGGAGCTCCGTCAAAGGACGCCGTGAGCCCAG | 5 | glgA1- |
| | | | | dw/R/SacI |
| | MK-13 | GAGGACGTCCACAGCGGCTTTGACTGGATCG | 2 | glgA1- |
| | | | | up/F/AatII |
| | MK-14 | GAGGGTACCGAGTTTACCGAGGTGGATTTCGCC | 3 | glgA1- |
| | | | | up/R/KpnI |
| | MK-15 | GACCATGGCACAACGGCATATTGGATTGC | 40 | glgB1- |
| | | | | up/NcoI |
| | MK-16 | GAGAATTCGCTGTCGGCATCTTTGATC | 41 | glgB1- |
| | | | | up/EcoRI |

5GB1 GenBank/EMBL under the accession numbers AOTL01000000 and KB45575
and KB455576.

| | | | |
|---|---|---|---|
| Glycogen | AP-90 | TGGATGCATATGCTGCAGCTGAGTTCGGGCAAG | AP90_glgA1KO_ |
| synthase, glgA1 | | GTACAGA | UP_Fwd |
| METBUDRAFT_3833 | AP-91 | GAACTCTCCAGGCAGTCAAAATTCGGTTCTAG | AP91_glgA1KO_ |
| | | | UP_Rev |
| | AP-92 | TTTGACTGCCTGGAGAGTTCGGTAACTCGATTG | AP92_glgA1KO_ |
| | | | DWN_Fwd |
| | AP-93 | CCGGTTAACACGCGTACGTATGTGGAATTACCG | AP93_glgA1KO_ |
| | | GAAGGCA | DWN_Rev |
| Glycogen | AP-94 | TGGATGCATATGCTGCAGCTAGTTGGGGCGAAC | AP94_glgA2KO_ |
| synthase, glgA2 | | AAGGCTA | UP_Fwd |
| METBUDRAFT_3841 | AP-95 | ATCGGCCTCTATTTATCATCCCGCTGCCCTTAG | AP95_glgA2KO_ |
| | | | UP_Rev |
| | AP-96 | GATGATAAATAGAGGCCGATTGATGAGGAT | AP96_glgA2KO_ |
| | | | DWN_Fwd |
| | AP-97 | CCGGTTAACACGCGTACGTAGCAAATAAACGAG | AP97_glg2KO_ |
| | | GCGTATC | DWN_Rev |
| PEP synthase, | AP-9 | GAGGAATTCCGCCAAGCATACTCCACACC | pps-UP-F- |
| pps | | | EcoRI |
| METBUDRAFT_0376 | AP-10 | GAGGGTACCCTTCACGCATGATTAACACCC | pps-UP-R- |
| | | | KpnI |
| | AP-11 | GAGGGGCCCATAATCCTGAGCAGCCGGAG | pps-Dwn-F- |
| | | | ApaI |
| | AP-12 | GAGGAGCTCCTGCAACAACATAAAGCCGAC | pps-Dwn-R- |
| | | | SacI |
| Sucrose-6- | AP-110 | ATTGGTACCATGGATGCATATGCTGCAGCTACGC | AP110_spsKO_ |
| phosphate | | TGCTCTAAATACCTTG | UP_Fwd |
| Synthase, sps | | | |
| METBUDRAFT_2515 | | | |
| Lactate | AB-7 | ATATGAGTAAACTTGGTCTGACAGTTACCACCTT | ldh-up-fwd |
| dehydrogenase | | CGCCGATTTGGTTTCT | |
| ldh | AB-8 | ATCTTTCAGCTTTGGCATTCTAAATCCCGTTCGC | ldh-up-rev |
| METBUDRAFT_3726 | | C | |
| | AB-9 | ACGGGATTTAGAATGCCAAAGCTGAAAGATGAA | ldh-dwn-fwd |
| | | GG | |
| | AB-10 | CGTGCATCACGACACCGACAACCTGCACATTCG | ldh-dwn-rev |
| | | ATTCTAATGAGCCTGTTC | |
| Acetate kinase | AB-3 | ATATGAGTAAACTTGGTCTGACAGTTACCAATTT | ack-up-fwd |
| ack | | ATTCAAACTGCAACCG | |
| METBUDRAFT_1552 | AB-4 | AACGTGTTGAAGAATAAACCAGCATATCCCTAG | ack-up-rev |
| | | CAG | |
| | AB-5 | GGGATATGCTGCTGGTTTATTCTTCAACACGTTA | ack-dwn-fwd |
| | | GGTCCC | |
| | AB-6 | CGTGCATCACGACACCGACAACCTGCACATGGG | ack-dwn-rev |
| | | TGAATGGGTGGATGAAT | |
| Methylenetetrahy | mtdB1_ | TGGATGCATATGCTGCAGCTTCTATCGAAAGCA | mtdb12-up- |
| dromethanopterin | LF_F | ACACCAGGAACC | fwd |
| dehydrogenase | mtdB1&2_ | CAGCATTGATGCGAGCAGTAGATCATCTTGCACT | mtdb12-up- |
| mtdB1B2 | LF_R | AAGCCGCGTA | rev |
| METBUDRAFT_ | mtdB1&2_ | TACGCGGCTTAGTGCAAGATGATCTACTGCTCGC | mtdb12- |
| 1893-1894 | RF_F | ATCAATGCTG | dwn-fwd |
| | mtdB2_ | CCGGTTAACACGCGTACGTAGTGATATTCAGGTT | mtdb12- |
| | RF_R | GTGGCGGT | dwn-rev |

20Z GENEMBL under accession numbers FO082060 and FO082061

| | | | |
|---|---|---|---|
| Sucrose-6- | SB-1 | TTAGATCTCGGCCAAAATTGGCGATGAGTTGGT | sps-up-F |
| phosphate | SB-2 | TACCATGGACGATGACGGCAGCCGGCTTATCGA | sps-ud-R |
| synthase, sps | | C | |
| MALCv4_0614 | SB-3 | TTCCGCGGTCGGCAATGGCAATTTTAAAGA | sps-dw-F |
| | SB-4 | TAGAGCTCGCCCAATCCGGTTCGATTTCA | sps-dw-R |
| Amylosucrase, | SB-5 | GAGACGTCGCTCAACCGACTCATTGACG | ams-up-F |
| alpha-amylase, | SB-6 | GTCAGCTGGGATGAATCGGCCTCATTCG | ams-up-R |
| ams | SB-7 | GAGGGCCCGTGGAATAGTAGCGCTAAAC | amp-dw-f |
| MALCv4_0617 | SB-8 | GAGAGCTCGATCAAAATCCGGCGTCGGGGGTCG | amp-dw-r |
| glgABC1- | MK-7 | GAGACGTCGCTCAACCGACTCATTGACG | glgA2-dw- |
| glgABC2 | | | AatII |
| MEALZv4_4028360_ | MK-8 | GTCAGCTGGGATGAATCGGCCTCATTCG | glgA2-dw- |
| 4048136 | | | PvuII |
| | MK-9 | GAGGGCCCGTGGAATAGTAGCGCTAAAC | glgA1-up- |
| | | | ApaI |
| | MK-10 | GAGAGCTCGATCAAAATCCGGCGTCGGGGGTCG | glgA1-up- |
| | | | Sac |
| | MK-11 | GAGGGGCCCGTGGAATAGTAGTGCTAAACAAT | glgA1- |
| | | | dw/F/ApaI |
| | MK-12 | GAGGAGCTCCGTCAAAGGACGCCGTGAGCCCAG | glgA1- |
| | | | dw/R/SacI |
| | MK-13 | GAGGACGTCCACAGCGGCTTTGACTGGATCG | glgA1- |
| | | | up/F/AatII |
| | MK-14 | GAGGGTACCGAGTTTACCGAGGTGGATTTCGCC | glgA1- |
| | | | up/R/KpnI |
| | MK-15 | GACCATGGCACAACGGCATATTGGATTGC | glgB1- |
| | | | up/NcoI |
| | MK-16 | GAGAATTCGCTGTCGGCATCTTTGATC | glgB1- |
| | | | up/EcoRI |
| Acetate kinase | MK-17 | CAACCGAACACCGAACATCCTC | ask/UP-F- |
| MALCv4_2853 | | | AatII |
| | MK-18 | AAGCTGAAGCATCTTGCTAATCAGG | ask/UP-R- |
| | | | KpnI |
| | MK-19 | GGCTGTGCTTGATAAAGATCACGC | ask/DW-F- |
| | | | SacII |
| | MK-20 | GACGATTGCGCGGCTGTGTTGG | ask/DW-R- |
| | | | HpaI |
| Lactate | MK-21 | CAGGATGCGTGCCGAAATAAG | Ldh/UP-F- |
| dehydrogenase | | | AatII |
| MALCv4_0534 | MK-22 | GAAGAACCGACATGGCCGG | Ldh/UP-R- |
| | | | NcoI |
| | MK-23 | GGAGTAGGTGAAAGGTGAAAGG | Ldh/Dw-F- |
| | | | SacII |
| | MK-24 | GGCAGGGTAAGGCGGCGAG | Ldh/Dw-R- |
| | | | SacI |

Upstream and downstream fragments were PCR amplified, cloned into pCR2.1, and then subcloned into pCM184. Each construct was verified by sequencing. Alternatively, PCR amplified upstream and downstream fragments were inserted into a kanamycin-resistant version of the sucrose counterselection vector pCM433 (Marx 2008). Resulting vectors were introduced into a donor strain E. coli S17-1 via standard transformation procedure (Sambrook et al., 1989). The donor strain grown on LB-agar medium supplemented with appropriate antibiotic and the recipient Methylomicrobium strain grown on NMS2-agar medium were mixed in a donor:recipient ratio of 1:2, and plated on the optimized mating medium (Ojala et al., 2010). Plates were incubated at 30° C. under methane:air atmosphere (25:75) for 48 h, and cells were transferred from a mating medium onto selective plates. Rifamycin, high pH and 3% salinity were applied for counter-selection against the donor cells. The Kan^{r} recombinants were selected and re-plated onto new plates. In the case of mutants constructed using the sucrose counter-selection vector for unmarked alleic exchange, the Kan^{r} recombinants were plated on NMS2 medium containing 2.5% sucrose and subsequently checked for kanamycin sensitivity. The identity of the double-crossover mutants was verified by diagnostic PCR with primers specific to the insertion sites. Constructed strains include those listed in Table 7.

TABLE 7
Constructed Strains

| Strains | Description | Parental strain |
|---|---|---|
| M. alcaliphilum | Rif^{R} derivative | 20Z wild type |
| 20ZR | | |
| M. alcaliphilum | High conjugation efficiency | |
| 20ZRor [symbol] | strai [symbol] | |
| M. buryatense | Rif^{R} derivative, | 5G wild type |
| 5GB1 | with a mutation in rpoS-gene | |
| | (MBURv2_50058), which | |
| | has a 309 base pair insertion | |
| | that results in a stop codon | |
| | after 218 aa | |
| M. buryatense | The strain lacks an endogenous | 5GB1 |
| 5GB1S | 82 kb plasmid | |
| pAWP45 | ΔglgA1 | 5GB1 |
| pAWP47 | ΔglgA2 | 5GB1 |
| pAWP45/47 | ΔglgA1, ΔglgA2 | 5GB1 |
| pAWP14 | Δpps::kan | 5GB1 |
| pAB2 | Δsps | 5GB1 |
| pAWP68 | ΔfadE | 5GB1 |
| pAB7 | Δack | 5GB1 |
| pAB8 | Δldh | 5GB1 |
| pRL5 | ΔmtdB1B2 | 5GB1 |
| pBS1 | Δsps::kan | 20Z |
| pBS2 | Δams::kan | 20Z |
| pBS3 | ΔglgABC1manBQamyC::kan | 20ZR |
| pBS4 | Δack::kan | 20ZR |
| pBS5 | ΔglgAB2amyAC | 20ZR |
| pBS6 | ΔglgABC1-glgABC2 | 20ZR |
| pBS7 | Δldh::kan | 20ZR |

[symbol]

indicates data missing or illegible when filed

Characterization of glycogen accumulation in Methylomicrobium alcaliphilum 20Z and Methylomicrobium buryatense 5GB1 wild type and mutant strains demonstrated that down-regulation of genes involved in glycogen or sucrose biosynthesis results in reduction of intracellular glycogen as shown in Tables 8 and 10.

TABLE 8
Glycogen accumulation* in Methylomicrobium alcaliphilum 20Z wild
type and mutant strains grown at different concentrations of salt.

| | Glycogen | | | |
|---|---|---|---|---|
| Salt concentration in | ng/mg of cells | | | |
| growth medium | WT | Δ sps | Δ ams | Δ glgABC1manBQamyC |
| 1% NaCl | 1 | 5 | 31 | 2 |
| 3% NaCl | 177 | 157 | 2 | 8 |
| 5% NaCl | 103 | 80 | 20 | 100 |

*Glycogen concentrations were measured by using Glycogen Assay Kit (Abcam) in accordance with the manufacturer instructions.

TABLE 10
Glycogen accumulation* in Methylomicrobium buryatense
5GB1 wild type and mutant strains.

| Strain | Glycogen content (% cell dry weight) |
|---|---|
| 5GB1 | 0.20 ± 0.05 |
| Δsps | 0.22 ± 0.04 |
| Δ glgA1 | 0.23 ± 0.02 |
| Δ glgA2 | 0.20 ± 0.04 |
| Δ glgA1/A2 | Not detected |

**Glycogen concentrations in potassium hydroxide cell extracts were measured by using Glycogen Assay Kit (Abcam) in accordance with the manufacturer instructions.

Each modification disclosed herein results in a modified Group I MB that has at least one of the following characteristics:

(a) lipid content of 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50% wherein lipid content is measured by gravimetric assay after solvent (chloroform:methanol) extraction;
(b) carbon conversion efficiency of 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%,74%, 75%, 76%,77%, 78%, 79%, 80%, 81%, 82%, 83%, 84%, 85%, 86%, 87%, 88%, 89% or 90% wherein carbon conversion efficiency is percentage of total carbon incorporated into cell material and is calculated using equation: CCE=([C mol in biomass]/[C mol of substrate consumed])*100; and/or
(c) ability to generate 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5 or 3.6 g total fuel precursor lipids (FFAs+phospholipids+fatty acid esters)/L fermentation broth/hr of substrate.

### REFERENCES

Angelé, B., Kirchner, K. 1980. Chem Eng Sci. 35(10): 2089-2091.
Anthony, C. 1982. The biochemistry of methylotrophs. London. New York: Acad. Press 431p.
Bao, B., El-Halwagi, M. M., Elbashir, N. O. 2010. Fuel Processing Technology 91:703-713.
Beck D A, Hendrickson E L, Vorobev A, Wang T, Lim S, Kalyuzhnaya M G, Lidstrom M E, Hackett M, Chistoserdova L. 2011. J Bacteriol. 193(18):4758-65.
Bligh, E. G., Dyer, W. J. 1959. Can J Biochem Phys 37, 911-917.
Bouwens, S. M., Vissers, J. P. R., et al. 1988. J Catal. 112(2): 401-10.
Chistoserdova, L., Kalyuzhnaya M. G., M. E. Lidstrom. 2009. Annu Rev Microbiol. 63:477-99.
Christie, W. W. 2005. Lipid analysis. Bridgewater, N.J.: The Oily Press, PJ Barnes &Associates.
Clarke, A. J., Sarabia, V., et al. 1991. Anal Biochem. 199. 68p.
Cvejic, J. H., Bodrossy, L., et al. 2000. FEMS Microbiol Lett. 182: 361-5
Dalton H. 2005. Philos Trans R Soc Lond B Biol Sci. 360:1207-22
Davis, D., Lok, C. M., et al. 2009. PCT Publication No. WO2009095711.
Dedysh, S. N., Panikov, et al. 1998. Science. 282: 281-4.
Economides M. J. 2005. World Energy. 8(1): 136-40.
Fan J., Yan C., A. C., et al., 2012. Plant Cell Physiol. May 28. [Epub ahead of print]
Fang, J., Barcelona, M. J., Semrau, J. D. 2000. FEMS Microbiol Lett 189: 67-72.
Guarnieri, M. T., Nag, A., et al., 2011. PLoS One 6:e25851.
Guckert, J. B., Cooksey, K. E., Jackson, L. L. 1988. J Microbiol Methods 8: 139-149.
Hanson, R. S., T. E. Hanson 1996. Microbiol. Rev. 60 (2): 439-71.
Hara, A., Radin, N. S. 1978. Anal Biochem. 90: 420-6.
Hu, Q., Sommerfeld, M., Jarvis, E., et al. 2008. Plant Journal. 54: 621-639.
Huber, G. W., O'Connor, P., Corma, A., Sauvanaud, L. 2007. J Catal. 257: 136-71
Kaluzhnaya, M. G., Khmelenina V. N., et al. 2001. Syst Appl Microbiol. 24: 166-76
Kalyuzhnaya et al. 2013 Nature Comms Doi: 10.1038/ncomss3785
Kalyuzhnaya, M. G., Beck, D. A. C., Chistoserdova, L. 2011. Methods Enzymol. 495: 81-98.
Kalscheuer, R., Stölting T., & A. Steinbüchel. 2006. Microbiol. 152: 2529-36.
Khadem, A. F., A. Pol, A. Wieczorek, et al. 2011. J Bacteriol. 193: 4438-46.
Khmelenina, V. N., Kalyuzhnaya M. G., et al. 1999. Arch. Microbiol, 172: 321-29.
Koritala, S. 1975. J Am Oil Chemist's Soc. 52(7): 240-3
Kubicka, D., Kaluza, L. 2010. Appl Catal A:Gen. 372:199-208
Laurens, L. M. L., Quinn, M., VanWychen, S., et al. 2012. Anal Bioanal Chem. 403: 167-78.
Leak, D. J., Dalton, H. 1986. Appl Microbiol Biotech. 23:477-481
Lidstrom, M. E. 2006. In The Prokaryotes, pp. 618-634. New York: Springer-Verlag.
Liu X, Sheng J, Curtiss R 3rd. 2011. PNAS 108:6899-904.
Marx and Lidstrom 2002 Biotechniques 5:1062-7
Marx 2008 BMC Res Notes 1:1
McCall, M. J., Kocal J. A., et al. 2009. U.S. Pat. No. 803,968B2.
Merkle J., A., Ball H. 2001. U.S. Pat. No. 6,217,926.
Murrell, J. C., Jettel, M. S. M. 2009. Environ Microbiol Rep. 1(5): 279-84.
Myllyoja, J. 2007. U.S. Pat. No. 05,014,426.0
Nádasdi, L., and Joó, F. 1999. Chimica Acta. 293(2): 218-22.
Nagle, N., Lemke, P., 1990. Appl Biochem Biotechnol. 24(1): 355-361.
Ojala D. S., Beck, D. A. C., Kalyuzhnaya, M. G. 2011. Methods Enzymol. 495: 99-118
Olah G.A., Goeppert, A., et al. 2006. Beyond Oil and Gas: the Methanol Economy. 290pp Oxford Catalyst. 2012.
Palkovits, R., Antonietti, M., et al. 2009. Angew Chem Int Ed. 48: 6908-12.
Scott, D., Best, D. J., Higgins I. J. 1981. Biotech Lett, 3: 641-4.
Shoda, M., S. Nagai, S. Aiba. 1975. J. appl. Chem Biotechnol. 25:305-318.
Starostina, N. G., Pashkova, N. I., Tsiomenko, A. B. 1998. Biochemistry (RAS). 63(10): 1122-5.
Stehen, E. J., Kang, Y., et al. 2010. Nature. 463:559-62.
Templeton, D. W., Quinn, M., et al., 2012. J Chromatogr. (submitted).
Van Dien, S. J., Lidstrom, M. E. 2002. Biotechnol Bioeng 78: 296-312.
Veazey M. W. 2012. E&P news. Rigzone. Available on the world wide web at rigzone.com/news/article.asp?a_id=116784
Vigh, L., Horváth, I., et al., 1987. Biochimica et Biophysica Acta (BBA). 921(2): 167-74.
Vuilleumier, S. , Khmelenina, V. N. at al., 2012. J. Bacteriol. 194(2): 551-2.
Winder R. 2010. Chemistry and Industry. World Bank and The Global Gas Flaring Reduction Partnership (GGFR) 2008.
Yang S, Sadilek M, Lidstrom M E. 2010. J. Chromatogr A. 1217(47):7401-10.

### Example 2

### Genetically Tractable Variant Strains of Methanotrophic Bacteria and Exemplary Methods of Making

Aerobic methanotrophic bacteria, including Methylomicrobium bacteria, have generated interest because they utilize methane as a sole source of carbon and energy.

A bottleneck for working with industrially relevant methanotrophic bacteria, including Methylomicrobium buryatense and Methylomicrobium alcaliphillium strains, are the low frequencies at which genetic material can be introduced via conjugation. There is presently an unmet need for a method of generating genetically tractable variants of methanotrophic bacteria. Specifically, there is an unmet need for methods of generating genetically tractable methanotrophic bacteria and for the genetically tractable methanotrophic bacteria themselves.

### Genetic Tractability

Genetic tractability was measured by conjugating either pVK100 or pCM66 into the Methylomicrobium buryatense strains using an E. coli S17-1 donor strain, and counting the number of antibiotic-resistant colonies formed per plate. This was done in triplicate and the numbers reported are the mean number of colonies±standard deviation.

For pVK100 conjugations, the relative number colonies per plate was calculated as the mean of: (number of 5GB1 or 5GB1S colonies divided by the mean number of 5GB1 colonies per plate).

Relative genetic tractability was calculated as the mean of: (number of 5GB1S colonies divided by the sum of the mean number of 5GB1 colonies per plate plus the mean number of 5GB1S colonies per plate)

TABLE 12
Genetic tractability (pVK100)

| | Mean # | Relative # | Relative Genetic |
|---|---|---|---|
| | Colonies/Plate | Colonies/Plate | Tractability |
| 5GB1 | 48 ± 21 | 1 ± 0.4 | 4.9 × 10⁻⁵ ± 2.2 × 10⁻⁵ |
| 5GB1S | 982800 ± 801506 | 20475 ± 16698 | 1.0 ± 0.8 |

Relevant ranges:

3,000-50,000 fold increase in # colonies/plate for pVK100 conjugated into 5GB1 vs 5GB1S, A relative genetic tractability of 0.2-1.0 for 5GB1S compared to 5GB1 using pVK100.

TABLE 13
Genetic tractability (pCM66)

| | Mean # | Relative Genetic |
|---|---|---|
| | Colonies/Plate | Tractability |
| 5GB1 | 0 ± 0 | 0 ± 0 |
| 5GB1S | 13 ± 1 | 1 ± 0.1 |

Relevant range:

A relative genetic tractability of 0.9-1.0 for 5GB1S compared to 5GB1 using pCM66.

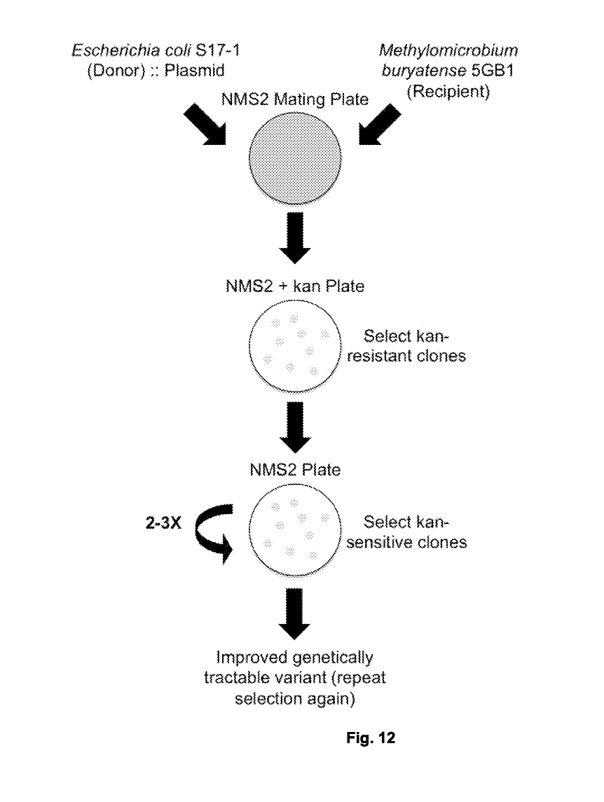

A variant of M. buryatense 5GB1 was selected that is more amenable to genetic manipulation. This selection was carried out by mating a version of the IncP-based broad host range plasmid pVK100 into 5GB1 using an E. coli S17-1 donor (FIG. 12) (Knauf & Nester, 1982). Once kanamycin-resistant 5GB1::pVK100 colonies were obtained, these were passaged 3 times on non-selective NMS2 plates to enable loss of the plasmid. Following plasmid loss as determined by kanamycin sensitivity, a variant of the smaller IncP-based broad host range vector pCM66 was mated into this new strain to begin a second round of selection (Marx & Lidstrom, 2001).

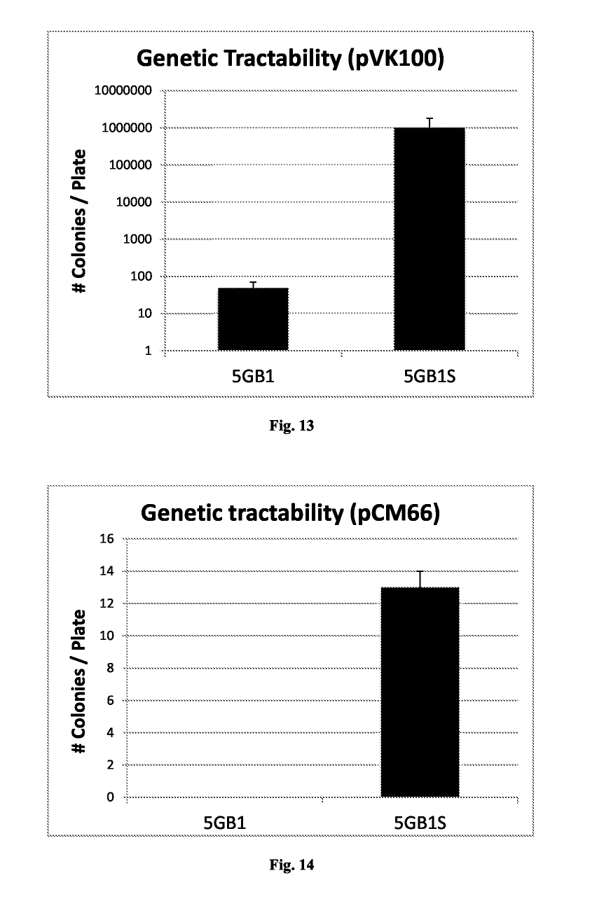

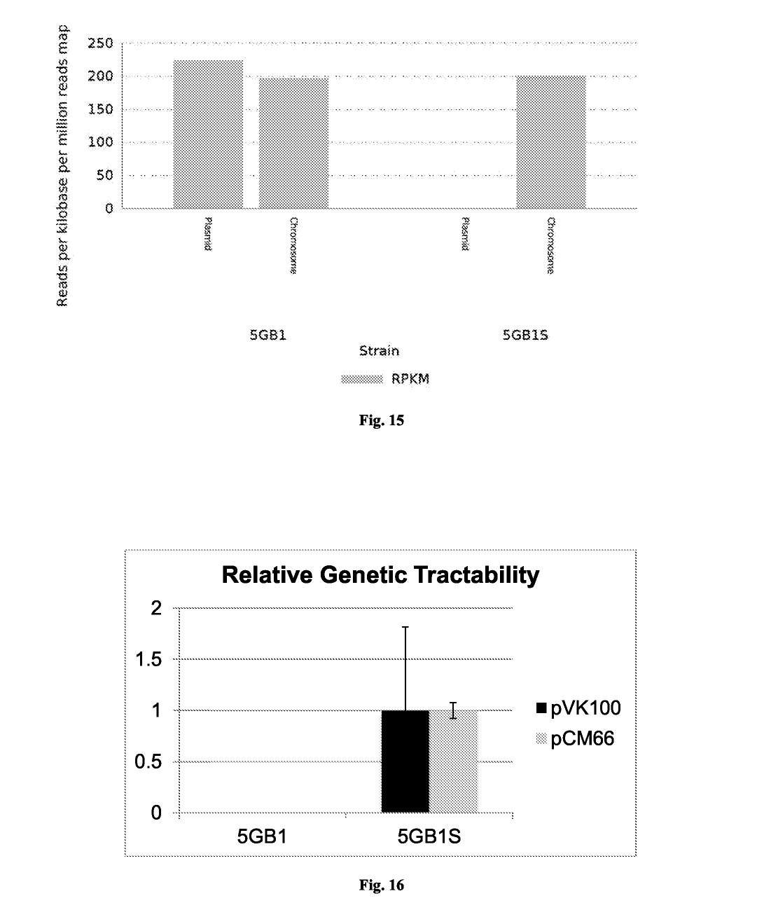

After curing out this second plasmid, the resulting improved genetically tractable 5GB1S strain accepts pVK100 at a frequency several orders of magnitude higher than the parent strain, 5GB1 (FIG. 13). In addition, smaller trimmed variants of IncP plasmids such as pCM66 are also successfully transferred (FIG. 14), greatly improving heterologous expression capabilities in M. buryatense. This includes the use of modern cloning techniques such as Gibson cloning that benefit from smaller vector backbone sizes (Gibson et al., 2009).

Sequencing of the genomes of 5GB1 and 5GB1S has revealed that the 5GB1S strain has lost the endogenous 82 kb plasmid found in M. buryatense (Khmelenina et al., 2013). Without wishing to be bound by theory, it is thought that replication and/or conjugation functions on this endogenous plasmid interfere with replication and/or conjugation of a heterologous plasmid. Therefore, loss of this endogenous plasmid results in a more genetically tractable variant of M. buryatense and this strain will serve as a promising platform for industrial methane biocatalysis.

### Example 3

### Pyruvate Flux Into Mutants vs. Wild-Type M. buryatense 5GB1

Methods

M. buryatense 5GB1 wild type and mutants were pre-cultured in a 250 ml vial with 50 ml of NMS2 medium (Ojala et al. 2010) using ¹²C methane as sole carbon source and energy source. ¹³C methane was introduced to the vial when the OD₆₆₀ reached 0.6 to 0.8, and the samples were incubated at room temperature. 12 ml each for two replicates per vial were harvested at time point 1 min, 5 min, 10 min, 20 min, 40 min and 1 hour using fast filtration. The filter was then frozen in liquid nitrogen and lyophilized. Hot water extraction method was used for metabolite extraction, and the same was further concentrated by lyophilization. Dried samples were then reconstituted in 50 μl of H₂O and separated on a Zic-pHILIC column using LC-MS/MS for metabolite and isotopomer detection. Isotopomer ratio data for pyruvate were analyzed in MassLynx. Time course data were then fitted using exponential decay in R for each replicate to obtain the rate constant k.

Results

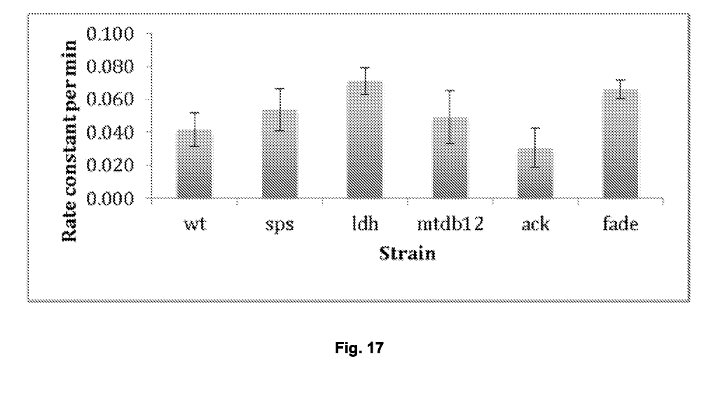

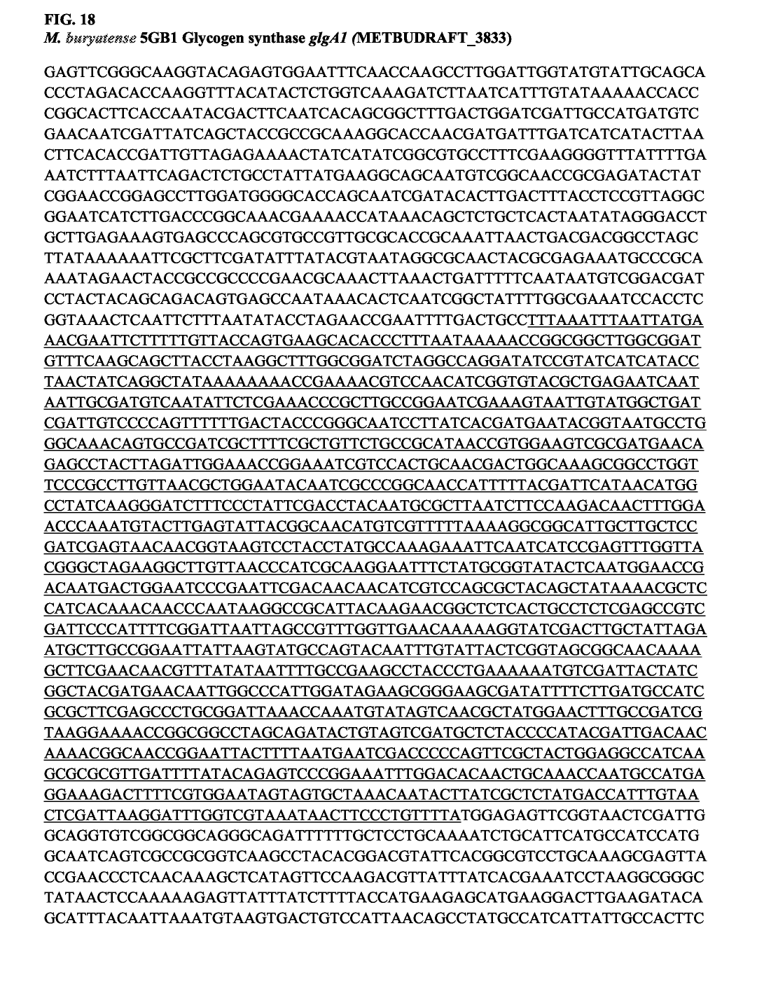

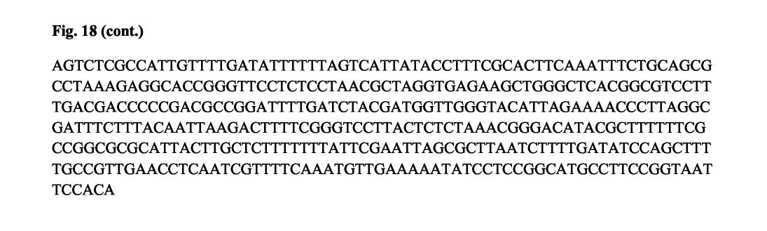

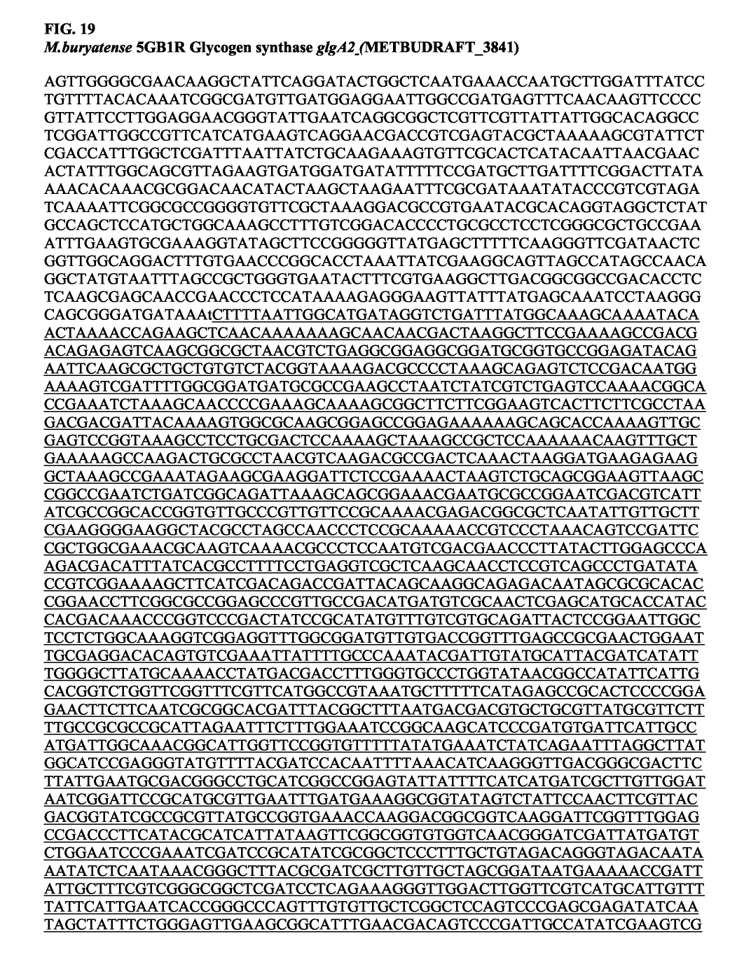

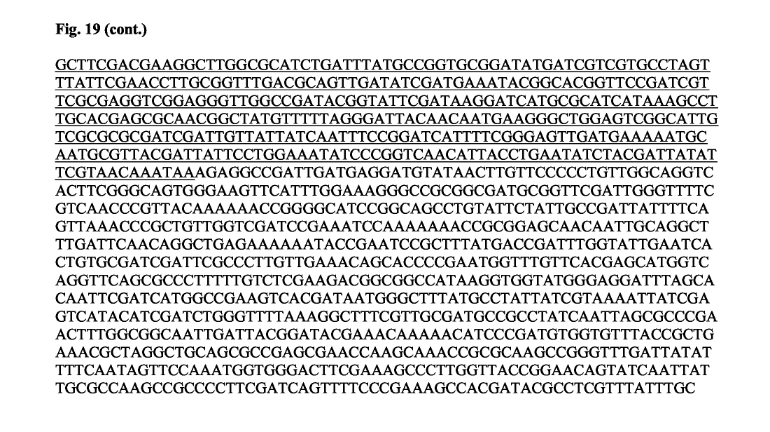

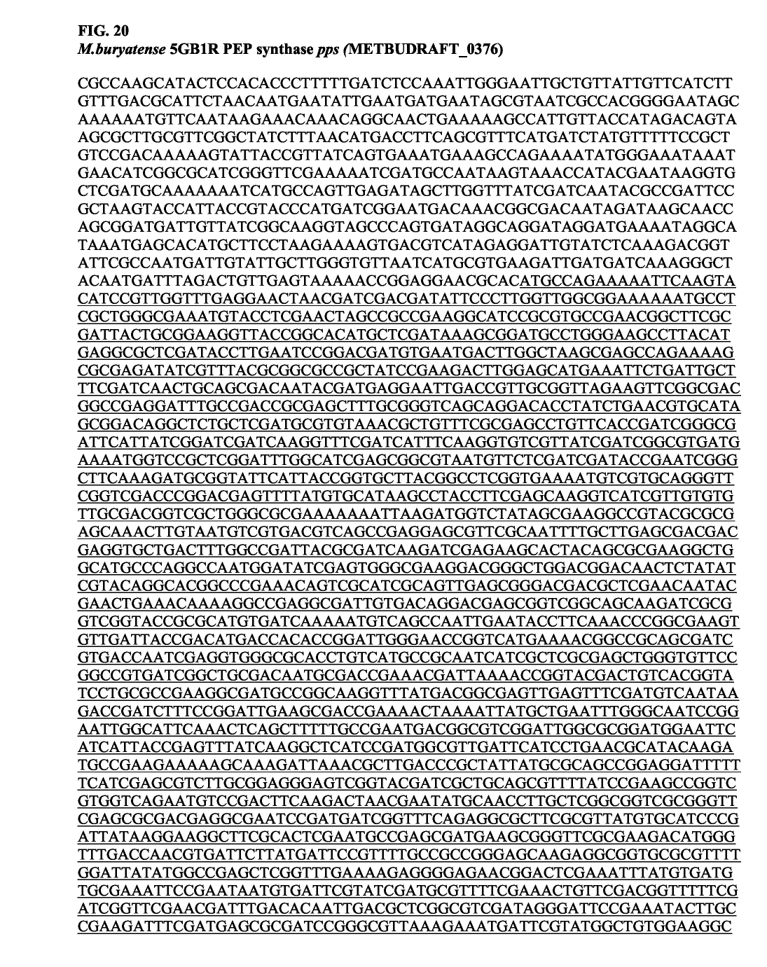

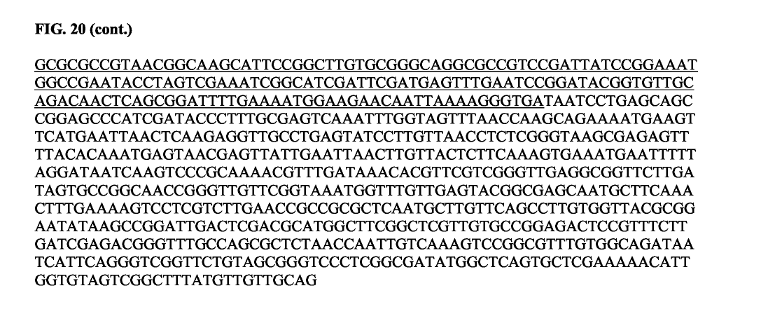

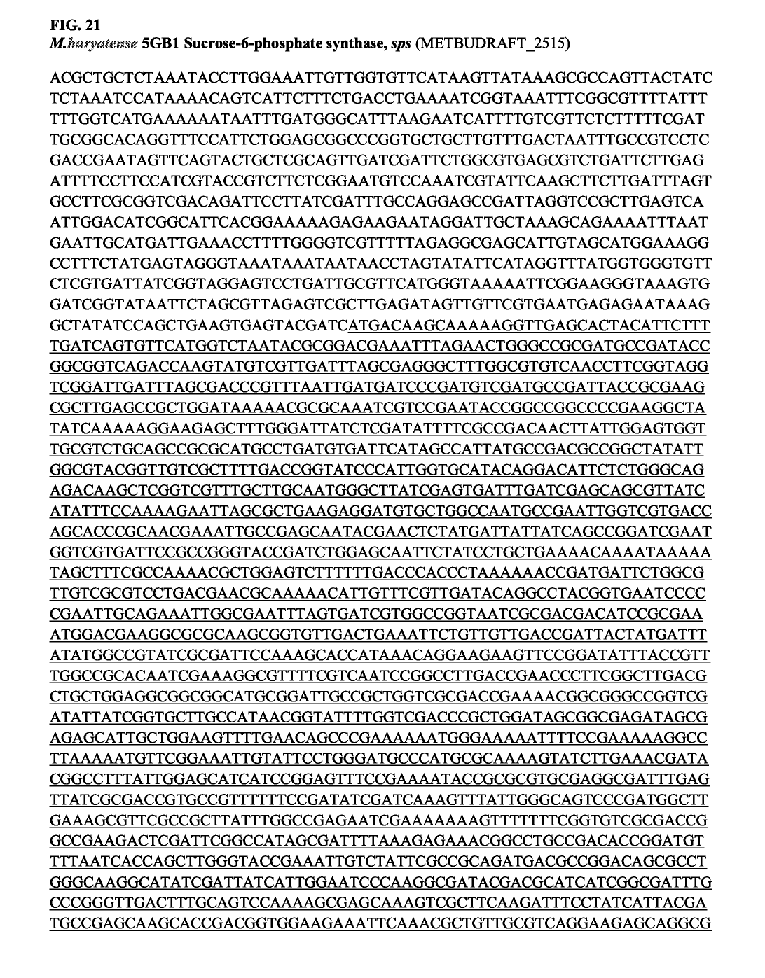

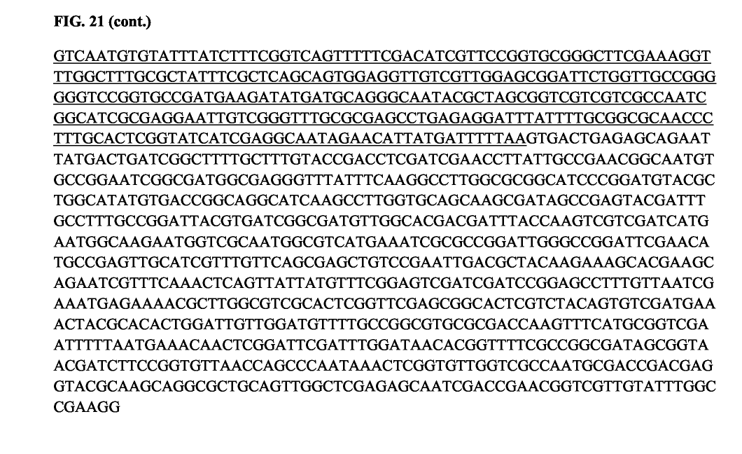

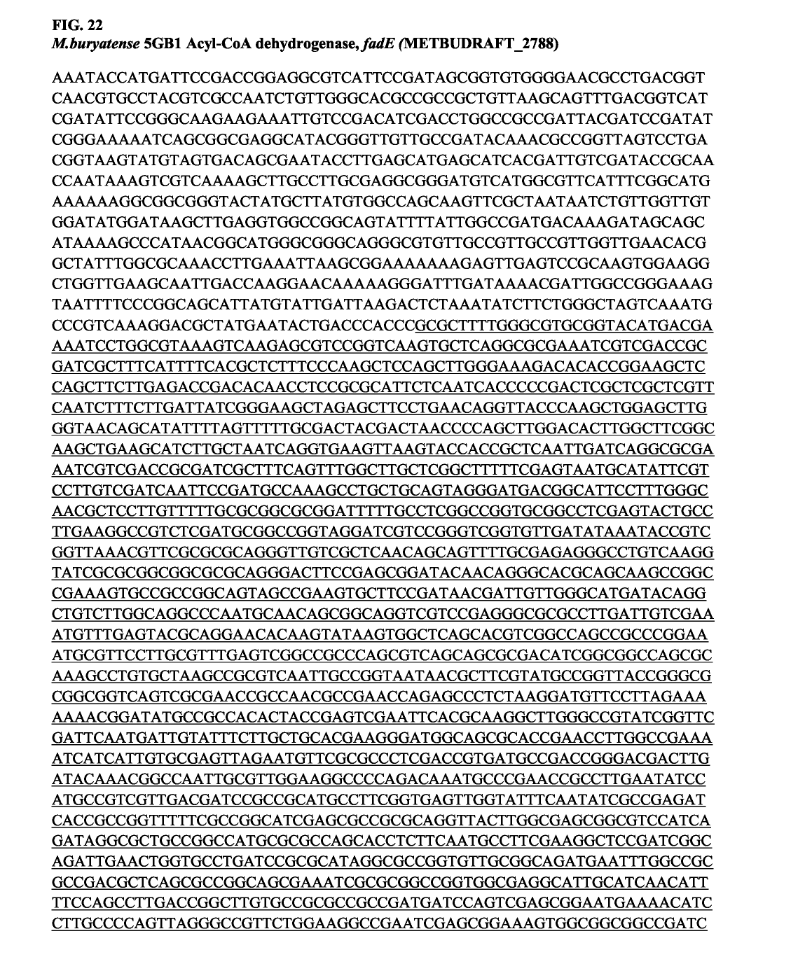

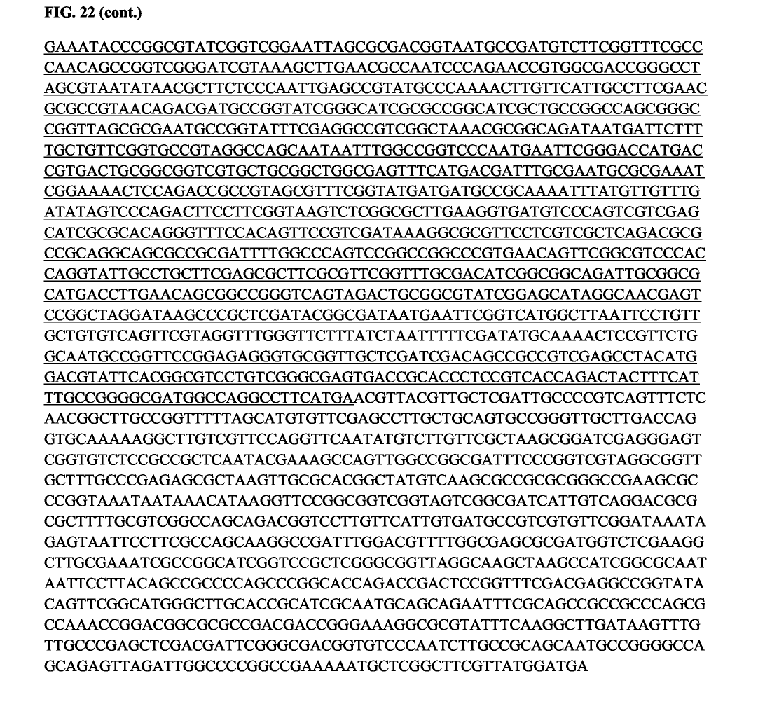

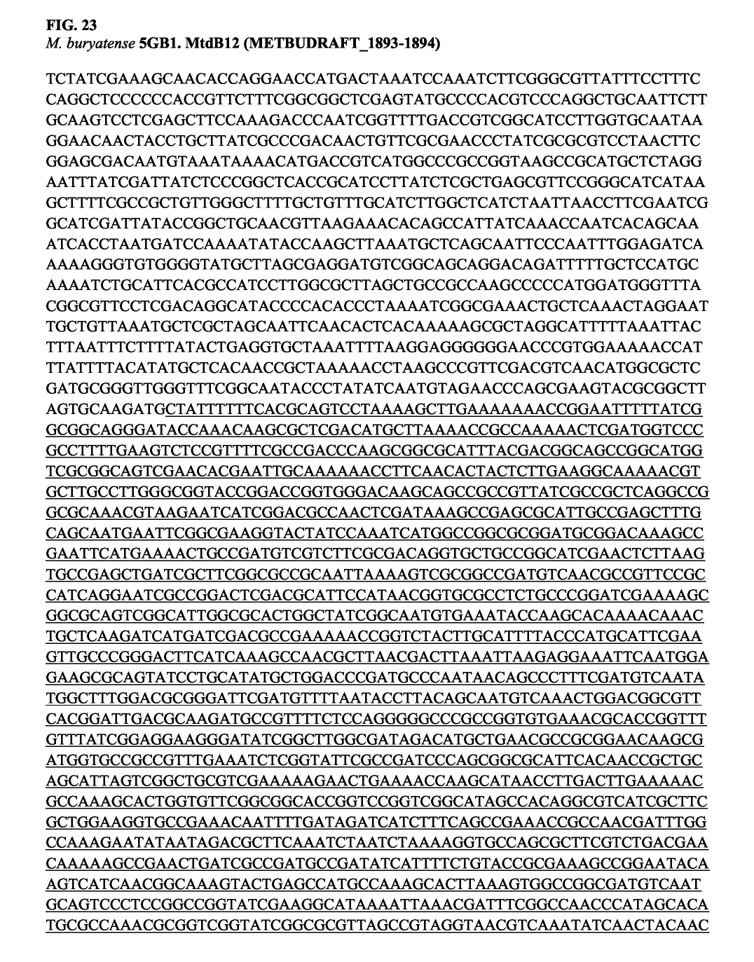

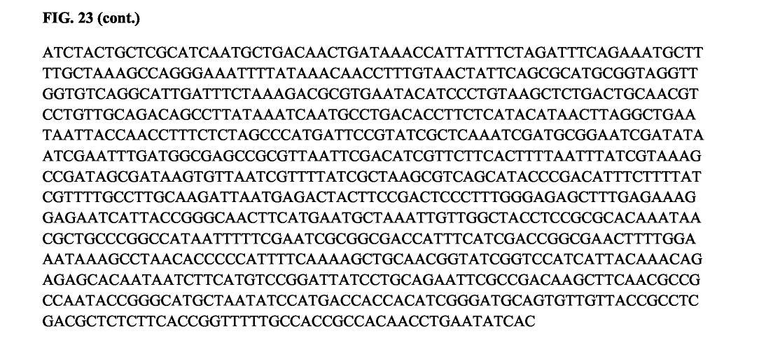

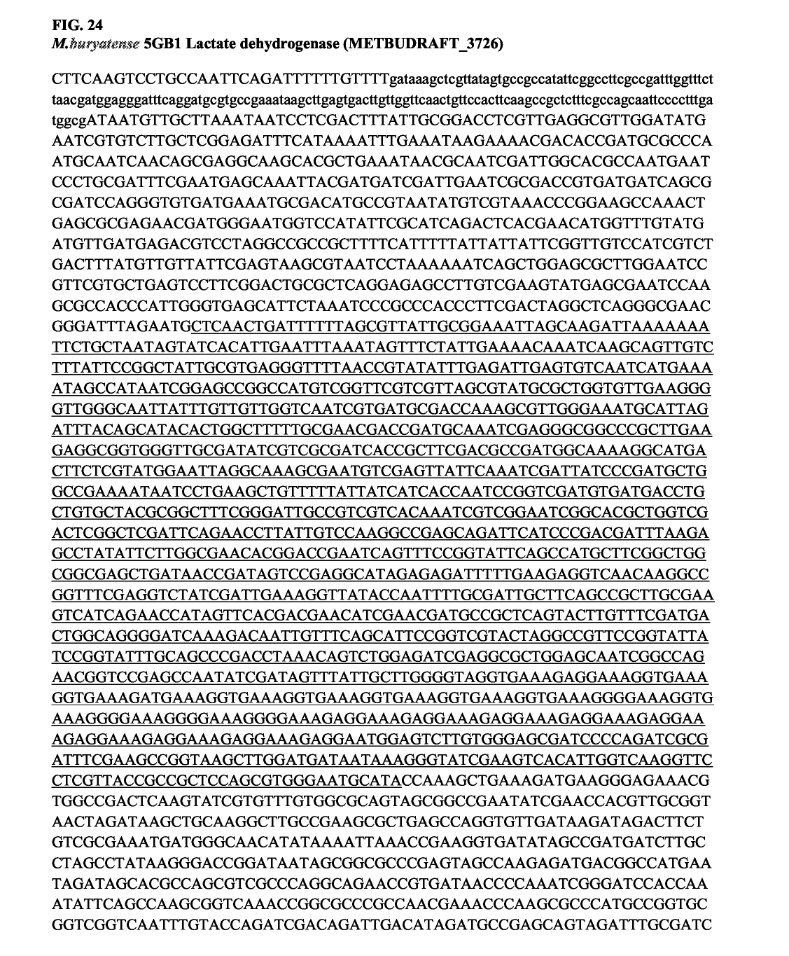

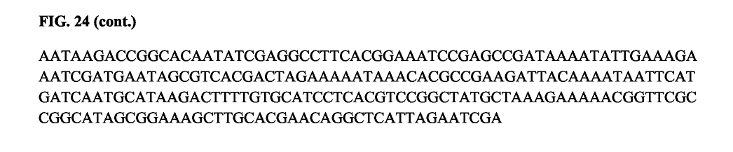

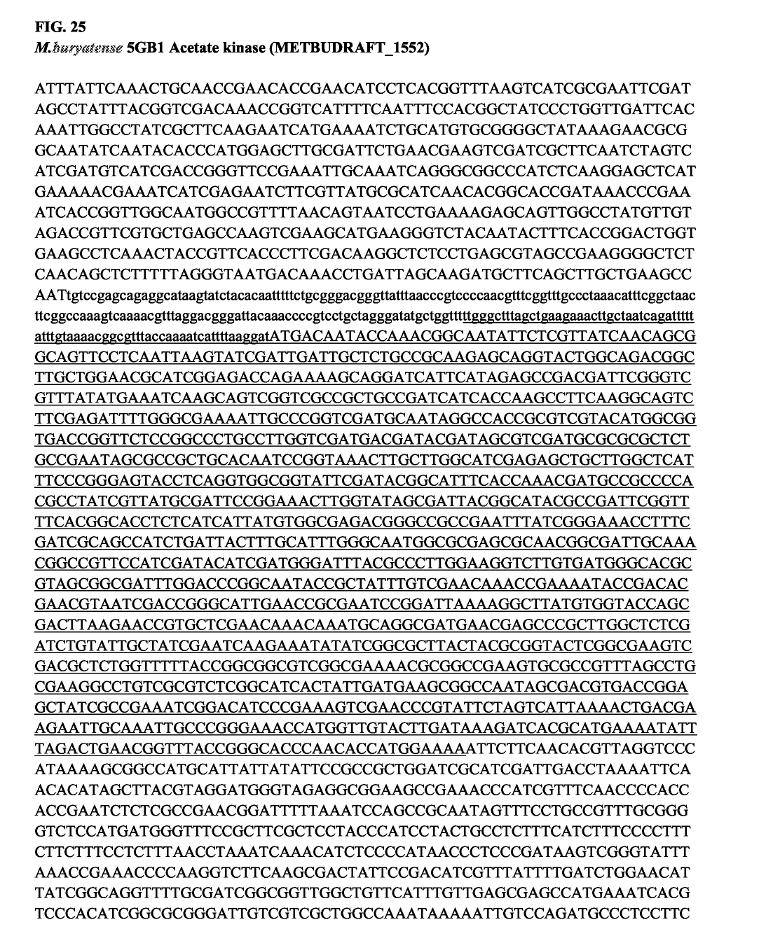

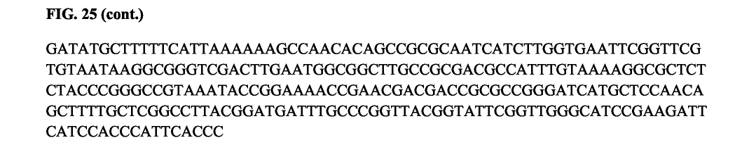

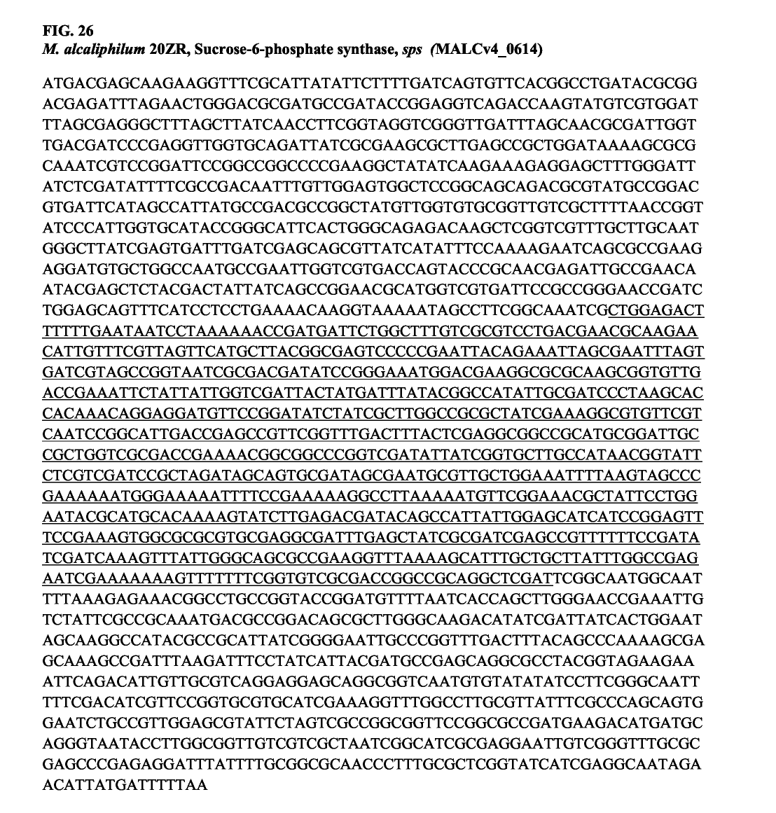

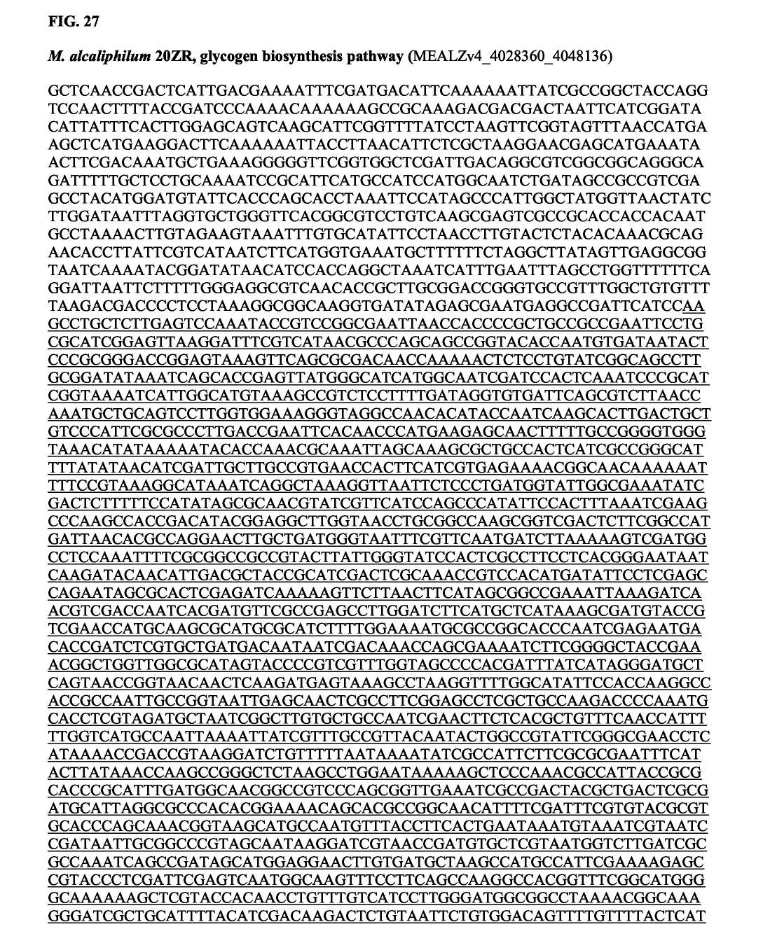

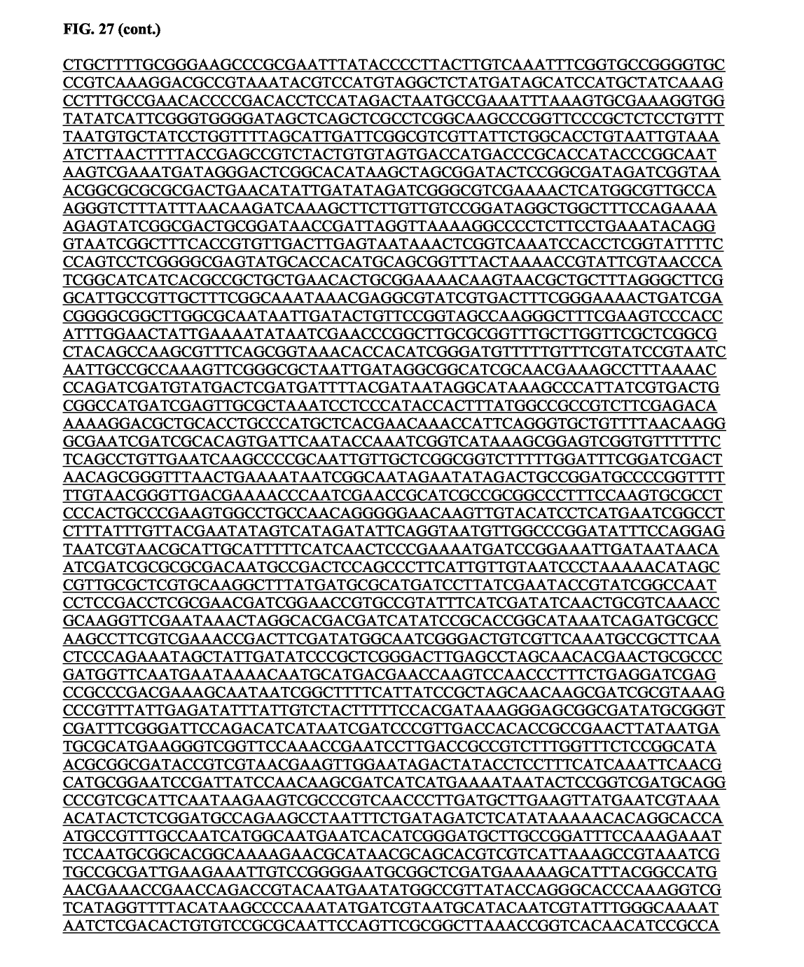

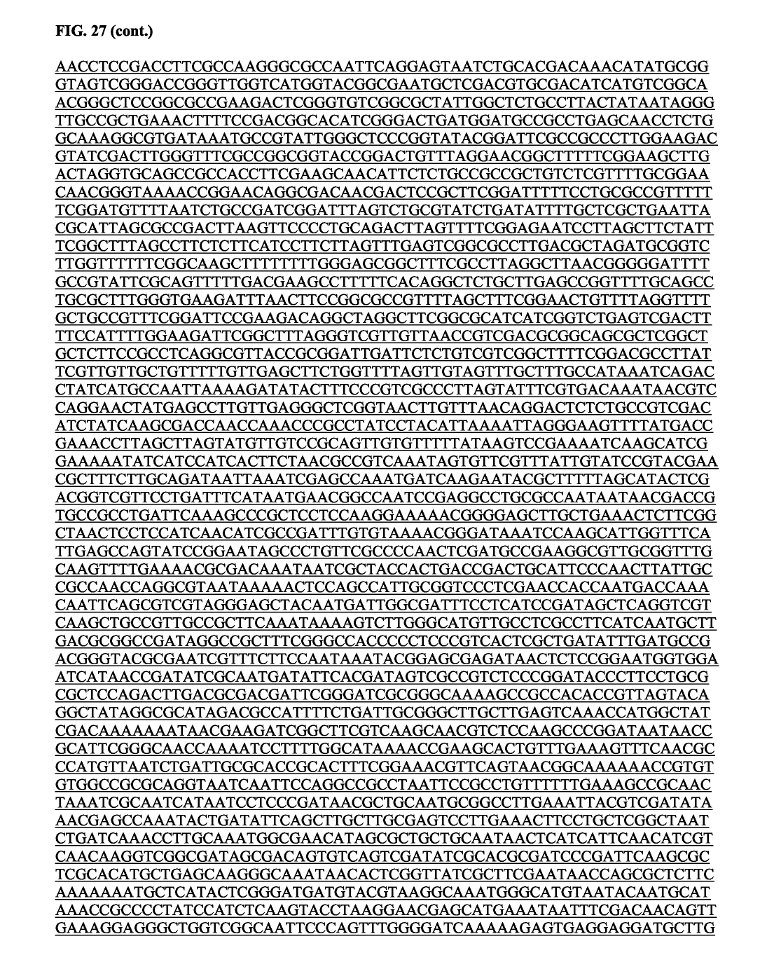

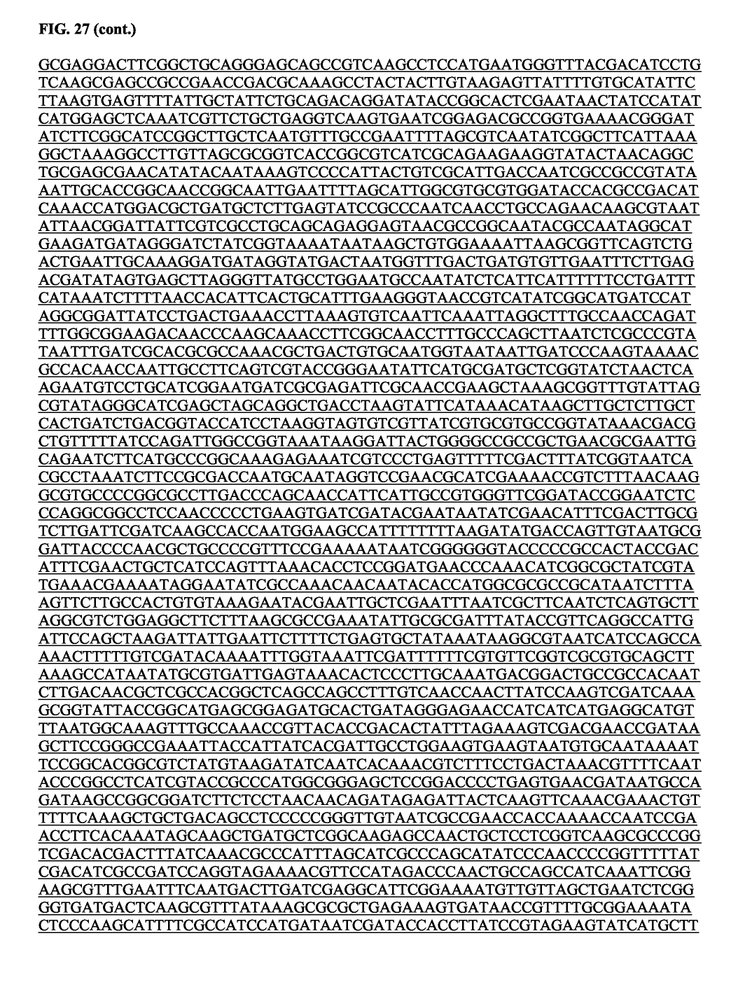

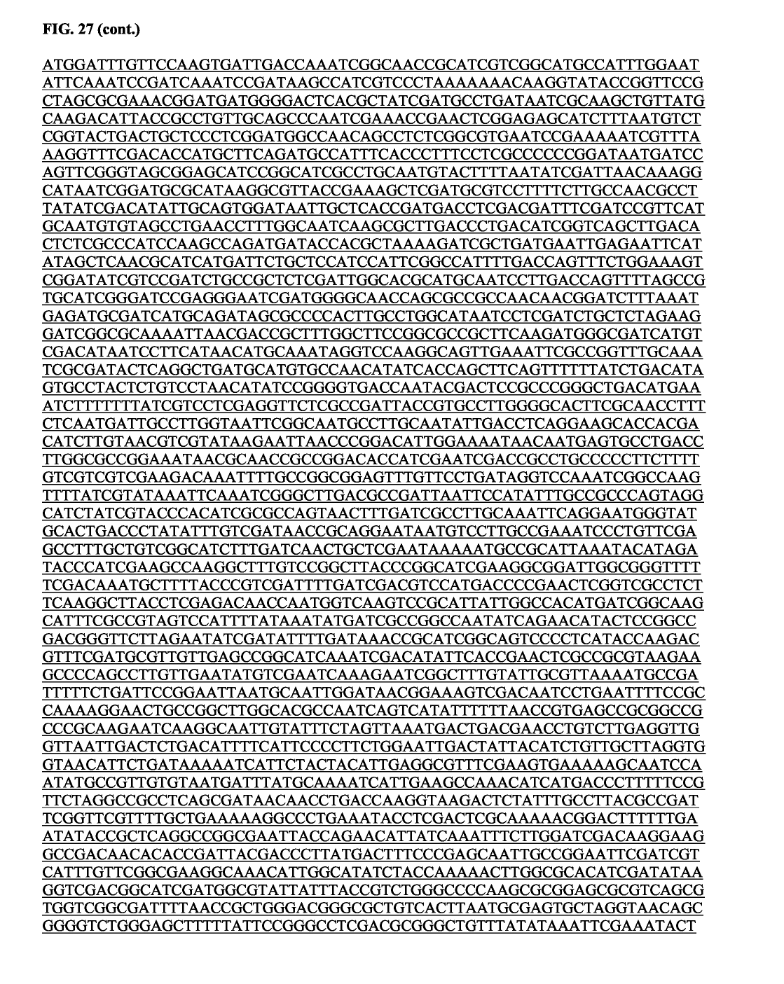

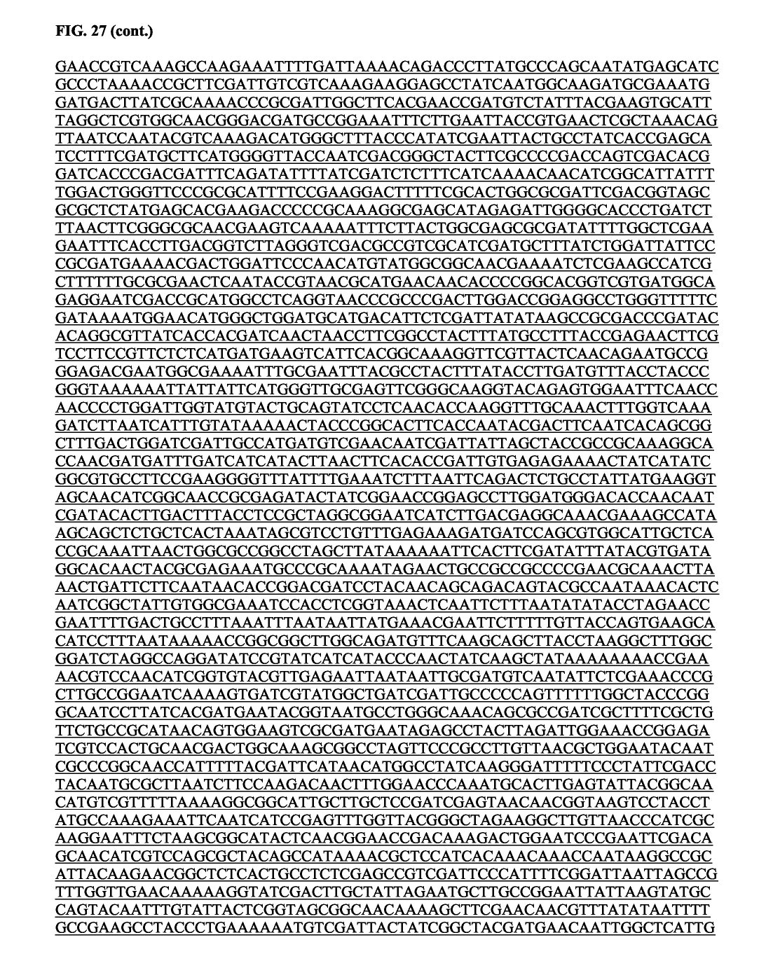

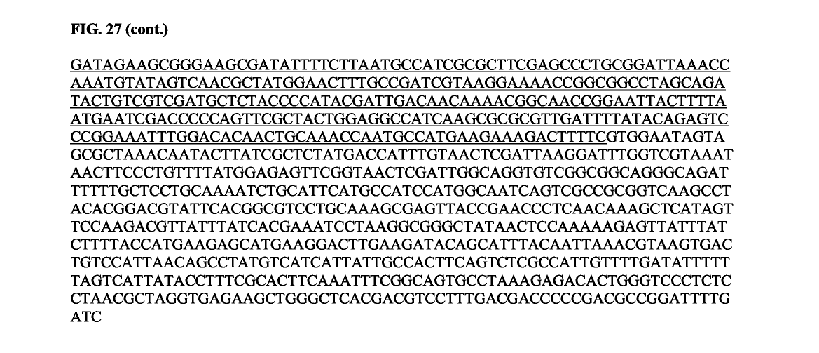

As shown in FIG. 17, the rate of flux to pyruvate was increased in the sps, ldh, and fadE mutants.

### Example 4

### Genetically Modified Strains: Pyruvate Excretion

Cultivation and growth parameters. M. alcaliphilum 20Z cells were grown using a mineral salts medium (Ojala et al. 2010) in bioreactor cultures (fed-batch or chemostat; 1L working volume in a two liter bench top BioFlo 110 modular bioreactors, New Brunswick Scientific, Edison, N.J.). Cells were grown at 28-30° C. Optical density of cell cultures was measured on a Beckman DU® 640B spectrophotometer in plastic 1.5 mL cuvettes with a 1 cm path length. Chemostat cultures maintained a steady-state optical density at 600 nm (OD₆₀₀) of approximately 5.0±0.5. The dilution rate was 0.12 h⁻¹ for aerobic cultures (influent gas mixture—5% CH₄: 3.5% O₂ balanced with N₂, pH (9.0) was controlled by the automatic addition of 1N NaOH. Agitation was kept at 500-750 rpm. Samples of inflow and outflow gases were either collected daily in triplicates for gas analysis or were analyzed immediately every 15 min using a SRI 8610C Gas Chromatograph connected to bioreactor unit. To estimate the concentration of pyruvate excreted into growth medium, 50 ml samples were collected. Cells were separated by centrifugation (15 min at 2,700×g), filtration via 0.2 μm filter units followed by ultrafiltration through Amicon@Ultra 3K filters. NMR analyses of the culture media were submitted to the Northwest Metabolomics Research Center (NW-MRC) for analyses. Generated strains and their phenotypes are shown in Table 11.

### REFERENCES

Gibson, D. G., Young, L., Chuang, R.-Y., Venter, J. C., Hutchison, C. A., & Smith, H. O. (2009). Enzymatic assembly of DNA molecules up to several hundred kilobases. Nature Methods, 6(5), 343-345.

Khmelenina, V. N., Beck, D. A. C., Munk, C., Davenport, K., Daligault, H., Erkkila, T., et al. (2013). Draft Genome Sequence of Methylomicrobium buryatense Strain 5G, a Haloalkaline-Tolerant Methanotrophic Bacterium. Genome Announcements, 1(4).

Knauf, V. C. & Nester, E. W. (1982). Wide host range cloning vectors: a cosmid clone bank of an Agrobacterium Ti plasmid. Plasmid, 8(1), 45-54.

Marx, C. J. & Lidstrom, M. E. (2001). Development of improved versatile broad-host-range vectors for use in methylotrophs and other Gram-negative bacteria. Microbiology, 147(8), 2065-2075.

Davison, J., Genetic Tools for Pseudomondas, Rhizobia, and Other Gram-Negative Bacteria, BioTechniques 32:386-401 (February 2002).

Lale et al., Methods in Molecular Biology, vol. 765, pages 327-343, (2011)

### Example 5

### Pyruvate Flux Into Mutants vs. Wild-Type M. buryatense 5GB1

TABLE 11

| | | | | | Increase in |
|---|---|---|---|---|---|
| | | Locus | | Observed phenotype | pyruvate |
| Gene(s) | Strain | tag(s) | Rationale | (results) | flux? |
| Glycogen | Methylomicrobium | MBURv2_{—} | Decrease | No change in growth | Not tested |
| synthase 1 | buryatense 5GB1 | 210177 | glycogen | rate; 60 ± 13% decrease | |
| (glgA1) | | | production | in glycogen | |
| Glycogen | Methylomicrobium | MBURv2_{—} | Decrease | No change in growth | Not tested |
| synthase 2 | buryatense 5GB1 | 210185 | glycogen | rate; 32 ± 19% decrease | |
| (glgA2) | | | production | in glycogen | |
| Glycogen | Methylomicrobium | | | No change in growth | Not tested |
| synthase | buryatense 5GB1 | | | rate; 87 ± 4% decrease | |
| (glgA1-glgA2) | | | | in glycogen | |
| Acyl-CoA | Methylomicrobium | MBURv2_{—} | Decrease | No change in growth | Pyruvate |
| dehydrogenase | buryatense 5GB1 | 190114 | β-oxidation | rate. No increase in | flux |
| (fadE) | | | of fatty acids | lipid content. | increased |
| | | | | | 57% |
| Sucrose-6- | Methylomicrobium | MBURv2_{—} | Decrease | No change in growth | Pyruvate |
| phosphate | buryatense 5GB1 | 130613 | glycogen/ | rate. | flux |
| synthase (sps) | | | exopolysaccharide | | increased |
| | | | production | | 29% |
| Lactate | Methylomicrobium | MBURv2_{—} | Increase | No change in growth | Pyruvate |
| dehydrogenase | buryatense 5GB1 | 210062 | pyruvate | rate. | flux |
| 1 (ldh1) | | | pool | | increased |
| | | | | | 69% |
| Acetate kinase | Methylomicrobium | MBURv2_{—} | Increase | No change in growth | No increase |
| (ack) | buryatense 5GB1 | 60338 | pyruvate | rate. | in pyruvate |
| | | | pool | | flux |
| Glycogen | Methylomicrobium | MALCv4_{—} | Decrease | No change in growth | Not tested |
| synthase | alcaliphilum 20Z | 3507. | glycogen | rate; Data are shown in | |
| cluster | | MALCv4_{—} | production | Table 8 | |
| 1(glgABC1manBQamyC) | | 3508 | | | |
| Glycogen | Methylomicrobium | MALCv4_{—} | Decrease | Not tested | Not tested |
| synthase | alcaliphilum 20Z | 3502- | glycogen | | |
| cluster 2 | | MALCv4_{—} | production | | |
| (glgAB2amyAC) | | 3503- | | | |
| | | MALCv4_{—} | | | |
| | | 3504 | | | |
| Glycogen | Methylomicrobium | MALCv4_{—} | Decrease | Not tested | Not tested |
| synthase | alcaliphilum 20Z | 3502. | glycogen | | |
| clusters 1 + 2 | | MALCv4_{—} | production | | |
| (glgABC1- | | 3503. | | | |
| glgABC2) | | MALCv4_{—} | | | |
| | | 3504. | | | |
| | | MALCV4_{—} | | | |
| | | 3505. | | | |
| | | MALCv4_{—} | | | |
| | | 3506. | | | |
| | | MALCv4_{—} | | | |
| | | 3507. | | | |
| | | MALCv4_{—} | | | |
| | | 3508 | | | |
| Sucrose-6- | Methylomicrobium | MALCv4_{—} | Decrease | No sucrose | Excretion of |
| phosphate | alcaliphilum 20Z | 0614 | sucrose/ | accumulation is | pyruvate |
| synthase (sps) | | | glycogen/ | observed. Increased | was |
| | | | exopolysaccharide | excretion of | observed, |
| | | | production | fermentation products. | 1.6 +/− 0.3 |
| | | | | | uM/g CDW; |
| | | | | | shows |
| | | | | | increased |
| | | | | | pyruvate |
| | | | | | production |
| Lactate | Methylomicrobium | MALCv4_{—} | Increase | No change in growth | Excretion of |
| dehydrogenase | alcaliphilum 20Z | 0534 | pyruvate | rate. Reduced | pyruvate |
| (ldh) | | | pool | production of acetate. | was |
| | | | | Excretion of glycerate | observed, |
| | | | | up to 34 uM/g CDW | 0.91 +/− 0.33 |
| | | | | | uM/g CDW; |
| | | | | | shows |
| | | | | | increased |
| | | | | | pyruvate |
| | | | | | production |
| Acetate kinase | Methylomicrobium | MALCv4_{—} | Increase | No change in growth | Excretion of |
| (ack) | alcaliphilum 20Z | 2853 | pyruvate | rate. Reduced | pyruvate |
| | | | pool | production of acetate. | was |
| | | | | Excretion of ethanol up | observed, |
| | | | | to 0.1 mM/gCDW | 1.82 +/− 0.23 |
| | | | | | uM/g CDW; |
| | | | | | shows |
| | | | | | increased |
| | | | | | pyruvate |
| | | | | | production |

1. An engineered methanotrophic bacterium, the bacterium comprising a genetic alteration causing a modulation selected from the group consisting of:
an increase in the conversion of methane to pyruvate and/or AcCoA;
a decrease in the activity of a pathway that diverts formate and/or pyruvate from fatty acid biosynthesis;
a decrease in lipid degradation activity; and
an increase in fatty ester production.

2. The bacterium of claim 1, wherein the bacterium comprises a genetic alteration causing an increase in the conversion of methane to pyruvate and/or AcCoA.

3. The bacterium of claim 1, further comprising a genetic alteration causing a decrease in the activity of a pathway that diverts formate and/or pyruvate from fatty acid biosynthesis.

4. The bacterium of claim 1, wherein the bacterium has an increased pyruvate flux.

5. The bacterium of claim 1, wherein the genetic alteration is selected from Table 2 or Table 9.

6. The bacterium of claim 1, wherein the genetic alteration is selected from the group consisting of:
an alteration resulting in an increase in the expression or activity of a gene selected from the group consisting of:
pmoCAB(methane monoxygenase); pyk1 (pyruvate kinase); accABC(acetyl-CoA carboxylase); ppc (PEP carboxylase); ftfL (formyltetrahydrofolate); mtdA (methylenetetrahydrofolate dehydrogenase); and fch (formyltetrahydrofolate cyclohydrogenase).
the introduction of an exogenous or ectopic ppc (PEP carboxylase); atfA (actyltransferase WS/DGAT); or tesA gene;
an alteration resulting in a decrease in the expression or activity of a gene selected from the group consisting of:
fdsABCD, fdhAB (formate dehydrogenase); gnd (gluconate-6-phosphate dehydrogenase); glgC (ADP-glucose pyrophosphorylase); glgA (glycogen synthase); glgB (glycogen branching enzyme); pps (phosphoenolpyruvate); fae(formaldehyde activating enzyme), mch (methenyltetrahydromethanopterin cyclohydrolase); mtdB (methylenetetrahydromethanopterin dehydrogenase); sps (sucrose phosphate synthase); ldh (lactate dehydrogenase); and ack (acetate kinase).

7. The bacterium of claim 6, wherein the genetic alteration is an alteration resulting in a decrease in the expression or activity of a gene selected
from the group consisting of:
gnd (gluconate-6-phosphate dehydrogenase); glgC (ADP-glucose pyrophosphorylase); glgA (glycogen synthase); glgB (glycogen branching enzyme); pps (phosphoenolpyruvate); mtdB (methylenetetrahydromethanopterin dehydrogenase); sps (sucrose phosphate synthase); ldh (lactate dehydrogenase); and ack (acetate kinase).

8. The bacterium of claim 1, wherein the bacterium is selected from the group consisting of:
Methylomicrobium spp.; Methylmonas spp.; Group I methanotrophic bacterium; Methylomicrobium alcahphilum; M alcahphilum 20ZR; M. buryatenase; M. buryatenase 5GB1; Methylomonas sp. LW13; Methylmonas MK1; Methylomonas sp.11b.

9. An engineered methanotrophic bacterium, the bacterium comprising a genetic alteration which modulates the expression of a gene product as specified in Table 9.

10. The bacterium of claim 9, wherein the bacterium has an increased pyruvate flux.

11. The bacterium of claim 9, wherein the bacterium is selected from the group consisting of:
Methylomicrobium spp.; Methylmonas spp.; Group I methanotrophic bacterium; Methylomicrobium alcaliphilum; M. alcaliphilum 20ZR; M. buryatenase; M. buryatenase 5GB1; Methylomonas sp. LW13; Methylmonas MK1; Methylomonas sp.11b.

12. A method of engineering a methanotrophic bacterium to increase pyruvate flux, the method comprising genetically altering a methanotrophic bacterium to cause a modulation selected from the group consisting of:
an increase in the conversion of methane to pyruvate and/or AcCoA;
a decrease in the activity of a pathway that diverts formate and/or pyruvate from fatty acid biosynthesis;
a decrease in lipid degradation activity; and
an increase in fatty ester production.

13. The method of claim 12, wherein the bacterium comprises a genetic alteration causing an increase in the conversion of methane to pyruvate and/or AcCoA.

14. The method of claim 12, further comprising a genetic alteration causing a decrease in the activity of a pathway that diverts formate and/or pyruvate from fatty acid biosynthesis.

15. The method of claim 12, wherein the bacterium has an increased pyruvate flux.

16. The method of claim 12, wherein the genetic alteration is selected from Table 2 or Table 9.

17. The method of claim 12, wherein the genetic alteration is selected from the group consisting of:
an alteration resulting in an increase in the expression or activity of a gene selected from the group consisting of:
pmoCAB(methane monoxygenase); pyk1 (pyruvate kinase); accABC(acetyl-CoA carboxylase); ppc (PEP carboxylase); ftfL (formyltetrahydrofolate); mtdA (methylenetetrahydrofolate dehydrogenase); and fch (formyltetrahydrofolate cyclohydrogenase).
the introduction of an exogenous or ectopic ppc (PEP carboxylase); atfA (actyltransferase WS/DGAT); or tesA gene;
an alteration resulting in a decrease in the expression or activity of a gene selected from the group consisting of:
fdsABCD, fdhAB (formate dehydrogenase); gnd (gluconate-6-phosphate dehydrogenase); glgC (ADP-glucose pyrophosphorylase); glgA (glycogen synthase); glgB (glycogen branching enzyme); pps (phosphoenolpyruvate); fae(formaldehyde activating enzyme), mch (methenyltetrahydromethanopterin cyclohydrolase); mtdB (methylenetetrahydromethanopterin dehydrogenase); sps (sucrose phosphate synthase); ldh (lactate dehydrogenase); and ack (acetate kinase).

18. The method of claim 17, wherein the genetic alteration of a gene is an alteration resulting in a decrease in the expression or activity of a gene selected
from the group consisting of:
gnd (gluconate-6-phosphate dehydrogenase); glgC (ADP-glucose pyrophosphorylase); glgA (glycogen synthase); glgB (glycogen branching enzyme); pps (phosphoenolpyruvate); mtdB (methylenetetrahydromethanopterin dehydrogenase); sps (sucrose phosphate synthase); ldh (lactate dehydrogenase); and ack (acetate kinase).

19. The method of claim 12, wherein the bacterium is selected from the group consisting of:
Methylomicrobium spp.; Methylmonas spp.; Group I methanotrophic bacterium; Methylomicrobium alcahphilum; M. alcahphilum 20ZR; M. buryatenase; M. buryatenase 5GB1; Methylomonas sp. LW13; Methylmonas MK1; Methylomonas sp.11b.

20. The method of claim 12, wherein the method further comprises measuring the catabolism of methane to pyruvate.

21. A method of increasing the flux of carbon from methane to pyruvate, the method comprising treating a methanotrophic bacterium to alter the expression or activity of a gene product as specified in Table 9.

22. The method of claim 21, wherein the method further comprises measuring the catabolism of methane to pyruvate.

23. A method of producing carbon catabolic products from methane, the method comprising contacting a bacterium of claim 1 with methane under conditions suitable for carbon catabolism.

24. The method of claim 23, wherein the carbon catabolic product is selected from the group consisting of:
lipids; fatty acids; fatty acid esters; free fatty acids; phospholipids.

25. The method of claim 23, wherein the method further comprises measuring the catabolism of methane to pyruvate.

26. The method of claim 23, further comprising the step of isolating the carbon catabolic product.

27. The method of claim 23, wherein the carbon catabolic product is a lipid.

28. A method of fixing methane carbon in pyruvate, the method comprising contacting a bacterium of claim 1 with methane under conditions suitable for methane catabolism.

29. The method of claim 28, wherein the method further comprises measuring the catabolism of methane to pyruvate.
